(19) United States
(12) Patent Application Publication
(10) Pub. No.: US 2026/0284005 A1
(43) Pub. Date: Sep. 24, 2026

(54) INHIBITING MITOGEN-ACTIVATED PROTEIN (MAP)/ERK KINASE (MEK)1 AND MEK2 AND RELATED METHODS OF TREATMENT

(71) Applicant: Immuneering Corporation, Cambridge, MA (US)

(72) Inventors: Brett Matthew Hall, San Diego, CA (US); Peter John King, San Diego, CA (US); Praveen Nair, San Diego, CA (US)

(21) Appl. No.: 19/475,944

(22) PCT Filed: Apr. 16, 2024
(86) PCT No.: PCT/US2024/024818

§ 371 Date: Oct. 16, 2025

Related U.S. Application Data
(63) Continuation-in-part of application No. PCT/US2024/018246, filed on Mar. 1, 2024.
(60) Provisional application No. 63/496,630, filed on Apr. 17, 2023.
(60) Provisional application No. 63/506,011, filed on Jun. 2, 2023.
(60) Provisional application No. 63/509,944, filed on Jun. 23, 2023.
(60) Provisional application No. 63/589,592, filed on Oct. 11, 2023.
(60) Provisional application No. 63/589,598, filed on Oct. 11, 2023.
(60) Provisional application No. 63/564,794, filed on Mar. 13, 2024.
(60) Provisional application No. 63/564,815, filed on Mar. 13, 2024.
(60) Provisional application No. 63/574,531, filed on Apr. 4, 2024.
(60) Provisional application No. 63/631,732, filed on Apr. 9, 2024.

(51) Int. Cl. A61K 31/37; A61K 31/506; A61P 35/00; A61P 35/04

(52) U.S. Cl. CPC A61K 31/37; A61K 31/506; A61P 35/00; A61P 35/04

(57) ABSTRACT

Methods of treating cancer, including RAS-mutated cancers and certain RAF-mutated cancers, are provided, comprising the administration of a therapeutically effective amount of dual-MEK inhibitor with a short plasma half-life that inhibits both the phosphorylation of mitogen-activated protein kinase kinase 1 or 2 (pMEK) and the phosphorylation of mitogen-activated protein kinase 1 or 2 (pERK), wherein the dual-MEK inhibitor is administered to the patient in certain dose amounts that achieves a Cmax effective to inhibit pERK and pMEK and with an interval between consecutive doses that is at least about certain times the plasma half-life of the dual-MEK inhibitor.

### INCORPORATION BY REFERENCE TO ANY PRIORITY APPLICATIONS

This patent application is a U.S. National Phase of International Application PCT/US2024/024818, filed Apr. 16, 2024 and claims the benefit of, and priority to, Patent Cooperation Treaty (PCT) Patent Application No. PCT/US2024/018246, filed Mar. 1, 2024, and U.S. Provisional Patent Application No. 63/496,630, filed Apr. 17, 2023; U.S. Provisional Patent Application No. 63/506,011, filed Jun. 2, 2023; U.S. Provisional Patent Application No. 63/509,944, filed Jun. 23, 2023; U.S. Provisional Patent Application No. 63/589,592, filed Oct. 11, 2023; U.S. Provisional Patent Application No. 63/589,598, filed Oct. 11, 2023; U.S. Provisional Patent Application No. 63/564,794, filed Mar. 13, 2024; U.S. Provisional Patent Application No. 63/564,815, filed Mar. 13, 2024; U.S. Provisional Patent Application No. 63/574,531, filed Apr. 4, 2024; and U.S. Provisional Patent Application No. 63/631,732, filed Apr. 9, 2024; each of which are incorporated by reference herein in their entirety.

### TECHNICAL FIELD

The present disclosure relates to the fields of chemistry and medicine. More particularly, the present disclosure relates to methods of treatment of certain forms of cancer with MEK inhibitor therapies.

### BACKGROUND

In all cells, signaling pathways govern how cells regulate themselves as well as direct activities in relation to other cells in the body. One of the most commonly altered signaling pathways in cancer is the Mitogen Activated Protein Kinase (MAPK) pathway. RAS is a family of related oncogenes found upstream in the MAPK pathway that codes for four highly related protein isoforms, HRAS, NRAS, KRAS4A and KRAS4B. In many solid tumors, the MAPK pathway is inappropriately activated, often through mutations in the key members of the pathway, including RAS. When RAS is switched “on” through the activation of the membrane-bound receptor tyrosine kinase, or RTK, the MAPK pathway functions to drive cell proliferation, differentiation, survival, and a variety of other cellular functions that are critical for the formation of tumors. There are three RAS genes: kirsten-RAS (KRAS), neuroblastoma-RAS (NRAS), and harvey-RAS (HRAS), encoding four RAS proteins (KRAS4A, KRAS4B, NRAS, and HRAS). Among RAS genes, KRAS could encode two proteins (KRAS4A and KRAS4B) due to alternative RNA splicing.

In many solid tumors, the MAPK pathway is inappropriately activated, often through mutations in the key members of the pathway, including RAS. When RAS is switched “on” through the activation of the membrane-bound receptor tyrosine kinase, or RTK, the MAPK pathway functions to drive cell proliferation, differentiation, survival, and a variety of other cellular functions that are critical for the formation of tumors. Oncogenic RAS family mutations have been found in about one quarter of all human cancers, however cancers characterized by a RAS mutation are challenging to treat using known therapies. In addition, RAS mutation is a negative predictor of cancer patient survival; cancer patients have much poorer prognostic outcomes (or shorter overall survival) when their cancer has a RAS mutation. Thus, there is a dire unmet need for a more effective treatment for cancer associated with oncogenic RAS gene mutation.

Through widespread adaptation of molecular profiling, it is now recognized that up to one in two cancer patients harbor tumors which are inappropriately activated through the MAPK pathway. Many of these patients display tumors with activation mutations in RAS or RAF, which lie upstream of MEK and ERK. Because inappropriate activation of the MAPK pathway supports many of the core tumor capabilities described above, efforts to create new therapeutics to target this pathway have been a high priority in cancer drug research. However, to date nearly all therapeutics that target the MAPK pathway have been limited to narrow patient subpopulations and have struggled to balance tolerability, durability of response, and efficacy. Nearly all targeted therapeutics against the MAPK pathway have been designed for sustained pathway suppression, which has resulted in on-target drug-related toxicities that limit clinical durability and potential drug-drug combinations.

Activating mutations of RAS and/or RAF in the MAPK pathway are observed in approximately 30% of all cancer patients, and inappropriate activation of this pathway is observed in up to 50% of all tumors and represents one of the most highly utilized signaling pathways in oncology. In aggressive solid tumors of the pancreas, skin, lungs and colon, mutations in RAS and/or RAF are even more common. For example, approximately 40% of lung cancers and approximately 95% of pancreatic cancers are due to RAS and/or RAF mutations, and approximately 50% of melanomas, 40% of thyroid and 30% of ovarian tumors display activation mutations, mainly, in BRAF but also in ARAF and CRAF (RAF1). To date, mitogen-activated extracellular signal-regulated kinase (MEK) inhibitors approved by the Food and Drug Administration (FDA) have been ineffective at treating RAS mutant tumors likely attributable to well-known mechanisms of resistance, including C-isomer of rapidly accelerated fibrosarcoma (CRAF)-mediated MEK (re)activation, or CRAF-bypass.

Traditional targeted oncology drug development generally seeks to chronically sustain pathway inhibition, by prioritizing drugs with long to moderate half-lives and dosing them at intervals that maintain a sufficient level of drug, even at trough, to maintain target occupancy. This prevailing chronic ablation approach can cause on-target drug-related toxicity and limit clinical durability as a result of drug holidays, treatment discontinuation, and/or adaptive resistance. To date, FDA-approved MEK inhibitors have been ineffective at treating RAS mutant tumors when compared to BRAF mutant tumors because of a well-known mechanism of resistance, CRAF-mediated MEK reactivation, also known as CRAF-bypass. In addition, a well-known limitation of current FDA approved MEK inhibitors are high rates of serious drug-related adverse events, or SAE's, most often observed in over 50% of treated patients, which leads to drug intolerability. The longer half-life of these drugs (e.g., up to 2 to 4 days) with once per day or less frequent dosing, or moderate half-life (e.g., 3 to 6 hours) with increased dosing frequency, contributes to high rates of adverse events because these drugs systemically circulate for an extended period of time harming healthy normal cells, which also rely on the pathway for survival.

Developing novel therapeutics to effectively, broadly and safely target this pathway may provide clinical benefit in large patient populations with significant unmet needs. In addition, although this pathway represents one of the most active areas in cancer drug discovery and development, targeted therapeutics that more effectively and safely normalize, but not chronically ablate, ERK signaling may uncouple drug activity and tolerability, while optimizing both. There remains an unmet medical need for methods of treating certain forms of cancer activated through the MAPK pathway, including methods of treatment to cyclically disrupt the ERK and MEK with a MEK inhibiting compound having a shorter half-life than prior “first generation” MEK inhibitor compounds such as trametinib, cobimetinib, binimetinib, and selumetinib. In addition, there is a clear unmet medical need and limited treatment options available to patients with RAS-mutated solid tumors.

Previous MEK inhibitors have been approved for BRAF-mutated lung cancer and melanoma; however, to reduce cytoreduction in the malignant clones, they must sustain the effective target occupancy across the dosing interval, and this chronic pathway ablation can be associated with on-target toxicities in normal cells. First-generation MEK inhibitors were designed to prevent phosphorylation of ERK (i.e., a surrogate for activation) by MEK through therapeutic engagement of an allosteric pocket adjacent to the ATP binding site, but first-generation compounds fail to control MEK phosphorylation at the RAF activation domain (e.g., Serine 218 and Serine 222 on MEK1), which is the initial step of MEK activation. Following treatment of RAS mutant cells, an increased accumulation of cytoplasmic pMEK contributes, in part, to the dogma that MEK inhibitors must sustain chronic target occupancy in order to inhibit the MAPK pathway effectively. However, chronic MEK inhibition leads to narrow therapeutic windows, significant class-effect toxicities and acquired or adaptive resistance of tumor cells. All of the first generation MEK inhibitors are designed for prolonged systemic circulation, either through longer half-life (e.g., up to 2 to 4 days), or moderate half-life (e.g., 3 to 6 hours) combined with increased dosing frequency. In either case, the prolonged systemic circulation contributes to high rates of adverse events by harming healthy normal cells, which also rely on the MAPK pathway. While significant advances have been made to better understand the underlying biology of the MAPK pathway, drugs designed to counteract these perturbations in the signaling pathway continue to be hindered by excess drug-related toxicity, short-lived durability of clinical response, and limited numbers of biomarker positive patients for mutation-specific drugs.

There remains an unmet medical need for methods of treating certain forms of cancer activated through the MAPK pathway, including methods of treatment to cyclically disrupt the ERK and MEK with a MEK inhibiting compound having a shorter half-life than prior “first generation” MEK inhibitor compounds such as trametinib, cobimetinib, binimetinib, and selumetinib.

### SUMMARY OF THE DISCLOSURE

Provided herein are methods of treating cancer comprising administering to a subject in need thereof Compound A in a daily dose of 320 mg, or 240 mg. In some embodiments, the daily dose is 320 mg of Compound A once per day (QD). In some embodiments, the daily dose is 240 mg of Compound A once per day (QD). In some embodiments, the dose can be administered orally. In some embodiments, the dose can be administered as a single daily dose. In some embodiments, the subject is administered Compound A for at least one months, or at least three months, or at least six months. Compound A can be administered as a pharmaceutically acceptable salt of Compound A, in an amount providing a total of 320 mg or 240 mg of Compound A per day, such as a once daily (QD) dose of 320 mg or 240 mg of Compound A to a subject in need thereof. In some embodiments, a method of treating cancer in a patient in need thereof, comprises administering Compound A

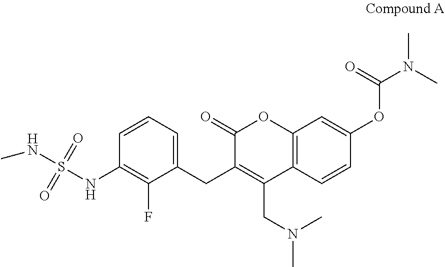

or a pharmaceutically acceptable salt thereof to the patient a dose providing 320 mg or 240 mg of Compound A once daily to the patient in need thereof for the treatment of the cancer. In some embodiments, a total of 320 mg of Compound A is orally administered (po) once daily (QD) to treat the cancer. In some embodiments, a total of 320 mg of Compound A is orally administered (po) once daily (QD) to treat the cancer. In some embodiments, a total of 240 mg of Compound A is orally administered (po) once daily (QD) to treat the cancer.

In some embodiments, the cancer is a RAS-mutated cancer. In some embodiments, the cancer is a BRAF-mutated cancer. In some embodiments, the patient is an adult and the cancer has a RAS mutation or a RAF mutation. In some embodiments, the cancer has a KRAS, HRAS or NRAS mutation. In some embodiments, cancer has a BRAF mutation. In some embodiments, the cancer has a KRAS mutation. In some embodiments, the KRAS mutation is a G12C, G12D, G12A, G12R, G12S, G12V, Q61 or G13 mutation. In some embodiments, the KRAS mutation is a G12D mutation. In some embodiments, the KRAS mutation is a G12V mutation. In some embodiments, the cancer has a KRAS mutation. In some embodiments, the KRAS mutation is a G12C, G12D, G12A, G12R, G12S, G12V, Q61 or G13 mutation. In some embodiments, the KRAS mutation is a G12D mutation. In some embodiments, the KRAS mutation is a G12V mutation.

In some embodiments, the patient is diagnosed with pancreatic cancer. In some embodiments, the patient is diagnosed with lung cancer. In some embodiments, the patient is diagnosed with melanoma. In some embodiments, the cancer is pancreatic ductal adenocarcinoma (PDAC). In some embodiments, the cancer optionally has a KRAS G12V, G12D or G12R mutation, and the method further comprises (a) treating the PDAC with one or more chemotherapy agents selected from the group consisting of gemcitabine, capecitabine, docetaxel and nab-paclitaxel, or (b) treating the PDAC with one or more chemotherapy agents selected from the group consisting of oxaliplatin, 5-fluorouracil and irinotecan.

In some embodiments, the cancer is non-small cell lung cancer (NSCLC). In some embodiments, the cancer is a KRAS G12C, G12D, G12A, G12R, G12V, Q61, or G13 mutated NSCLC. In some embodiments, the cancer is a KRAS G12C mutated NSCLC, And the method further comprises treating the NSCLC with a G12C inhibitor. In some embodiments, the G12C inhibitor is sotorasib or adagrasib.

In some embodiments, the cancer is melanoma. In some embodiments, Compound A is administered to the patient in need thereof in combination with an immune checkpoint inhibitor (ICI). In some embodiments, the ICI is an anti-PD-1, anti-PD-L1 or anti-CTLA-4 ICI. In some embodiments, the ICI is nivolumab, atezolizumab, ipilimumab, pembrolizumab, durvalumab, or cemiplimab. In some embodiments, the melanoma has a NRAS, HRAS or KRAS mutation. In some embodiments, the melanoma has a NRAS Q61R, Q61K, Q61H, G13R, G12D, or G13D mutation, or a HRAS Q61K mutation or a KRAS G12C, G12D, G12A, G12R, G12S, G12V, or Q61 mutation. In some embodiments, the method further comprises administering Compound A to the patient who has previously received an immune checkpoint inhibitor (ICI) for the treatment of the melanoma. In some embodiments, the immune checkpoint inhibitor is an anti-PD-1, anti-PD-L1 or anti-CTLA-4 ICI. In some embodiments, the ICI is ipilimumab, pembrolizumab, nivolumab, or relatlimab. In some embodiments, the method further comprises administering Compound A in combination with a BRAF inhibitor to the patient in need thereof for the treatment of the melanoma. In some embodiments, the BRAF inhibitor is encorafenib and the melanoma is an unresectable or metastatic BRAF V600E or V600K mutated melanoma.

In some embodiments, the cancer is selected from the group consisting of: pancreatic cancer with a KRAS G12C, G12V, G12D or G12R mutation; lung cancer with a KRAS G12C, G12D, Q61K; or G13C mutation or a NRAS Q61K or Q61L mutation; or a ALK S553A, R317T or G103R mutation; or an EGFR T790M, L858R or E746_A750del mutation; colorectal cancer with a KRAS G12V, G12D, or A146T mutation; or a BRAF V600E or G596R mutation; melanoma with a BRAF V600E mutation; acute myeloid leukemia (AML) with a KRAS Amp, G12C, or A146V mutation; or a NRAS G12C, G12D, Q61L, Q61P, or Q61D mutation; or multiple myeloma (MM) with a KRAS G12A, or G12C mutation or a NRAS G12C, or Q61H mutation.

In some embodiments, an oral dosage form of Compound A comprising Compound A or a pharmaceutically acceptable salt and a pharmaceutically acceptable excipients to form the oral unit dosage form. In some embodiments, the pharmaceutically acceptable excipients comprise an intra granulation excipients comprising a microcrystalline cellulose, a crospovidone, a hydroxypropyl cellulose; and an extra granulation excipient comprising Compound A. In some embodiments, the unit dosage form comprises a total of 120 mg or 160 mg of Compound A. In some embodiments, the unit dosage form is a tablet.

In some embodiments, this disclosure provides uses of Compound A or a pharmaceutically acceptable salt thereof as a dual MEK 1/2 inhibitor. In some embodiments, this disclosure provides use of Compound A or a pharmaceutically acceptable salt thereof to treat a cancer. In some embodiments, the cancer is a KRAS, NRAS or HRAS mutated cancer, a BRAF mutated cancer or an EGFR mutated cancer.

The subjects administered Compound A in the methods disclosed herein are diagnosed with a form cancer. The cancer can be a RAS mutated form of cancer, such as a KRAS, HRAS or NRAS mutated cancer. In some embodiments, the cancer is pancreatic cancer, melanoma or non-small cell lung cancer. In some cases, the cancer is colorectal cancer. In some cases, the cancer is pancreatic cancer. In some embodiments, the subject is one who, prior to start of therapy with Compound A, had undergone at least one (e.g., at least two) other systemic cancer therapy.

In some embodiments, a subject administered Compound A for at least a month does not exhibit any grade 3 or grade 4 adverse events associated with Compound A therapy. In some cases, the subject does not exhibit any grade 3 or grade 4 adverse events associated with Compound A therapy after at least three months of administration of Compound A. In some embodiments, the subject exhibits an at least stable disease after administration with Compound A. In some cases, the subject exhibits an at least partial response after administration with Compound A.

In some embodiments, methods of therapeutically treating pancreatic cancer, melanoma and non-small cell lung cancer are provided, where the method comprises administering a dual-MEK inhibitor to a subject in need thereof. In some embodiments, methods of therapeutically treating a cancer characterized by expressing a mutant form of a RAS protein comprises administering a dual-MEK inhibitor to a subject in need thereof. In some embodiments, methods of therapeutically treating pancreatic cancer are provided, including treatment of pancreatic cancer characterized by expressing a mutant form of a RAS protein, where the method comprises administering a dual-MEK inhibitor to a subject in need thereof. In some embodiments, methods of therapeutically treating melanoma are provided, including treatment of pancreatic cancer characterized by expressing a mutant form of a RAS or RAF protein, where the method comprises administering a dual-MEK inhibitor to a subject in need thereof. In some embodiments, methods of therapeutically treating pancreatic cancer are provided, including treatment of lung cancer (e.g., non-small cell lung cancer) characterized by expressing a mutant form of a RAS protein, where the method comprises administering a dual-MEK inhibitor to a subject in need thereof. In some embodiments, methods of therapeutically treating a cancer characterized by expressing a mutant form of a RAS protein comprises administering a dual-MEK inhibitor to a subject in need thereof.

In some embodiments, methods of treatment comprise the administration of a dual-MEK inhibitor to achieve deep cyclic inhibition of the MAPK pathway. Deep cyclic inhibition is a therapeutic approach for treatment of certain forms of cancer that aims to deprive tumor cells of the sustained proliferative signaling required for rapid growth, while sparing healthy cells through a cadenced, normalized level of signaling. In some embodiments, deep cyclic inhibition can be achieved by administering a dual-MEK inhibitor having sufficiently high pharmacodynamic inhibition of pERK (e.g., 90%+) and sufficiently short plasma half-life (e.g., 2 hours) at sequential dose intervals that are about seven or more times the plasma half-life (e.g., once daily).

In some embodiments, methods of treatment provide methods of administering a dual-MEK inhibitor having a high Cmax and a short plasma drug half-life at sequential doses that are administered at a dose interval (i.e., time between doses) that is greater than the plasma drug half-life. For example, in some embodiments, a method of treatment comprises administering a dual-MEK inhibitor on a dosing schedule providing a Cmax that achieves sufficiently high pharmacodynamic (PD) inhibition (e.g. 90% or greater) with sequential doses of the dual-MEK inhibitor being administered at intervals that are at least about 7-10 times the duration of the plasma drug half-life.

In some embodiments, methods provided herein comprise the use of and/or a method of treatment comprising administering Compound A or a pharmaceutically acceptable salt thereof to a subject in need thereof,

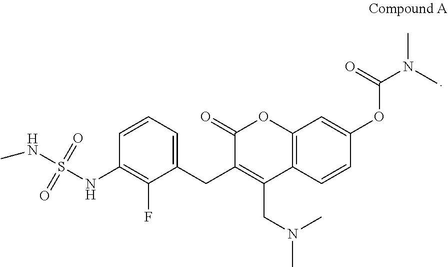

Compound A can be administered to rapidly reach a high Cmax and has a sufficiently short half-life to achieve a deep cyclic inhibition pathway for the treatment of patients with RAS-mutated solid tumors.

Compound A has an extremely short half-life, which allows for chronic dosing while maintaining a near-zero drug trough between doses on a 24-hour cycle, and this unique pharmacokinetic profile is expected to improve tolerability by inhibiting MAPK signaling cyclically rather than ablating it chronically. Compound A is characterized by a sufficiently short plasma half-life to reduce sustained pathway inhibition.

Unlike previous MEK inhibitors, Compound A rapidly reaches maximal drug exposure while achieving negligible drug trough levels within 12- to 24-hour cycles. This allows Compound A to be dosed chronically on a daily schedule to drive deep, cyclic inhibition of the MAPK pathway. By also engaging the RAF activation loop on MEK, Compound A prevents RAF-mediated MEK activation and further disrupts KSR. By designing Compound A to rapidly reach a high Cmax and have a short half-life to inhibit the activation of ERK while preventing RAF-mediated pathway reactivation, Compound A presents a novel approach to targeting MEK that may allow for broader activity and improved tolerability in patients with RAS-mutated solid tumors when compared with currently approved MEK inhibitors. Preclinical studies demonstrate well-tolerated anti-tumor activity profiles of Compound A in both the RAS- and RAF-mutant tumor setting.

Compound A is a dual MEK1/2 inhibitor useful to inhibit the activation of ERK while preventing RAF-mediated pathway reactivation. In some embodiments, Compound A is useful for the treatment of certain forms of cancer. In some embodiments, a method of treating a patient with a RAS-mutated or RAF-mutated cancer, comprises administering a therapeutically effective amount of Compound A to a patient in need thereof. In some embodiments, a method of treating a patient with a RAS-mutated or RAF-mutated cancer, comprises orally (p.o.) administering a therapeutically effective amount of Compound A to a patient in need thereof. In some embodiments, a method of treating a patient with a RAS-mutated or RAF-mutated cancer, comprises orally (p.o.) administering a therapeutically effective amount of Compound A once per day (QD) to a patient in need thereof. In some embodiments, Compound A is an oral, dual MEK1/2 inhibitor useful as monotherapy and in combination with approved agents for the treatment of solid tumors.

In some embodiments, methods of treatment comprise orally administering Compound A once-daily to a patient in need thereof in a total daily dose of 160 mg to treat certain forms of cancer. In some embodiments, methods of treatment comprise orally administering Compound A once-daily to a patient in need thereof in a total daily dose of 240 mg to treat certain forms of cancer. In some embodiments, methods of treatment comprise orally administering Compound A once-daily to a patient in need thereof in a total daily dose of 320 mg to treat certain forms of cancer.

In some embodiments, a dual-MEK inhibitor can be administered to solid tumor patients, specifically those with inappropriate activation of the MAPK pathway. Compound A has been specifically designed to provide deep cyclic inhibition of the MAPK pathway with a once-daily oral dosing cadence. Compound A was designed to overcome MAPK-feedback loops and to have an intentionally short plasma half-life, which is believed could enable it to provide deep cyclic inhibition. Without wishing to be bound by theory, it is believed that Compound A has the potential to provide broader therapeutic activity and an improved tolerability profile relative to known MEK inhibitors and that Compound A has the potential to target patients with solid tumors driven by any mutation in KRAS, NRAS, or HRAS. Compound A is currently being evaluated as a monotherapy in a Phase 1/2a clinical trial.

In some embodiments, Compound A is useful for the treatment of patients with certain forms of cancer, including pancreatic, melanoma, colorectal and non-small cell lung cancer, or NSCLC, caused by mutations of RAS. Methods of treatment comprising the administration of Compound A for the treatment of patients diagnosed with certain forms of cancer are provided. In some embodiments, Compound A is administered as a once-daily oral monotherapy for the treatment of RAS mutant tumors. In some embodiments, methods of administering Compound A can achieve a pharmacokinetic profile in a patient that is characterized by sufficiently high Cmax to provide stronger inhibition of the MAPK pathway than has been observed with first and second generation MEK inhibitors, followed by a near-zero drug trough.

The disclosure also provides the discovery that Compound A was observed to bind to mitogen-activated protein kinase kinase, or MEK, and acts as a highly selective inhibitor of mitogen-activated protein kinase, or ERK, activation (i.e., phosphorylation), with a “Dual MEK” function that is designed to block the CRAF-bypass feedback to prevent MAPK pathway reactivation. Compound A is also designed to prevent RAF-mediated activation of MEK, such as CRAF-bypass, by engagement of the RAF activation loop on MEK, and at elevated levels further disrupt the kinase suppressor of RAS 1 and 2, or KSR.

Compound A was evaluated in a range of human and murine solid tumor models, including those with activating MAPK pathway mutations in KRAS, NRAS, HRAS, BRAF, NF1 and KSR. For example, Compound A was evaluated in the MIA PaCa-2 (KRAS G12C PDAC), SK-MEL-2 (NRAS Q61R melanoma), A549 (KRAS G12S NSCLC) and Colon-26 (KRAS G12D CRC) mouse tumor models. Compound A was compared head-to-head to current FDA-approved MEK and BRAF inhibitors in KRAS-, NRAS- and BRAF-mutated solid tumor rodent models representing lung, colon, pancreas and skin cancers. In each of these studies, Compound A demonstrated tumor stasis or regression while maintaining body weight.

In some embodiments, methods of treatment comprising administering a therapeutically effective amount of Compound A to treat broad populations of solid tumor patients, specifically those with inappropriate activation of the MAPK pathway such as patients with solid tumors driven by any mutation in KRAS, NRAS, or HRAS.

In some embodiments, a method of treating a patient with a RAS- or RAF-mutated cancer, comprises administering a therapeutically effective amount of a compound having the structure of Compound A:

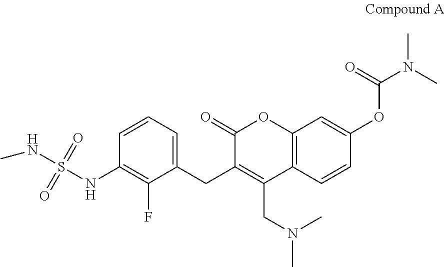

or a pharmaceutically acceptable salt thereof, to the patient in need thereof.

In some embodiments, Compound A is administered to the patient (a) in a dose amount that achieves a Cmax effective to inhibit pERK and pMEK and (b) with an interval between consecutive doses that is at least about seven times the plasma half-life of Compound A. In some embodiments, the method comprises administering Compound A to the patient as a monotherapy to treat the cancer. In some embodiments, a method of treating a patient with a RAS- or RAF-mutated cancer, comprising administering a therapeutically effective amount of a compound having the structure of Compound A or a pharmaceutically acceptable salt thereof, to the patient in need thereof, wherein the cancer expresses a KRAS mutation. In some embodiments, a method of treating a patient with a RAS- or RAF-mutated cancer, comprising administering a therapeutically effective amount of a compound having the structure of Compound A or a pharmaceutically acceptable salt thereof, to the patient in need thereof, wherein the cancer expresses a RAF mutation. In some embodiments, the Compound A is administered orally to the patient in need thereof.

In some embodiments, a method of treatment comprises administering to a patient total of 320 mg per day (QD) of Compound A to a patient in need thereof as a monotherapy for the treatment of a RAS- or RAF-mutated cancer. In some embodiments, a method of treatment comprises administering to a patient total of 240 mg or 320 mg per day (QD) of Compound A to a patient in need thereof in combination with another therapeutic agent for the treatment of a RAS- or RAF-mutated cancer.

### BRIEF DESCRIPTION OF THE DRAWINGS

FIGS. 1A and 1B are graphs summarizing results obtained from preclinical testing of Compound A in various xenograft tumor models (tumor volume), for pancreatic cancer (KRAS-G12C and KRAS-G12V), melanoma (NRAS-G61R), non-small cell lung cancer (NSCLC) (KRAS-G12S), and colorectal cancer (KRAS-G12D).

FIGS. 2A and 2B are graphs showing tumor pharmacodynamic transcriptome data from a head-to-head comparison of Compound A against selumetinib using a Colon-26 syngeneic tumor model.

FIG. 3 is a graph showing results from a head-to-head comparison of Compound A against four FDA-Approved MEK Inhibitors Using a A549 Tumor Cell Model, showing Compound A prevented downstream activation of ERK (↓ pERK) and inhibited activation of MEK (↓ pMEK).

FIG. 4A is a graph showing the tumor Pharmacokinetic and Pharmacodynamic (pERK) activity of Compound A in the Colon-26 animal model.

FIG. 4B is a graph showing the tumor pharmacodynamic transcriptome activity of Compound A in the Colon-26 animal model.

FIGS. 5A-C are graphs showing the head-to-head comparison of Compound A against binimetinib and selumetinib using a Colon-26 syngeneic tumor model (tumor volume).

FIGS. 6A and 6B are graphs showing data from head-to-head comparison of Compound A against binimetinib+/− encorafenib using an A549 xenograft tumor model (tumor volume).

FIGS. 7A and 7B are graphs showing data from the head-to-head comparison of Compound A against binimetinib+/− encorafenib using a A375 xenograft tumor model (tumor volume).

FIG. 8A are graphs showing data from the head-to-head comparison of Compound A+/− sotorasib (AMG-510) using a MIA PaCa-2 Xenograft Tumor Model (Tumor Volume).

FIG. 8B are graphs showing data from the head-to-head comparison of Compound A, sotorasib (AMG-510) and adagrasib using a CAPAN-2 Xenograft Tumor Model (Tumor Volume).

FIG. 9 are graphs showing results from a head-to-head comparison of tumor volume measured with Compound A against binimetinib in a NRAS-Q61R SK-MEL-2 melanoma xenograft study (Tumor Volume).

FIG. 10 is a graph showing data obtained from an evaluation of Compound A as compared to vehicle using a SK-MEL-2 xenograft tumor model (Tumor Volume).

FIG. 11A is a graph showing 3D-TGA Compound A dose responses calculated for sensitive tumor models.

FIG. 11B is a graph showing 3D-TGA Compound A dose responses calculated for intermediate tumor models.

FIG. 11C is a graph showing 3D-TGA Compound A dose responses calculated for resistant tumor models.

FIG. 12A is graph showing the mapping of biomarker sensitive patient populations (GENIE) for pancreatic cancer.

FIG. 12B is graph showing the mapping of biomarker sensitive patient populations (GENIE) for melanoma cancer.

FIG. 12C is graph showing the mapping of biomarker sensitive patient populations (GENIE) for lung cancer.

FIG. 12D is graph showing the mapping of biomarker sensitive patient populations (GENIE) for colorectal cancer.

FIG. 12E is graph showing the mapping of biomarker sensitive patient populations (GENIE) for prostate cancer.

FIG. 12F is graph showing the mapping of biomarker sensitive patient populations (GENIE) for breast cancer.

FIG. 12G is graph showing the mapping of biomarker sensitive patient populations (GENIE) for ovarian cancer.

FIG. 12H is graph showing the mapping of biomarker sensitive patient populations (GENIE) for glioblastoma cancer.

FIG. 13 is a table showing response and non response identified in analysis of 132 tumor models using humanized 3D-TGA.

FIG. 14A is a graph showing the pharmacokinetics (PK) and pharmacodynamics (PD) for pERK for Compound A in two human patients (3 and 4) diagnosed with colorectal cancer with a NRAS-Q61K mutation, after administration of 160 mg of Compound A administered orally (po) once daily (QD) (Dose Level III).

FIG. 14B is a graph showing the pharmacokinetics (PK) and pharmacodynamics (PD) for phosphorylated MEK (pMEK) for Compound A in two human patients (3 and 4) diagnosed with colorectal cancer with a NRAS-Q61K mutation, after administration of 160 mg of Compound A administered orally (po) once daily (QD) (Dose Level III).

FIG. 14C is a graph showing the modeling of PK and binding kinetics (BK) for Compound A in two human patients (3 and 4) diagnosed with colorectal cancer with a NRAS-Q61K mutation, after administration of 160 mg of Compound A administered orally (po) once daily (QD) (Dose Level III).

FIG. 14D is a graph showing PK/PD (pERK) for Compound A from patient 3 after receiving 160 mg Compound A administered orally (po) once daily (QD) (cycle 1, day 15).

FIG. 14E is a graph showing PK/PD (pMEK) for Compound A from patient 3 after receiving 160 mg Compound A administered orally (po) once daily (QD) (cycle 1, day 15).

FIG. 14F is a graph showing PK/PD (pERK) for Compound A from patient 4 after receiving 160 mg Compound A administered orally (po) once daily (QD) (cycle 1, day 15).

FIG. 14G is a graph showing PK/PD (pMEK) for Compound A from patient 4 after receiving 160 mg Compound A administered orally (po) once daily (QD) (cycle 1, day 15).

FIG. 15A is a graph showing PK/PD (pERK) for Compound A from patient 1 after receiving 40 mg Compound A administered orally (po) once daily (QD) (cycle 1, day 1).

FIG. 15B is a graph showing PK/PD (pMEK) for Compound A from patient 1 after receiving 40 mg Compound A administered orally (po) once daily (QD) (cycle 1, day 1).

FIG. 16A is a graph showing PK/PD (pERK) for Compound A from patients after receiving 80 mg Compound A administered orally (po) once daily (QD) (cycle 1, day 1).

FIG. 16B is a graph showing PK/PD (pMEK) for Compound A from patient 2 after receiving 80 mg Compound A administered orally (po) once daily (QD) (cycle 1, day 1).

FIG. 17A is a graph showing PK/PD (pERK) for Compound A from patient 5 after receiving 320 mg administered orally (po) once daily (QD) po (cycle 1, day 1).

FIG. 17B is a graph showing PK/PD (pMEK) for Compound A from patient 5 after receiving 320 mg administered orally (po) once daily (QD) po (cycle 1, day 1).

FIG. 17C is a graph showing PK/PD (pERK) for Compound A from patient 5 after receiving 320 mg administered orally (po) once daily (QD) po (cycle 1, day 15).

FIG. 17D is a graph showing PK/PD (pMEK) for Compound A from patient 5 after receiving 320 mg administered orally (po) once daily (QD) po (cycle 1, day 15).

FIG. 18A is a graph showing PK/PD (pERK) for Compound A from patient 6 after receiving 320 mg administered orally (po) once daily (QD) po (cycle 1, day 1).

FIG. 18B is a graph showing PK/PD (pMEK) for Compound A from patient 6 after receiving 320 mg administered orally (po) once daily (QD) po (cycle 1, day 1).

FIG. 18C is a graph showing PK/PD (pERK) for Compound A from patient 6 after receiving 320 mg administered orally (po) once daily (QD) po (cycle 1, day 15).

FIG. 18D is a graph showing PK/PD (pMEK) for Compound A from patient 6 after receiving 320 mg administered orally (po) once daily (QD) po (cycle 1, day 15).

FIG. 19A is a graph showing PK/PD (pERK) for Compound A from patient 7 after receiving 320 mg administered orally (po) once daily (QD) po (cycle 1, day 1).

FIG. 19B is a graph showing PK/PD (pMEK) for Compound A from patient 7 after receiving 320 mg administered orally (po) once daily (QD) po (cycle 1, day 1).

FIG. 19C is a graph showing PK/PD (pERK) for Compound A from patient 7 after receiving 320 mg administered orally (po) once daily (QD) po (cycle 1, day 15).

FIG. 19D is a graph showing PK/PD (pMEK) for Compound A from patient 7 after receiving 320 mg administered orally (po) once daily (QD) po (cycle 1, day 15).

FIG. 20A is a graph showing plasma concentration (PK) for Compound A from patient 13 after receiving 240 mg Compound A administered orally (po) once daily (QD) at cycle 1, day 1 and at cycle 1, day 15.

FIG. 20B is a graph showing PK/PD (pERK) for Compound A from patient 13 after receiving 240 mg Compound A administered orally (po) once daily (QD) at cycle 1, day 1 and at cycle 1, day 15.

FIG. 20C is a graph showing plasma concentration (PK) for Compound A from patient 16 after receiving 320 mg Compound A administered orally (po) once daily (QD) at cycle 1, day 1 and at cycle 1, day 15.

FIG. 20D is a graph showing PK/PD (pERK) for Compound A from patient 16 after receiving 320 mg Compound A administered orally (po) once daily (QD) at cycle 1, day 1 and at cycle 1, day 15.

FIG. 21A is a schematic diagram of an end-to-end translational modeling approach in support of Compound A.

FIG. 21B is a table providing certain Phase 1 clinical trial summary parameters for Compound A.

FIG. 22A is a graph showing 3D-TGA Compound A dose responses calculated for sensitive tumor models.

FIG. 22B is a graph showing 3D-TGA Compound A dose responses calculated for intermediate tumor models.

FIG. 22C is a graph showing 3D-TGA Compound A dose responses calculated for resistant tumor models.

FIG. 23 is a schematic depicting examples of various therapeutic applications of Compound A both as monotherapy and Compound A in combination with other therapies, for the treatment of certain RAS and RAF mutations.

FIG. 24A is a kernel density plot of probability of predicted response for entire cohort (projected onto the negative y-axis), and biomarker+ subset (positive y-axis) for colorectal cancer.

FIG. 24B is a kernel density plot of probability of predicted response for entire cohort (projected onto the negative y-axis), and biomarker+ subset (positive y-axis) for melanoma.

FIG. 25A is a graph showing data obtained from an evaluation of Compound A as compared to vehicle using a HT-29 BRAF^{V600E} Colorectal Cancer (CRC) xenograft tumor model in athymic nude mice (Tumor Volume).

FIG. 25B is a graph showing data obtained from an evaluation of Compound A and encorafenib, as compared to vehicle using a HT-29 BRAF^{V600E} Colorectal Cancer (CRC) xenograft tumor model in athymic nude mice (Tumor Volume).

FIG. 26A is a graph showing data obtained from an evaluation of binimetinib or encorafenib individually, as compared to vehicle using a HT-29 BRAF^{V600E} Colorectal Cancer (CRC) xenograft tumor model in athymic nude mice (Tumor Volume).

FIG. 26B is a graph showing data obtained from an evaluation of binimetinib and encorafenib combined, as compared to vehicle using a HT-29 BRAF^{V600E} Colorectal Cancer (CRC) xenograft tumor model in athymic nude mice (Tumor Volume).

FIG. 27A is a dose response curves obtained in the humanized 3D Tumor Growth Assay (3D-TGA) with Compound A±gemcitabine with MIA PaCa-2 cells.

FIG. 27B is a dose response curves obtained in the humanized 3D Tumor Growth Assay (3D-TGA) with Compound A±gemcitabine with Panc 03-27 (KRAS G12V) cells.

FIG. 27C is a dose response curves obtained in the humanized 3D Tumor Growth Assay (3D-TGA) with Compound A±gemcitabine with Panc1 (KRAS-G12D) cells.

FIG. 28A is a dose response curves obtained in the humanized 3D Tumor Growth Assay (3D-TGA) with Compound A±paclitaxel with MIA PaCa-2 cells.

FIG. 28B is a dose response curves obtained in the humanized 3D Tumor Growth Assay (3D-TGA) with Compound A±paclitaxel with Panc 03-27 (KRAS G12V) cells.

FIG. 28C is a dose response curves obtained in the humanized 3D Tumor Growth Assay (3D-TGA) with Compound A±paclitaxel with Panc1 (KRAS-G12D) cells.

FIG. 29 illustrates a distribution of RAS^{mut} across patients in the GENIE (v13.0) database.

FIG. 30A illustrates mutation profiles across pancreatic cancer indications in GENIE (v13) patient database.

FIG. 30B illustrates mutation profiles across lung cancer indications in GENIE (v13) patient database.

FIG. 30C illustrates mutation profiles across melanoma cancer indications in GENIE (v13) patient database.

FIG. 30D illustrates mutation profiles across colorectal cancer indications in GENIE (v13) patient database.

FIG. 31A illustrates a representative subset of compound A dose responses for sensitive (IC₅₀<1 micromolar) cell lines shown in the table below the graph.

FIG. 31B illustrates a representative subset of compound A dose responses for intermediate (IC₅₀≥1 and >25% reduction at 10 micromolar) cell lines shown in the table below the graph.

FIG. 31C illustrates a representative subset of compound A dose responses for resistant (not susceptible or intermediate, and otherwise resistant in 3D-TGA response) cell lines shown in the table below the graph.

FIG. 32 illustrates a table of humanized 3D-TGA response based on tumor tissue and RAS mutation status.

FIG. 33 illustrates a table of KRAS G12 variant and Associated 3D-TGA response category.

FIG. 34A is a graph showing the RAS mutations reported at enrollment (N=41) for phase 1 human clinical trial patients treated with Compound A.

FIG. 34B is a graph showing cancer diagnosis for the patients reported at enrollment (N=41) for phase 1 human clinical trial patients treated with Compound A.

FIG. 35 is a graph showing pharmacokinetic (PK) and pharmacodynamic (PD) data obtained from patients treated with Compound A.

FIG. 36 is a graph of model-predicted response for patient profiles in pancreatic adenocarcinoma (PAAD) in GENIE 15.0.

FIG. 37A is a graph showing data obtained from an evaluation of Compound A (A), gemcitabine (GEM) or nab-paclitaxel (PAC) individually or in the specified combinations, as compared to vehicle using a MIA PaCa-2 KRAS G12C mutant xenograft tumor model in athymic nude mice (Tumor Volume).

FIG. 37B is a graph showing a magnified portion of the graph in FIG. 37A, showing data obtained from an evaluation of Compound A (A), gemcitabine (GEM) or nab-paclitaxel (PAC) individually or in the specified combinations, as compared to vehicle using a MIA PaCa-2 KRAS G12C mutant xenograft tumor model in athymic nude mice (Tumor Volume).

FIG. 38A is a graph showing data obtained from an evaluation of Compound A (A), and 5-fluorouracil (5-FU) individually or in combination, as compared to vehicle using a MIA PaCa-2 KRAS G12C mutant xenograft tumor model in athymic nude mice (Tumor Volume).

FIG. 38B is a graph showing a magnified portion of the graph in FIG. 38A, showing data obtained from an evaluation of Compound A (A), and 5-fluorouracil (5-FU) individually or in combination, as compared to vehicle using a MIA PaCa-2 KRAS G12C mutant xenograft tumor model in athymic nude mice (Tumor Volume).

FIG. 39A is a graph showing the results form an enrichment analysis of recurrently-mutated genes under continuous treatment in protein-protein interaction hubs for nab-paclitaxel, showing the number of mice per treatment group and a scatter plot of the corresponding odds ratio.

FIG. 39B is a graph showing the results form an enrichment analysis of recurrently-mutated genes under continuous treatment in protein-protein interaction hubs for gemcitabine, showing the number of mice per treatment group and a scatter plot of the corresponding odds ratio.

FIG. 39C is a graph showing the results form an enrichment analysis of recurrently-mutated genes under continuous treatment in protein-protein interaction hubs for 5-fluorouracil, showing the number of mice per treatment group and a scatter plot of the corresponding odds ratio.

FIG. 39D is a graph showing the results form an enrichment analysis of recurrently-mutated genes under continuous treatment in protein-protein interaction hubs for Compound A, showing the number of mice per treatment group and a scatter plot of the corresponding odds ratio.

FIG. 39E is a graph showing the results form an enrichment analysis of recurrently-mutated genes under continuous treatment in protein-protein interaction hubs for a combination of Compound A, gemcitabine and nab-paclitaxel, showing the number of mice per treatment group and a scatter plot of the corresponding odds ratio.

FIG. 39F is a graph showing the results form an enrichment analysis of recurrently-mutated genes under continuous treatment in protein-protein interaction hubs for a combination of Compound A, and 5-fluorouracil (5-FU), showing the number of mice per treatment group and a scatter plot of the corresponding odds ratio.

FIG. 40 is a graph showing 3D-TGA Compound A dose responses calculated for PaCaDD-137 cells.

### DETAILED DESCRIPTION

In some aspects, the present disclosure provides methods for administering a dual-MEK inhibitor characterized by a high maximum plasma concentration (Cmax) and a short plasma drug half-life. In some embodiments, the method includes sequential doses that are administered at a dose interval (i.e., time between doses) that is greater than the plasma drug half-life. For example, in some embodiments, a method of treatment includes administering a dual-MEK inhibitor on a dosing schedule providing a Cmax that achieves sufficiently high pharmacodynamic (PD) inhibition (e.g. 90% or greater) with sequential doses of the dual-MEK inhibitor being administered at intervals that are about 7-18 times the duration of the plasma drug half-life.

In some embodiments, the dual MEK inhibitor is Compound A and the method of treatment includes administering Compound A once-daily to a patient in need thereof, for the treatment of a solid tumor harboring RAS mutations.

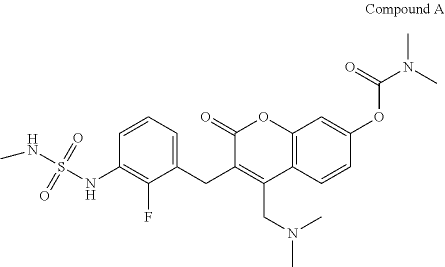

In certain embodiments, Compound A acts as a potent, dual-MEK inhibitor that uniquely inhibits both MEK and ERK enzymes. Without being limited to any particular theory, it is noted that the inhibition mechanism of Compound A is theorized to involve allosteric inhibition of MEK by targeting a site adjacent to the ATP binding pocket, which in turn inhibits ERK downstream. This compound exhibits a short plasma half-life and achieves a nearly zero steady-state trough concentration, facilitating deep, cyclic inhibition of the MAPK pathway. Compound A is chemically defined with a molecular weight of 506.55 g/mol and a molecular formula of C₂₃H27FN₄O₆S, and can be chemically named as 4-((dimethylamino)methyl)-3-(2-fluoro-3-((N-methylsulfamoyl)amino)benzyl)-2-oxo-2H-chromen-7-yl dimethylcarbamate. The method for synthesizing Compound A is detailed in Example 1.

Furthermore, Compound A may be distinguished from first-generation MEK inhibitors by its ability to block both the phosphorylation of MEK and the downstream phosphorylation of ERK (i.e., dual MEK), thereby disrupting both MEK activation by RAF and further engagement with the RAF activation loop on MEK. In contrast, first-generation inhibitors primarily inhibit MEK's ability to activate ERK without affecting MEK phosphorylation via RAF, which can lead to CRAF-mediated bypass activation of MEK. Phosphorylation of MEK through the RAF activation pathway is left unimpeded. Depleting intracellular pools of pERK is believed to remove the negative feedback signal on CRAF, which in turn is believed to hyperactivate phosphorylated MEK (pMEK) through CRAF leading to CRAF bypass. Preclinical models suggest that the administration of Compound A leads to high drug concentration peaks (Cmax) followed by a rapid decrease to near-zero trough levels within a 12- to 24-hour period, necessitating between 7 to 18 half-lives between doses. This pattern allows for a novel mechanism of action that avoids continuous suppression, potentially reducing toxicity and enhancing the durability of response in RAS-mutant tumors.

Unlike first-generation MEK inhibitors, such as cobimetinib, binimetinib, and selumetinib, which maintain prolonged MAPK pathway suppression and exhibit dose-limiting toxicities, Compound A presents an improved profile by allowing MAPK signaling recovery between doses. This results from its short half-life (1.2 to 1.3 hours in rodents and approximately 2 hours in humans), leading to cyclic, rather than chronic, inhibition of the pathway. Although second-generation MEKi, which includes VS-6766 (CH5126766), display mechanistic resistance to pathway reactivation, they also sustain suppression of MAPK pathway activity through chronic occupancy of MEK1 and MEK2 and, as such, share similar class-effect toxicities to first-generation MEKi. Selumetinib has a human plasma half-life of 5.3-7.2 h and a tmax of 1-1.5 h, which results in a sustained, non-zero drug trough with BID dosing (≤2.3 half-lives per BID cycle) and chronic ablation of MAPK signaling. Binimetinib has a shorter human plasma half-life of 3.5 h and tmax of 1.6 h but this still does not allow for a near-zero drug trough (≤3.4 half-lives per BID cycle). Conversely, cobimetinib has a long human plasma half-life of 44 h and tmax of 2.4 h (≤0.55 half-lives per cycle) while trametinib, with its long human plasma half-life of 96 hours has virtually no trough in exposure (≤0.25 half-lives per cycle).

In preclinical studies, Compound A inhibited MEK and ERK across a wide range of human and murine solid tumor models, including those with activating mutations in KRAS, NRAS, HRAS and BRAF. In addition, in head-to-head preclinical studies, Compound A was evaluated in murine-based KRAS, NRAS, and BRAF mutant solid tumor models representing lung, colon, pancreas and skin cancer, and observed tumor stasis or regression with insignificant body weight loss, or BWL, when compared to certain FDA-approved MEK inhibitors at reported human dose equivalent dose and schedules. For example, in head-to-head preclinical studies, Compound A was evaluated in murine-based KRAS, NRAS, and BRAF mutant solid tumor models representing lung (i.e., A549), colon (i.e., Colon-26), pancreas (i.e., MIA PaCa-2 and CAPAN-2) and skin cancer (i.e., A375 and SK-MEL-2), and observed superior antitumor activity with insignificant body weight loss, or BWL, when compared to certain FDA-approved MEK inhibitors and superior to non-inferior antitumor activity when compared to certain FDA-approved KRAS-G12C and BRAF inhibitors at reported human dose equivalent dose and schedules.

In primary pharmacodynamic assays, Compound A inhibited pERK across multiple tumor cells with half maximal inhibitory concentrations (IC₅₀) values ranging from 24.2 nM to 83.2 nM, with the most sensitive models being predominantly melanoma and pancreatic cancer cell lines that harbor activating MAPK pathway mutations in K isomer of rat sarcoma (KRAS), N isomer of rat sarcoma (NRAS), H isomer of rat sarcoma (HRAS), BRAF, neurofibromatosis type 1 (NF1), and/or kinase suppressor of Ras (KSR). Corresponding reductions in pMEK were observed with treatment of cells with 100 nM Compound A, confirming that Compound A was not sensitive to the MAPK pathway feedback reactivation events in RAS mutant models, such as CRAF-bypass.

Compound A has been evaluated in a wide range of human and murine solid tumor models, including those with activating MAPK pathway mutations in KRAS, NRAS, HRAS, BRAF, NF1, and KSR. Among others, Compound A was investigated in MIA PaCa-2 (KRAS^{G12C} PDAC), Capan-2 (KRAS^{G12V} PDAC), SK-MEL-2 (NRASQ^{61R} melanoma), A549 (KRAS^{G}12S NSCLC) and Colon-26 (KRAS^{G12D} CRC) mouse tumor models at various dose ranges. Significant tumor reduction was observed at all doses and in all models, lending evidence that Compound A activity is not dependent upon specific RAS mutations or RAS paralogues.

In head-to-head nonclinical studies, Compound A was compared to certain current FDA-approved MEK, BRAF and KRAS^{G12C} inhibitors in KRAS-, NRAS-, and BRAF-mutated solid tumor rodent models representing lung, colon, pancreas, and skin cancers; in each study, it demonstrated tumor stasis or regression while maintaining body weight. For instance, Compound A (100 and 150 mg/kg BID) was compared to the commercially available KRAS inhibitors sotorasib (30 and 100 mg/kg QD) and adagrasib (30 and 100 mg/kg QD) in the Capan-2 (KRAS^{G12D}) model, with Compound A showing efficacy in a non-KRAS^{G}12C model. In the KRAS^{G12C} MIA Paca-2 pancreatic model, similar tumor growth inhibition was observed between Compound A and sotorasib, and tumor regressions with improved durability of response (DoR) were demonstrated when Compound A and sotorasib were used in combination.

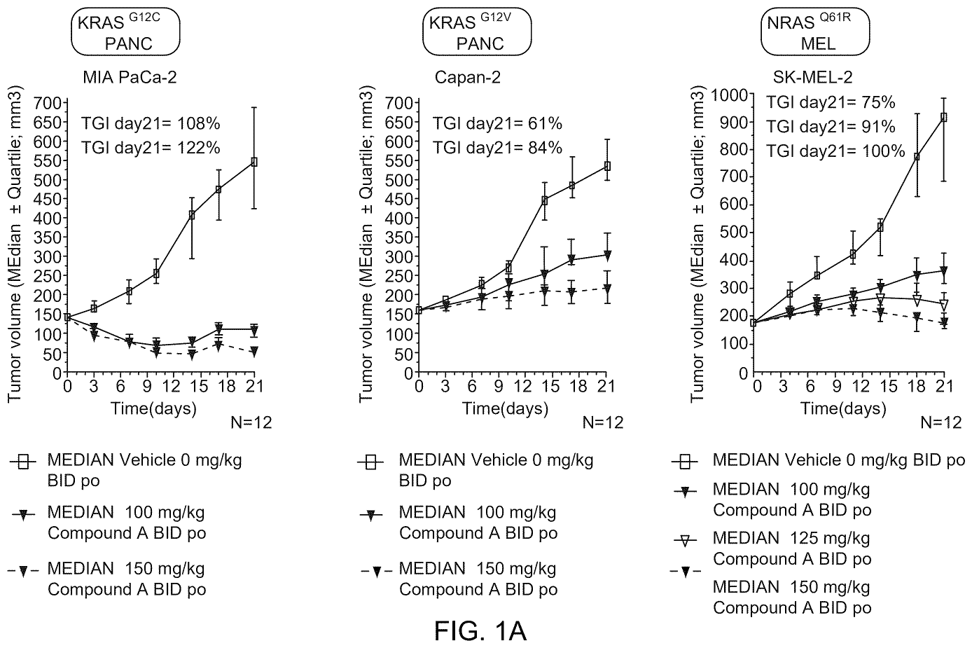

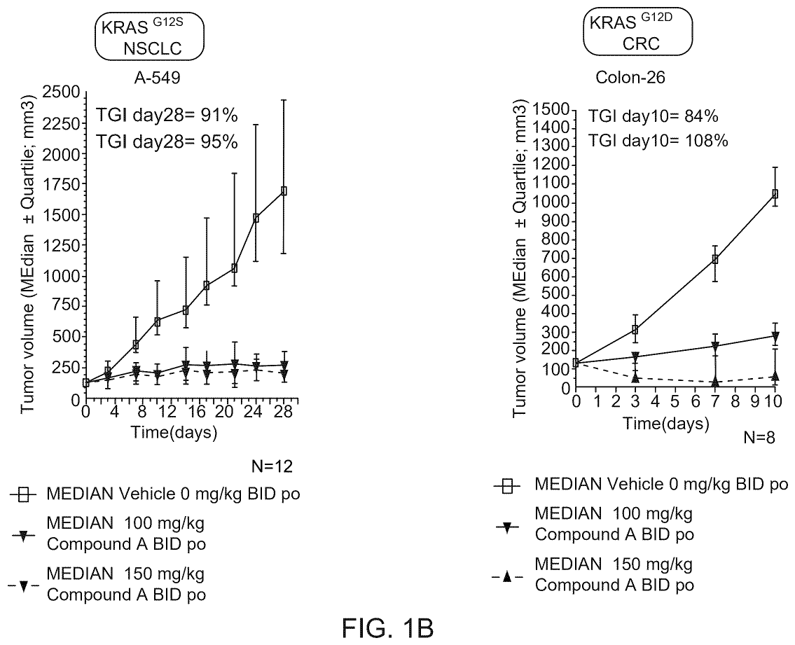

FIGS. 1A and 1B are a summary of results obtained from preclinical testing of Compound A in various xenograft tumor models, for pancreatic cancer (KRAS-G12C and KRAS-G12V), melanoma (NRAS-Q61R), non-small cell lung cancer (NSCLC) (KRAS-G12S), and colorectal cancer (KRAS-G12D). Compound A was well tolerated with a median body weight loss ≤3-6% of baseline at top doses. Maximum effective dose range in mice (plasma t1/2=1.3 hours) is 100 mg/kg to 150 mg/kg BID po. CRC=colorectal cancer, NSCLC=non-small cell lung cancer; MEL—melanoma; PANC=pancreatic cancer. Tumor Growth Inhibition; TGI %=[1−(Ti−T₀)/(Ci−C₀)]×100; T=treatment groups; C=control groups. Capan-2 PANC model (0% TGI for sotorasib and adagrasib at top doses).

In some embodiments, methods of treating a patient with a RAS-mutated cancer are provided. The method can include the administration of a therapeutically effective amount of a dual-MEK inhibitor inhibiting both the phosphorylation of mitogen-activated protein kinase kinase MEK1 and MEK2 (MEK) and the phosphorylation of mitogen-activated protein kinase (pERK), wherein the dual-MEK inhibitor is administered to the patient (a) in a dose amount that achieves a Cmax effective to inhibit pERK and MEK and (b) with an interval between consecutive doses that is at least about seven times the plasma half-life of the dual-MEK inhibitor. In some embodiments, the Cmax measured on the day of initial administration of the dual-MEK inhibitor may include at least 2,000 ng/mL. In some embodiments, the dual-MEK inhibitor may include at least about 1 micromolar dual-MEK inhibitor free fraction. In some embodiments, the Cmin may include about 0.1 micromolar free fraction of the dual-MEK inhibitor and may occur more than about 6 hours after the administration of the dual-MEK inhibitor. For example, the Cmin of about 0.1 micromolar free fraction occurs about 6-8 hours after the administration of the dual-MEK inhibitor, or later. In some embodiments, the interval between consecutive doses of the dual-MEK inhibitor that is about seven to eighteen times the plasma half-life of the dual-MEK inhibitor. In some embodiments, the dual-MEK inhibitor is Compound A or a pharmaceutically acceptable salt thereof.

In some embodiments, a method of treatment includes administering a dual-MEK inhibitor having a high Cmax and a short plasma drug half-life at sequential doses that are administered at a dose interval (i.e., time between doses) that is greater than the plasma drug half-life. For example, in some embodiments, a method of treatment comprises administering a dual-MEK inhibitor on a dosing schedule providing a Cmax that achieves sufficiently high pharmacodynamic (PD) inhibition (e.g. 90% or greater) with sequential doses of the dual-MEK inhibitor being administered at intervals that are about 7-18 times the duration of the plasma drug half-life.

In some embodiments, methods of treatment comprise the administration of a dual-MEK inhibitor to achieve deep cyclic inhibition of the MAPK pathway. Deep cyclic inhibition is a therapeutic approach for treatment of certain forms of cancer that aims to deprive tumor cells of the sustained proliferative signaling required for rapid growth, while sparing healthy cells through a cadenced, normalized level of signaling. In some embodiments, deep cyclic inhibition can be achieved by administering a dual-MEK inhibitor having sufficiently high pharmacodynamic inhibition of pERK (e.g., ~90%) and sufficiently short plasma half-life (e.g., ~2 hours) at sequential dose intervals that are about seven or more times the plasma half-life (e.g., once or twice daily).

In some embodiments, the cancer is pancreatic ducutal adenocarcinoma (PDAC). Preclinical mouse models in PDAC, non-small cell lung cancer, melanoma, and colorectal cancer demonstrated tumor reduction across all dose levels and in all models. Additionally, Compound A showed greater reductions in tumor volume in head-to-head comparisons with approved MEK inhibitors. In some embodiments, the cancer is colorectal cancer (CRC).

In some embodiments, the cancer has a RAS mutation that is a KRAS G12D or G12V mutation or a NRAS Q61L or Q61K mutation. In some embodiments, the cancer has a KRAS G12D mutation. In some embodiments, the cancer has a KRAS G12V mutation. In some embodiments, the cancer has a NRAS Q61L mutation. In some embodiments, the cancer has a NRAS Q61K mutation.

In some embodiments, the cancer is selected from the group consisting of: NRAS-mutated melanoma, KRAS-mutated PDAC, KRAS-mutated NSCLC and KRAS-mutated, and APC-wildtype CRC. In some embodiments, the cancer is NRAS-mutated melanoma. In some embodiments, the cancer is KRAS-mutated PDAC. In some embodiments, the cancer is KRAS-mutated NSCLC. In some embodiments, the cancer is KRAS-mutated CRC. In some embodiments, the cancer is APC-wildtype CRC. In some embodiments, the cancer is pancreatic cancer with a KRAS G12C mutation. In some embodiments, the cancer is pancreatic cancer with a G12D mutation. In some embodiments, the cancer is pancreatic cancer with a G12V mutation. In some embodiments, the cancer is colorectal cancer with a NRAS Q61L mutation. In some embodiments, the cancer is colorectal cancer with a NRAS Q61K mutation. In some embodiments, the cancer is colorectal cancer with a KRAS G12D mutation. In some embodiments, the cancer is melanoma with a NRAS Q61R mutation. In some embodiments, the cancer is melanoma with a KRAS G12S mutation. In some embodiments, the cancer melanoma with a BRAF V600E mutation. In some embodiments, the cancer is non-small cell lung cancer (NSCLC) with a KRAS G12S mutation.

In some embodiments, the dual-MEK inhibitor is Compound A and may be administered to the patient once daily in a dose amount of about 40 mg to treat a RAS mutated cancer. In some embodiments, the dual-MEK inhibitor is Compound A and may be administered to the patient once daily in a dose amount of 60 mg to treat a RAS mutated cancer. In some embodiments, the dual-MEK inhibitor is Compound A and may be administered to the patient once daily in a dose amount of about 80 mg to treat a RAS mutated cancer. In some embodiments, the dual-MEK inhibitor is Compound A administered and may be to the patient once daily in a dose amount of about 100 mg to treat a RAS mutated cancer. In some embodiments, the dual-MEK inhibitor is Compound A and may be administered to the patient once daily in a dose amount of about 120 mg to treat a RAS mutated cancer. In some embodiments, the dual-MEK inhibitor is Compound A and may be administered to the patient once daily in a dose amount of about 140 mg to treat a RAS mutated cancer. In some embodiments, the dual-MEK inhibitor is Compound A and may be administered to the patient once daily in a dose amount of about 160 mg to treat a RAS mutated cancer. In some embodiments, the dual-MEK inhibitor is Compound A and may be administered to the patient once daily in a dose amount of about 180 mg to treat a RAS mutated cancer. In some embodiments, the dual-MEK inhibitor is Compound A and may be administered to the patient once daily in a dose amount of about 200 mg to treat a RAS mutated cancer. In some embodiments, the dual-MEK inhibitor is Compound A and may be administered to the patient once daily in a dose amount of about 240 mg to treat a RAS mutated cancer. In some embodiments, the dual-MEK inhibitor is Compound A and may be administered to the patient once daily in a dose amount of about 260 mg to treat a RAS mutated cancer. In some embodiments, the dual-MEK inhibitor is Compound A and may be administered to the patient once daily in a dose amount of about 280 mg to treat a RAS mutated cancer. In some embodiments, the dual-MEK inhibitor is Compound A and may be administered to the patient once daily in a dose amount of about 300 mg to treat a RAS mutated cancer. In some embodiments, the dual-MEK inhibitor is Compound A and may be administered to the patient once daily in a dose amount of about 320 mg to treat a RAS mutated cancer. In some embodiments, the dual-MEK inhibitor is Compound A and may be administered to the patient once daily in a dose amount of about 340 mg to treat a RAS mutated cancer. In some embodiments, the dual-MEK inhibitor is Compound A and may be administered to the patient once daily in a dose amount of about 360 mg to treat a RAS mutated cancer. In some embodiments, the dual-MEK inhibitor is Compound A and may be administered to the patient once daily in a dose amount of about 380 mg to treat a RAS mutated cancer. In some embodiments, the dual-MEK inhibitor is Compound A and may be administered to the patient once daily in a dose amount of about 400 mg to treat a RAS mutated cancer. In some embodiments, the dual-MEK inhibitor is Compound A and may be administered to the patient once daily in a dose amount of about 420 mg to treat a RAS mutated cancer. In some embodiments, the dual-MEK inhibitor is Compound A and may be administered to the patient once daily in a dose amount of about 440 mg to treat a RAS mutated cancer. In some embodiments, the dual-MEK inhibitor is Compound A and may be administered to the patient once daily in a dose amount of about 460 mg to treat a RAS mutated cancer. In some embodiments, the dual-MEK inhibitor is Compound A and may be administered to the patient once daily in a dose amount of about 480 mg to treat a RAS mutated cancer. In some embodiments, the dual-MEK inhibitor is Compound A and may be administered to the patient once daily in a dose amount of about 500 mg to treat a RAS mutated cancer.

In some embodiments, the dual-MEK inhibitor is Compound A and may be administered to the patient once daily in a dose of about 40 mg, 50 mg, 60 mg, 70 mg, 80 mg, 90 mg, 100 mg, 110 mg 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, or ranges including and/or spanning the aforementioned values. In some embodiments, the dual-MEK inhibitor is Compound A and may be administered to the patient once daily in a dose of about 40 mg to about 500 mg to treat a RAS mutated cancer. In some embodiments, the dual-MEK inhibitor is Compound A administered to the patient once daily in a dose of about 40 mg to about 160 mg to treat a RAS mutated cancer. In some embodiments, the dual-MEK inhibitor is Compound A administered to the patient once daily in a dose of about 40 mg to about 80 mg to treat a RAS mutated cancer. In some embodiments, the dual-MEK inhibitor is Compound A and may be administered to the patient once daily in a dose amount of 40 mg to about 320 mg to treat a RAS mutated cancer. In some embodiments, the dual-MEK inhibitor is Compound A and may be administered to the patient once daily in a dose of about 80 to about 500 mg to treat a RAS mutated cancer. In some embodiments, the dual-MEK inhibitor is Compound A and may be administered to the patient once daily in a dose of about 80 to about 320 mg to treat a RAS mutated cancer. In some embodiments, the dual-MEK inhibitor is Compound A and may be administered to the patient once daily in a dose of about 80 to about 160 mg to treat a RAS mutated cancer. In some embodiments, the dual-MEK inhibitor is Compound A and may be administered to the patient once daily in a dose amount of about 80 mg to about 120 mg to treat a RAS mutated cancer. In some embodiments, the dual-MEK inhibitor is Compound A and may be administered to the patient once daily in a dose of about 100 to about 500 mg to treat a RAS mutated cancer. In some embodiments, the dual-MEK inhibitor is Compound A and may be administered to the patient once daily in a dose of about 100 to about 320 mg to treat a RAS mutated cancer. In some embodiments, the dual-MEK inhibitor is Compound A and may be administered to the patient once daily in a dose of about 100 to about 160 mg to treat a RAS mutated cancer. In some embodiments, the dual-MEK inhibitor is Compound A and may be administered to the patient once daily in a dose amount of about 100 mg to about 120 mg to treat a RAS mutated cancer. In some embodiments, the dual-MEK inhibitor is Compound A and may be administered to the patient once daily in a dose of about 120 to about 500 mg to treat a RAS mutated cancer. In some embodiments, the dual-MEK inhibitor is Compound A and may be administered to the patient once daily in a dose of about 120 to about 320 mg to treat a RAS mutated cancer. In some embodiments, the dual-MEK inhibitor is Compound A and may be administered to the patient once daily in a dose of about 120 to about 160 mg to treat a RAS mutated cancer. In some embodiments, the dual-MEK inhibitor is Compound A and may be administered to the patient once daily in a dose amount of about 120 mg to about 140 mg to treat a RAS mutated cancer. In some embodiments, the dual-MEK inhibitor is Compound A and may be administered to the patient once daily in a dose of about 160 to about 500 mg to treat a RAS mutated cancer. In some embodiments, the dual-MEK inhibitor is Compound A and may be administered to the patient once daily in a dose amount of about 160 mg to about 320 mg to treat a RAS mutated cancer. In some embodiments, the dual-MEK inhibitor is Compound A and may be administered to the patient once daily in a dose of about 160 to about 240 mg to treat a RAS mutated cancer. In some embodiments, the dual-MEK inhibitor is Compound A and may be administered to the patient once daily in a dose of about 160 to about 180 mg to treat a RAS mutated cancer.

In some embodiments, Compound A may be formulated as a pharmaceutical composition using a pharmaceutically acceptable excipients and administered by a variety of routes. In some embodiments, such compositions are for oral administration. Such pharmaceutical compositions and processes for preparing them are well known in the art. (See, e.g., Remington: The Science and Practice of Pharmacy, L. V. Allen, Editor, 22nd Edition, Pharmaceutical Press, 2012).

In a particular embodiment, the pharmaceutical composition comprises 4-((dimethylamino)methyl)-3-(2-fluoro-3-((N-methylsulfamoyl)amino)benzyl)-2-oxo-2H-chromen-7-yl dimethylcarbamate or a pharmaceutically acceptable salt or hydrate thereof, and a pharmaceutically acceptable carrier.

In some embodiments, to prepare a pharmaceutical composition for oral delivery, 0.1 mg to 120 mg of a compound of an embodiment is mixed with 750 mg of starch. The mixture is incorporated into an oral dosage unit, such as a hard gelatin capsule, or 0.1 mg to 120 mg of compound is granulated with binder solution such as starch solution along with suitable diluents such as microcrystalline cellulose or like, disintegrants such as croscarmellose sodium, dry the resultant mixture and add lubricant and compress into tablet which is suitable for oral administration.

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g., intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. In some embodiments, the pharmaceutical composition can be a medicament.

In some embodiments, pharmaceutical compositions comprising Compound A or pharmaceutically acceptable salts thereof can be formulated for oral administration. For example, a compound provided herein can be combined with suitable compendial excipients to form an oral unit dosage form, such as a capsule or tablet, containing a target dose of Compound A. The drug product can be prepared by first manufacturing Compound A as an active pharmaceutical ingredient (API), followed by roller compaction/milling with intragranular excipients and blending with extra granular excipients. A Drug Product can contain Compound A as the API and excipient components in a tablet in a desired dosage strength of Compound A. The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include Compound A as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this disclosure, active compounds can be given per se or as a pharmaceutical composition containing, for example, 0.1 to 99.5% (or, for example, 0.5 to 90%) of an active ingredient in combination with a pharmaceutically acceptable carrier.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present disclosure may contain conventional pharmaceutical carriers and/or auxiliary agents. In some embodiments, the pharmaceutical compositions according to the present disclosure may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

In some embodiments, an oral dosage form of Compound A is provided, such as the oral dosage form disclosed in Example 11. In some embodiments, an oral dosage form such as a tablet or capsule can contain Compound A or a pharmaceutically acceptable salt thereof as the API and one or more excipients such as a dry binder, tablet disintegrant, an absorbent, filler or diluent, a lubricant, anti-adherent, binder, thickening agent, viscosity-increasing agent, coating agent and film former. In some embodiments, an oral dosage form such as a tablet or capsule can contain 20 mg to 160 mg of Compound A or a pharmaceutically acceptable salt thereof as the API and one or more intra-granulation excipients such as a dry binder, tablet disintegrant, an absorbent, filler or diluent, a lubricant, anti-adherent, binder, thickening agent, viscosity-increasing agent, coating agent and film former. In some embodiments, an oral dosage form such as a tablet or capsule can contain 20 mg, 120 mg or 160 mg of Compound A or a pharmaceutically acceptable salt thereof as the API and one or more intra-granulation excipients such as a dry binder, tablet disintegrant, an absorbent, filler or diluent, a lubricant, anti-adherent, binder, thickening agent, viscosity-increasing agent, coating agent and film former and one or more extra granulation excipients such as additional Compound A API granulate and a lubricant or film forming agent.

In some embodiments, an oral dosage form such as a tablet or capsule can contain 20 mg, 120 mg or 160 mg of Compound A or a pharmaceutically acceptable salt thereof as the API and one or more intra-granulation excipients such as microcrystalline cellulose, crospovidone Type A, and hydroxypropyl cellulose, and one or more extra-granulation excipients such as API granulate of Compound A and magnesium stearate. In some embodiments, an oral dosage form such as a tablet or capsule can contain about 20% w/w of Compound A or a pharmaceutically acceptable salt thereof as the API and about 60-70% w/w microcrystalline cellulose, about 5% w/w crospovidone Type A, and about 10% w/w hydroxypropyl cellulose, and one or more extra-granulation excipients such as API granulate of Compound A (95-99.5% w/w) and magnesium stearate (0.5-5% w/w).

Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

In certain aspects, therapeutic methods or uses are provided herein for treating, preventing, or ameliorating a disease or condition in a subject. Some embodiments relate to the use of a compound or composition described herein to treat, prevent, or ameliorate cancer associated with an oncogenic RAS mutation. Some embodiments relate to the use of a compound or composition as described herein to treat cancer characterized by expressing a mutant form of RAS protein in a subject. Some embodiments relate to the use of a compound or composition as described herein to inhibit the proliferation of a cell having a RAS mutation. Some embodiments relate to the use of a compound or composition as described herein to induce apoptosis in a cell having a RAS mutation. Some embodiments relate to the use of a compound or composition as described herein to inhibit the progression of cancer that is characterized by expressing a mutant form of RAS protein in a subject. In some embodiments, the RAS protein is a KRAS protein. In some embodiments, the RAS protein is an NRAS protein. In some embodiments, the RAS protein is an HRAS protein.

### Detecting RAS-Mutated Cancer

In some embodiments, patients are selected for treatment with Compound A based on the presence of RAS mutation such as a KRAS G12C mutation in plasma or tumor specimens. If no mutation is detected in a plasma specimen, test tumor tissue. Additional information on FDA-approved tests for the detection of a KRAS G12C mutation is available at: https://www.fda.gov/CompanionDiagnostics. FDA approved diagnostics for identifying patients with a RAS-mutated cancer include the companion diagnostics provided in Table 1 below.

TABLE 1

| | Indication | | |
|---|---|---|---|
| Diagnostic Name | Sample Type | Biomarker | Biomarker(s) (Details) |
| Agilent Resolution ctDx | Non-Small Cell | KRAS | KRAS G12C |
| FIRST assay | Lung Cancer | | |
| (Resolution Bioscience, | (NSCLC)—Plasma | | |
| Inc.) | | | |
| cobas KRAS Mutation | Colorectal | KRAS | Mutations in codons 12 and 13 of |
| Test (Roche Molecular | Cancer—Tissue | | KRAS gene |
| Systems, Inc.) | | | |
| cobas KRAS Mutation | Colorectal | KRAS | Mutations in codons 12 and 13 of |
| Test (Roche Molecular | Cancer—Tissue | | KRAS gene |
| Systems, Inc.) | | | |
| CRCDx RAS Mutation | Colorectal Cancer | KRAS and | KRAS wild-type biomarkers (the |
| Detection Assay Kit | (CRC)—Tissue | NRAS | absence of mutations in exons 2, 3, or |
| (EntroGen, Inc.) | | | 4) and NRAS wild-type biomarkers |
| | | | (the absence of mutations in exons 2, |
| | | | 3, or 4) |
| FoundationOne CDx | Colorectal | KRAS | KRAS wild-type (absence of |
| (Foundation Medicine, | Cancer—Tissue | | mutations in codons 12 and 13) |
| Inc.) | | | |
| FoundationOne CDx | Colorectal | KRAS and | KRAS wild-type (absence of |
| (Foundation Medicine, | Cancer—Tissue | NRAS | mutations in exons 2, 3, and 4) and |
| Inc.) | | | NRAS wild type (absence of |
| | | | mutations in exons 2, 3, and 4) |
| Guardant360 CDx | Non-Small Cell | KRAS | G12C |
| (Guardant Health, Inc.) | Lung Cancer | | |
| | (NSCLC)—Plasma | | |
| ONCO/Reveal Dx Lung | Colorectal | KRAS | KRAS wild-type (absence of |
| & Colon Cancer Assay | Cancer—Tissue | | mutations in codons 12 and 13) |
| (O/RDx-LCCA) (Pillar | | | |
| Biosciences, Inc.) | | | |
| ONCO/Reveal Dx Lung | Colorectal | KRAS | KRAS wild-type (absence of |
| & Colon Cancer Assay | Cancer—Tissue | | mutations in codons 12 and 13) |
| (O/RDx-LCCA) (Pillar | | | |
| Biosciences, Inc.) | | | |
| therascreen KRAS RGQ | Colorectal | KRAS | G12A, G12D, G12R, G12C, G12S, |
| PCR Kit (Qiagen | Cancer—Tissue | | G12V, G13D |
| Manchester, Ltd.) | | | |
| therascreen KRAS RGQ | Non-Small Cell | KRAS | G12C |
| PCR Kit (Qiagen | Lung Cancer | | |
| Manchester, Ltd.) | (NSCLC)—Tissue | | |
| therascreen KRAS RGQ | Colorectal | KRAS | G12A, G12D, G12R, G12C, G12S, |
| PCR Kit (Qiagen | Cancer—Tissue | | G12V, G13D |
| Manchester, Ltd.) | | | |
| therascreen KRAS RGQ | Colorectal | KRAS | KRAS wild-type (absence of |
| PCR Kit (Qiagen | Cancer—Tissue | | mutations in codons 12 and 13) |
| Manchester, Ltd.) | | | |
| therascreen KRAS RGQ | Non-Small Cell | KRAS | KRAS G12C |
| PCR Kit (Qiagen | Lung Cancer | | |
| Manchester, Ltd.) | (NSCLC)—Tissue | | |
| xT CDx (Tempus Labs, | Colorectal Cancer | KRAS | KRAS wild-type (absence of |
| Inc.) | (CRC)—Tissue | | mutations in codons 12 or 13) |
| | (Matching | | |
| | Blood/Saliva) | | |
| xT CDx (Tempus Labs, | Colorectal Cancer | KRAS and | KRAS wild-type (absence of |
| Inc.) | (CRC)—Tissue | NRAS | mutations in exons 2, 3, or 4) and |
| | (Matching | | NRAS wild-type (absence of |
| | Blood/Saliva) | | mutations in exons 2, 3, or 4) |

In some embodiments, RAS mutations can be detected by next generation sequencing (NGS) techniques. For example, molecular diagnostics of HRAS/KRAS and NRAS targeted gene panels can be obtained by NGS techniques. Next generation sequencing of amplification products can be performed on the Ion Torrent Personal Genome Machine and analyzed with the Torrent Suite Software. Reference DNA sequences used for these genes can be found at https://www.ncbi.nlm.nih.gov/refseq/rsg/. This mutation panel is designed to detect targeted mutations only. Confirmation of these mutations is performed by traditional or real time PCR followed by Sanger sequencing, fluorescent melting curve analysis and/or pyrosequencing (CPT codes: 81210 (BRAF), 81275 (KRAS codons 12 and 13 in exon 2), 81276 (KRAS codon 61 in exon 3), 81403 (HRAS), 81311 (NRAS)).

### Immune Checkpoint Inhibitors

In some embodiments, Compound A is administered to treat a cancer such as melanoma that has been previously treated with one or more immune checkpoint inhibitor. In some embodiments, Compound A is administered in combination with one or more immune checkpoint inhibitor to treat a cancer such as melanoma.

A review describing immune checkpoint pathways and the blockade of such pathways with immune checkpoint inhibitor compounds is provided by Pardoll in Nature Reviews Cancer (April 2012), pages 252-264, which is incorporated herein by reference in its entirety. Immune check point inhibitor compounds display anti-tumor activity by blocking one or more of the endogenous immune checkpoint pathways that downregulate an anti-tumor immune response. The inhibition or blockade of an immune checkpoint pathway typically involves inhibiting a checkpoint receptor and ligand interaction with an immune checkpoint inhibitor compound to reduce or eliminate the down regulation signal and resulting diminishment of the anti-tumor response.

The term “immune checkpoint inhibitor” or “ICI,” as used herein, refers to a molecule (e.g., small molecule, peptide, polypeptide, protein, antibody, antibody fragment and the like) that acts as an inhibitor (antagonist) of an immune checkpoint pathway. Inhibition of a pathway can include blockade of the pathway through binding to a receptor or signaling molecule that is part of the immune checkpoint pathway.

In some embodiments of the present disclosure, the immune checkpoint inhibitor compound inhibits the signaling interaction between an immune checkpoint receptor and the corresponding ligand of the immune checkpoint receptor. The immune checkpoint inhibitor compound can act by blocking activation of the immune checkpoint pathway by inhibition (antagonism) of an immune checkpoint receptor (some examples of receptors include CTLA-4, PD-1, LAG-3, TIM-3, BTLA, and KIR) or by inhibition of a ligand of an immune checkpoint receptor (some examples of ligands include PD-L1 and PD-L2). In such embodiments, the effect of the immune checkpoint inhibitor compound is to reduce or eliminate down regulation of certain aspects of the immune system anti-tumor response in the tumor microenvironment.

The Programmed Death 1 (PD-1) protein is an inhibitory member of the extended CD28/CTLA-4 family of T cell regulators (Okazaki et al. (2002) Cuff Opin Immunol 14: 391779-82; Bennett et al. (2003) J. Immunol. 170:711-8; which are incorporated herein by reference in their entirety). Other members of the CD28 family include CD28, CTLA-4, ICOS and BTLA. PD-1 is suggested to exist as a monomer, lacking the unpaired cysteine residue characteristic of other CD28 family members. PD-1 is expressed on activated B cells, T cells, and monocytes.

The PD-1 gene encodes a 55 kDa type I transmembrane protein (Agata et al. (1996) Int Immunol. 8:765-72, which is incorporated herein by reference in its entirety). Although structurally similar to CTLA-4, PD-1 lacks the MYPPY motif that is important for B7-1 and B7-2 binding. Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43; which are incorporated herein by reference in their entirety). Both PD-L1 and PD-L2 are B7 homologs that bind to PD-1, but do not bind to other CD28 family members. PD-L1 is abundant in a variety of human cancers (Dong et al. (2002) Nat. Med. 8:787-9, which is incorporated herein by reference in its entirety).

PD-1 is known as an immunoinhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745; which are incorporated herein by reference in their entirety). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100; which are incorporated herein by reference in their entirety). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat′l. Acad. Sci. USA 99:12293-7; Brown et al. (2003) J. Immunol. 170:1257-66; which are incorporated herein by reference in their entirety).

The immune checkpoint receptor cytotoxic T-lymphocyte associated antigen 4 (CTLA-4) is expressed on T-cells and is involved in signaling pathways that reduce the level of T-cell activation. It is believed that CTLA-4 can downregulate T-cell activation through competitive binding and sequestration of CD80 and CD86. In addition, CTLA-4 has been shown to be involved in enhancing the immunosuppressive activity of TReg cells.

The immune checkpoint receptor programmed death 1 (PD-1) is expressed by activated T-cells upon extended exposure to antigen. Engagement of PD-1 with its known binding ligands, PD-L1 and PD-L2, occurs primarily within the tumor microenvironment and results in downregulation of anti-tumor specific T-cell responses. Both PD-L1 and PD-L2 are known to be expressed on tumor cells. The expression of PD-L1 and PD-L2 on tumors has been correlated with decreased survival outcomes.

The immune checkpoint receptor T cell membrane protein 3 (TIM-3) is expressed on Th1 and Tc1 cells, but not other T-cells. Interaction of TIM-3 with its ligand, galectin-9, produces a Th1 cell death signal. TIM-3 has been reported to play a role in maintaining T-cell exhaustion and blockade of TIM-3 has been shown to restore activity to exhausted T-cells.

The immune checkpoint receptor B- and T-lymphocyte attenuator (BTLA) receptor is expressed on both resting and activated B-cells and T-cells. Activation of BTLA when combined with its ligand HVEM (herpes virus entry mediator) results in downregulation of both T-cell activation and proliferation. HVEM is expressed by certain tumors (e.g., melanoma) and tumor-associated endothelial cells.

The immune checkpoint receptors known as killer cell immunoglobulin-like receptors (KIR) are a polymorphic family of receptors expressed on NK cells and some T-cells and function as regulators of immune tolerance associated with natural killer (NK) cells. Blocking certain KIR receptors with inhibitor compounds can facilitate the destruction of tumors through the increased activity of NK cells.

In some embodiments of the present disclosure, the immune checkpoint inhibitor compound is a small organic molecule (molecular weight less than 1000 daltons), a peptide, a polypeptide, a protein, an antibody, an antibody fragment, or an antibody derivative. In some embodiments, the immune checkpoint inhibitor compound is an antibody. In some embodiments, the antibody is a monoclonal antibody, specifically a human or a humanized monoclonal antibody.

Monoclonal antibodies, antibody fragments, and antibody derivatives for blocking immune checkpoint pathways can be prepared by any of several methods known to those of ordinary skill in the art, including but not limited to, somatic cell hybridization techniques and hybridoma, methods. Hybridoma generation is described in Antibodies, A Laboratory Manual, Harlow and Lane, 1988, Cold Spring Harbor Publications, New York, which is incorporated herein by reference in its entirety. Human monoclonal antibodies can be identified and isolated by screening phage display libraries of human immunoglobulin genes by methods described for example in U.S. Pat. Nos. 5,223,409, 5,403,484, 5,571,698, 6,582,915, and 6,593,081, which are incorporated herein by reference in their entirety. Monoclonal antibodies can be prepared using the general methods described in U.S. Pat. No. 6,331,415 (Cabilly), which is incorporated herein by reference in its entirety.

As an example, human monoclonal antibodies can be prepared using a XenoMouse™ (Abgenix, Freemont, Calif.) or hybridomas of B cells from a XenoMouse. A XenoMouse is a murine host having functional human immunoglobulin genes as described in U.S. Pat. No. 6,162,963 (Kucherlapati), which is incorporated herein by reference in its entirety.

Methods for the preparation and use of immune checkpoint antibodies are described in the following illustrative publications. The preparation and therapeutic uses of anti-CTLA-4 antibodies are described in U.S. Pat. No. 7,229,628 (Allison), U.S. Pat. No. 7,311,910 (Linsley), and U.S. Pat. No. 8,017,144 (Korman), which are incorporated herein by reference in their entirety. The preparation and therapeutic uses of anti-PD-1 antibodies are described in U.S. Pat. No. 8,008,449 (Korman) and U.S. Patent Application No. 2011/0271358 (Freeman), which are incorporated herein by reference in their entirety. The preparation and therapeutic uses of anti-PD-L1 antibodies are described in U.S. Pat. No. 7,943,743 (Korman), which is incorporated herein by reference in its entirety. The preparation and therapeutic uses of anti-TIM-3 antibodies are described in U.S. Pat. No. 8,101,176 (Kuchroo) and U.S. Pat. No. 8,552,156 (Tagayanagi), which are incorporated herein by reference in their entirety. The preparation and therapeutic uses of anti-LAG-3 antibodies are described in U.S. Patent Application No. 2011/0150892 (Thudium) and International Publication Number WO2014/008218 (Lonberg), which are incorporated herein by reference in their entirety. The preparation and therapeutic uses of anti-KIR antibodies are described in U.S. Pat. No. 8,119,775 (Moretta), which is incorporated herein by reference in its entirety. The preparation of antibodies that block BTLA regulated inhibitory pathways (anti-BTLA antibodies) are described in U.S. Pat. No. 8,563,694 (Mataraza), which is incorporated herein by reference in its entirety.

In some embodiments, the one or more immune checkpoint inhibitor is an inhibitor of PD-1, PD-L1, or CTLA-4. In some embodiments, the immune checkpoint inhibitor is a PD-1 inhibitor. In some embodiments, the immune checkpoint inhibitor is a binding ligand of PD-L1. In some embodiments, the immune checkpoint inhibitor is a PD-L1 inhibitor. In some embodiments, the immune checkpoint inhibitor is a CTLA-4 inhibitor.

In some embodiments, the one or more immune checkpoint inhibitor as described herein includes a first immune checkpoint inhibitor and a second immune checkpoint inhibitor, wherein the first immune checkpoint inhibitor is different from the second immune checkpoint inhibitor. In some embodiments, the first and the second immune checkpoint inhibitor are independently an inhibitor of PD-1, PD-L1 or CTLA-4. In some embodiments, the first immune checkpoint inhibitor is a PD-1 inhibitor, and the second immune checkpoint inhibitor is a CTLA-4 inhibitor.

In some embodiments, the immune checkpoint inhibitor is pembrolizumab, nivolumab, cemiplimab, atezolizumab, avelumab, pembrolizumab, pidilizumab, ipilimumab, BMS 936559, relatlimab, durvalumab, or any combinations thereof. In some embodiments, the one or more immune checkpoint inhibitor may include an anti-PD-1 HuMAbs can be selected from 17D8, 2D3, 4H1, 5C4 (also referred to herein as nivolumab), 4A11, 7D3 and 5F4, all of which are described in U.S. Pat. No. 8,008,449, which is incorporated herein by reference in its entirety. In some embodiments, the anti-PD-1 HuMAbs can be selected from 3G10, 12A4 (also referred to herein as BMS-936559), 10A5, 5F8, 10H10, 1B12, 7H1, 11E6, 12B7, and 13G4, all of which are described in U.S. Pat. No. 7,943,743, which is incorporated herein by reference in its entirety.

In some embodiments, the one or more immune checkpoint inhibitor may be incorporated in a pharmaceutically acceptable formulation. In some embodiments, the one or more immune checkpoint inhibitor is incorporated in a pharmaceutically acceptable aqueous formulation. Examples of acceptable aqueous formulations include isotonic buffered and pH 4.5-8 adjusted saline solutions such as Lactated Ringer's Solution and the like.

In some embodiments, the immune checkpoint inhibitor compound is incorporated in a pharmaceutically acceptable liposome formulation, wherein the formulation is a passive or targeted liposome formulation. Examples of methods for the preparation of suitable liposome formulations of antibodies are described U.S. Pat. No. 5,399,331 (Loughrey), U.S. Pat. No. 8,304,565 (Wu) and U.S. Pat. No. 7,780,882 (Chang), which are incorporated herein by reference in their entirety.

In some embodiments, the one or more immune checkpoint inhibitor may be an antibody. In some embodiments, the antibody is a dry, lyophilized solid that is reconstituted with an aqueous reconstitution solvent prior to use. In some embodiments, the antibody is incorporated in a pharmaceutically acceptable formulation and the pharmaceutically acceptable formulation is injected directly into a tumor. In some embodiments, the immune checkpoint inhibitor antibody is incorporated in a pharmaceutically acceptable formulation and the pharmaceutically acceptable formulation is injected into the peritumoral region surrounding a tumor. The peritumoral region may contain antitumor immune cells. In some embodiments, the antibody is incorporated in a pharmaceutically acceptable formulation and the pharmaceutically acceptable formulation is administered by intravenous injection or infusion. In some embodiments, the immune checkpoint inhibitor antibody is incorporated in a pharmaceutically acceptable formulation and the pharmaceutically acceptable formulation is administered by subcutaneous injection or intradermal injection. In some embodiments, the antibody is incorporated in a pharmaceutically acceptable formulation and the pharmaceutically acceptable formulation is administered by intraperitoneal injection or lavage.

The precise amount of immune checkpoint inhibitor compound incorporated in a particular method or therapeutic combination of the disclosure may vary according to factors known in art such as for example, the physical and clinical status of the subject, the method of administration, the content of the formulation, the physical and chemical nature of the immune checkpoint inhibitor compound, the intended dosing regimen or sequence. Those of ordinary skill in the art, however, can readily determine the appropriate amount with due consideration of such factors.

### Definitions

The term “pharmaceutical composition,” as used herein, refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier,” as used herein, refers to a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent,” as used herein, refers to chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable,” as used herein, refers to a carrier or diluent that does not abrogate the biological activity and properties of the compound.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

As used herein, “alkyl” refers to a straight or branched hydrocarbon chain fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

The term “about,” as used herein, refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. When a value is preceded by the term about, the component is not intended to be limited strictly to that value, but it is intended to include amounts that vary from the value.

The term “pharmaceutically acceptable carrier” or “pharmaceutically acceptable excipient,” as used herein, includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The term “pharmaceutically acceptable salt,” as used herein, refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C1-C7 alkylamine, cyclohexylamine, triethanolamine, ethylenediamine, and salts with amino acids such as arginine, lysine, and the like.

The term “subject,” as used herein, refers to a human or a non-human mammal, e.g., a dog, a cat, a mouse, a rat, a cow, a sheep, a pig, a goat, a non-human primate or a bird, e.g., a chicken, as well as any other vertebrate or invertebrate.

### Additional Embodiments

In some embodiments, one or more of following methods are also provided.

A-1. A method of treating a patient with a RAS-mutated cancer, comprising administering a therapeutically effective amount of a dual-MEK inhibitor inhibiting both phosphorylation of mitogen-activated protein kinase kinase 1 and 2 (pMEK) and phosphorylation of mitogen-activated protein kinase 1 and 2 (pERK), wherein the dual-MEK inhibitor is administered to the patient (a) in a dose amount that achieves a Cmax effective to inhibit pERK and pMEK and (b) with an interval between consecutive doses that is at least about seven times the plasma half-life of the dual-MEK inhibitor.

A-2. The method of embodiment A-1, wherein the Cmax measured on day 1 of administering the dual-MEK inhibitor is at least 2,000 ng/mL.

A-3. The method of embodiment A-2, wherein the dual-MEK inhibitor has at least about 1 micromolar dual-MEK inhibitor free fraction.

A-4. The method of any one of embodiments A-1 to A-3, wherein a Cmin of over about 0.1 micromolar occurs longer than about 6 hours after administering of the dual-MEK inhibitor in a free fraction form.

A-5. The method of any one of embodiments A-1 to A-3, wherein a Cmin of over about 0.1 micromolar occurs longer than about 6-8 hours after administering of the dual-MEK inhibitor in a free fraction form.

A-6. The method of any one of embodiments A-1 to A-5, wherein the interval between consecutive doses of the dual-MEK inhibitor is about seven to eight times the plasma half-life of the dual-MEK inhibitor.

A-7. The method of any one of embodiments A-1 to A-6, wherein the dual-MEK inhibitor is Compound A

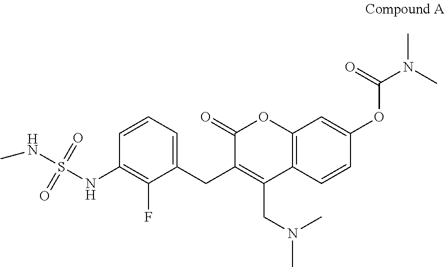

or a pharmaceutically acceptable salt thereof.

A-8. The method of any one of embodiments A-1 to A-7, wherein the RAS-mutated cancer is selected from the group consisting of: RAS-mutated melanoma, RAS-mutated PDAC, RAS-mutated NSCLC and RAS-mutated CRC, where RAS is any activation mutation in KRAS, NRAS or HRAS.

A-9. The method of any one of embodiments A-1 to A-7, wherein the RAS-mutated cancer is a KRAS G12D or G12V mutation or a NRAS Q61L or Q61K mutation.

A-10. The method of any one of embodiments A-1 to A-7, wherein the RAS-mutated cancer is pancreatic cancer with a KRAS G12C, G12D or G12V mutation.

A-11. The method of any one of embodiments A-1 to A-7, wherein the RAS-mutated cancer is colorectal cancer with a NRAS Q61L or Q61K mutation or colorectal cancer with a KRAS G12D mutation.

A-12. The method of any one of embodiments A-1 to A-7, wherein the RAS-mutated cancer is RAS-mutated melanoma with a NRAS Q61R mutation, or RAS-mutated melanoma with a BRAF V600E mutation.

A-13. The method of any one of embodiments A-1 to A-7, wherein the RAS-mutated cancer is non-small cell lung cancer (NSCLC) with a KRAS G12S mutation.

A-14. The method of any one of embodiments A-1 to A-13, wherein the dual-MEK inhibitor is Compound A administered to the patient once daily in a dose amount of about 240 mg or about 320 mg.

A-15. The method of embodiment A-14, wherein the dual-MEK inhibitor is Compound A administered to the patient once daily in a dose amount of about 240 mg.

A-16. The method of embodiment A-14, wherein the dual-MEK inhibitor is Compound A administered to the patient once daily in a dose amount of about 320 mg.

A-17. The method of any one of embodiments A-14 to A-16, wherein Compound A is administered orally to the patient in need thereof, for at least 28 consecutive days.

A-18. The method of embodiment A-17, wherein the patient is diagnosed with pancreatic cancer with a KRAS G12D mutation.

A-19. The method of embodiment A-17, wherein the patient is diagnosed with colorectal cancer with a KRAS G12V mutation.

A-20. The method of embodiment A-17, wherein the patient is diagnosed with colorectal cancer with a NRAS Q61L mutation.

A-21. The method of embodiment A-17, wherein the patient is diagnosed with colorectal cancer with a NRAS Q61K mutation.

A-22. A method of treating a patient with a cancer with a KRAS G12D or G12V mutation, the method consisting of orally administering to the patient in need thereof a therapeutically effective amount of Compound A,

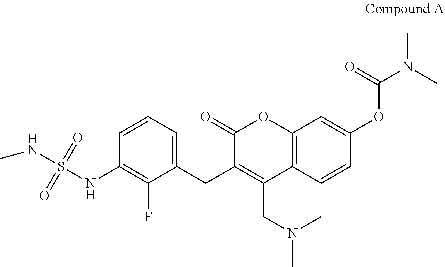

or a pharmaceutically acceptable salt thereof, once daily as a monotherapy.

A-23. The method of embodiment A-22, wherein the patient is diagnosed with pancreatic cancer with a KRAS G12D or G12V mutation.

A-24. The method of embodiment A-22, wherein the patient is diagnosed with colorectal cancer with a KRAS G12D or G12V mutation.

A-25. The method of any one of embodiments A-22 to A-24, wherein the therapeutically effective amount is a dose of 160 mg of Compound A orally once daily as a monotherapy.

A-26. The method of any one of embodiments A-22 to A-24, wherein the therapeutically effective amount is a dose of 240 mg of Compound A orally once daily as a monotherapy.

A-27. The method of any one of embodiments A-22 to A-24, wherein the therapeutically effective amount is a dose of 320 mg of Compound A orally once daily as a monotherapy.

A-28. A method of treating a patient with a cancer with a KRAS G12D or G12V mutation or a cancer with a NRAS Q61L or Q61K mutation, the method consisting of orally administering to the patient in need thereof a total dose of 160 mg of Compound A once daily to treat the cancer,

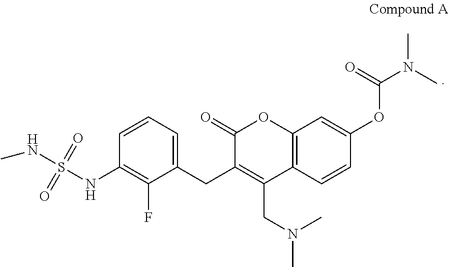

A-29. A method of treating a patient with a cancer with a KRAS G12D or G12V mutation or a cancer with a NRAS Q61L or Q61K mutation, the method consisting of orally administering to the patient in need thereof a total dose of 240 mg of Compound A once daily to treat the cancer,

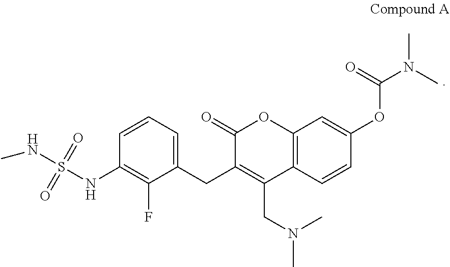

A-30. A method of treating a patient with a cancer with a KRAS G12D or G12V mutation or a cancer with a NRAS Q61L or Q61K mutation, the method consisting of orally administering to the patient in need thereof a total dose of 320 mg of Compound A once daily to treat the cancer,

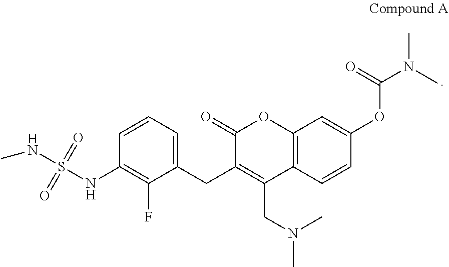

A-31. The method of any one of embodiments A-28 to A-30, wherein Compound A is administered as a monotherapy.

A-32. The method of embodiment A-31, wherein the cancer has a KRAS G12D mutation.

A-33. The method of embodiment A-31, wherein the cancer is pancreatic cancer with a KRAS G12D mutation.

A-34. The method of embodiment A-31, wherein the cancer has a KRAS G12V mutation.

A-35. The method of embodiment A-31, wherein the cancer is colorectal cancer with a KRAS G12V mutation.

A-36. The method of embodiment A-31, wherein the cancer with a NRAS Q61L mutation.

A-37. The method of embodiment A-31, wherein the cancer is colorectal cancer with a NRAS Q61L mutation.

A-38. The method of embodiment A-31, wherein the cancer has a NRAS Q61K mutation.

A-39. The method of embodiment A-31, wherein the cancer is colorectal cancer with a NRAS Q61K mutation.

A-40. A method of treating a patient with a cancer with a mutation in KRAS, NRAS, or HRAS, the method consisting of orally administering to the patient in need thereof a total dose of 160 mg of Compound A once daily to treat the cancer,

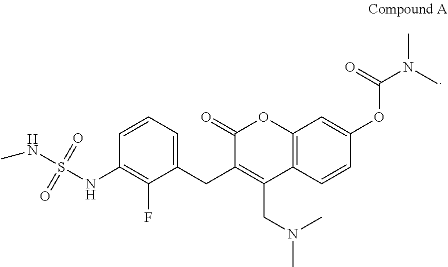

A-41. A method of treating a patient with a cancer with a mutation in KRAS, NRAS, or HRAS, the method consisting of orally administering to the patient in need thereof a total dose of 240 mg of Compound A once daily to treat the cancer,

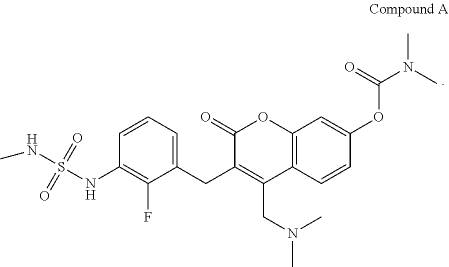

A-42. A method of treating a patient with a cancer with a mutation in KRAS, NRAS, or HRAS, the method consisting of orally administering to the patient in need thereof a total dose of 320 mg of Compound A once daily to treat the cancer,

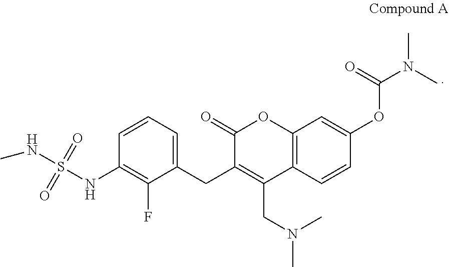

A-43. The method of any one of embodiments A-40 to A-42, wherein the cancer has a KRAS mutation.

A-44. The method of any one of embodiments A-40 to A-42, wherein the cancer has a KRAS G12C, G12D or G12S mutation.

A-45. The method of any one of embodiments A-43 to A-44, wherein the cancer is pancreatic cancer.

A-46. The method of any one of embodiments A-40 to A-42, wherein the cancer has a NRAS mutation.

A-47. The method of any one of embodiments A-40 to A-42, wherein the cancer has a NRAS Q61L or Q61K mutation.

A-48. The method of any one of embodiments A-46 to A-47, wherein the cancer is colorectal cancer.

A-49. The method of any one of embodiments A-40 to A-42, wherein the cancer has a HRAS mutation.

A-50. The method of any one of embodiments A-40 to A-49, wherein Compound A is administered as a monotherapy dual-MEK inhibitor.

A-51. A method of treating a patient with a RAS-mutated cancer, comprising administering once-daily orally a therapeutically effective amount of a dual-MEK inhibitor having at least about 1 micromolar dual-MEK inhibitor in free fraction, wherein the dual-MEK inhibitor inhibits both the phosphorylation of mitogen-activated protein kinase kinase 1 and 2 (pMEK) and the phosphorylation of mitogen-activated protein kinase 1 and 2 (pERK), wherein the dual-MEK inhibitor is administered to the patient (a) in a dose amount that achieves a Cmax, wherein, the Cmax is measured on day one of administering the dual-MEK inhibitor, wherein the Cmax at least 2,000 ng/mL and (b) with an interval between consecutive doses of the dual-MEK inhibitor is about seven to eight times a plasma half-life of the dual-MEK inhibitor.

A-52. The method of embodiment A-51, wherein the plasma half-life of the dual-MEK inhibitor in humans is 1-3 hours.

A-53. The method of embodiment A-51, wherein the plasma half-life of the dual-MEK inhibitor in humans is about 2 hours.

A-54. The method of any one of embodiments A-51 to A-53, wherein the dual-MEK inhibitor has a Cmin of over about 0.1 micromolar occurs 6-8 hours after administering the dual-MEK inhibitor free fraction.

A-55. The method of any one of embodiments A-1 to A-6 or A-51 to A-54, wherein the dual-MEK inhibitor is administered for at least 15 consecutive days.

A-56. The method of any one of embodiments A-1 to A-6 or A-51 to A-55, wherein the dual-MEK inhibitor is administered for at least 28 consecutive days.

A-57. A method of treating a patient with a RAS-mutated cancer, the method comprising orally administering to the patient in need thereof a total dose of 240 mg of Compound A once daily to the patient in need thereof, to treat the cancer

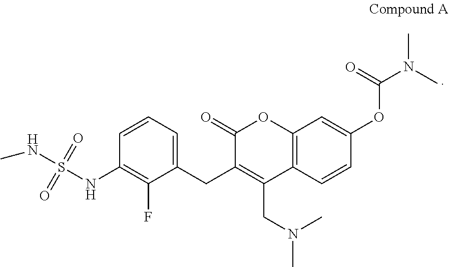

A-58. A method of treating a patient with a RAS-mutated cancer, the method comprising orally administering to the patient in need thereof a total dose of 320 mg of Compound A once daily to the patient in need thereof, to treat the cancer

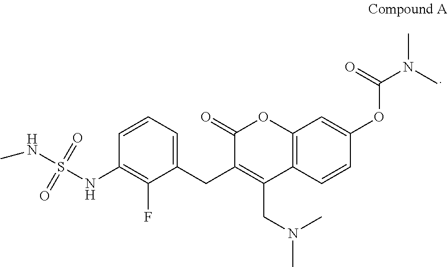

A-59. The method of any one of embodiments A-7 to A-50 or A-57 to A-58, wherein the dual-MEK inhibitor is administered for at least 15 consecutive days.

A-60. The method of any one of embodiments A-7 to A-50 or A-57 to A-58, wherein the dual-MEK inhibitor is administered for at least 28 consecutive days.

A-61. The method of any one of embodiments A-1 to A-7, or A-51 to A-60, wherein the RAS-mutated cancer is any RAS activated mutation in KRAS, NRAS or HRAS.

A-62. The method of embodiment A-61, wherein the RAS-mutated cancer is any RAS activated mutation in KRAS.

A-63. The method of embodiment A-62, wherein the RAS-mutated cancer has a mutation at KRAS G12C.

A-64. The method of embodiment A-62, wherein the RAS-mutated cancer has a mutation at KRAS G12D).

A-65. The method of embodiment A-62, wherein the RAS-mutated cancer has a mutation at KRAS G12V.

A-66. The method of any one of embodiments A-62 to A-65, wherein the cancer is pancreatic cancer.

A-67. The method of embodiment A-61, wherein the RAS-mutated cancer has a mutation at any activation mutation in NRAS.

A-68. The method of embodiment A-67, wherein the RAS-mutated cancer has a mutation at NRAS Q61L.

A-69. The method of embodiment A-67, wherein the RAS-mutated cancer has a mutation at NRAS Q61K.

A-70. The method of embodiment A-67, wherein the RAS-mutated cancer has a mutation at NRAS Q61R.

A-71. The method of any one of embodiments A-64, 68 or 69, wherein the cancer is colorectal cancer.

A-72. The method of embodiment A-70, wherein the cancer is melanoma.

A-73. The method of embodiment A-62, wherein the RAS-mutated cancer has a mutation at KRAS G12S.

A-74. The method of embodiment A-73, wherein the cancer is NSCLC.

A-75. The method of embodiment A-61, wherein the RAS-mutated cancer is any activation mutation in HRAS.

A-76. A method of treating cancer, the method comprising administering orally once-daily a therapeutically effective amount of a dual-MEK inhibitor having at least about 1 micromolar dual-MEK inhibitor in free fraction, wherein the dual-MEK inhibitor inhibits both the phosphorylation of mitogen-activated protein kinase kinase 1 and 2 (pMEK) and the phosphorylation of mitogen-activated protein kinase 1 and 2 (pERK), wherein the dual-MEK inhibitor is administered to the patient (a) in a dose amount that achieves a Cmax, wherein, the Cmax is measured on day one of administering the dual-MEK inhibitor, wherein the Cmax is at least 2,000 ng/mL and (b) with an interval between consecutive doses of the dual-MEK inhibitor is about seven to eight times a plasma half-life of the dual-MEK inhibitor.

A-77. The method of embodiment A-76, wherein the plasma half-life of the dual-MEK inhibitor in humans is 1-3 hours.

A-78. The method of embodiment A-77, wherein the plasma half-life of the dual-MEK inhibitor in humans is about 2 hours.

A-79. The method of any one of embodiments A-76 to A-78, wherein the dual-MEK inhibitor has a Cmin of over about 0.1 micromolar occurs 6-8 hours after administering the dual-MEK inhibitor free fraction.

A-80. The method of any one of embodiments A-76 to A-79, wherein the dual-MEK inhibitor is administered for at least 15 consecutive days.

A-81. The method of any one of embodiments A-76 to A-80, wherein the dual-MEK inhibitor is administered for at least 28 consecutive days.

A-82. A method of treating cancer in a patient, the method comprising orally administering to the patient in need thereof a total dose of 240 mg of Compound A once daily to the patient in need thereof, to treat the cancer

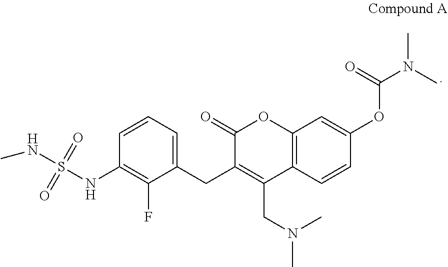

A-83. A method of treating cancer in a patient, the method comprising orally administering to the patient in need thereof a total dose of 320 mg of Compound A once daily to the patient in need thereof, to treat the cancer

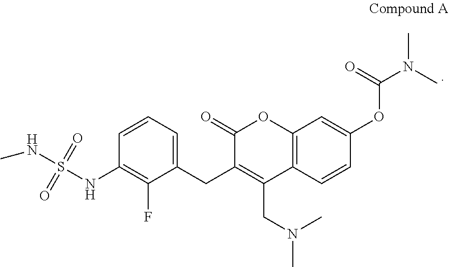

A-84. The method of any one of embodiments A-76 to A-83, wherein the cancer has a MAPK mutation selected from the group consisting of: KRAS-G12S, BRAF-V600E, NRAS-Q61R, KRAS-G12C, NRAS-Q61K, or KRAS-G12D.

A-85. The method of embodiment A-84, wherein the cancer is human lung adenocarcinoma.

A-86. The method of embodiment A-84, wherein the cancer is human malignant melanoma.

A-87. The method of embodiment A-84, wherein the cancer is human NSCLC.

A-88. The method of embodiment A-84, wherein the cancer is human colon carcinoma.

A-89. The method of any one of embodiments A-76 to A-83, wherein the cancer has a KRAS, BRAF, NRAS, or MAPK mutation.

A-90. The method of embodiment A-89, wherein the cancer has a KRAS-G12S mutation.

A-91. The method of embodiment A-89, wherein the cancer has a BRAF-V600E mutation.

A-92. The method of embodiment A-89, wherein the cancer has a NRAS-Q61R mutation.

A-93. The method of embodiment A-89, wherein the cancer has a KRAS-G12C mutation.

A-94. The method of embodiment A-89, wherein the cancer has a NRAS-Q61K mutation.

A-95. The method of embodiment A-89, wherein the cancer has a KRAS-G12D mutation.

A-96. The method of embodiment A-17, wherein the patient is diagnosed with pancreatic cancer with a KRAS G12V mutation.

A-97. The method of embodiment A-17, wherein the patient is diagnosed with colorectal cancer with a KRAS G12S mutation.

A-98. The method of embodiment A-88, wherein the cancer has a V600E mutation.

A-99. A method of treating a patient with a cancer with a BRAF mutation, the method consisting of orally administering to the patient in need thereof a therapeutically effective amount of Compound A,

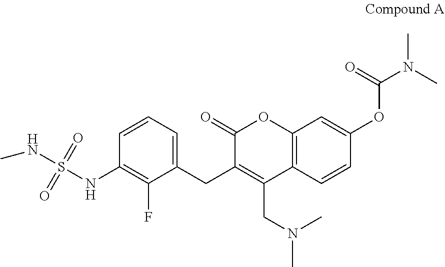

or a pharmaceutically acceptable salt thereof, in combination with a therapeutically effective amount of encorafenib.

A-100. The method of embodiment A-99, wherein the cancer is colorectal cancer (CRC).

A-101. The method of embodiment A-100, wherein the CRC is metastatic CRC.

A-102. The method of any one of embodiments A-99 to A-101, wherein the cancer is a cancer with a BRAF V600E mutation.

A-103. The method of any one of embodiments A-99 to A-102, wherein the cancer is a cancer with a BRAF V600E mutation.

A-104. The method of embodiment A-103, wherein the cancer is not wild-type BRAF CRC.

A-105. The method of any one of embodiments A-103 to A-104, wherein the therapeutically effective amount of encorafenib is 300 mg orally once daily.

A-106. The method of embodiment A-105, wherein the method further comprises the administration of cetuximab in combination with the encorafenib.

A-107. The method of embodiment A-99, wherein the cancer is melanoma.

A-108. The method of embodiment A-107, wherein the melanoma is unresectable or metastatic melanoma.

A-109. The method of any one of embodiments A-107 to A-108, wherein the cancer is a cancer with a BRAF V600E or BRAF V600K mutation.

A-110. The method of any one of embodiments A-107 to A-109, wherein the cancer is a melanoma cancer with a BRAF V600E mutation.

A-111. The method of embodiment A-110, wherein the cancer is not wild-type BRAF melanoma.

A-112. The method of any one of embodiments A-107 to A-111, wherein the therapeutically effective amount of encorafenib is 450 mg orally once daily.

A-113. The method of embodiment A-112, wherein the method further comprises the administration of cetuximab in combination with the binimetinib.

A-114. The method of any one of embodiments A-99 to A-113, wherein the encorafenib is administered with or without food.

A-115. The method of any one of embodiments A-99 to A-115, wherein the encorafenib is administered as capsules each having a total of 75 mg of encorafenib.

A-116. A method of treating a patient with pancreatic cancer with a RAS mutation, the method consisting of orally administering to the patient in need thereof a therapeutically effective amount of Compound A,

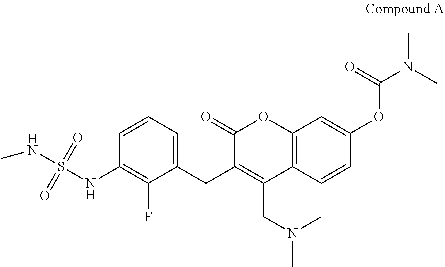

or a pharmaceutically acceptable salt thereof, in combination with a therapeutically effective amount of a nucleoside metabolic inhibitor.

A-117. The method of embodiment A-116, wherein the nucleoside metabolic inhibitor is gemcitabine administered as follows: 1000 mg/m² over 30 minutes once weekly for the first 7 weeks, then one week rest, then once weekly for 3 weeks on a 28-day cycle.

A-118. The method of embodiment A-117, wherein the gemcitabine and Compound A are the only drugs administered for the treatment of the pancreatic cancer.

A-119. The method of any one of embodiments A-116 to A-118, wherein the pancreatic cancer is pancreatic cancer with a KRAS mutation.

A-120. The method of embodiment A-119, wherein the pancreatic cancer is pancreatic cancer with a KRAS G12 mutation.

A-121. The method of embodiment A-119, wherein the pancreatic cancer is pancreatic cancer with a KRAS G12V mutation.

A-122. The method of embodiment A-119, wherein the pancreatic cancer is pancreatic cancer with a KRAS G12D mutation.

A-123. The method of any one of embodiments A-116 to A-122, wherein the pancreatic cancer is adenocarcinoma of the pancreas.

A-124. A method of treating a patient with pancreatic cancer with a RAS mutation, the method consisting of orally administering to the patient in need thereof a therapeutically effective amount of Compound A,

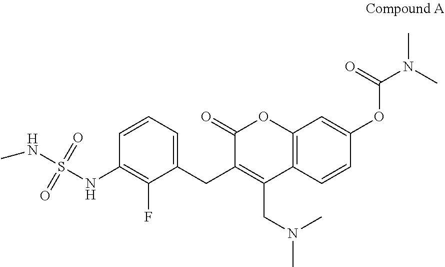

or a pharmaceutically acceptable salt thereof, in combination with a therapeutically effective amount of a microtubule inhibitor.

A-125. The method of embodiment A-124, wherein the microtubule inhibitor is administered in combination with a nucleoside metabolic inhibitor.

A-126. The method of embodiment A-125, wherein the microtubule inhibitor is paclitaxel.

A-127. The method of any one of embodiments A-124 to A-125, wherein the nucleoside metabolic inhibitor is gemcitabine.

A-128. The method of any one of embodiments A-124 to A-127, wherein the microtubule inhibitor is paclitaxel administered as follows: 25 mg/m² intravenously over 30-40 minutes on Days 1, 8, and 15 of each 28-day cycle; administer gemcitabine on Days 1, 8, and 15 of each 28-day cycle immediately after gemcitabine administration.

A-129. The method of any one of embodiments A-125 to A-128, wherein the microtubule inhibitor, the nucleoside metabolic inhibitor and Compound A are the only drugs administered for the treatment of the pancreatic cancer.

A-130. The method of any one of embodiments A-124 to A-129, wherein the pancreatic cancer is pancreatic cancer with a KRAS mutation.

A-131. The method of embodiment A-130, wherein the pancreatic cancer is pancreatic cancer with a KRAS G12 mutation.

A-132. The method of embodiment A-130, wherein the pancreatic cancer is pancreatic cancer with a KRAS G12V mutation.

A-133. The method of embodiment A-130, wherein the pancreatic cancer is pancreatic cancer with a KRAS G12D mutation.

A-134. The method of any one of embodiments A-124 to A-133, wherein the pancreatic cancer is adenocarcinoma of the pancreas.

In some embodiments, one or more of following methods are also provided.

B-98. A method of treating a patient diagnosed with a cancer with a RAS mutation, the method comprising orally administering to the patient in need thereof a therapeutically effective amount of Compound A,

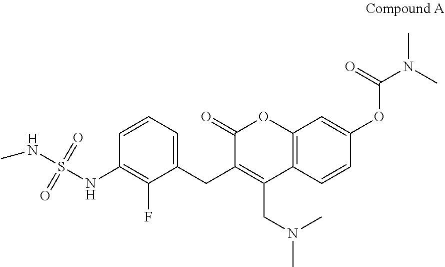

or a pharmaceutically acceptable salt thereof, once daily;
wherein the patient is diagnosed with a cancer selected from the group consisting of pancreatic cancer (PANC), colorectal cancer (CRC), lung adenocarcinoma (LUAD), melanoma (MEL), uterine serous carcinoma (USC), ampullary adenocarcinoma (AmpCA), mesonephric adenocarcinoma (MNAC), cholangiocarcinoma (CHOL), or lung squamous cell carcinoma (LUSC).

B-99. The method of embodiment B-98, wherein the patient is diagnosed with a cancer having a RAS mutation selected from a KRAS mutation or a NRAS mutation.

B-100. The method of embodiment B-99, wherein the patient is diagnosed with a KRAS mutation.

B-101. The method of embodiment B-99, wherein the patient is diagnosed with a KRAS G12 mutation.

B-102. The method of embodiment B-101, wherein the patient is diagnosed with a KRAS G12 mutation selected from the group consisting of: G12V, G12D, G12R, G12A, G12S.

B-103. The method of embodiment B-99, wherein the patient is diagnosed with a KRAS Q61 mutation.

B-104. The method of embodiment B-103, wherein the patient is diagnosed with a KRAS Q61 mutation selected from the group consisting of: Q61H, Q61L and Q61R.

B-105. The method of embodiment B-99, wherein the patient is diagnosed with a NRAS mutation.

B-106. The method of embodiment B-105, wherein the patient is diagnosed with a NRAS mutation.

B-107. The method of embodiment B-105, wherein the patient is diagnosed with a NRAS Q61 mutation.

B-108. The method of embodiment B-105, wherein the patient is diagnosed with a NRAS Q61L or Q61K mutation.

B-109. The method of embodiment B-105, wherein the patient is diagnosed with a NRAS G12 mutation.

B-110. The method of embodiment B-105, wherein the patient is diagnosed with a NRAS G12S mutation.

B-111. The method of embodiment B-105, wherein the patient is diagnosed with a NRAS G13 mutation.

B-112. The method of embodiment B-105, wherein the patient is diagnosed with a NRAS G13R mutation.

B-113. The method of any one of embodiments B-98 to B-112, wherein Compound A or a pharmaceutically acceptable salt thereof is orally administered to the patient in need thereof in an amount providing a total dose of 320 mg of Compound A once per day (QD).

B-114. The method of any one of embodiments B-98 to B-112, wherein Compound A or a pharmaceutically acceptable salt thereof is orally administered to the patient in need thereof in an amount providing a total dose of 320 mg of Compound A once per day (QD) as a monotherapy to treat the cancer.

B-115. The method of any one of embodiments B-98 to B-112, wherein Compound A or a pharmaceutically acceptable salt thereof is orally administered to the patient in need thereof in an amount providing a total dose of 240 mg of Compound A once per day (QD).

B-116. A method of treating a patient diagnosed with a cancer with a KRAS or NRAS mutation, the method comprising orally administering Compound A to the patient in need thereof,

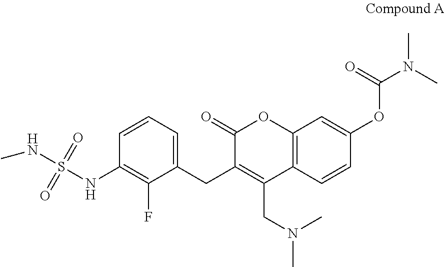

or a pharmaceutically acceptable salt thereof, once per day (QD) in an amount providing 240 mg or 320 mg of Compound A once per day;
wherein the patient is diagnosed with a cancer selected from the group consisting of pancreatic cancer (PANC), colorectal cancer (CRC), lung adenocarcinoma (LUAD), melanoma (MEL), uterine serous carcinoma (USC), ampullary adenocarcinoma (AmpCA), mesonephric adenocarcinoma (MNAC), cholangiocarcinoma (CHOL), or lung squamous cell carcinoma (LUSC); or wherein the patient is diagnosed with a cancer having a RAS mutation selected from KRAS G12V, KRAS G12D, KRAS G12R, KRAS G12A, KRAS G12S, KRAS Q61H, KRAS Q61L, KRAS Q61R, NRAS G12S, NRAS G13R, NRAS GQ61K, and NRAS Q61L.

B-117. The method of any one of embodiments B-98 to B-112, wherein the patient is diagnosed with a cancer having a RAS mutation selected from KRAS G12V, KRAS G12D, KRAS G12R, KRAS G12A, KRAS G12S, KRAS Q61H, KRAS Q61L, KRAS Q61R, NRAS G12S, NRAS G13R, NRAS GQ61K, and NRAS Q61L.

B-118. The method of any one of embodiments B-116 to B-117, wherein Compound A or a pharmaceutically acceptable salt thereof is orally administered to the patient in need thereof in an amount providing a total dose of 320 mg of Compound A once per day (QD).

B-119. The method of any one of embodiments B-116 to B-117, wherein Compound A or a pharmaceutically acceptable salt thereof is orally administered to the patient in need thereof in an amount providing a total dose of 240 mg of Compound A once per day (QD).

B-120. The method of any one of embodiments B-116 to B-119, wherein the patient is diagnosed with pancreatic cancer (PANC).

B-121. The method of any one of embodiments B-116 to B-119, wherein the patient is diagnosed with melanoma (MEL).

B-122. The method of any one of embodiments B-116 to B-119, wherein the patient is diagnosed with lung cancer.

B-123. The method of any one of embodiments B-116 to B-119, wherein the patient is diagnosed with lung squamous cell carcinoma (LUSC).

B-124. The method of any one of embodiments B-116 to B-119, wherein the patient is diagnosed with lung adenocarcinoma (LUAD).

B-125. The method of any one of embodiments B-116 to B-119, wherein the patient is diagnosed with non-small cell lung cancer (NSCLC).

In some embodiments, one or more of following methods are also provided.

B-126. A method of treating a patient diagnosed with pancreatic cancer comprising orally administering to the patient in need thereof a therapeutically effective amount of Compound A,

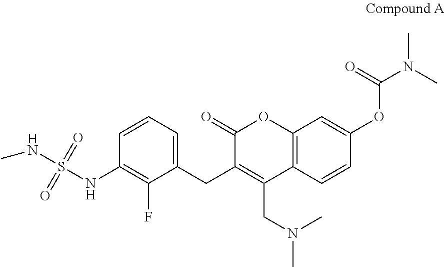

or a pharmaceutically acceptable salt thereof.

B-127. The method of embodiment B-135, wherein the pancreatic cancer is adenocarcinoma of the pancreas.

B-128. The method of any one of embodiments B-135 to B-136, wherein the therapeutically effective amount of Compound A is an amount of Compound A or a pharmaceutically acceptable salt thereof that provides a total dose of 240 mg or 320 mg of Compound A once daily (QD) to the patient in need thereof.

B-129. The method of any one of embodiments B-135 to B-136, wherein the therapeutically effective amount of Compound A is an amount of Compound A or a pharmaceutically acceptable salt thereof that provides a total dose of 240 mg of Compound A once daily (QD) to the patient in need thereof.

B-130. The method of any one of embodiments B-135 to B-136, wherein the therapeutically effective amount of Compound A is an amount of Compound A or a pharmaceutically acceptable salt thereof that provides a total dose of 320 mg of Compound A once daily (QD) to the patient in need thereof.

B-131. The method of any one of embodiments B-135 to B-139, wherein the method further comprises administering gemcitabine to the patient.

B-132. The method of embodiment 140, wherein the method further comprises administering nab-paclitaxel to the patient.

B-133. The method of any one of embodiments B-135 to B-139, wherein the method further comprises administering a FOLFIRINOX regimen to the patient.

B-134. The method of any one of embodiments B-135 to B-139, wherein the method further comprises administering a modified FOLFIRINOX regimen to the patient.

B-135. The method of any one of embodiments B-135 to B-139, wherein the method further comprises administering oxaliplatin, irinotecan and fluorouracil to the patient.

B-136. The method of embodiment B-144, wherein the oxaliplatin, irinotecan and fluorouracil are administered once every 14 days to the patient, while Compound A is administered once daily to the patient.

In some embodiments, one or more of following methods are also provided.

A method of treating a patient diagnosed with a cancer, the method comprising orally administering Compound A to the patient in need thereof,

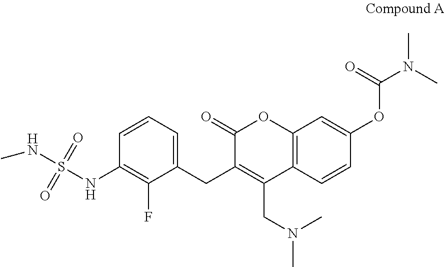

or a pharmaceutically acceptable salt thereof, once per day (QD) in an amount providing 240 mg or 320 mg of Compound A once per day;
wherein the patient is diagnosed with a cancer having a BRAF mutation.

A method of treating a patient diagnosed with a cancer, the method comprising orally administering Compound A to the patient in need thereof,

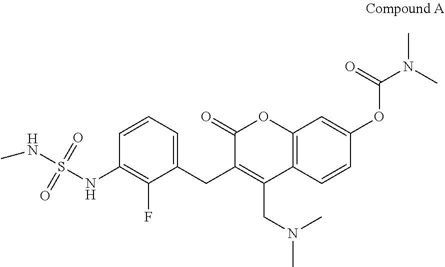

or a pharmaceutically acceptable salt thereof, once per day (QD) in an amount providing 240 mg or 320 mg of Compound A once per day;
wherein the patient is diagnosed with a cancer having a BRAF mutation. selected from the group consisting of:

| | |
|---|---|
| V600E/V600E(2) | V471F |
| V600K | K601Q |
| V600R | Q257R |
| V600L | T599R |
| V600D | V487_P492delinsA |
| CLASS 2 TOTAL | L597S |
| G469A | G464A |
| K601E | K499E |
| G469V | L505H |
| G469R | K601T |
| G464V | V600_K601delinsEN |
| L597R | V600_S605delinsEISRWR |
| K601N | L505F |
| E586K | L485_P490delinsY |
| T599dup | I463S |
| L597Q | CLASS 3 TOTAL |
| V600_K601delinsE | D594G |
| L485F | D594N |
| N486_P490del | G466V |
| G464R | N581S |
| G466E | D594A |
| G596R | D594E |
| N581I | D594H |
| G466R | F595L |
| G466A | D287H |
| S467L | D594V |
| G469E | G596C |
| N581Y | T599A |
| K483E | N581K |

A method of treating a patient diagnosed with a cancer, the method comprising orally administering Compound A to the patient in need thereof,

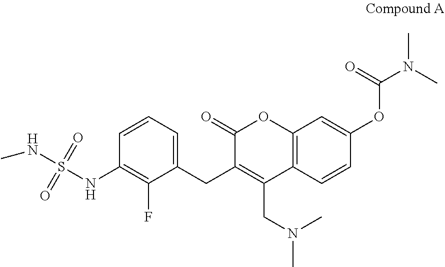

or a pharmaceutically acceptable salt thereof, once per day (QD) in an amount providing 240 mg or 320 mg of Compound A once per day;
wherein the patient is diagnosed with a cancer having a Class I BRAF mutation.

A method of treating a patient diagnosed with a cancer, the method comprising orally administering Compound A to the patient in need thereof,

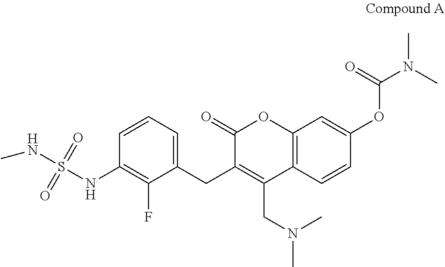

or a pharmaceutically acceptable salt thereof, once per day (QD) in an amount providing 240 mg or 320 mg of Compound A once per day;
wherein the patient is diagnosed with a cancer having a Class I BRAF mutation in the activation loop.

A method of treating a patient diagnosed with a cancer, the method comprising orally administering Compound A to the patient in need thereof,

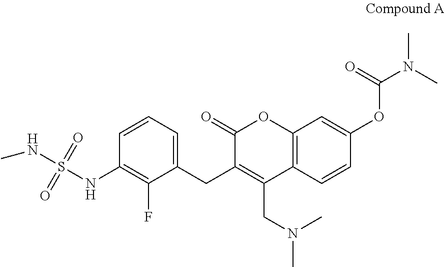

or a pharmaceutically acceptable salt thereof, once per day (QD) in an amount providing 240 mg or 320 mg of Compound A once per day;
wherein the patient is diagnosed with a cancer having a BRAF mutation selected from the group consisting of V600D, V600E, V600K, V600L, V600M, and V600R.

A method of treating a patient diagnosed with a cancer, the method comprising orally administering Compound A to the patient in need thereof,

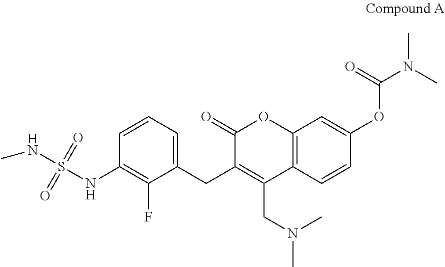

or a pharmaceutically acceptable salt thereof, once per day (QD) in an amount providing 240 mg or 320 mg of Compound A once per day;
wherein the patient is diagnosed with a cancer having a Class II BRAF mutation.

A method of treating a patient diagnosed with a cancer, the method comprising orally administering Compound A to the patient in need thereof,

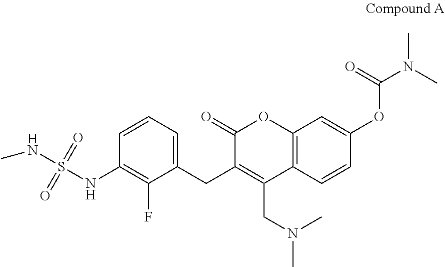

or a pharmaceutically acceptable salt thereof, once per day (QD) in an amount providing 240 mg or 320 mg of Compound A once per day;
wherein the patient is diagnosed with a cancer having a Class II BRAF mutation in the activation segment.

A method of treating a patient diagnosed with a cancer, the method comprising orally administering Compound A to the patient in need thereof,

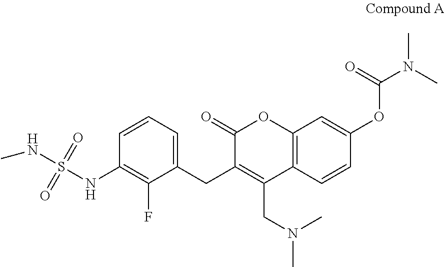

or a pharmaceutically acceptable salt thereof, once per day (QD) in an amount providing 240 mg or 320 mg of Compound A once per day;
wherein the patient is diagnosed with a cancer having a BRAF mutation selected from the group consisting of: L597Q, L597R, L597S, L597V, T599R, T599dup, K601E, K601N, K601Q, K601T.

A method of treating a patient diagnosed with a cancer, the method comprising orally administering Compound A to the patient in need thereof,

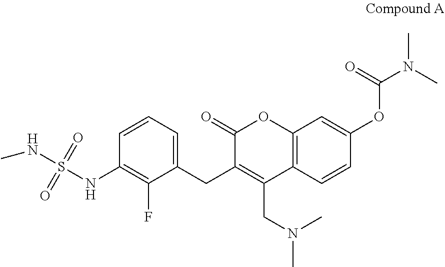

or a pharmaceutically acceptable salt thereof, once per day (QD) in an amount providing 240 mg or 320 mg of Compound A once per day;
wherein the patient is diagnosed with a cancer having a Class II BRAF mutation in the P-loop.

A method of treating a patient diagnosed with a cancer, the method comprising orally administering Compound A to the patient in need thereof,

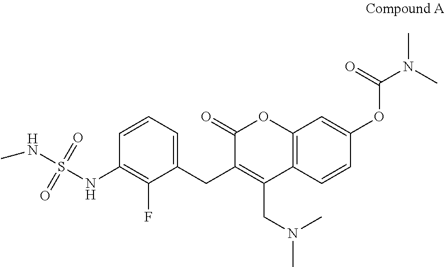

or a pharmaceutically acceptable salt thereof, once per day (QD) in an amount providing 240 mg or 320 mg of Compound A once per day;
wherein the patient is diagnosed with a cancer having a BRAF mutation selected from the group consisting of: G464A, G464E, G464R, G464V, G469A, G469R, G469V, and V471F.

A method of treating a patient diagnosed with a cancer, the method comprising orally administering Compound A to the patient in need thereof,

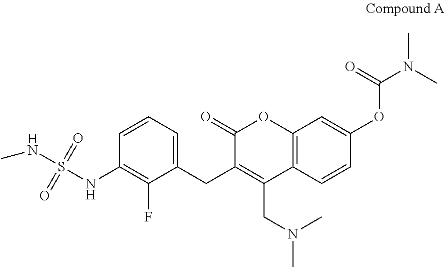

or a pharmaceutically acceptable salt thereof, once per day (QD) in an amount providing 240 mg or 320 mg of Compound A once per day;
wherein the patient is diagnosed with a cancer having a BRAF mutation selected from the group consisting of: Q257R, I463S, L485F, L485_P490delinsY, N486_P490del, V487_P492delinsA, K499E, L505F, L505H, E586K, V600_K601delinsE, V600_K601delinsEN, and V600_S605delinsEISRWR.

A method of treating a patient diagnosed with a cancer, the method comprising orally administering Compound A to the patient in need thereof,

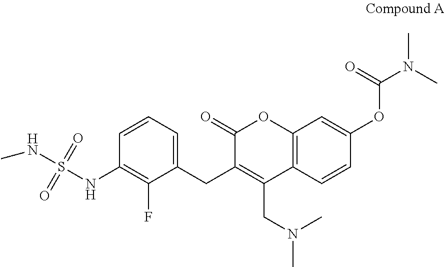

or a pharmaceutically acceptable salt thereof, once per day (QD) in an amount providing 240 mg or 320 mg of Compound A once per day;
wherein the patient is diagnosed with a cancer having a Class III BRAF mutation.

A method of treating a patient diagnosed with a cancer, the method comprising orally administering Compound A to the patient in need thereof,

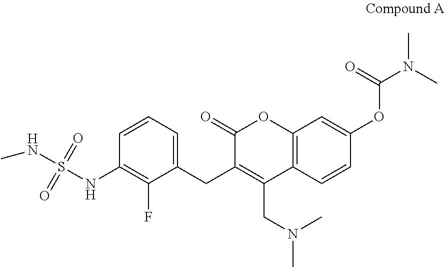

or a pharmaceutically acceptable salt thereof, once per day (QD) in an amount providing 240 mg or 320 mg of Compound A once per day;
wherein the patient is diagnosed with a cancer having a Class III activation segment BRAF mutation.

A method of treating a patient diagnosed with a cancer, the method comprising orally administering Compound A to the patient in need thereof,

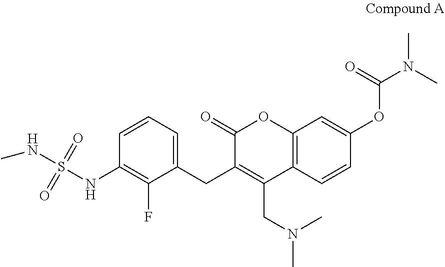

or a pharmaceutically acceptable salt thereof, once per day (QD) in an amount providing 240 mg or 320 mg of Compound A once per day;
wherein the patient is diagnosed with a cancer having a BRAF mutation selected from the group consisting of: T599A.

A method of treating a patient diagnosed with a cancer, the method comprising orally administering Compound A to the patient in need thereof,

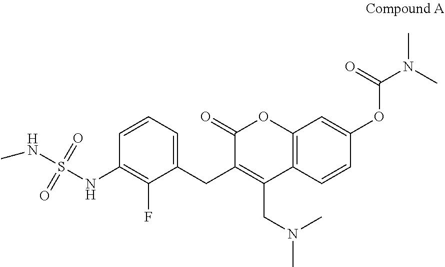

or a pharmaceutically acceptable salt thereof, once per day (QD) in an amount providing 240 mg or 320 mg of Compound A once per day;
wherein the patient is diagnosed with a cancer having a Class III P-loop BRAF mutation.

A method of treating a patient diagnosed with a cancer, the method comprising orally administering Compound A to the patient in need thereof,

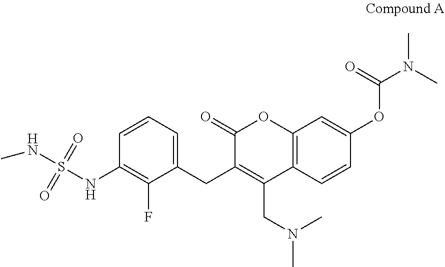

or a pharmaceutically acceptable salt thereof, once per day (QD) in an amount providing 240 mg or 320 mg of Compound A once per day;
wherein the patient is diagnosed with a cancer having a BRAF mutation selected from the group consisting of: G466A, G466E, G466R, G466V, S467L, and G469E.

A method of treating a patient diagnosed with a cancer, the method comprising orally administering Compound A to the patient in need thereof,

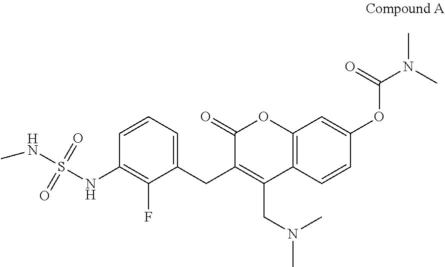

or a pharmaceutically acceptable salt thereof, once per day (QD) in an amount providing 240 mg or 320 mg of Compound A once per day;
wherein the patient is diagnosed with a cancer having a Class III DGF motif BRAF mutation.

A method of treating a patient diagnosed with a cancer, the method comprising orally administering Compound A to the patient in need thereof,

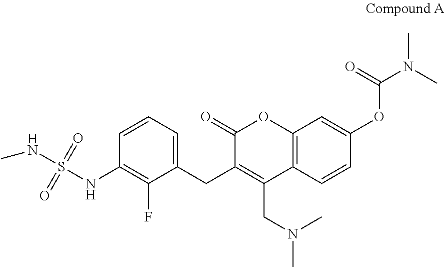

or a pharmaceutically acceptable salt thereof, once per day (QD) in an amount providing 240 mg or 320 mg of Compound A once per day;
wherein the patient is diagnosed with a cancer having a BRAF mutation selected from the group consisting of: D594A, D594E, D594G, D594H, D594N, D594V and F595L.

A method of treating a patient diagnosed with a cancer, the method comprising orally administering Compound A to the patient in need thereof,

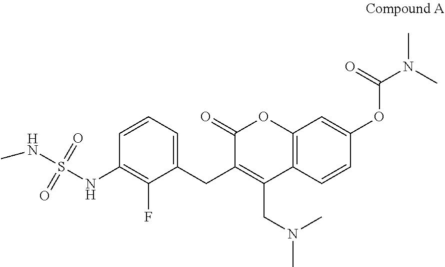

or a pharmaceutically acceptable salt thereof, once per day (QD) in an amount providing 240 mg or 320 mg of Compound A once per day;
wherein the patient is diagnosed with a cancer having a Class III catalytic loop BRAF mutation.

A method of treating a patient diagnosed with a cancer, the method comprising orally administering Compound A to the patient in need thereof,

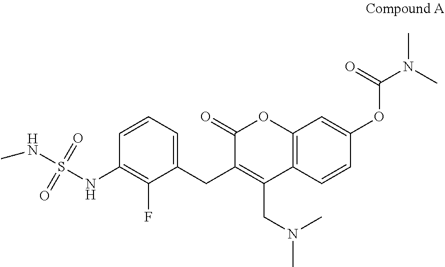

or a pharmaceutically acceptable salt thereof, once per day (QD) in an amount providing 240 mg or 320 mg of Compound A once per day;
wherein the patient is diagnosed with a cancer having a BRAF mutation selected from the group consisting of: N581I, N581K, N581S and N581Y.

A method of treating a patient diagnosed with a cancer, the method comprising orally administering Compound A to the patient in need thereof,

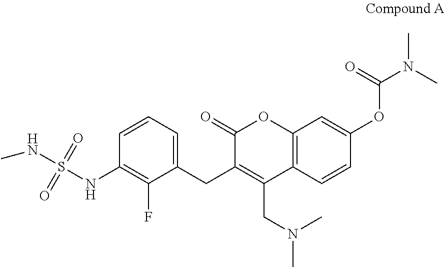

or a pharmaceutically acceptable salt thereof, once per day (QD) in an amount providing 240 mg or 320 mg of Compound A once per day;
wherein the patient is diagnosed with a cancer having a BRAF mutation selected from the group consisting of: D287H, V459L and K483E.

A method of treating a patient diagnosed with a cancer, the method comprising orally administering Compound A to the patient in need thereof,

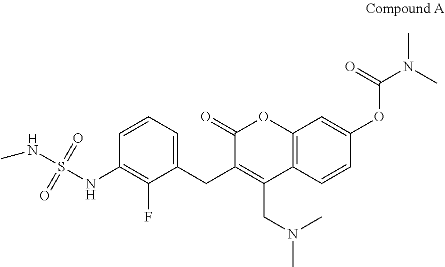

or a pharmaceutically acceptable salt thereof, once per day (QD) in an amount providing 240 mg or 320 mg of Compound A once per day;
wherein the patient is diagnosed with a cancer selected from the group consisting of:

| | |
|---|---|
| Melanoma | Bladder cancer |
| Thyroid carcinoma | Ovarian surface epithelial carcinomas |
| Multiple myeloma | Prostatic adenocarcinoma |
| Small intestinal malignancies | Uveal melanoma |
| Colorectal adenocarcinoma | Non-melanoma, non-Merkel skin cancers |
| Cancer of unknown primary | Small cell lung cancer |
| Non-small cell lung cancer | Uterine cancers |
| Cholangiocarcinoma | Cervical cancer |
| Low grade glioma | Soft tissue tumors |
| Others | Esophageal and esophagogastric junction |
| High grade glioma | Breast carcinoma |
| Others | Kidney cancer |
| Lymphoma | Head and neck cancers |
| Gastric adenocarcinoma | |
| Neuroendocrine tumors | |
| Pancreatic adenocarcinoma | |

In some embodiments, one or more of following methods are also provided.

C-1. A method of treating a patient with a RAS-mutated cancer, comprising administering a therapeutically effective amount of a dual-MEK inhibitor inhibiting both phosphorylation of mitogen-activated protein kinase kinase 1 and 2 (pMEK) and phosphorylation of mitogen-activated protein kinase 1 and 2 (pERK), wherein the dual-MEK inhibitor is administered to the patient (a) in a dose amount that achieves a Cmax effective to inhibit pERK and pMEK and (b) with an interval between consecutive doses that is at least about seven times the plasma half-life of the dual-MEK inhibitor.

C-2. The method of embodiment C-1, wherein the Cmax measured on day 1 of administering the dual-MEK inhibitor is at least 2,000 ng/mL.

C-3. The method of embodiment C-2, wherein the dual-MEK inhibitor has at least about 1 micromolar dual-MEK inhibitor free fraction.

C-4. The method of any one of embodiments C-1 to C-3, wherein a Cmin of over about 0.1 micromolar occurs longer than about 6 hours after administering of the dual-MEK inhibitor in a free fraction form.

C-5. The method of any one of embodiments C-1 to C-3, wherein a Cmin of over about 0.1 micromolar occurs longer than about 6-8 hours after administering of the dual-MEK inhibitor in a free fraction form.

C-6. The method of any one of embodiments C-1 to C-5, wherein the interval between consecutive doses of the dual-MEK inhibitor is about seven to eight times the plasma half-life of the dual-MEK inhibitor.

C-7. The method of any one of embodiments C-1 to C-6, wherein the dual-MEK inhibitor is Compound A

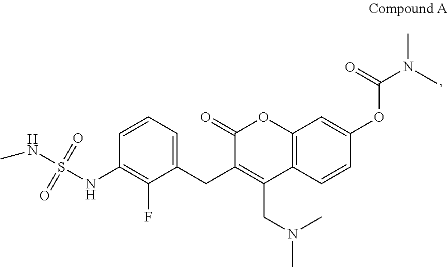

or a pharmaceutically acceptable salt thereof.

C-8. The method of any one of embodiments C-1 to C-7, wherein the RAS-mutated cancer is selected from the group consisting of: RAS-mutated melanoma, RAS-mutated PDAC, RAS-mutated NSCLC and RAS-mutated CRC, where RAS is any activation mutation in KRAS, NRAS or HRAS.

C-9. The method of any one of embodiments C-1 to C-7, wherein the RAS-mutated cancer is a KRAS G12D or G12V mutation or a NRAS Q61L or Q61K mutation.

C-10. The method of any one of embodiments C-1 to C-7, wherein the RAS-mutated cancer is pancreatic cancer with a KRAS G12C, G12D or G12V mutation.

C-11. The method of any one of embodiments C-1 to C-7, wherein the RAS-mutated cancer is colorectal cancer with a NRAS Q61L or Q61K mutation or colorectal cancer with a KRAS G12D mutation.

C-12. The method of any one of embodiments C-1 to C-7, wherein the RAS-mutated cancer is a RAS-mutated melanoma with a NRAS Q61R mutation, or a RAS-mutated melanoma with a BRAF V600E mutation.

C-13. The method of any one of embodiments C-1 to C-7, wherein the RAS-mutated cancer is non-small cell lung cancer (NSCLC) with a KRAS G12S mutation.

C-14. The method of any one of embodiments C-1 to C-13, wherein the dual-MEK inhibitor is Compound A administered to the patient once daily in a dose amount of about 240 mg or about 320 mg.

C-15. The method of embodiment C-14, wherein the dual-MEK inhibitor is Compound A administered to the patient once daily in a dose amount of about 240 mg.

C-16. The method of embodiment C-14, wherein the dual-MEK inhibitor is Compound A administered to the patient once daily in a dose amount of about 320 mg.

C-17. The method of any one of embodiments C-14 to C-16, wherein Compound A is administered orally to the patient in need thereof, for at least 28 consecutive days.

C-18. The method of embodiment C-17, wherein the patient is diagnosed with pancreatic cancer with a KRAS G12D mutation.

C-19. The method of embodiment C-17, wherein the patient is diagnosed with colorectal cancer with a KRAS G12V mutation.

C-20. The method of embodiment C-17, wherein the patient is diagnosed with colorectal cancer with a NRAS Q61L mutation.

C-21. The method of embodiment C-17, wherein the patient is diagnosed with colorectal cancer with a NRAS Q61K mutation.

C-22. A method of treating a patient with a cancer with a KRAS G12D or G12V mutation, the method consisting of orally administering to the patient in need thereof a therapeutically effective amount of Compound A,

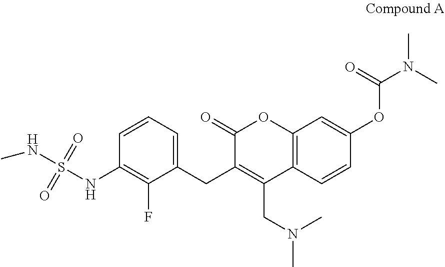

or a pharmaceutically acceptable salt thereof, once daily as a monotherapy.

C-23. The method of embodiment C-22, wherein the patient is diagnosed with pancreatic cancer with a KRAS G12D or G12V mutation.

C-24. The method of embodiment C-22, wherein the patient is diagnosed with colorectal cancer with a KRAS G12D or G12V mutation.

C-25. The method of any one of embodiments C-22 to C-24, wherein the therapeutically effective amount is a dose of 160 mg of Compound A orally once daily as a monotherapy.

C-26. The method of any one of embodiments C-22 to C-24, wherein the therapeutically effective amount is a dose of 240 mg of Compound A orally once daily as a monotherapy.

C-27. The method of any one of embodiments C-22 to C-24, wherein the therapeutically effective amount is a dose of 320 mg of Compound A orally once daily as a monotherapy.

C-28. A method of treating a patient with a cancer with a KRAS G12D or G12V mutation or a cancer with a NRAS Q61L or Q61K mutation, the method consisting of orally administering to the patient in need thereof a total dose of 160 mg of Compound A once daily to treat the cancer,

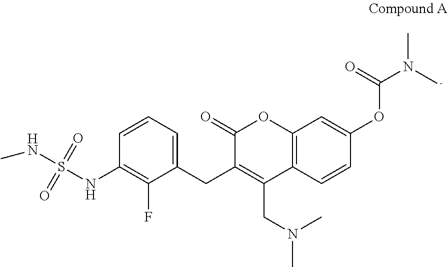

C-29. A method of treating a patient with a cancer with a KRAS G12D or G12V mutation or a cancer with a NRAS Q61L or Q61K mutation, the method consisting of orally administering to the patient in need thereof a total dose of 240 mg of Compound A once daily to treat the cancer,

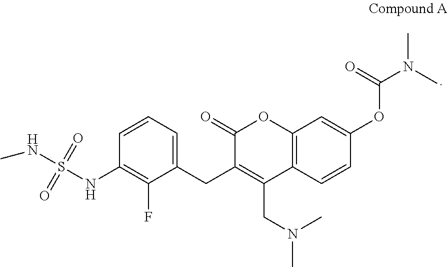

C-30. A method of treating a patient with a cancer with a KRAS G12D or G12V mutation or a cancer with a NRAS Q61L or Q61K mutation, the method consisting of orally administering to the patient in need thereof a total dose of 320 mg of Compound A once daily to treat the cancer,

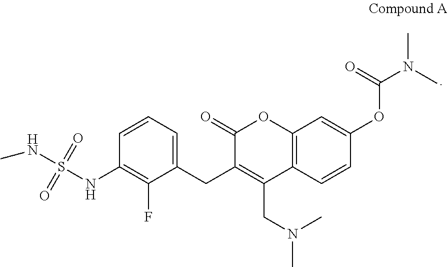

C-31. The method of any one of embodiments C-28 to C-30, wherein Compound A is administered as a monotherapy.

C-32. The method of embodiment C-31, wherein the cancer has a KRAS G12D mutation.

C-33. The method of embodiment C-31, wherein the cancer is pancreatic cancer with a KRAS G12D mutation.

C-34. The method of embodiment C-31, wherein the cancer has a KRAS G12V mutation.

C-35. The method of embodiment C-31, wherein the cancer is colorectal cancer with a KRAS G12V mutation.

C-36. The method of embodiment C-31, wherein the cancer with a NRAS Q61L mutation.

C-37. The method of embodiment C-31, wherein the cancer is colorectal cancer with a NRAS Q61L mutation.

C-38. The method of embodiment C-31, wherein the cancer has a NRAS Q61K mutation.

C-39. The method of embodiment C-31, wherein the cancer is colorectal cancer with a NRAS Q61K mutation.

C-40. A method of treating a patient with a cancer with a mutation in KRAS, NRAS, or HRAS, the method consisting of orally administering to the patient in need thereof a total dose of 160 mg of Compound A once daily to treat the cancer,

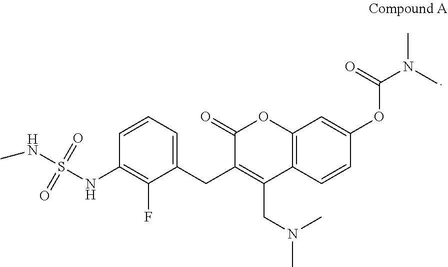

C-41. A method of treating a patient with a cancer with a mutation in KRAS, NRAS, or HRAS, the method consisting of orally administering to the patient in need thereof a total dose of 240 mg of Compound A once daily to treat the cancer,

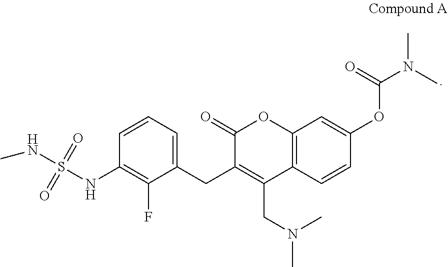

C-42. A method of treating a patient with a cancer with a mutation in KRAS, NRAS, or HRAS, the method consisting of orally administering to the patient in need thereof a total dose of 320 mg of Compound A once daily to treat the cancer,

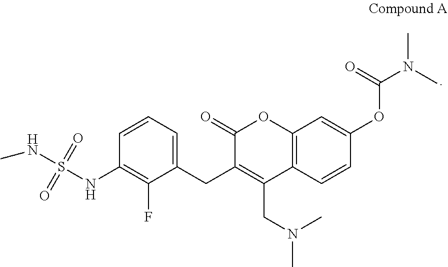

C-43. The method of any one of embodiments C-40 to C-42, wherein the cancer has a KRAS mutation.

C-44. The method of any one of embodiments C-40 to C-42, wherein the cancer has a KRAS G12C, G12D or G12S mutation.

C-45. The method of any one of embodiments C-43 to C-44, wherein the cancer is pancreatic cancer.

C-46. The method of any one of embodiments C-40 to C-42, wherein the cancer has a NRAS mutation.

C-47. The method of any one of embodiments C-40 to C-42, wherein the cancer has a NRAS Q61L or Q61K mutation.

C-48. The method of any one of embodiments C-46 to C-47, wherein the cancer is colorectal cancer.

C-49. The method of any one of embodiments C-40 to C-42, wherein the cancer has a HRAS mutation.

C-50. The method of any one of embodiments C-40 to C-49, wherein Compound A is administered as a monotherapy dual-MEK inhibitor.

C-51. A method of treating a patient with a RAS-mutated cancer, comprising administering once-daily orally a therapeutically effective amount of a dual-MEK inhibitor having at least about 1 micromolar dual-MEK inhibitor in free fraction, wherein the dual-MEK inhibitor inhibits both the phosphorylation of mitogen-activated protein kinase kinase 1 and 2 (pMEK) and the phosphorylation of mitogen-activated protein kinase 1 and 2 (pERK), wherein the dual-MEK inhibitor is administered to the patient (a) in a dose amount that achieves a Cmax, wherein, the Cmax is measured on day one of administering the dual-MEK inhibitor, wherein the Cmax at least 2,000 ng/mL and (b) with an interval between consecutive doses of the dual-MEK inhibitor is about seven to eight times a plasma half-life of the dual-MEK inhibitor.

C-52. The method of embodiment C-51, wherein the plasma half-life of the dual-MEK inhibitor in humans is 1-3 hours.

C-53. The method of embodiment C-51, wherein the plasma half-life of the dual-MEK inhibitor in humans is about 2 hours.

C-54. The method of any one of embodiments C-51 to C-53, wherein the dual-MEK inhibitor has a Cmin of over about 0.1 micromolar occurs 6-8 hours after administering the dual-MEK inhibitor free fraction.

C-55. The method of any one of embodiments C-1 to C-6 or C-51 to C-54, wherein the dual-MEK inhibitor is administered for at least 15 consecutive days.

C-56. The method of any one of embodiments C-1 to C-6 or C-51 to C-55, wherein the dual-MEK inhibitor is administered for at least 28 consecutive days.

C-57. A method of treating a patient with a RAS-mutated cancer, the method comprising orally administering to the patient in need thereof a total dose of 240 mg of Compound A once daily to the patient in need thereof, to treat the cancer

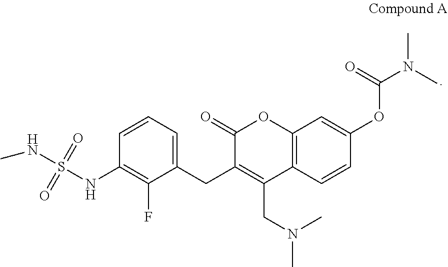

C-58. A method of treating a patient with a RAS-mutated cancer, the method comprising orally administering to the patient in need thereof a total dose of 320 mg of Compound A once daily to the patient in need thereof, to treat the cancer

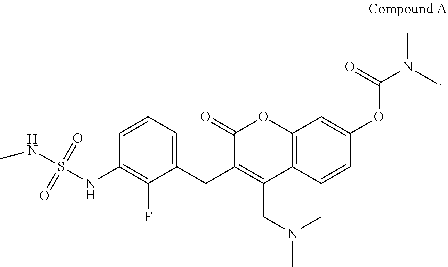

C-59. The method of any one of embodiments C-7 to C-50 or C-57 to C-58, wherein the dual-MEK inhibitor is administered for at least 15 consecutive days.

C-60. The method of any one of embodiments C-7 to C-50 or C-57 to C-58, wherein the dual-MEK inhibitor is administered for at least 28 consecutive days.

C-61. The method of any one of embodiments C-1 to C-7, or C-51 to C-60, wherein the RAS-mutated cancer is any RAS activated mutation in KRAS, NRAS or HRAS.

C-62. The method of embodiment C-61, wherein the RAS-mutated cancer is any RAS activated mutation in KRAS.

C-63. The method of embodiment C-62, wherein the RAS-mutated cancer has a mutation at KRAS G12C.

C-64. The method of embodiment C-62, wherein the RAS-mutated cancer has a mutation at KRAS G12D.

C-65. The method of embodiment C-62, wherein the RAS-mutated cancer has a mutation at KRAS G12V.

C-66. The method of any one of embodiments C-62 to C-65, wherein the RAS-mutated cancer is pancreatic cancer.

C-67. The method of embodiment C-61, wherein the RAS-mutated cancer has the RAS activated mutation at any activation mutation in NRAS.

C-68. The method of embodiment C-67, wherein the RAS-mutated cancer has the RAS activated mutation at NRAS Q61L.

C-69. The method of embodiment C-67, wherein the RAS-mutated cancer has the RAS activated mutation at NRAS Q61K.

C-70. The method of embodiment C-67, wherein the RAS-mutated cancer has the RAS activated mutation at NRAS Q61R.

C-71. The method of any one of embodiments C-64, C-68 or C-69, wherein the RAS-mutated cancer is colorectal cancer.

C-72. The method of embodiment C-70, wherein the RAS-mutated cancer is melanoma.

C-73. The method of embodiment C-62, wherein the RAS-mutated cancer has a mutation at KRAS G12S.

C-74. The method of embodiment C-73, wherein the RAS-mutated cancer is NSCLC.

C-75. The method of embodiment C-61, wherein the RAS-mutated cancer is any activation mutation in HRAS.

C-76. A method of treating cancer, the method comprising administering orally once-daily a therapeutically effective amount of a dual-MEK inhibitor having at least about 1 micromolar dual-MEK inhibitor in free fraction, wherein the dual-MEK inhibitor inhibits both the phosphorylation of mitogen-activated protein kinase kinase 1 and 2 (pMEK) and the phosphorylation of mitogen-activated protein kinase 1 and 2 (pERK), wherein the dual-MEK inhibitor is administered to the patient (a) in a dose amount that achieves a Cmax, wherein, the Cmax is measured on day one of administering the dual-MEK inhibitor, wherein the Cmax is at least 2,000 ng/mL and (b) with an interval between consecutive doses of the dual-MEK inhibitor is about seven to eight times a plasma half-life of the dual-MEK inhibitor.

C-77. The method of embodiment C-76, wherein the plasma half-life of the dual-MEK inhibitor in humans is 1-3 hours.

C-78. The method of embodiment C-77, wherein the plasma half-life of the dual-MEK inhibitor in humans is about 2 hours.

C-79. The method of any one of embodiments C-76 to C-78, wherein the dual-MEK inhibitor has a Cmin of over about 0.1 micromolar occurs 6-8 hours after administering the dual-MEK inhibitor free fraction.

C-80. The method of any one of embodiments C-76 to C-79, wherein the dual-MEK inhibitor is administered for at least 15 consecutive days.

C-81. The method of any one of embodiments C-76 to C-80, wherein the dual-MEK inhibitor is administered for at least 28 consecutive days.

C-82. A method of treating cancer in a patient, the method comprising orally administering to the patient in need thereof a total dose of 240 mg of Compound A once daily to the patient in need thereof, to treat the cancer

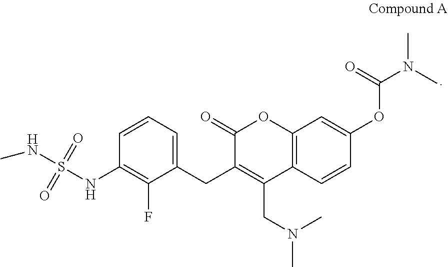

C-83. A method of treating cancer in a patient, the method comprising orally administering to the patient in need thereof a total dose of 320 mg of Compound A once daily to the patient in need thereof, to treat the cancer

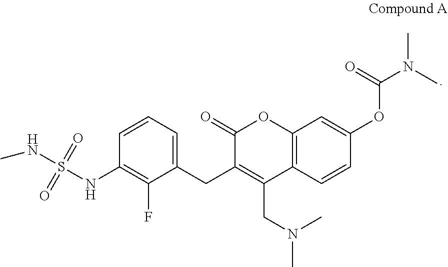

C-84. The method of any one of embodiments C-76 to C-83, wherein the cancer has a MAPK mutation selected from the group consisting of: KRAS-G12S, BRAF-V600E, NRAS-Q61R, KRAS-G12C, NRAS-Q61K, or KRAS-G12D.

C-85. The method of embodiment C-84, wherein the cancer is human lung adenocarcinoma.

C-86. The method of embodiment C-84, wherein the cancer is human malignant melanoma.

C-87. The method of embodiment C-84, wherein the cancer is human NSCLC.

C-88. The method of embodiment C-84, wherein the cancer is human colon carcinoma.

C-89. The method of any one of embodiments C-76 to C-83, wherein the cancer has a KRAS, BRAF, NRAS, or MAPK mutation.

C-90. The method of embodiment C-89, wherein the cancer has a KRAS-G12S mutation.

C-91. The method of embodiment C-89, wherein the cancer has a BRAF-V600E mutation.

C-92. The method of embodiment C-89, wherein the cancer has a NRAS-Q61R mutation.

C-93. The method of embodiment C-89, wherein the cancer has a KRAS-G12C mutation.

C-94. The method of embodiment C-89, wherein the cancer has a NRAS-Q61K mutation.

C-95. The method of embodiment C-89, wherein the cancer has a KRAS-G12D mutation.

C-96. The method of embodiment C-17, wherein the patient is diagnosed with pancreatic cancer with a KRAS G12V mutation.

C-97. The method of embodiment C-17, wherein the patient is diagnosed with colorectal cancer with a KRAS G12S mutation.

C-98. The method of embodiment C-88, wherein the cancer has a V600E mutation.

C-99. A method of treating a patient with a cancer with a BRAF mutation, the method consisting of orally administering to the patient in need thereof a therapeutically effective amount of Compound A,

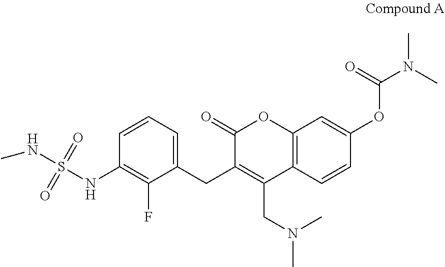

or a pharmaceutically acceptable salt thereof, in combination with a therapeutically effective amount of encorafenib.

C-100. The method of embodiment C-99, wherein the cancer is colorectal cancer (CRC).

C-101. The method of embodiment C-100, wherein the CRC is metastatic CRC.

C-102. The method of any one of embodiments C-99 to C-101, wherein the cancer is a cancer with a BRAF V600E mutation.

C-103. The method of any one of embodiments C-99 to C-102, wherein the cancer is a cancer with a BRAF V600E mutation.

C-104. The method of embodiment C-103, wherein the cancer is not wild-type BRAF CRC.

C-105. The method of any one of embodiments C-103-104, wherein the therapeutically effective amount of encorafenib is 300 mg orally once daily.

C-106. The method of embodiment C-105, wherein the method further comprises the administration of cetuximab in combination with the encorafenib.

C-107. The method of embodiment C-99, wherein the cancer is melanoma.

C-108. The method of embodiment C-107, wherein the melanoma is unresectable or metastatic melanoma.

C-109. The method of any one of embodiments C-107 to C-108, wherein the cancer is a cancer with a BRAF V600E or BRAF V600K mutation.

C-110. The method of any one of embodiments C-107 to C-109, wherein the cancer is a melanoma cancer with a BRAF V600E mutation.

C-111. The method of embodiment C-110, wherein the cancer is not wild-type BRAF melanoma.

C-112. The method of any one of embodiments C-107 to C-111, wherein the therapeutically effective amount of encorafenib is 450 mg orally once daily.

C-113. The method of embodiment C-112, wherein the method further comprises the administration of cetuximab in combination with the binimetinib.

C-114. The method of any one of embodiments C-99 to C-113, wherein the encorafenib is administered with or without food.

C-115. The method of any one of embodiments C-99 to C-115, wherein the encorafenib is administered as capsules each having a total of 75 mg of encorafenib.

C-116. A method of treating pancreatic cancer with a RAS mutation, the method consisting of orally administering to a patient in need thereof a therapeutically effective amount of Compound A,

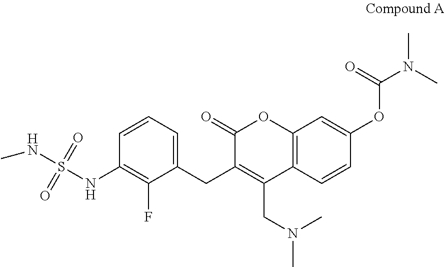

or a pharmaceutically acceptable salt thereof, in combination with a therapeutically effective amount of a nucleoside metabolic inhibitor.

C-117. The method of embodiment C-116, wherein the nucleoside metabolic inhibitor is gemcitabine administered as follows: 1000 mg/m2 over 30 minutes once weekly for the first 7 weeks, then one week rest, then once weekly for 3 weeks on a 28-day cycle.

C-118. The method of embodiment C-117, wherein the gemcitabine and Compound A are the only drugs administered for the treatment of the pancreatic cancer.

C-119. The method of any one of embodiments C-116-118, wherein the pancreatic cancer is pancreatic cancer with a KRAS mutation.

C-120. The method of embodiment C-119, wherein the pancreatic cancer is pancreatic cancer with a KRAS G12 mutation.

C-121. The method of embodiment C-119, wherein the pancreatic cancer is pancreatic cancer with a KRAS G12V mutation.

C-122. The method of embodiment C-119, wherein the pancreatic cancer is pancreatic cancer with a KRAS G12D mutation.

C-123. The method of any one of embodiments C-116 to C-122, wherein the pancreatic cancer is adenocarcinoma of the pancreas.

C-124. A method of treating pancreatic cancer with a RAS mutation, the method consisting of orally administering to a patient in need thereof a therapeutically effective amount of Compound A,

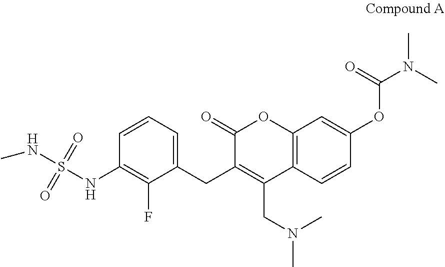

or a pharmaceutically acceptable salt thereof, in combination with a therapeutically effective amount of a microtubule inhibitor.

C-125. The method of embodiment C-124, wherein the microtubule inhibitor is administered in combination with a nucleoside metabolic inhibitor.

C-126. The method of embodiment C-125, wherein the microtubule inhibitor is paclitaxel.

C-127. The method of any one of embodiments C-124 to C-125, wherein the nucleoside metabolic inhibitor is gemcitabine.

C-128. The method of any one of embodiments C-124 to C-127, wherein the microtubule inhibitor is paclitaxel administered as follows: 25 mg/m² intravenously over 30-40 minutes on Days 1, 8, and 15 of each 28-day cycle; administer gemcitabine on Days 1, 8, and 15 of each 28-day cycle immediately after gemcitabine administration.

C-129. The method of any one of embodiments C-125 to C-128, wherein the microtubule inhibitor, the nucleoside metabolic inhibitor and Compound A are the only drugs administered for the treatment of the pancreatic cancer.

C-130. The method of any one of embodiments C-124 to C-129, wherein the pancreatic cancer is pancreatic cancer with a KRAS mutation.

C-131. The method of embodiment C-130, wherein the pancreatic cancer is pancreatic cancer with a KRAS G12 mutation.

C-132. The method of embodiment C-130, wherein the pancreatic cancer is pancreatic cancer with a KRAS G12V mutation.

C-133. The method of embodiment C-130, wherein the pancreatic cancer is pancreatic cancer with a KRAS G12D mutation.

C-134. The method of any one of embodiments C-124 to C-133, wherein the pancreatic cancer is adenocarcinoma of the pancreas.

C-135. A method of treating pancreatic cancer comprising orally administering to the patient in need thereof a therapeutically effective amount of Compound A,

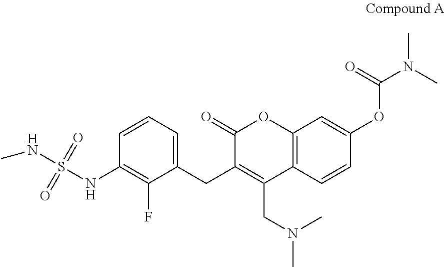

or a pharmaceutically acceptable salt thereof.

C-136. The method of embodiment C-135, wherein the pancreatic cancer is adenocarcinoma of the pancreas.

C-137. The method of any one of embodiments C-135 to C-136, wherein the therapeutically effective amount of Compound A is an amount of Compound A or a pharmaceutically acceptable salt thereof that provides a total dose of 240 mg or 320 mg of Compound A once daily (QD) to the patient in need thereof.

C-138. The method of any one of embodiments C-135 to C-136, wherein the therapeutically effective amount of Compound A is an amount of Compound A or a pharmaceutically acceptable salt thereof that provides a total dose of 240 mg of Compound A once daily (QD) to the patient in need thereof.

C-139. The method of any one of embodiments C-135 to C-136, wherein the therapeutically effective amount of Compound A is an amount of Compound A or a pharmaceutically acceptable salt thereof that provides a total dose of 320 mg of Compound A once daily (QD) to the patient in need thereof.

C-140. The method of any one of embodiments C-135 to C-139, wherein the method further comprises administering gemcitabine to the patient.

C-141. The method of embodiment C-140, wherein the method further comprises administering nab-paclitaxel to the patient.

C-142. The method of any one of embodiments C-135 to C-139, wherein the method further comprises administering a FOLFIRINOX regimen to the patient.

C-143. The method of any one of embodiments C-135 to C-139, wherein the method further comprises administering a modified FOLFIRINOX regimen to the patient.

C-144. The method of any one of embodiments C-135 to C-139, wherein the method further comprises administering oxaliplatin, irinotecan and fluorouracil to the patient.

C-145. The method of embodiment C-144, wherein the oxaliplatin, irinotecan and fluorouracil are administered once every 14 days to the patient, while Compound A is administered once daily to the patient.

C-146. A method of treating pancreatic cancer comprising orally administering to a patient in need thereof a therapeutically effective amount of Compound A,

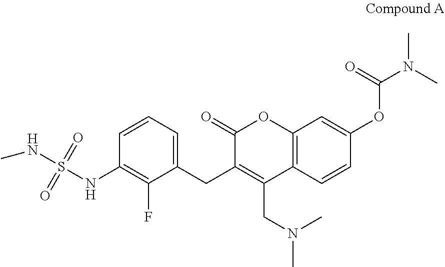

or a pharmaceutically acceptable salt thereof, in combination with a chemotherapy regimen.

C-147. The method of embodiment C-146, wherein the chemotherapy regimen is a gemcitabine-based regimen.

C-148. The method of embodiment C-147, wherein the gemcitabine-based regimen is selected from the gemcitabine-based regimens listed in Table 7.

C-149. The method of embodiment C-146, wherein the chemotherapy regimen is a fluoropyrimidine-based regimen.

C-150. The method of embodiment C-147, wherein the fluoropyrimidine-based regimen is selected from the fluoropyrimidine-based regimens listed in Table 8A.

C-151. The method of embodiment C-147, wherein the fluoropyrimidine-based regimen comprises the administration of 5-fluorouracil.

C-152. The method of any one of embodiments C-147 to C-151, wherein the fluoropyrimidine-based regimen comprises the administration of oxaliplatin.

C-153. The method of any one of embodiments C-147 to C-151, wherein the fluoropyrimidine-based regimen comprises the administration of capecitabine.

C-154. The method of any one of embodiments C-147 to C-151, wherein the fluoropyrimidine-based regimen comprises the administration of leucovorin.

C-155. The method of any one of embodiments C-147 to C-151, wherein the fluoropyrimidine-based regimen comprises the administration of irinotecan.

C-156. The method of embodiment C-147, wherein the fluoropyrimidine-based regimen comprises the administration of 5-fluorouracil, oxaliplatin and leucovorin.

C-157. The method of embodiment C-147, wherein the fluoropyrimidine-based regimen comprises the administration of oxaliplatin and capecitabine.

C-158. The method of embodiment C-147, wherein the fluoropyrimidine-based regimen comprises the administration of 5-fluorouracil, oxaliplatin, irinotecan, and leucovorin.

C-159. The method of embodiment C-147, wherein the fluoropyrimidine-based regimen comprises the administration of liposomal irinotecan, leucovorin, and fluorouracil.

C-160. The method of any one of embodiments C-146 to C-159, wherein Compound A or a pharmaceutically acceptable salt thereof is administered in an amount providing 320 mg of Compound A to the patient per day.

C-161. The method of any one of embodiments C-146 to C-159, wherein Compound A or a pharmaceutically acceptable salt thereof is administered in an amount providing 240 mg of Compound A to the patient per day.

C-162. The method of any one of embodiments C-160 or C-161, wherein Compound A is administered to the patient only once per day (QD).

C-163. The method of embodiment C-162, wherein Compound A is orally administered to the patient (PO).

C-164. A method of treating pancreatic cancer comprising administering to a patient in need thereof a therapeutically effective amount of Compound A,

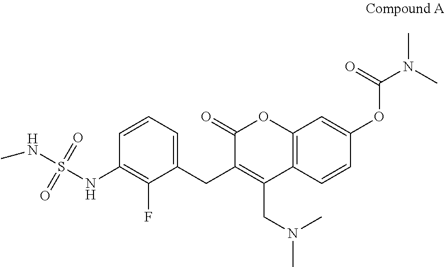

or a pharmaceutically acceptable salt thereof, in combination with nab-paclitaxel for the treatment of the pancreatic cancer.

C-165. The method of embodiment C-164, wherein Compound A or a pharmaceutically acceptable salt thereof is orally administered at a dose of 320 mg or 240 mg of Compound A once per day (QD).

C-166. The method of embodiment C-165, wherein the patient is diagnosed with pancreatic cancer having a KRAS mutation.

C-167. The method of embodiment C-166, wherein the KRAS mutation is a G12C mutation.

C-168. The method of embodiment C-166, wherein the KRAS mutation is a G12V mutation.

C-169. The method of embodiment C-166, wherein the KRAS mutation is a G12D mutation.

C-170. The method of embodiment C-166, wherein the KRAS mutation is a G12R mutation.

C-171. A method of treating pancreatic cancer comprising administering to a patient in need thereof a therapeutically effective amount of Compound A,

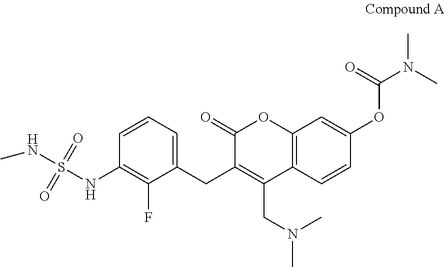

or a pharmaceutically acceptable salt thereof, in combination with 5-fluorouracil for the treatment of the pancreatic cancer.

C-172. The method of embodiment C-171, wherein Compound A or a pharmaceutically acceptable salt thereof is orally administered at a dose of 320 mg or 240 mg of Compound A once per day (QD).

C-173. The method of embodiment C-172, wherein the patient is diagnosed with pancreatic cancer having a KRAS mutation.

C-174. The method of embodiment C-173, wherein the KRAS mutation is a G12C mutation.

C-175. The method of embodiment C-173, wherein the KRAS mutation is a G12V mutation.

C-176. The method of embodiment C-173, wherein the KRAS mutation is a G12D mutation.

C-177. A method of treating colon cancer comprising orally administering to the patient in need thereof a therapeutically effective amount of Compound A,

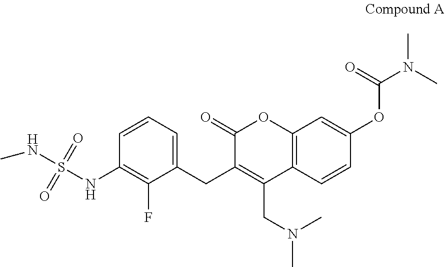

or a pharmaceutically acceptable salt thereof, in combination with gemcitabine for the treatment of the colon cancer.

C-178. The method of embodiment C-177, wherein Compound A or a pharmaceutically acceptable salt thereof is orally administered at a dose of 320 mg or 240 mg of Compound A once per day (QD).

C-179. The method of embodiment C-178, wherein the patient is diagnosed with pancreatic cancer having a KRAS mutation.

C-180. The method of embodiment C-179, wherein the KRAS mutation is a G12V mutation.

C-181. The method of embodiment C-179, wherein the KRAS mutation is a G12D mutation.

C-182. The method of embodiment C-179, wherein the KRAS mutation is a A146T mutation.

C-183. The method of embodiment C-178, wherein the patient is diagnosed with pancreatic cancer having a BRAF mutation.

C-184. The method of embodiment C-183, wherein the BRAF mutation is a V600E mutation.

C-185. A method of treating pancreatic cancer comprising administering to a patient in need thereof a therapeutically effective amount of Compound A,

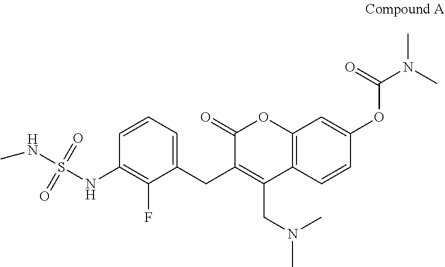

or a pharmaceutically acceptable salt thereof, in combination with oxaliplatin for the treatment of the pancreatic cancer.

C-186. A method of treating colon cancer comprising administering to a patient in need thereof a therapeutically effective amount of Compound A,

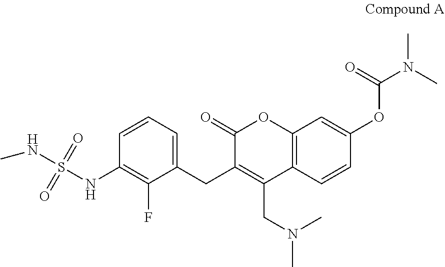

or a pharmaceutically acceptable salt thereof, in combination with oxaliplatin for the treatment of the colon cancer.

C-187. A method of treating lung cancer comprising administering to a patient in need thereof a therapeutically effective amount of Compound A,

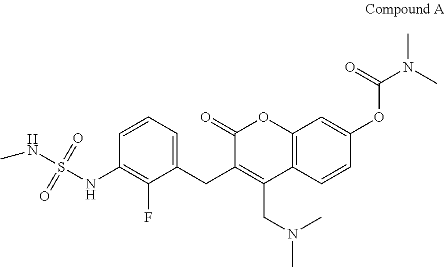

or a pharmaceutically acceptable salt thereof, in combination with oxaliplatin for the treatment of the lung cancer.

C-188. The method of any one of embodiments C-185 to C-187, wherein Compound A or a pharmaceutically acceptable salt thereof is orally administered at a dose of 320 mg or 240 mg of Compound A once per day (QD).

C-189. The method of embodiment C-188, wherein the patient is diagnosed with pancreatic cancer having a KRAS mutation.

C-190. The method of embodiment C-179, wherein the KRAS mutation is a G12C mutation.

C-191. The method of embodiment C-179, wherein the KRAS mutation is a G12V mutation.

C-192. The method of embodiment C-179, wherein the KRAS mutation is a G12D mutation.

C-193. The method of embodiment C-179, wherein the KRAS mutation is a G12S mutation.

C-194. A method of treating pancreatic cancer comprising administering to a patient in need thereof a therapeutically effective amount of Compound A,

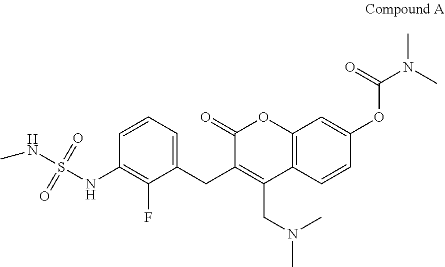

or a pharmaceutically acceptable salt thereof, in combination with irinotecan for the treatment of the pancreatic cancer.

C-195. A method of treating colon cancer comprising administering to a patient in need thereof a therapeutically effective amount of Compound A,

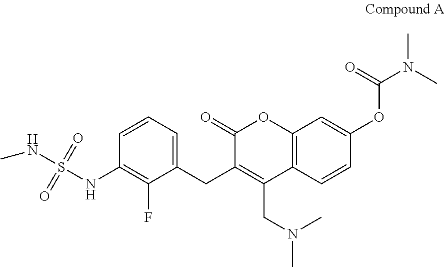

or a pharmaceutically acceptable salt thereof, in combination with irinotecan for the treatment of the colon cancer.

C-196. The method of any one of embodiments C-194 to C-195, wherein Compound A or a pharmaceutically acceptable salt thereof is orally administered at a dose of 320 mg or 240 mg of Compound A once per day (QD).

C-197. The method of embodiment C-196, wherein the patient is diagnosed with cancer having a KRAS mutation.

C-198. The method of embodiment C-197, wherein the KRAS mutation is a G12D mutation.

C-199. The method of embodiment C-197, wherein the KRAS mutation is a G12V mutation.

C-200. A method of treating pancreatic cancer comprising administering to a patient in need thereof a therapeutically effective amount of Compound A,

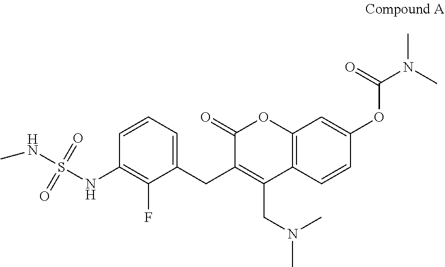

or a pharmaceutically acceptable salt thereof, in combination with a KRAS G12C inhibitor for the treatment of the pancreatic cancer.

C-201. A method of treating colon cancer comprising administering to the patient in need thereof a therapeutically effective amount of Compound A,

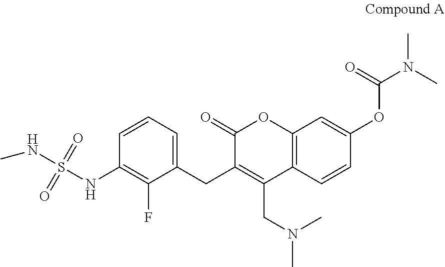

or a pharmaceutically acceptable salt thereof, in combination with a KRAS G12C inhibitor for the treatment of the colon cancer.

C-202. The method of any one of embodiments C-200 to C-201, wherein Compound A or a pharmaceutically acceptable salt thereof is orally administered at a dose of 320 mg or 240 mg of Compound A once per day (QD).

C-203. The method of embodiment C-202, wherein the patient is diagnosed with cancer having a KRAS mutation.

C-204. The method of embodiment C-197, wherein the KRAS mutation is a G12C mutation.

C-205. The method of any one of embodiments C-200 to C-204, wherein the KRAS G12C inhibitor is sotorasib.

C-206. The method of any one of embodiments C-200 to C-204, wherein the KRAS G12C inhibitor is adagrasib.

C-207. A method of treating pancreatic cancer comprising administering to the patient in need thereof a therapeutically effective amount of Compound A,

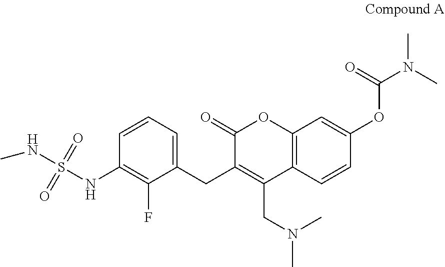

or a pharmaceutically acceptable salt thereof, in combination with a CDK-4/6 inhibitor for the treatment of the pancreatic cancer.

C-208. A method of treating lung cancer comprising administering to a patient in need thereof a therapeutically effective amount of Compound A,

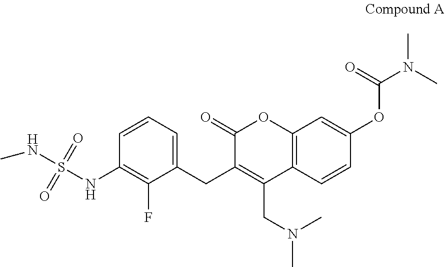

or a pharmaceutically acceptable salt thereof, in combination with a CDK-4/6 inhibitor for the treatment of the lung cancer.

C-209. A method of treating colon cancer comprising administering to a patient in need thereof a therapeutically effective amount of Compound A,

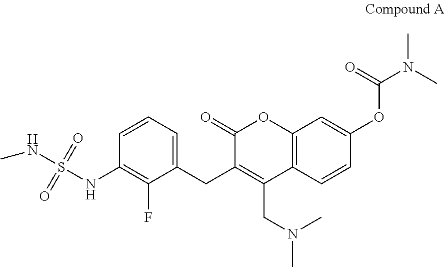

or a pharmaceutically acceptable salt thereof, in combination with a CDK-4/6 inhibitor for the treatment of the colon cancer.

C-210. The method of any one of embodiments C-207 to C-209, wherein Compound A or a pharmaceutically acceptable salt thereof is orally administered at a dose of 320 mg or 240 mg of Compound A once per day (QD).

C-211. The method of embodiment C-210, wherein the patient is diagnosed with cancer having a KRAS mutation.

C-212. The method of embodiment C-211, wherein the KRAS mutation is a G12C mutation.

C-213. The method of any one of embodiments C-207 to C-212, wherein the CDK-4/6 inhibitor is abemaciclib.

C-214. The method of any one of embodiments C-200 to C-204, wherein the KRAS G12C inhibitor is palbociclib.

C-215. A method of treating pancreatic cancer comprising administering to a patient in need thereof a therapeutically effective amount of Compound A,

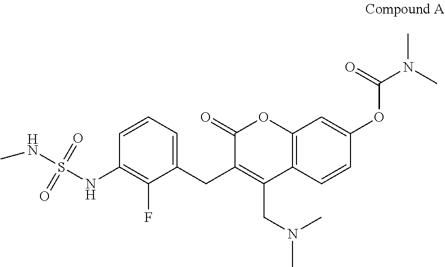

or a pharmaceutically acceptable salt thereof, in combination with an EGFR inhibitor for the treatment of the pancreatic cancer.

C-216. A method of treating a lung cancer comprising administering to a patient in need thereof a therapeutically effective amount of Compound A,

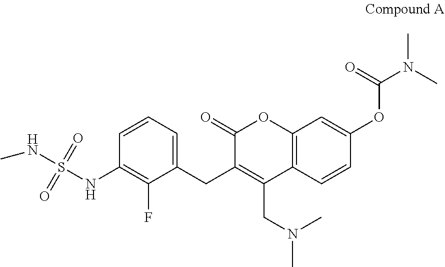

or a pharmaceutically acceptable salt thereof, in combination with an EGFR inhibitor for the treatment of the lung cancer.

C-217. A method of treating colon cancer comprising administering to a patient in need thereof a therapeutically effective amount of Compound A,

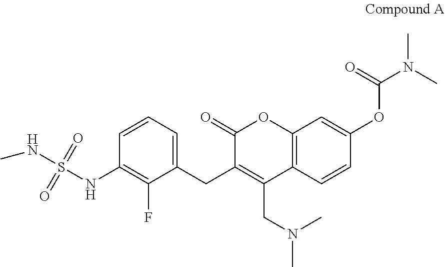

or a pharmaceutically acceptable salt thereof, in combination with an EGFR inhibitor for the treatment of the colon cancer.

C-218. The method of any one of embodiments C-215 to C-217, wherein Compound A or a pharmaceutically acceptable salt thereof is orally administered at a dose of 320 mg or 240 mg of Compound A once per day (QD).

C-219. The method of embodiment C-218, wherein the patient is diagnosed with cancer having a KRAS mutation.

C-220. The method of embodiment C-219, wherein the KRAS mutation is a G12C mutation.

C-221. The method of embodiment C-219, wherein the KRAS mutation is a G12V mutation.

C-222. The method of embodiment C-218, wherein the patient is diagnosed with cancer having a BRAF mutation.

C-223. The method of embodiment C-222, wherein the BRAF mutation is a G596R mutation.

C-224. The method of embodiment C-222, wherein the BRAF mutation is a V600E mutation.

C-225. The method of embodiment C-218, wherein the patient is diagnosed with cancer having an EGFR mutation.

C-226. The method of embodiment C-225, wherein the EGFR mutation is a T790M mutation.

C-227. The method of embodiment C-225, wherein the EGFR mutation is a L858R mutation.

C-228. The method of embodiment C-225, wherein the EGFR mutation is a E746 A750del mutation.

C-229. The method of any one of embodiments C-215 to C-228, wherein the EGFR inhibitor is afatinib.

C-230. The method of any one of embodiments C-215 to C-228, wherein the KRAS G12C inhibitor is erlotinib.

C-231. The method of any one of embodiments C-215 to C-228, wherein the KRAS G12C inhibitor is gefitinib.

C-232. The method of any one of embodiments C-215 to C-228, wherein the KRAS G12C inhibitor is osimertinib.

C-233. A method of treating melanoma comprising administering to a patient in need thereof a therapeutically effective amount of Compound A,

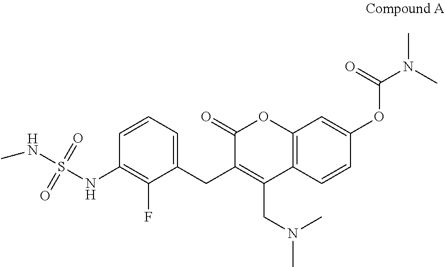

or a pharmaceutically acceptable salt thereof, in combination with a BRAF inhibitor for the treatment of the melanoma.

C-234. A method of treating colorectal cancer comprising administering to a patient in need thereof a therapeutically effective amount of Compound A,

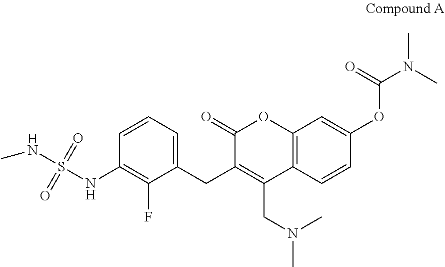

or a pharmaceutically acceptable salt thereof, in combination with a BRAF inhibitor for the treatment of the colorectal cancer.

C-235. The method of any one of embodiments C-233 to C-234, wherein Compound A or a pharmaceutically acceptable salt thereof is orally administered at a dose of 320 mg or 240 mg of Compound A once per day (QD).

C-236. The method of embodiment C-235, wherein the patient is diagnosed with cancer having a BRAF mutation.

C-237. The method of embodiment C-236, wherein the BRAF mutation is a V600E mutation.

C-238. The method of any one of embodiments C-233-237, wherein the BRAF inhibitor is encorafenib.

C-239. A method of treating lung cancer comprising administering to a patient in need thereof a therapeutically effective amount of Compound A,

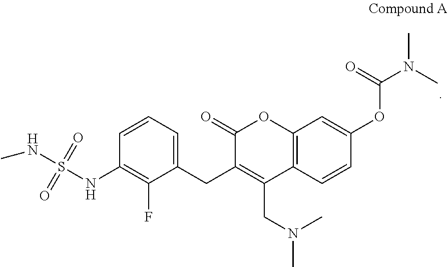

or a pharmaceutically acceptable salt thereof, in combination with an ALK inhibitor for the treatment of the lung cancer.

C-240. The method of embodiment C-239, wherein Compound A or a pharmaceutically acceptable salt thereof is orally administered at a dose of 320 mg or 240 mg of Compound A once per day (QD).

C-241. The method of embodiment C-240, wherein the patient is diagnosed with cancer having a NRAS mutation.

C-242. The method of embodiment C-241, wherein the NRAS mutation is a Q61K mutation.

C-243. The method of embodiment C-240, wherein the patient is diagnosed with cancer having a KRAS mutation.

C-244. The method of embodiment C-243, wherein the KRAS mutation is a G12V mutation.

C-245. The method of any one of embodiments C-239-244, wherein the patient is diagnosed with cancer having a ALK mutation.

C-246. The method of embodiment C-245, wherein the ALK mutation is a S553A mutation.

C-247. The method of embodiment C-245, wherein the ALK mutation is a G103R mutation.

C-248. The method of embodiment C-245, wherein the ALK mutation is a R317T mutation.

C-249. The method of any one of embodiments C-239 to C-248, wherein the ALK inhibitor is alectinib.

C-250. The method of any one of embodiments C-239 to C-248, wherein the ALK inhibitor is brigatinib.

C-251. The method of any one of embodiments C-239 to C-248, wherein the ALK inhibitor is crizotinib.

C-252. The method of any one of embodiments C-239 to C-248, wherein the ALK inhibitor is loratinib.

C-253. A method of treating AML comprising administering to a patient in need thereof a therapeutically effective amount of Compound A,

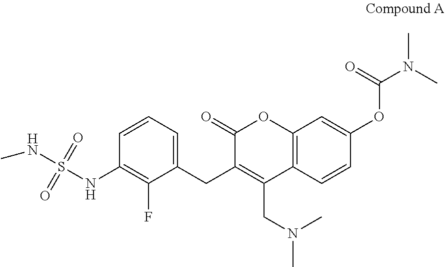

or a pharmaceutically acceptable salt thereof, in combination with azacitidine for the treatment of the AML.

C-254. The method of embodiment C-253, wherein Compound A or a pharmaceutically acceptable salt thereof is orally administered at a dose of 320 mg or 240 mg of Compound A once per day (QD).

C-255. The method of embodiment C-254, wherein the patient is diagnosed with cancer having a NRAS mutation.

C-256. The method of embodiment C-255, wherein the NRAS mutation is a Q61L mutation.

C-257. The method of embodiment C-255, wherein the NRAS mutation is a G12C mutation.

C-258. The method of embodiment C-255, wherein the NRAS mutation is a Q61P mutation.

C-259. The method of embodiment C-255, wherein the NRAS mutation is a G12D mutation.

C-260. The method of embodiment C-254, wherein the patient is diagnosed with cancer having a KRAS mutation.

C-261. The method of embodiment C-260, wherein the KRAS mutation is a A146V mutation.

C-262. A method of treating AML comprising administering to a patient in need thereof a therapeutically effective amount of Compound A,

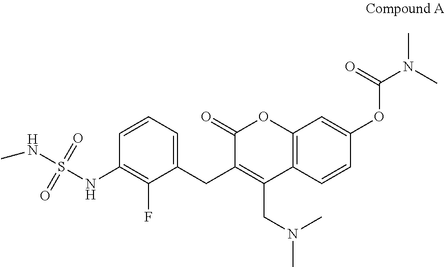

or a pharmaceutically acceptable salt thereof, in combination with decitabine for the treatment of the AML.

C-263. The method of embodiment C-262, wherein Compound A or a pharmaceutically acceptable salt thereof is orally administered at a dose of 320 mg or 240 mg of Compound A once per day (QD).

C-264. The method of embodiment C-263, wherein the patient is diagnosed with cancer having a NRAS mutation.

C-265. The method of embodiment C-264, wherein the NRAS mutation is a Q61L mutation.

C-266. The method of embodiment C-264, wherein the NRAS mutation is a G12D mutation.

C-267. The method of embodiment C-264, wherein the NRAS mutation is a Q61P mutation.

C-268. The method of embodiment C-264, wherein the NRAS mutation is a G12D mutation.

C-269. The method of embodiment C-263, wherein the patient is diagnosed with cancer having a KRAS mutation.

C-270. The method of embodiment C-269, wherein the KRAS mutation is a A146V mutation.

C-271. A method of treating AML comprising administering to a patient in need thereof a therapeutically effective amount of Compound A,

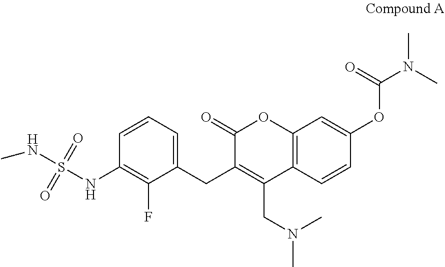

or a pharmaceutically acceptable salt thereof, in combination with daunorubicin for the treatment of the AML.

C-272. The method of embodiment C-271, wherein Compound A or a pharmaceutically acceptable salt thereof is orally administered at a dose of 320 mg or 240 mg of Compound A once per day (QD).

C-273. The method of embodiment C-272, wherein the patient is diagnosed with cancer having a NRAS mutation.

C-274. The method of embodiment C-273, wherein the NRAS mutation is a Q61L mutation.

C-275. The method of embodiment C-273, wherein the NRAS mutation is a G12D mutation.

C-276. The method of embodiment C-273, wherein the NRAS mutation is a Q61P mutation.

C-277. The method of embodiment C-272, wherein the patient is diagnosed with cancer having a KRAS mutation.

C-278. The method of embodiment C-277, wherein the KRAS mutation is a A146V mutation.

C-279. A method of treating AML comprising administering to a patient in need thereof a therapeutically effective amount of Compound A,

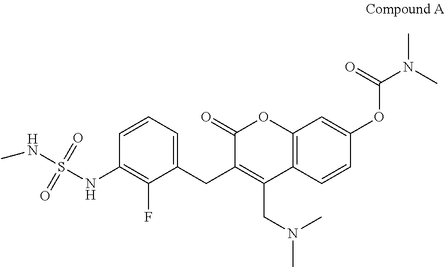

or a pharmaceutically acceptable salt thereof, in combination with bortezomib for the treatment of the AML.

C-280. The method of embodiment C-279, wherein Compound A or a pharmaceutically acceptable salt thereof is orally administered at a dose of 320 mg or 240 mg of Compound A once per day (QD).

C-281. The method of embodiment C-280, wherein the patient is diagnosed with cancer having a NRAS mutation.

C-282. The method of embodiment C-281, wherein the NRAS mutation is a G12C mutation.

C-283. The method of embodiment C-281, wherein the NRAS mutation is a Q61H mutation.

C-284. The method of embodiment C-280, wherein the patient is diagnosed with cancer having a KRAS mutation.

C-285. The method of embodiment C-284, wherein the KRAS mutation is a G12A mutation.

C-286. A method of treating pancreatic cancer comprising administering to a patient in need thereof a therapeutically effective amount of Compound A,

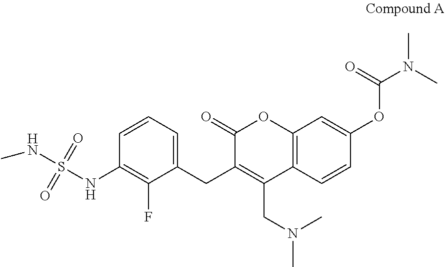

or a pharmaceutically acceptable salt thereof, in combination with FAK inhibitor for the treatment of the pancreatic cancer.

C-287. A method of treating lung cancer comprising administering to a patient in need thereof a therapeutically effective amount of Compound A,

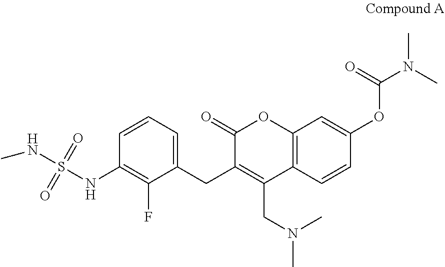

or a pharmaceutically acceptable salt thereof, in combination with FAK inhibitor for the treatment of the lung cancer.

C-288. A method of treating colon cancer comprising administering to a patient in need thereof a therapeutically effective amount of Compound A,

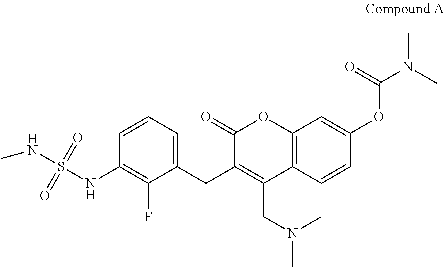

or a pharmaceutically acceptable salt thereof, in combination with FAK inhibitor for the treatment of the colon cancer.

C-289. The method of any one of embodiments C-286 to C-288, wherein Compound A or a pharmaceutically acceptable salt thereof is orally administered at a dose of 320 mg or 240 mg of Compound A once per day (QD).

C-290. The method of embodiment C-289, wherein the patient is diagnosed with cancer having a KRAS mutation.

C-291. The method of embodiment C-290, wherein the KRAS mutation is a G12C mutation.

C-292. The method of embodiment C-290, wherein the KRAS mutation is a G12D mutation.

C-293. The method of embodiment C-280, wherein the FAK inhibitor is ifebemetinib.

C-294. A method of treating pancreatic cancer comprising administering to a patient in need thereof a therapeutically effective amount of Compound A,

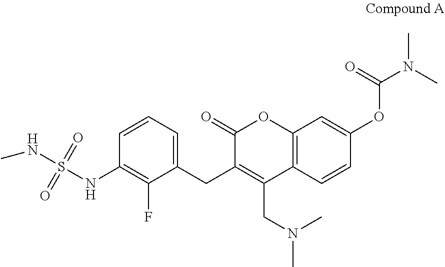

or a pharmaceutically acceptable salt thereof, in combination with PARP inhibitor for the treatment of the pancreatic cancer.

C-295. A method of treating lung cancer comprising administering to a patient in need thereof a therapeutically effective amount of Compound A,

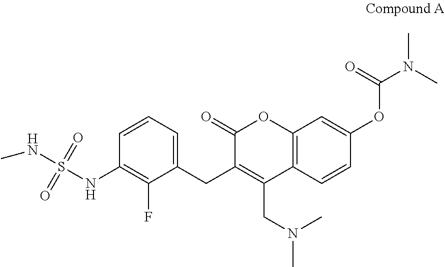

or a pharmaceutically acceptable salt thereof, in combination with PARP inhibitor for the treatment of the lung cancer.

C-296. A method of treating AML comprising administering to a patient in need thereof a therapeutically effective amount of Compound A,

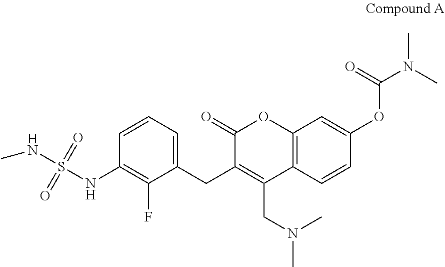

or a pharmaceutically acceptable salt thereof, in combination with PARP inhibitor for the treatment of the AML.

C-297. The method of any one of embodiments C-294-296, wherein Compound A or a pharmaceutically acceptable salt thereof is orally administered at a dose of 320 mg or 240 mg of Compound A once per day (QD).

C-298. The method of embodiment C-297, wherein the patient is diagnosed with cancer having a KRAS mutation.

C-299. The method of embodiment C-298, wherein the KRAS mutation is a G12C mutation.

C-300. The method of embodiment C-298, wherein the KRAS mutation is a G12V mutation.

C-301. The method of embodiment C-297, wherein the patient is diagnosed with cancer having a NRAS mutation.

C-302. The method of embodiment C-301, wherein the NRAS mutation is a Q61L mutation.

C-303. The method of embodiment C-301, wherein the NRAS mutation is a G12D mutation.

### EXAMPLES

Additional embodiments are disclosed in further detail in the following representative examples, which are not in any way intended to limit the scope of the claims. The following examples are provided for the guidance of the reader, and represent certain methods for making and using the compounds and compositions exemplified herein. These methods are not limiting, and it will be apparent that other routes may be employed to prepare these compounds. The compound numberings used in the synthetic schemes depicted below are meant for those specific schemes only, and should not be construed as or confused with same numberings in other sections of the application.

### Example 1

### Synthesis of Compound A

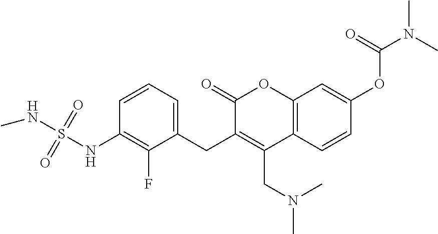

Compound A was prepared in 1 step:

4-(bromomethyl)-3-(2-fluoro-3-((N-methylsulfamoyl)amino)benzyl)-2-oxo-2H-chromen-7-yl dimethylcarbamate (22.22 g, 34.79 mmol) was suspended in methanol. Dimethylamine 2M was added and the formed reaction mixture was stirred until full conversion was observed. After full conversion the reaction was concentrated under reduced pressure. 1M HCl was added to the residue and the water layer was extracted with CH₂Cl₂. The water layer was made basic with solid Na₂CO₃. The basic water layer was extracted with CH₂Cl₂. The organic layer from the basic extraction was washed with brine, dried over Na₂SO₄, filtered and concentrated under reduced pressure to obtain the title compound (13.23 g, 25.7 mmol, yield: 74%) as a light yellow solid.

Yield: Compound A was isolated as a light yellow solid (74% over 1 step). Analysis: LCMS (Method T): t_{R}=1.53 min; m/z calculated for [M-H]⁺=507.2, found=507.2; ¹H NMR (400 MHz, DMSO) δ 9.38 (s, 1H), 8.08 (d, J=8.8 Hz, 1H), 7.28 (td, J=8.0, 1.6 Hz, 1H), 7.25-7.18 (m, 2H), 7.15 (dd, J=8.8, 2.4 Hz, 1H), 7.00 (t, J=7.9 Hz, 1H), 6.90-6.77 (m, 1H), 4.04 (s, 2H), 3.64 (s, 2H), 3.06 (s, 3H), 2.93 (s, 3H), 2.52 (d, J=4.9 Hz, 3H), 2.19 (s, 6H).

### Example 2

### Compound A Nonclinical Pharmacology

Compound A is a potent, non-adenosine 5′-Triphosphate (ATP) competitive selective dual MEK inhibitor with the biochemical inhibitory half-maximal inhibitory concentration (IC₅₀) for activated ERK of 24.2 nM to 83.2 nM across several cancer disease types. Compound A has been shown to have in vivo anti-tumor activity in mouse syngeneic and human xenograft models of activating MAPK pathway mutations in KRAS, NRAS, HRAS, BRAF, neurofibromatosis type 1 (NF1), and KSR, with the most sensitive being predominantly melanoma and pancreatic cancer cell lines that harbor activating MAPK pathway mutations in KRAS, NRAS, HRAS, BRAF, NF6, and KSR (Table 2).

TABLE 2
(IC₅₀ values for pERK in Multiple 2D Grown Cell Lines)

| Tumor cell line and MAPK | Key MAPK Activating | |
|---|---|---|
| mutation | Mutation(s) | IC₅₀ nM^{a} |
| A549 KRAS^{G12S} | KRAS: p.G12S, KSR1: Amplified, | 24.16 to 36.63 |
| Human lung adenocarcinoma | SOS1: Amplified | |
| A375 BRAF^{V600E} | BRAF: p.V600E: class1, | 37.65 to 83.19 |
| Human malignant melanoma | MAP2K2: Amplified | |
| SK-MEL-2 NRAS^{Q61R} | NRAS: p.Q61R, BRAF: Amplified, | 26.19 to 36.17 |
| Human pancreatic adenocarcinoma | RAF1: Amplifeid, | |
| | MAP2K2:Amplified | |
| MIA PaCa-2 KRAS^{G12C} | KRAS: p.G12C, NRAS: Amplified, | 49.39 |
| Human pancreatic adenocarcinoma | BRAF: Amplified, | |
| | MAP2K2: Amplified, | |
| | KSR1: Amplified | |
| NCI-H1299 NRAS^{Q61K} | KRAS: Amplified, NRAS: p.Q61K, | 66.46 |
| Human non-small cell lung cancer | BRAF: Amplified, | |
| | MAP2K2: Amplified | |
| SK-MEL-28 BRAF^{V600E} | KRAS: Amplified, | 29.86 |
| Human malignant melanoma | BRAF: p.V600E: class1; Amplified, | |
| | FAF1: Amplified, | |
| | MAPK1:Amplified, | |
| | SOS1: Amplified | |
| CT26 KRAS^{G12D} | KRAS: p.G12D (mouse cell line; | 46.26 |
| Murine colon carcinoma | known mutation) | |

2D = two dimensional;
Amp = amplified;
BRAF = B isomer of rapidly accelerated fibrosarcoma (RAF);
IC50 = 50% inhibitory concentration;
KRAS = K isomer of rat sarcoma (RAS);
KSR = kinase suppressor of Ras;
MAP2K2 = mitogen-activated protein kinase kinase 2;
MEL = melanoma;
NF1 = neurofibromatosis type 1;
NRAS = N isomer of ratserine/threonine kinase;
SOS1 = son of rat sarcoma (RAS);
RAF1 = Raf-1 proto-oncogene, serine/threonine kinase;
SOS1 = son of sevenless 1
^{a}Potency observed in cell-based assays (range shown when experiment was repeated)

Corresponding reductions in phosphorylated MEK (pMEK) were observed within 2-hours following treatment of cells with 100 nM Compound A, confirming that Compound A was not sensitive to the MAPK pathway feedback reactivation events, such as CRAF-bypass, that are a known limitation of first generation MEK inhibitors currently in clinical use.

### Example 3

### Preclinical Studies: Pharmacogenomics Data

In a pharmacogenomics study utilizing a colorectal KRAS G12D tumor model in BALB/c mice, downstream ERK inhibition of the MAPK pathway was evaluated after Compound A treatment.

To demonstrate the ability of Compound A to achieve deep, cyclic inhibition, the downstream transcriptomic activity following Compound A exposure (100 mg/kg BID PO) was evaluated and compared to selumetinib (100 mg/kg BID PO) in the Colon-26 model. RNA sequencing of tumors allowed for interrogation of the 20 most common genes associated with ERK-induced transcription. Analysis was performed at 2 h and 12 h post-dose after 18 days of chronic treatment administration.

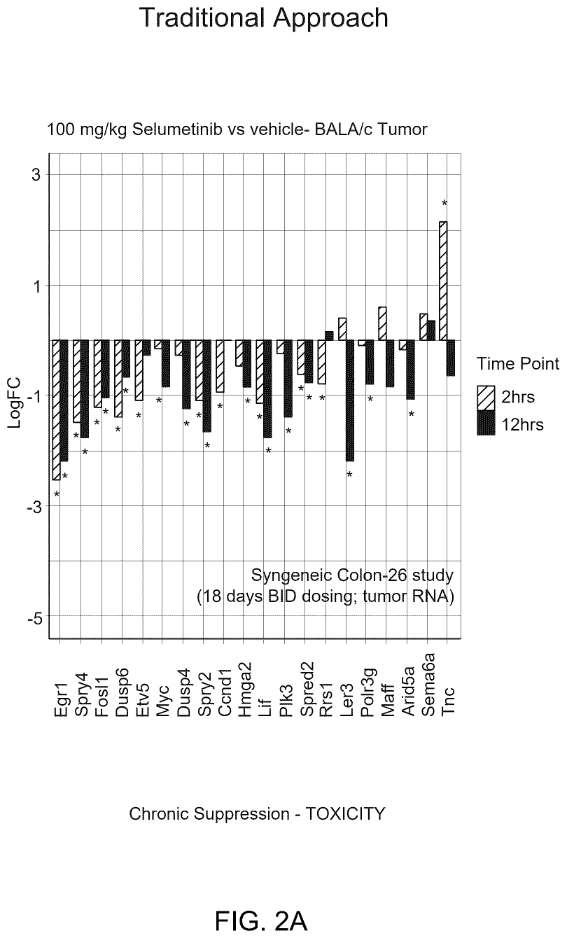

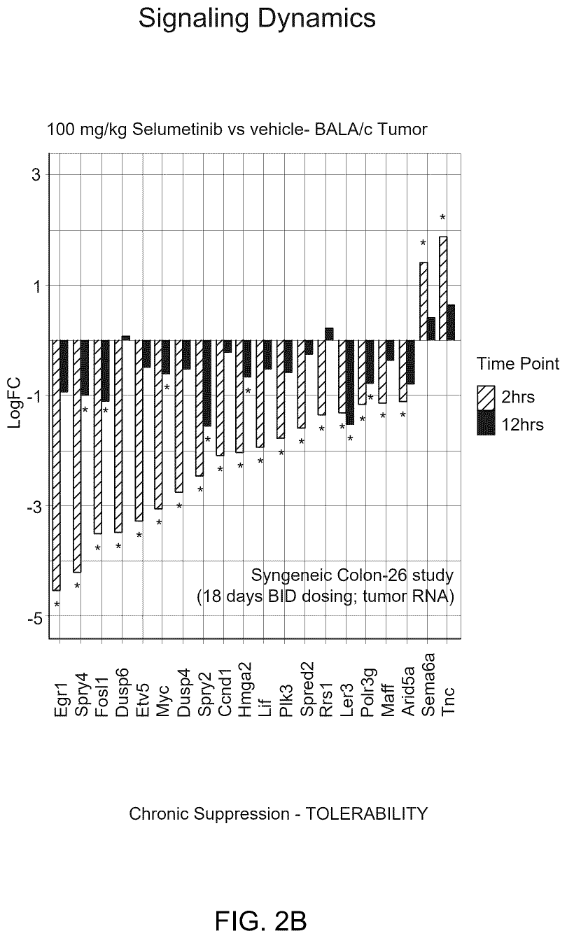

The vehicle, selumetinib and Compound A was orally administered twice a day at 100 mg/kg/dose, then harvested the tumors after 18 days of chronic treatment at 2 and 12 hours following the last drug dose to evaluate RNAseq changes. The tumors were collected across distinct BALB/c mice and RNAseq changes were evaluated using statistical analysis software. Consistent with Compound A's designed short plasma half-life, deep, cyclic inhibition of most of the top genes in the ERK transcriptome was observed, as noted by the differences of the dark and light blue bars, which suggests the potential for improved tolerability by allowing healthy normal cells to regenerate before the next dose is administered. For example, Erg1 and Spry4 were both downregulated over 16-fold at 2 hours after receiving the first dose on day 18 of the study, and at 12 hours after the first dose, which was prior to the second dose, both genes were approaching their baseline state when compared to vehicle treated tumors (FIG. 2A). In contrast to Compound A, deep cyclic inhibition by selumetinib was not observed, but rather sustained MAPK pathway suppression versus vehicle groups between the two timepoints on day 18 was observed (FIGS. 2A and 2B). The top 20 genes were a subset of a 52-gene signature for ERK signaling.

The data showed that, compared with selumetinib, Compound A resulted in a greater difference in transcriptomic activity at Cmax and at trough in most of the genes evaluated.

FIG. 2B is a graph showing data from a head-to-head comparison of Compound A against selumetinib using a Colon-26 syngeneic tumor model. The data shows deep cyclic inhibition of the ERK transcriptome.

### Example 4

### Preclinical Studies: Compound A in a KRAS Mutant NSCLC Tumor Model

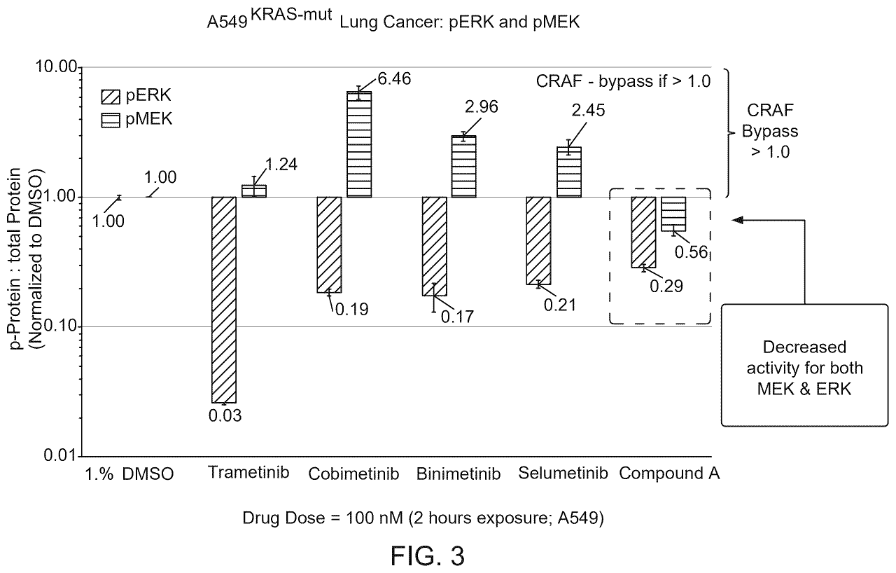

Compound A was evaluated head-to-head against four FDA-approved MEK inhibitors for CRAF-bypass resistance in a KRAS mutant NSCLC tumor model. The tumor cells with 100 nM of each drug were exposed for 2 hours and MEK and ERK activation levels were evaluated. Compound A was observed to reduce overall activity of the MAPK pathway at ERK and pathway reactivation at MEK through a decrease in MEK and ERK activation, resulting in CRAF-bypass resistance. In contrast, it was observed that all four FDA-approved MEK inhibitors displayed an increase in activated MEK, resulting in CRAF-bypass (FIG. 3).

As depicted in FIG. 3, while each MEK inhibitor effectively inhibited ERK, as evidenced by decreases in pERK (blue bars), the FDA-approved MEK inhibitor all demonstrated an increase in cellular pools of pMEK (red bars), indicative of a reactivation event through CRAF bypass. Only Compound A inhibited both pERK and pMEK in the RAS-mutated setting. Compound A acts as a dual MEK inhibitor.

FIG. 3 shows results from a head-to-head comparison of Compound A against four FDA-Approved MEK Inhibitors Using a A549 Xenograft Tumor Model, showing Compound A prevented downstream activation of ERK (↓ pERK) and inhibited activation of MEK (↓ pMEK).

### Example 5

### Preclinical Studies: Compound A in a Colon-26 Tumor Model

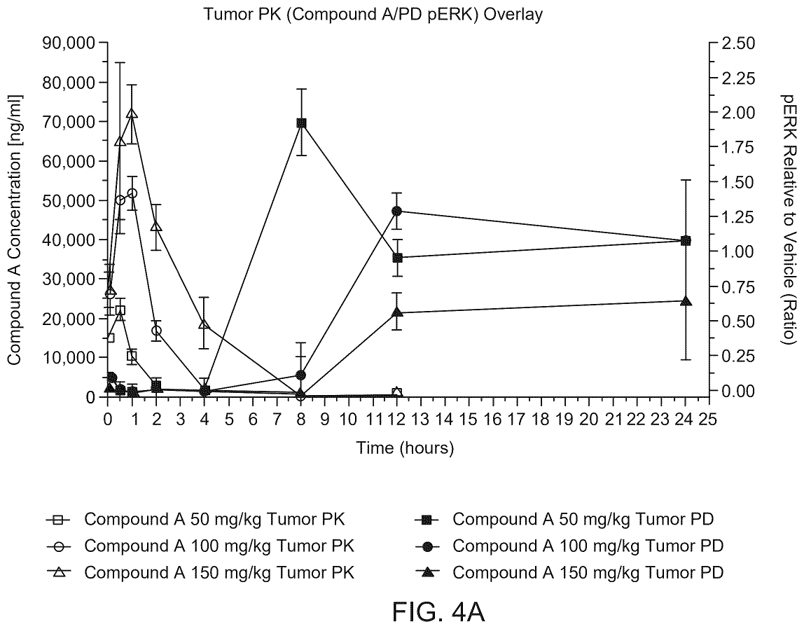

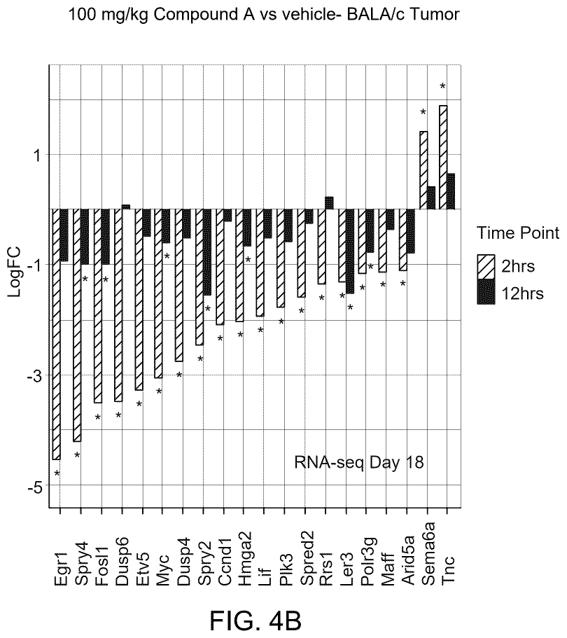

In the Colon-26 model, Compound A transiently inhibited activated pERK in a dose-dependent manner (FIG. 4A) at doses ranging from 12.5 to 150 mg/kg PO after a single dose. This suppression correlated with plasma and tumor exposures, showing that when levels dropped below 50% pERK inhibition (IC₅₀) levels compared to vehicle from in vitro 2D assays (24.16 nM to 83.90 nM) the attenuation was reversed. Further evidence supporting the selective deep cyclic inhibition was shown at the genomic level, where Compound A attenuated the expression of several ERK signaling pathway genes when dosed at 100 mg/kg BID PO for 18 days. As an example, on day 18 of the study Egr1 and Spry4 were both downregulated by over 16-fold, 2 hours after receiving the first dose (note: log 2FC scale), while at 12 hours after the first dose, representing the drug trough in a BID schedule, both genes were approaching their baseline state when compared to vehicle treated tumors (FIG. 4B).

Referring to FIG. 4A and FIG. 4B: Syngeneic Colon-26 (KRASG12D mutant) colorectal tumor bearing mice treated with Compound A. Composite graph (FIG. 4A) shows tumor PK and PD (pERK) quantified at the times indicated following a single PO dose. Top genes in the ERK transcriptome from tumor treated with 100 mg/kg BID PO Compound A (FIG. 4B) were expressed as log fold change (* adj p-value<0.05 vs. vehicle control). Tumors were taken at 2-hour (dark bars) or 12-hour (light bars) as last dose on day 18.

Compound A was evaluated head-to-head against binimetinib and selumetinib in an aggressive murine colorectal tumor model (i.e., Colon-26), which expresses mutant KRAS-G12D. Both binimetinib and selumetinib have relatively short half-lives (3.5 hr and 6.2 hr, respectively).

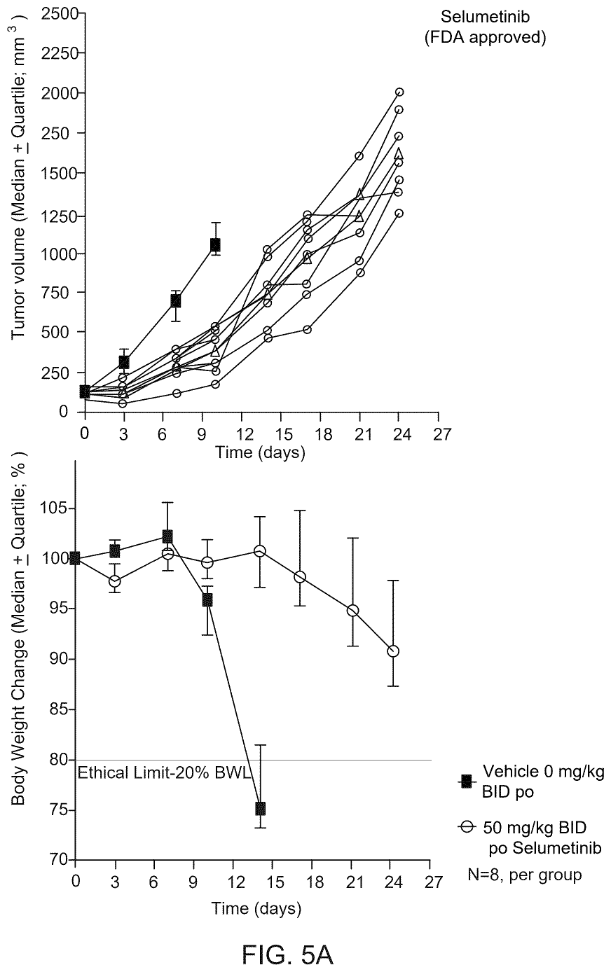

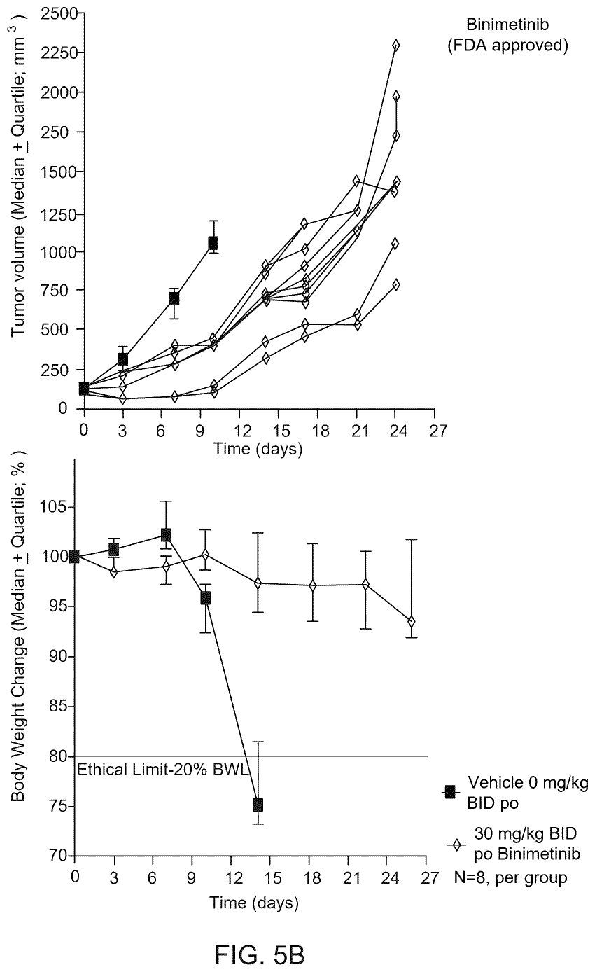

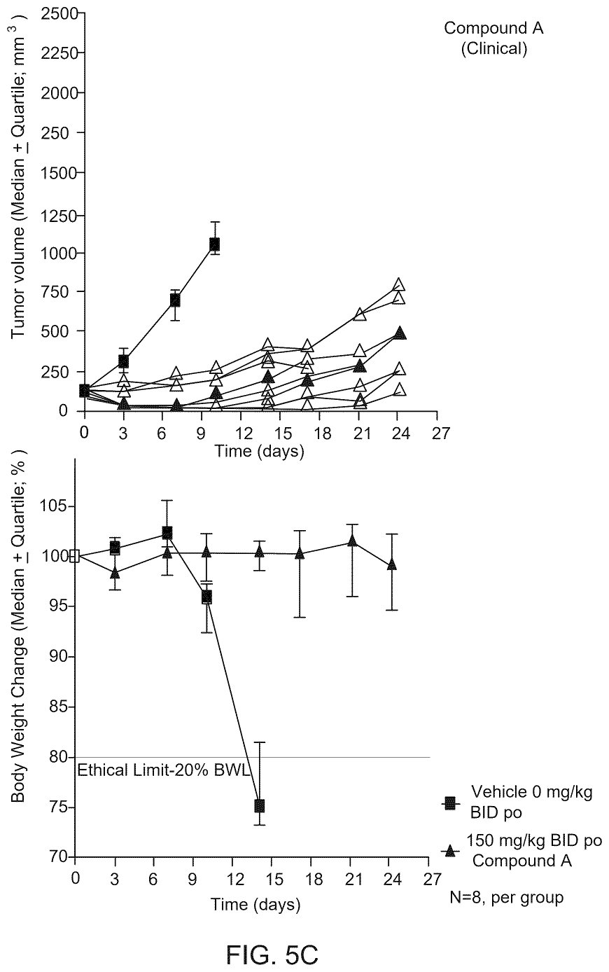

Compound A was observed to demonstrate greater tumor growth inhibition, where notably 5 of 8 mice experienced tumor regression during the first 10 days of dosing, as well as greater tolerability, evidenced by changes in BWL. In addition, Compound A was observed to have overall better durability of antitumor response as compared to the two FDA-approved MEK inhibitors, as demonstrated by significantly lower tumor volume, or TV, progression. This study demonstrated that Compound A as compared to binimetinib and selumetinib provided greater tumor inhibition, lower BWL and lower TV progression (as depicted in FIGS. 5A-C).

FIGS. 5A-C are graphs showing the head-to-head comparison of Compound A against binimetinib and selumetinib using a Colon-26 syngeneic tumor model (body weight loss and tumor volume observed). Colon-26 (KRAS-G12D) syngeneic colorectal tumor model in balb/c mice. Selumetinib and binimetinib were commercially purchased. Tumor Growth Inhibition; TGI %=[1−(Ti−T₀)/(Ci−C₀)]×100; T=treatment groups; C=control groups; expanded TGI formula vs. previous 1−[T/C)]×100% method.

### Example 6

### Preclinical Studies: Compound A in A549 NSCLC Xenograft Tumor Model

After observing the results of the Colon-26 tumor study, two follow-up in vivo studies were completed, where Compound A was evaluated head-to-head against binimetinib or encorafenib, a BRAF inhibitor, as monotherapy plus the combination of binimetinib with encorafenib in BALB/c mice tumor models with RAS and RAF mutations. It should be noted that when encorafenib is used to treat KRAS mutant tumors that are wild type for BRAF, it can paradoxically activate the MAPK pathway and antagonize the effects of binimetinib. In addition, the drug doses and schedules used for binimetinib and encorafenib in these studies were consistent with or exceeded what was provided in their NDAs to the FDA for human-relevant exposures in rodent models.

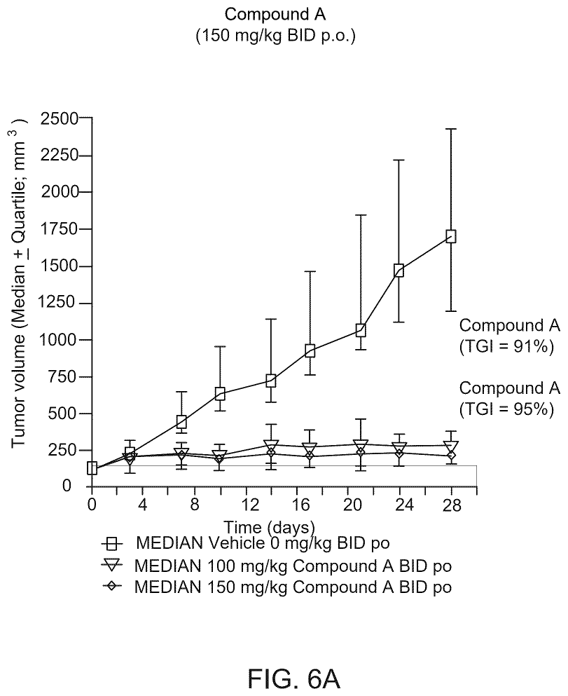

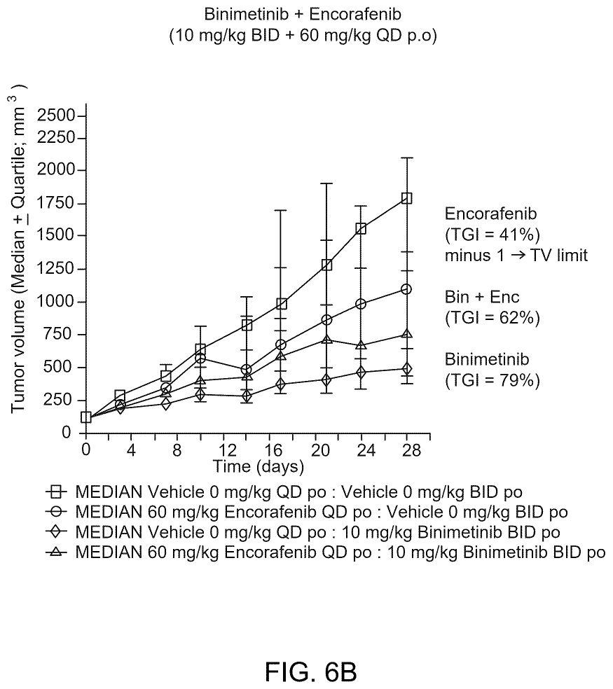

Compound A was evaluated head-to-head against binimetinib monotherapy and in combination with encorafenib in the KRAS G12S human NSCLC xenograft tumor model (i.e., A549). When comparing Compound A to binimetinib monotherapy, Compound A was observed to have greater tumor growth inhibition (FIGS. 6A and 6B). The observations of Compound A head-to-head against binimetinib alone and in combination with encorafenib, which was not considered relevant for a KRAS mutant, RAF wild-type tumor model, has been included in the figure below for comparison purposes.

FIGS. 6A and 6B are graphs showing data from head-to-head comparison of Compound A against binimetinib+/− encorafenib using an A549 xenograft tumor model (tumor volume). A549 (KRAS^{G}12S) NSCLC Xenograft Tumor Model in Athymic Nude Mice. Binimetinib and encorafenib were commercially purchased. Tumor Growth Inhibition; (TGI) %=[1−(Ti−T₀)/(Ci−C₀)]×100; T=treatment groups; C=control groups; expanded TGI formula vs. previously 1−[T/C]×100% method. Human Dose Equivalent (HDE) binimetinib=1-3 mg/kg BID.

### Example 7

### Preclinical Studies: Compound A in A375 (BRAF V600E)

### Melanoma Xenograft Tumor Model

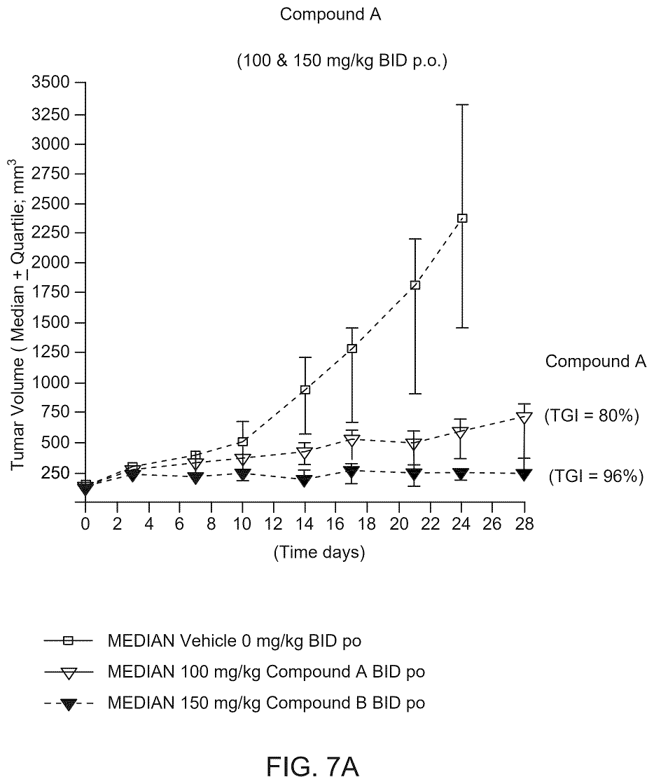

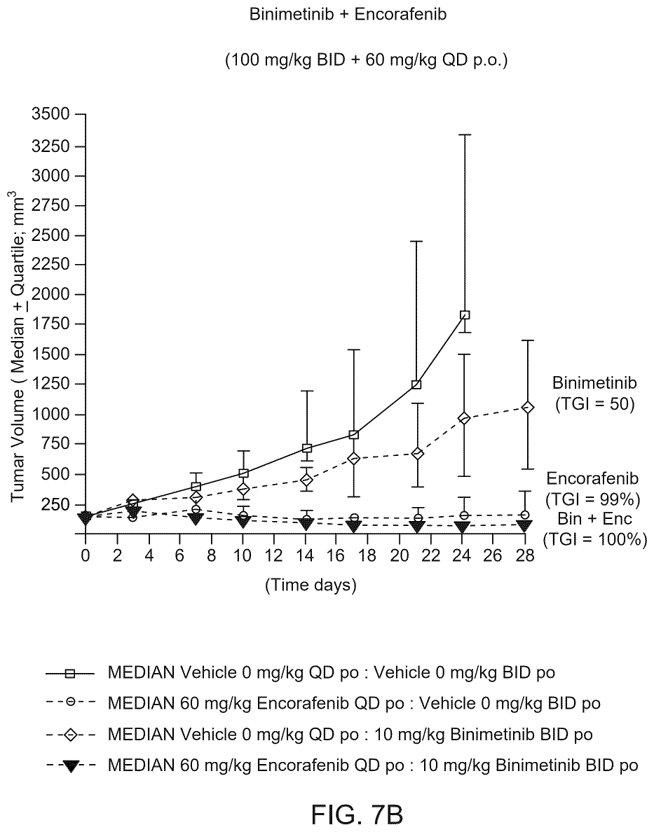

Compound A was evaluated head-to-head against binimetinib and encorafenib monotherapy and the combination of binimetinib with encorafenib in a BRAF V600E human melanoma xenograft model. It should be noted that the administered combination of binimetinib and encorafenib for BRAF mutant melanoma, such as BRAF V600E/K, is an FDA-approved combination. When comparing Compound A alone to binimetinib in combination with encorafenib, we observed that the combination therapy had greater tumor growth inhibition (FIG. 7A). However, Compound A was compared to binimetinib monotherapy, it was observed that Compound A had greater tumor growth inhibition (FIG. 7B).

FIGS. 7A and 7B are graphs showing data from the head-to-head comparison of Compound A against binimetinib+/− encorafenib using a A375 xenograft tumor model (tumor volume). A375 (BRAFV600E) Melanoma Xenograft Tumor Model in Athymic Nude Mice. Binimetinib and encorafenib were commercially purchased. Tumor Growth Inhibition; (TGI) %=[1−(Ti−T₀)/(Ci−C₀)]×100; T=treatment groups; C=control groups; expanded TGI formula vs. previously 1−[T/C]×100% method. Human Dose Equivalent (HDE) binimetinib=1-3 mg/kg BID.

### Example 8A

Preclinical studies: Compound A in MIA PaCa-2 (KRAS G12C) and Capan-2 (KRAS-G12V)

### Pancreatic Cancer Xenograft Tumor Models

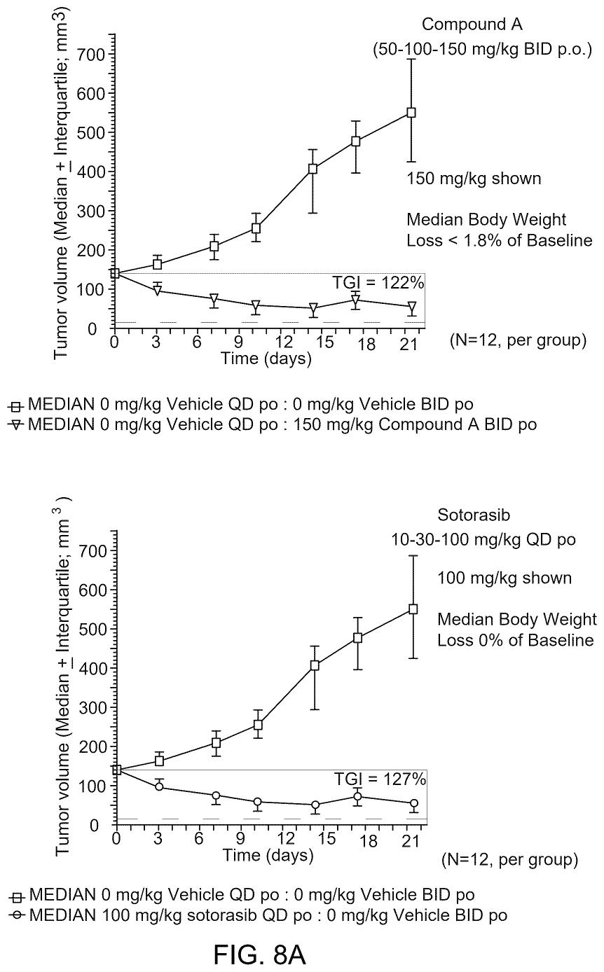

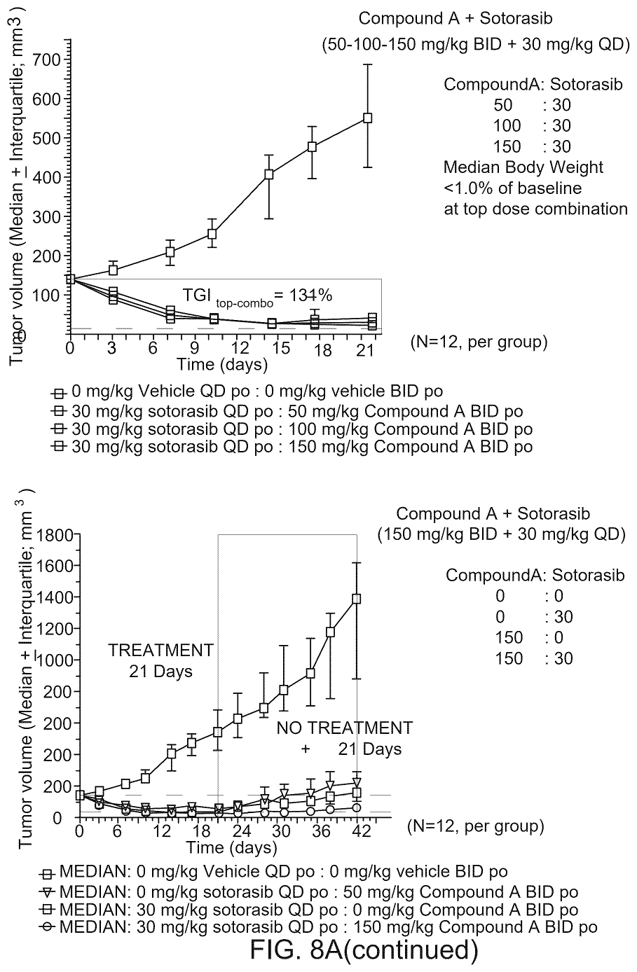

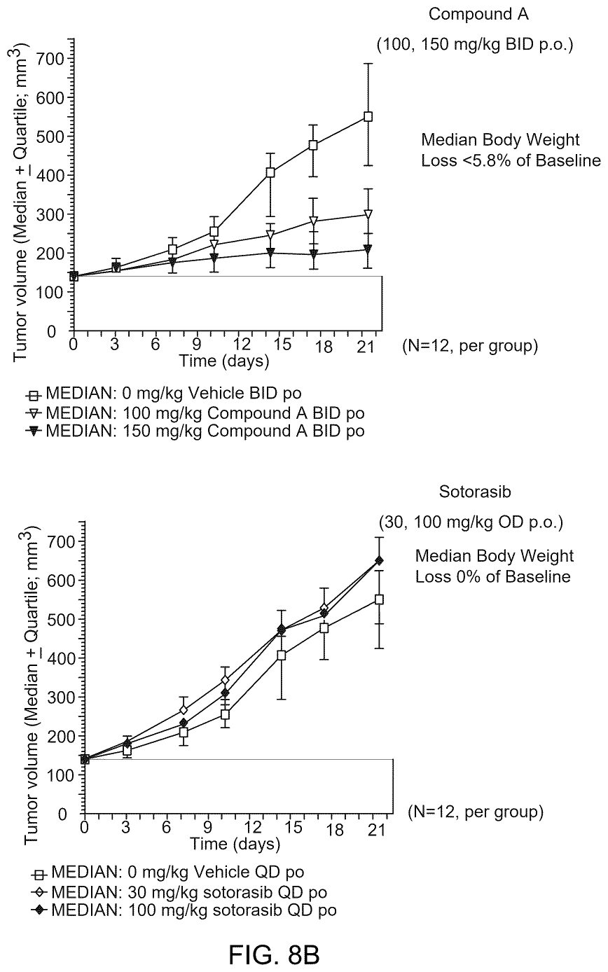

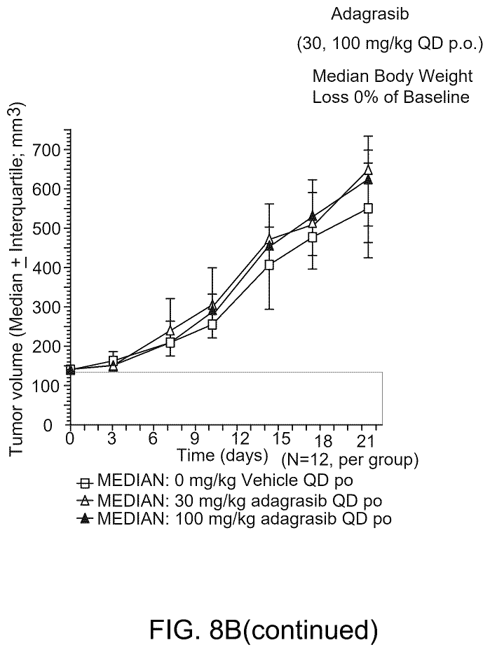

Compound A was evaluated head-to-head against sotorasib (AMG-510) and gemcitabine alone, and Compound A in combination with sotorasib, for 21 days in the KRAS G12C xenograft model (i.e., MIA PaCa-2 (KRAS) Pancreatic Xenograft Tumor Model in Athymic Nude Mice). Compound A as compared to sotorasib demonstrated tumor regression, both with insignificant BWL. Comparing Compound A alone, against sotorasib and in combination with sotorasib, tumor regressions with insignificant median BWL were observed (i.e., within 3% of baseline), which indicates activity, durability and tolerability of Compound A against a KRAS G12C mutant pancreatic cancer model. FIG. 8A shows data from the head-to-head Comparison of Compound A+/− sotorasib (AMG-510) using a MIA PaCa-2 Xenograft Tumor Model: Tumor Volume

Compound A was evaluated head-to-head against sotorasib (AMG-510) and adagrasib alone for 21 days in the Capan-2 (KRAS G12V) Pancreatic Xenograft Tumor Model in Athymic Nude Mice. As shown in FIG. 8B, Compound A demonstrated tumor regression as compared to sotorasib or adagrasib, with insignificant BWL.

In FIGS. 8A and 8B: Tumor Growth Inhibition (TGI) %=[1−(Ti−T₀)/(Ci−C₀)]×100; Expanded TGI formula vs. previous 1−[T/C]×100% method.

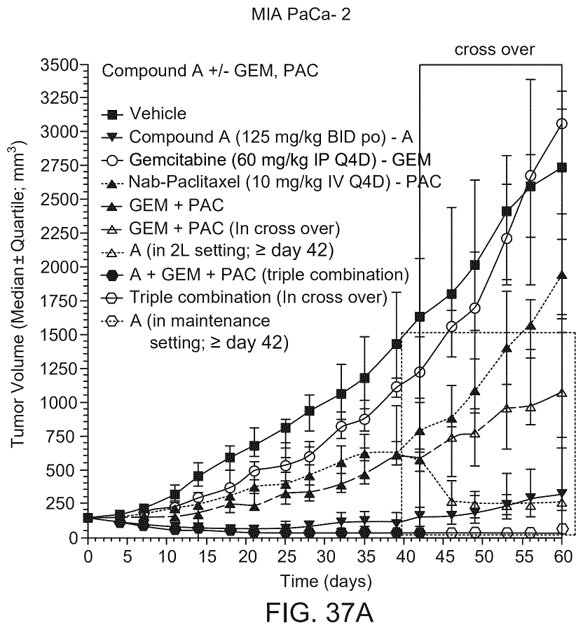

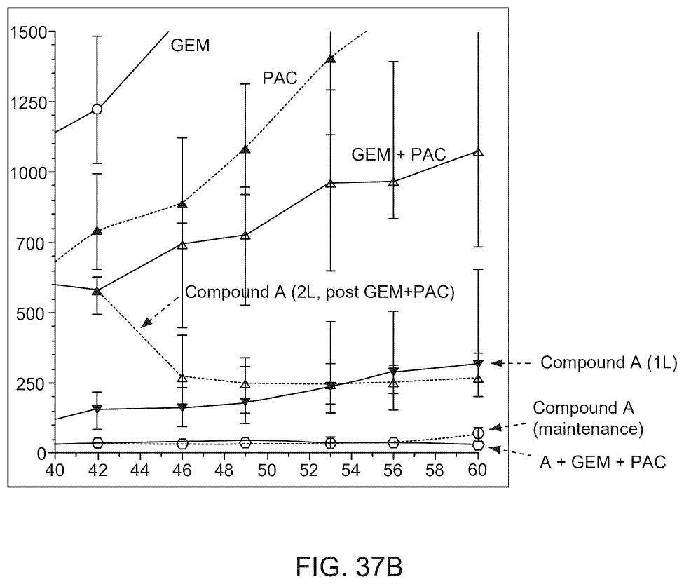

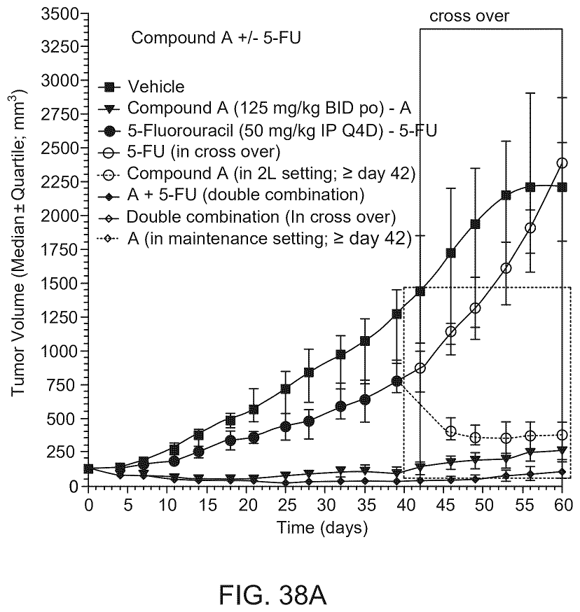

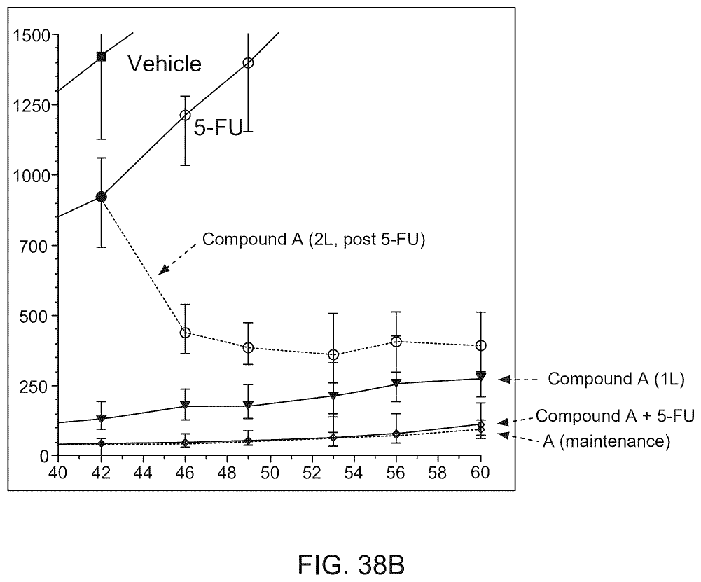

Compound A was evaluated in a MIA PaCa-2 Pancreatic (KRAS G12C) xenograft tumor models in athymic nude mice. Gemcitabine (antimetabolite), nab-paclitaxel (taxane) and fluorouracil (5-FU; antimetabolite) were commercially purchased. All studies started with 12 animals, per group. Mice within specific groups randomized for cross-over of treatments (≥day 42). Compound A+GEM (doublet combination) similar but slightly inferior to triple combination (A+GEM+PAC). FIG. 37A is a graph showing data obtained from an evaluation of Compound A (A), gemcitabine (GEM) or nab-paclitaxel (PAC) individually or in the specified combinations, as compared to vehicle using a MIA PaCa-2 KRAS G12C mutant xenograft tumor model in athymic nude mice (Tumor Volume). FIG. 37B is a graph showing a magnified portion of the graph in FIG. 37A, showing data obtained from an evaluation of Compound A (A), gemcitabine (GEM) or nab-paclitaxel (PAC) individually or in the specified combinations, as compared to vehicle using a MIA PaCa-2 KRAS G12C mutant xenograft tumor model in athymic nude mice (Tumor Volume). FIG. 38A is a graph showing data obtained from an evaluation of Compound A (A), and 5-fluorouracil (5-FU) individually or in combination, as compared to vehicle using a MIA PaCa-2 KRAS G12C mutant xenograft tumor model in athymic nude mice (Tumor Volume). FIG. 38B is a graph showing an magnified portion of the graph in FIG. 38A, showing data obtained from an evaluation of Compound A (A), and 5-fluorouracil (5-FU) individually or in combination, as compared to vehicle using a MIA PaCa-2 KRAS G12C mutant xenograft tumor model in athymic nude mice (Tumor Volume). The Bliss Combination Index (CI) and Synergy Score (SS) of Compound A was calculated in combination in vivo from above MIA PaCa-2 Tumor Xenograft Efficacy studies. The results are shown in Table 3A (Compound A in combination with 5-FU) and Table 3B (Compound A in combination with nab-paclitaxel and gemcitabine). The Twelve pancreatic tumor models were evaluated (3D-TGA model) in combinations of COMPOUND A with either gemcitabine, paclitaxel or 5-FU. 10 of 12 (83%) of the models showed additivity as assessed by ZIP synergy scores between −10 and +10 for each treatment. Additive models: MIA PaCa-2, PA-TU-8988S, CFPAC-1, HPAF-II, Panc 03.27, PSN-1, Panc 10.05, Capan-1, HPAC, PANC-1. Capan-2 and AsPC-1 failed to reach additive cutoffs for 5-FU alone.

TABLE 3A

| | AUC Combination | | AUC Synergy | | | |
|---|---|---|---|---|---|---|
| | Index (CI) +/− | | Score (SS) +/− | | | |
| | 95% Confid. Int. | | 95% Confid. Int. | | Synergy Call | |
| Comparison | Day 39 | Day 60 | Day 39 | Day 60 | Day 29 | Day 60 |
| 75 mg/kg PO | 0.30 | 0.34 | 8.60 | 2.50 | Synergistic | Additive |
| BID | [0.17-0.51] | [0.07-1.40] | [3.7-16.0] | [−1.00-6.40] | | |
| Compound A + | | | | | | |
| 50 mg/kg IP | | | | | | |
| Q4D 5-FU | | | | | | |
| 125 mg/kg PO | 0.30 | 0.47 | 3.00 | 0.41 | Synergistic | Additive |
| BID | [0.20-0.46] | [0.17-1.20] | [1.30-5.90] | [−0.09-1.20] | | |
| Compound A + | | | | | | |
| 50 mg/kg IP | | | | | | |
| Q4D 5-FU | | | | | | |

TABLE 3B

| | AUC Combination | | AUC Synergy | | | |
|---|---|---|---|---|---|---|
| | Index (CI) +/− | | Score (SS) +/− | | | |
| | 95% Confid. Int. | | 95% Confid. Int. | | Synergy Call | |
| Comparison | Day 39 | Day 60 | Day 39 | Day 60 | Day 29 | Day 60 |
| 60 mg/kg IP | 0.72 | 0.53 | 10.0 | 9.9 | A | A |
| Q4D GEM + | [0.43-1.10] | [0.16-1.30] | [−3.50-26.0] | [−4.60-26.0] | | |
| 10 mg/kg IV | | | | | | |
| Q4D PAC | | | | | | |
| 75 mg/kg PO | 0.21 | 0.28 | 12.0 | 4.00 | S | S |
| QD Cmpd A + | [0.14-0.30] | [0.14-0.57] | [7.30-19.0] | [1.40-8.50] | | |
| 60 mg/kg IP | | | | | | |
| Q4D GEM | | | | | | |
| 125 mg/kg PO | 0.18 | 0.12 | 4.50 | 1.00 | S | S |
| QD Cmpd A + | [0.12-0.27] | [0.042-0.36] | [2.60-7.70] | [0.39-2.10] | | |
| 60 mg/kg IP | | | | | | |
| Q4D GEM | | | | | | |
| 75 mg/kg PO | 0.23 | 0.22 | 4.40 | 0.83 | S | S |
| QD Cmpd A + | [0.14-0.39] | [0.06-0.96] | [2.00-8.40] | [0.02-2.80] | | |
| 60 mg/kg IP | | | | | | |
| Q4D GEM + | | | | | | |
| 10 mg/kg IV | | | | | | |
| Q4D PAC | | | | | | |
| 125 mg/kg PO | 0.41 | 0.83 | 1.20 | 0.04 | S | A |
| QD Cmpd A + | [0.21-0.77] | [0.21-3.60] | [0.28-2.90] | [−0.21-0.47] | | |
| 60 mg/kg IP | | | | | | |
| Q4D GEM + | | | | | | |
| 10 mg/kg IV | | | | | | |
| Q4D PAC | | | | | | |

GEM = gemcitabine;
PAC = nab-paclitaxel;
Cmpd A = Compound A
S = Synergistic;
A = Additive

### Example 8B

### Treatment-Acquired Mutations in MIA PaCa-2 (KRAS G12C)

### Pancreatic Cancer Xenograft Tumor Models

An analysis of treatment-acquired mutations in was performed in the mice treated with Compound A, 5-fluorouracil, gemcitabine and nab-paclitaxel.

DNA analysis and mutational enrichment analysis was performed. Tumor extracted from xenograft-induced mice was run through DNA isolation, library preparation, and Illumina exome sequencing via Crown Bio as previously described.

Then, a bioinformatics analysis was performed.

For whole exome sequencing data processing, the following workflow was implemented on DNAnexus. First, separation of reads into human and mouse sequence was conducted BBSplit (Bushnell B), where human reads were retained for subsequent analysis. Next, adaptor trimming was performed via BBDuk (Bushnell B). The adaptor-cleaved reads were then assessed via quality control method FastQC. Alignment was conducted on the adaptor-trimmed reads via BWA (Li 2013). Duplicate marking and base quality score recalibration was achieved via GATK (Poplin 2017), and single-sample somatic variant calling via MuTect2 (GATK package and using both a provided panel of normal samples and gnomAD from the GATK resource bundle) following the best-practices workflow. CNVkit (Talevich 2016) was used in a tumor-only fashion for copy-number calling.

For tertiary analysis of the mutation data, the resultant VCFs generated via MuTect2 were processed via the VCF2MAF pipeline (Kandoth 2020). The resultant MAF files were processed such that variants with a filter status of “PASS”, “clustered_events”, and “haplotype” were accepted. For mutation evaluation, if the mutation occurred in BRAF, it was compared to a literature-assessed list for classification into known Class 1, Class 2, or Class 3 mutations, it was considered damaging; if the mutation was in RAF1, nonsynonymous, and the affected locus was in either amino acids S257 or S2579, it was considered damaging; if the mutation was in ARAF and was a nonsynonymous mutation of S214, it was considered damaging; if the mutation was in KRAS, HRAS, or NRAS and a nonsynonymous substitution occurred in a known pathogenic residue (G12, G13, A59, G60, Q61, K117, A146), it was considered damaging. For all other genes, a mutation was considered damaging if it was in a known cancer hotspot, was annotated containing the description deleterious via SIFT, high impact variant consequence from Ensembl, present in a pfam or PANTHER domain, or was annotated containing the description “pathogenic” via CLIN_SIG (clinically significant annotation from dbSNP). Mutations were filtered by presence in at least one public patient dataset (GENIE, TCGA) and at least one public cell line dataset (CCLE, DepMap, SCMP).

A computational biology assessment was performed. Mutations were categorized by treatment class. Mice left untreated or treated with a vehicle served as the background group. Specific point mutations identified in these mice were excluded from subsequent analysis. To identify selection pressure, the remaining mutations were tallied, ensuring each mutated gene was counted only once per mouse (e.g., mutations in the same gene, YFG, such as point mutations c.A123C and c.T124C from a single mouse, were counted only once). The effect of drug selection was determined by tallying the frequency of gene mutations within each treatment group. The treatment groups were as follows:
Compound A: 75 treated with Compound A and 125 treated with Compound A
Gemcitabine (GEM): 60 treated with Gemcitabine and 60 treated with Gemcitabine+10 Nab-paclitaxel
Nab-paclitaxel (PAC): 10 treated with Nab-paclitaxel and 10 treated with Nab-paclitaxel+60 Gemcitabine
5-fluorouracil (5-FU): 50 treated with 5-Fluorouracil
Compound A+GEM+PAC: 75 treated with Compound A+60 Gemcitabine+10 Nab-paclitaxel and 125 treated with Compound A+60 Gemcitabine+10 Nab-paclitaxel
Compound A+5FU: 75 treated with Compound A+50 5-FU and 125 treated with Compound A+50 5-FU

To identify the most frequently mutated genes, thresholds were applied to each treatment group to capture mutations in at least 65 genes per group. These genes were further analyzed using Enrichr (Xie 2021), employing the Transcriptional Protein Hubs gene set. Volcano plots were generated, with the y-axis representing −log 10(adjusted p-value) and the x-axis representing the odds ratio.

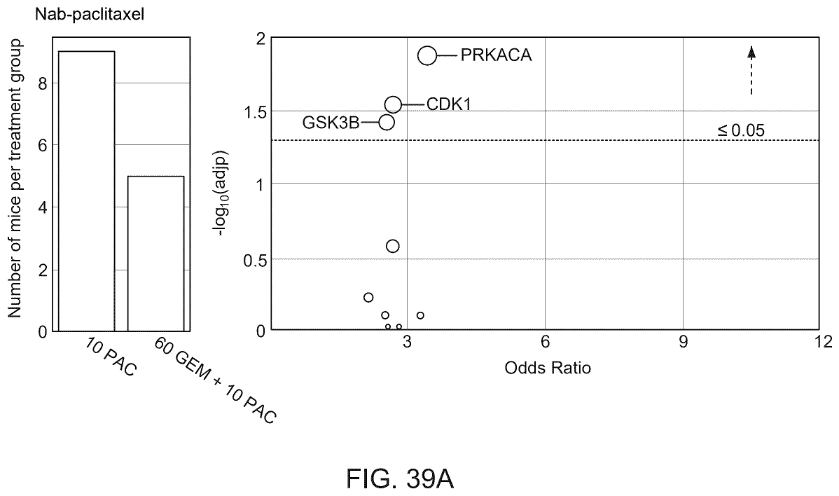

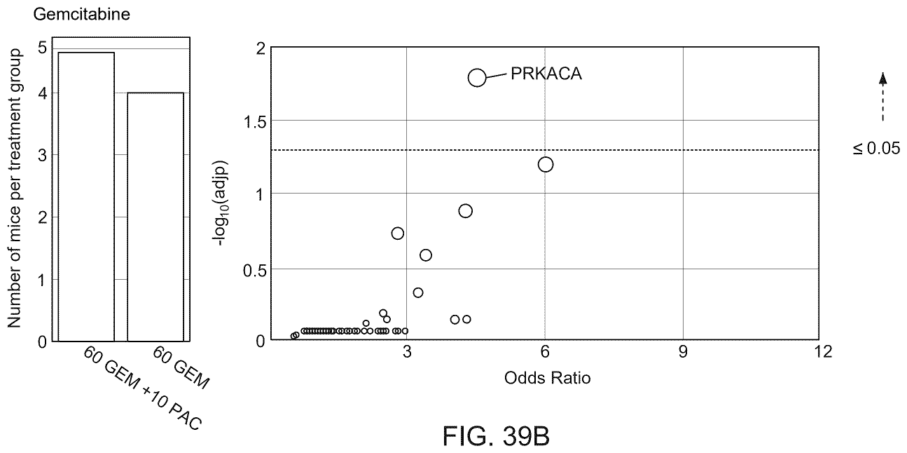

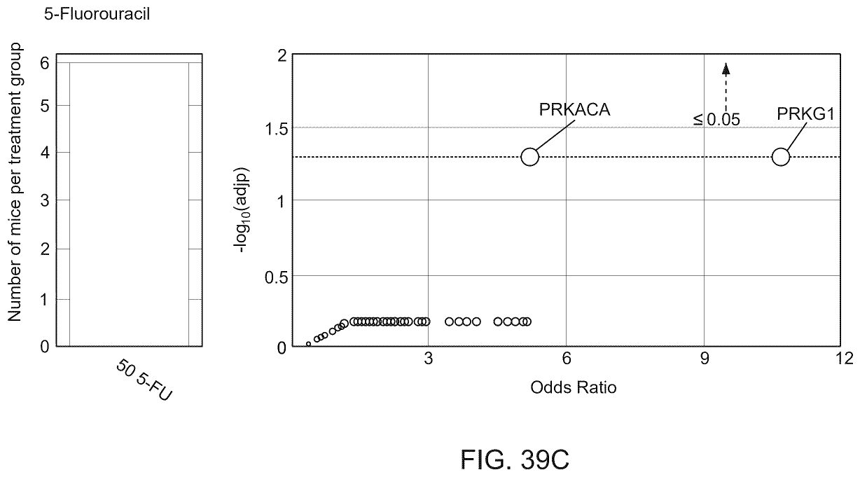

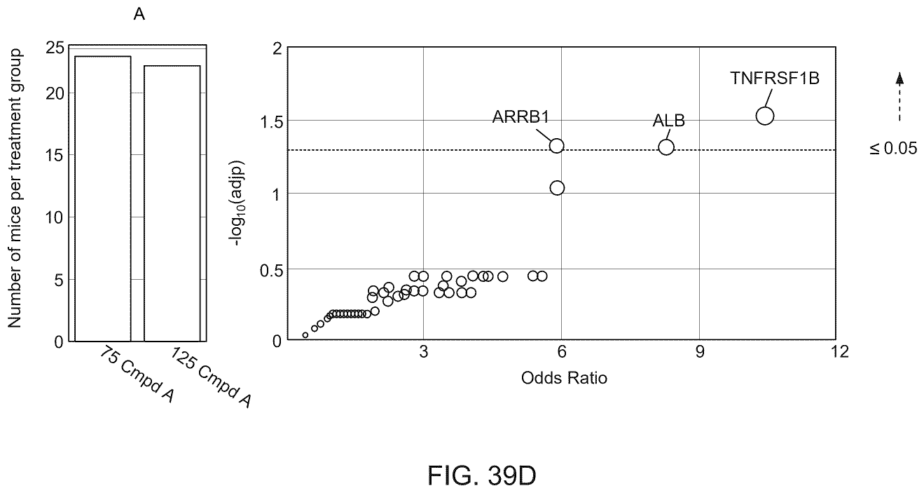

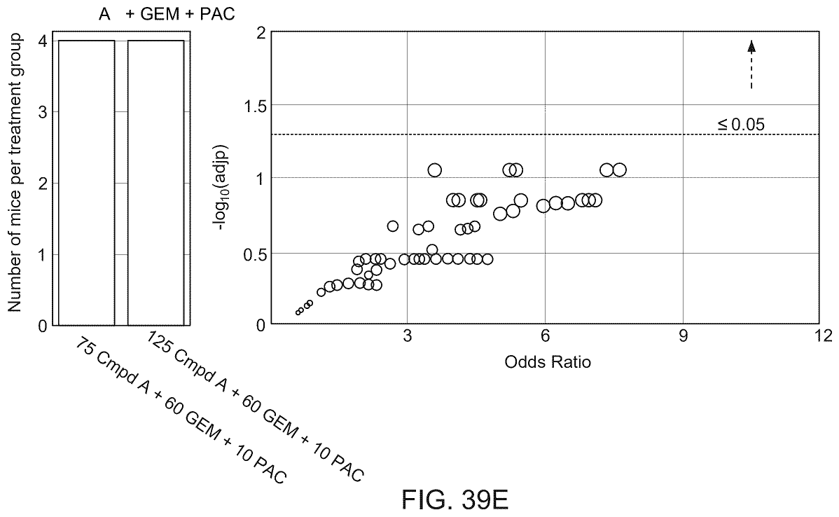

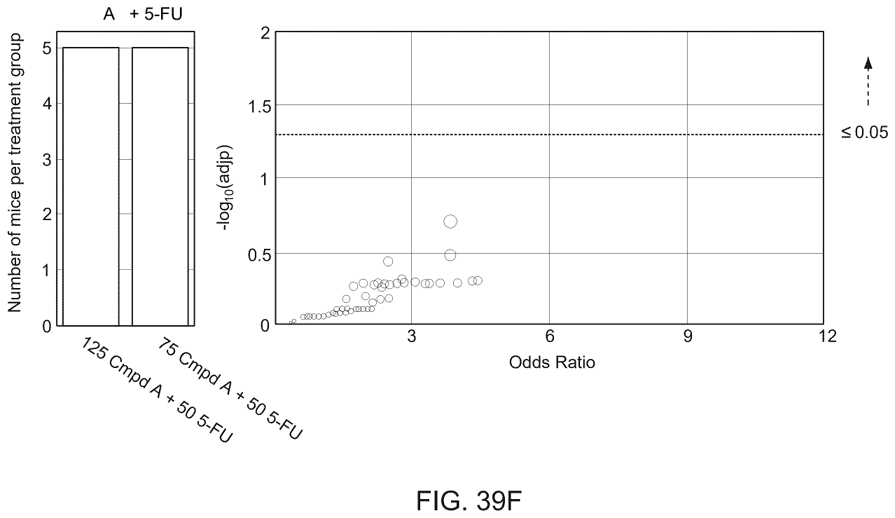

Results are shown in FIGS. 39A-39F. FIGS. 39A-F are graphs showing the results form an enrichment analysis of recurrently-mutated genes under continuous treatment in protein-protein interaction hubs for nab-paclitaxel (FIG. 39A), gemcitabine (FIG. 39B), 5-fluorouracil (FIG. 39C), Compound A (FIG. 39D), a combination of Compound A, gemcitabine and nab-paclitaxel (FIG. 39E) and a combination of Compound A and 5-fluorouracil (FIG. 39F), showing the number of mice per treatment group and a scatter plot of the corresponding odds ratio. These data show that PRKACA activates MAPK signaling via RAF; with 5-FU treatment or combination therapy using Nab-paclitaxel+Gemcitabine, tumors exhibit increased reliance on MAPK for survival and proliferation. Notably, this enrichment is absent in Compound A monotherapy or combinations. CDK1 mediates paclitaxel resistance, while GSK3B acts as a compensatory mechanism in paclitaxel treatment; co-treatment with a GSK3 inhibitor enhances paclitaxel efficacy. PRKG1 gene set itself is likely not a driver of resistance, but its component gene BRAF signals through MAPK. TNFRSF1B regulates PI3K-Akt, a MAPK-independent pro-survival pathway. ARRB1 facilitates ERK auto-phosphorylation in the absence of MEK.7

While pancreatic tumors display near universal activation of the RAS/MAPK pathway, not all tumors are addicted to this pathway alone. A goal of combination therapy is to drive deeper, more durable antitumor responses, and Compound A showed promising combination effects with GEM, PAC and 5-FU in multiple 3D-TGA pancreatic cancer models. Further, Compound A alone showed greater tumor growth inhibition (TGI) head-to-head versus single or combination chemotherapy in MIA PaCa-2. Combinations of Compound A plus standard-of-care chemotherapies demonstrated synergy in vivo and resulted in near complete responses in a majority of animals.

The ongoing Phase 2a clinical trial includes five arms, three of which focus on pancreatic cancer, where Compound A is being evaluated as both monotherapy and in combination with approved GEM and 5-FU containing chemotherapy combinations. These new in vitro, in vivo and ML modeling data further support an advancing translational roadmap for Compound A in pancreatic cancer.

### Example 9

### Compound A in SK-MEL-2 (NRAS Q61R) Melanoma Xenograft Tumor Model

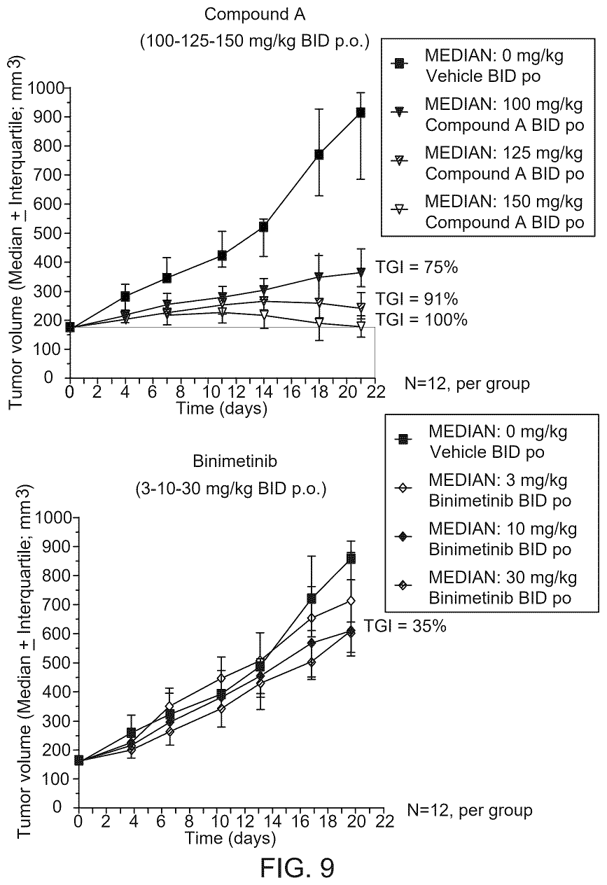

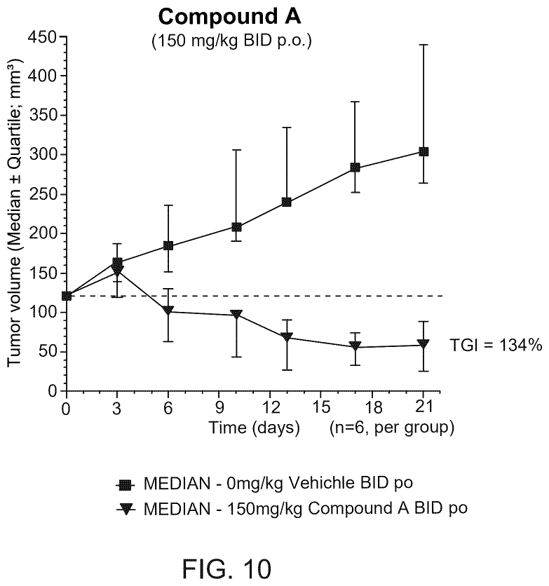

Compound A was evaluated as a monotherapy as compared to binimetinib for 21 days in the NRASQ61R mutant tumor model (i.e., SK-MEL-2). Superior tumor growth inhibition was observed relative to binimetinib. FIG. 9 shows results from a head-to-head comparison of tumor volume measured with Compound A against binimetinib in a NRAS-Q61R SK-MEL-2 melanoma xenograft study. Compound A as compared to binimetinib monotherapy demonstrated greater tumor growth inhibition (TGI), as shown in FIG. 9. Midcycle tumor regressions in mice treated with Compound A was observed, which demonstrated activity and durability of Compound A against an NRAS Q61R mutant melanoma cancer model (FIG. 10).

FIG. 10 is a graph showing data obtained from an evaluation of Compound A as compared to vehicle using a SK-MEL-2 xenograft tumor model (Tumor Volume).

A Capan-2 PDAC xenograft animal model was utilized to evaluate single agent activity of Compound A (100, 150 mg/kg BID p.o.) vs. sotorasib and adagrasib (30 and 100 mg/kg QD p.o. each) for 21 days treatment after tumors had reached volumes of 150 to 200 mm³. A head-to-head comparison in vivo demonstrated no Tumor Growth Inhibition (TGI) by sotorasib or adagrasib in KRAS-G12V mutant Capan-2 PDAC tumors, while Compound A prompted TGIs up to 84% at 150 mg/kg BID po.

### Example 10

### Humanized 3D Tumor Models

The humanized 3D tumor growth assay (3D-TGA) is an extracellular matrix (ECM)-based, 3D tumor model that more faithfully mimics the human tumor microenvironment (TME) with the goal of improving translational fidelity to patients. See Casneuf T, et al., “Interleukin-6 is a potential therapeutic target in interleukin-6 dependent, estrogen receptor-α-positive breast cancer,” Breast Cancer (Dove Med Press). 2016; 8:13-27; Onion D, et al. “3-Dimensional Patient-Derived Lung Cancer Assays Reveal Resistance to Standards-of-Care Promoted by Stromal Cells but Sensitivity to Histone Deacetylase Inhibitors,” Mol Cancer Ther. 2016 April; 15(4):753-63; and Sasser A K, et al., “Interleukin-6 is a potent growth factor for ER-alpha-positive human breast cancer,” FASEB J. 2007 Nov.; 21(13):3763-70, each incorporated herein by reference in their entirety. Disruption of the MAPK pathway has specifically demonstrated that 3D models more accurately predict certain aspects of in vivo tumor biology, especially when evaluating molecular activities of drug candidates that target the MAPK pathway (See Janes M R, et al., “Targeting KRAS Mutant Cancers with a Covalent G12C-Specific Inhibitor,” Cell, 2018 Jan. 25; 172(3):578-589.e17, incorporated herein by reference in its entirety). MEK inhibition in the 3D-TGA. MEK inhibition in the 3D-TGA is useful to compare tumor addiction levels to the MAPK pathway across many models and establish response biomarkers for Compound A.

Novel dual-MEK inhibitor Compound A is under clinical investigation for use in RAS-addicted solid tumors. Approved KRAS G12C inhibitors are available but cover a limited subset of patients. For example, the KRAS G12C substitution occurs in only 1-3% of pancreatic cancers. The response to Compound A was assessed across RAS mutant preclinical models to determine whether a preference was observed for mutation position, or for specific amino acid substitutions.

Response to Compound A was measured in a humanized 3D tumor growth assay across 132 tumor models. A subset (N=107) of the 132 models were “RAS Mutant” (including H/K/N isoforms). Seventy-five of these models have a reported RAS mutation, and all models are being mutationally profiled by whole exome sequencing, with the majority (~85%) completed to date. The RAS-mutant panel spans 12 tissue types and includes a subset of 30 confirmed KRAS G12 mutated cell lines drawn from three major indications: 12 pancreatic, 11 lung, and 7 colorectal cancer models. Based on the 3D assay, cell lines were classified into sensitive (IC50 less than 1 micromolar), intermediate (IC50 of at least 1 micromolar and greater than 25% reduction), and otherwise resistant to Compound A. The distribution of responses was then assessed across mutation position and amino acid substitutions. MAPK Normal additionally excluded models with BRAF (class 1/11) and GNAQ/GNA11 mutations. Patient-aligned tumor models represent models where the mutational profile mapped to the most frequent 95% of GENIE v 13.0 patients of the same indication.

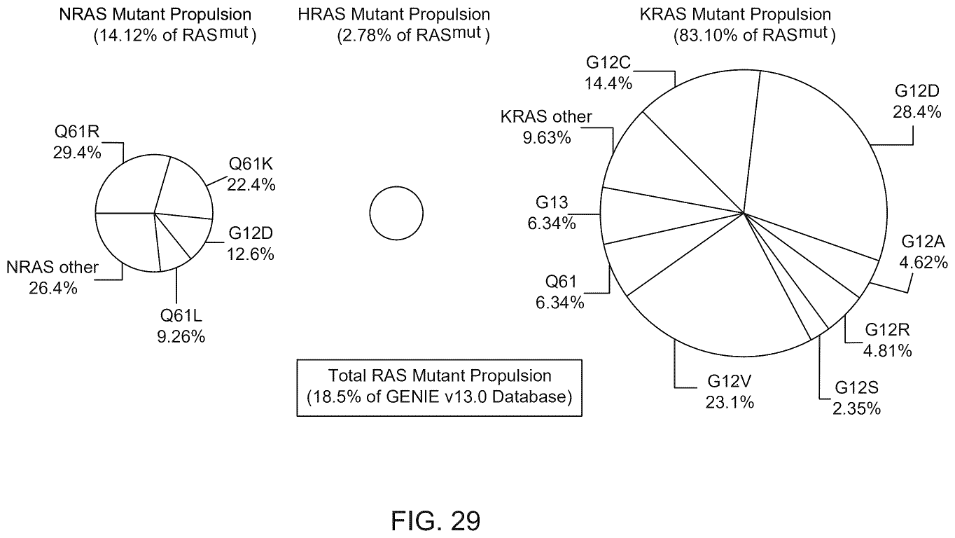

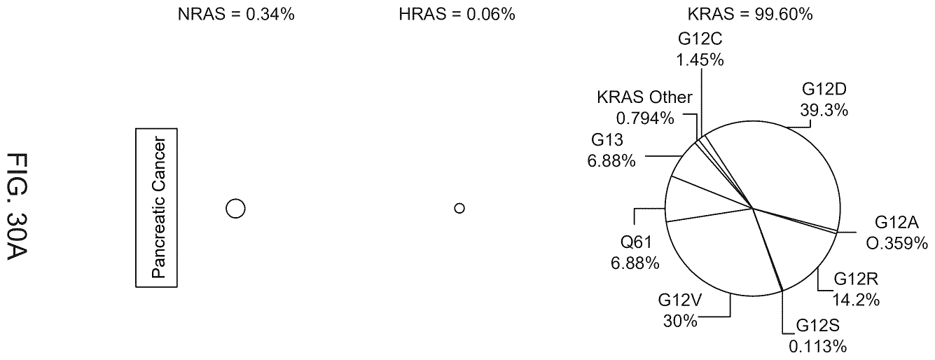

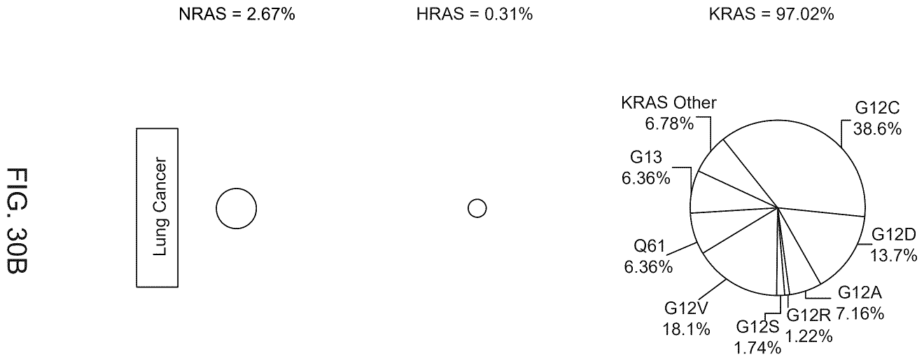

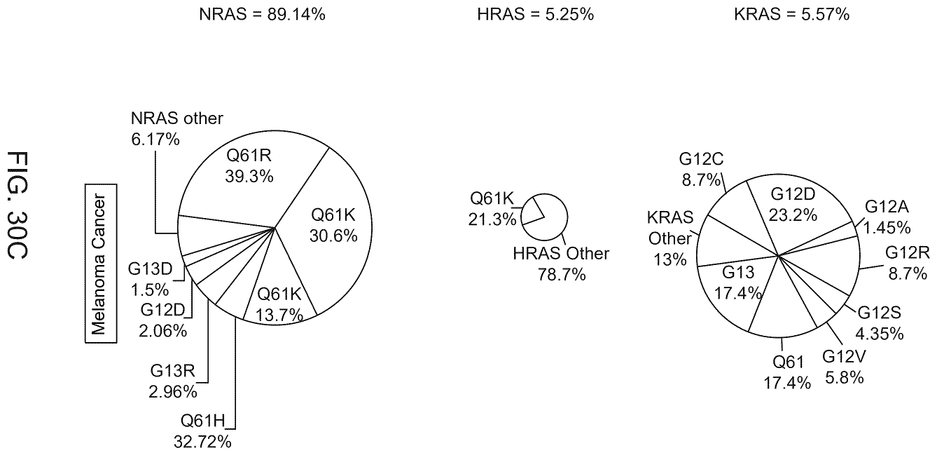

Across all RAS-mutant models, at least one model displayed response to Compound A for each observed mutation in K/N/HRAS. That is, no particular mutation position or amino acid substitution was exclusively found to confer resistance to drug exposure. FIG. 29 shows graphs representing the distribution of RAS mutations across patients in the GENIE database for NRAS mutant population (about 14% of the RAS mutations observed), the HRAS mutant population (about 3% of the RAS mutations observed), and the KRAS mutant population (about 83% of the RAS mutations observed).

Association of response with amino acid identity was further evaluated in a subset of lines for the most frequently altered residue in KRAS, G12. A distribution of responses was observed for each amino acid substitution. The G12 substitutions having at least four cell lines each total across the three indications included G12C (8 lines), D (5 lines), R (4 lines), and V (11 lines). Across cell lines for each of these substitutions, multiple response categories were observed. In each case, half or more lines fell into the intermediate category with the rest falling into one or both of the other response categories. For example, out of the 8 KRAS G12C lines, 6 showed intermediate response, 1 showed resistance, and 1 showed sensitivity. Examining these distributions together, no significant statistical relationship was seen between the amino acid substitution and response categories by Fisher's exact test.

Across 75 RAS-mutated cell lines, each mutation position or amino acid substitution was associated with sensitive or intermediate response in at least one line. Looking at the most commonly mutated position in KRAS, across 28 KRAS G12 mutated cell lines from three cancer indications, no significant preference was observed with respect to response to Compound A for a particular amino acid at G12. These observations suggest potential relevance of Compound A to a broad RAS-driven patient population.

Compound A, with Universal-RAS activity through Deep Cyclic Inhibition (DCI) of MEK, was evaluated in humanized 3D preclinical tumor models displaying diverse MAPK pathway activation events. Based on drug sensitivity and resistance profiles, a biomarker signature for Compound A was developed in order to project potential therapeutic response of cancer patients found in the AACR Project GENIE database.

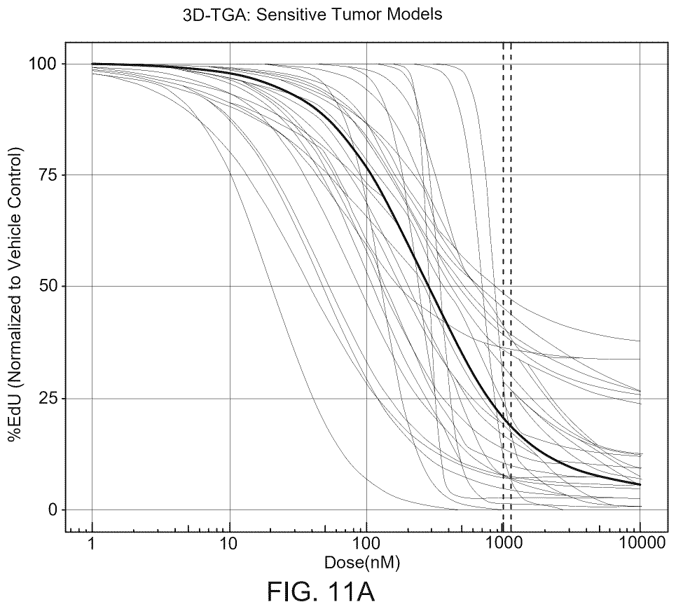

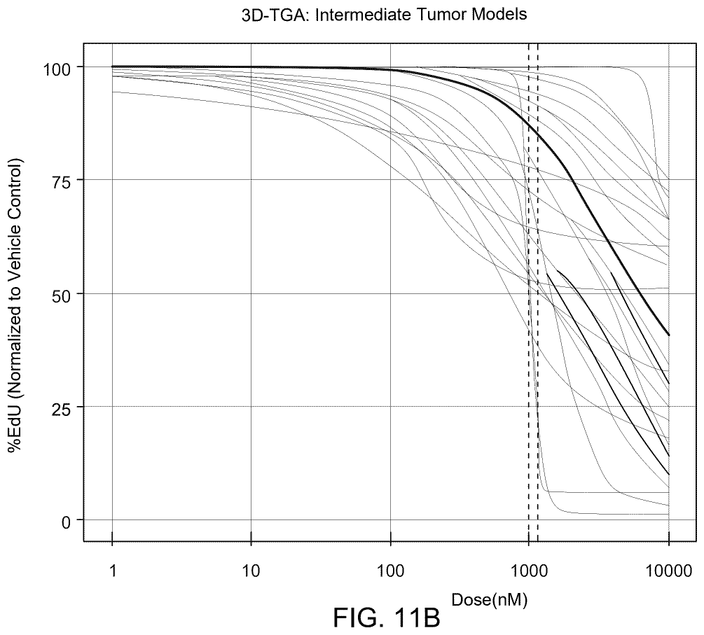

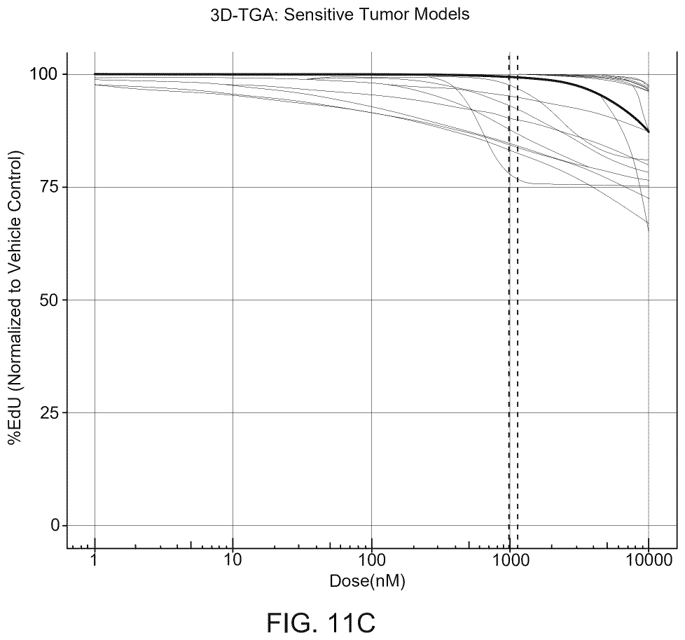

Humanized 3D preclinical models better predict in vivo tumor responses versus 2D culture and more accurately replicate biology of human tumors. Therefore, the antitumor activity of Compound A was evaluated in over 132 tumor models spanning 12 distinct tumor types in the humanized 3D tumor growth assay (3D-TGA) (see, FIGS. 11A-11C). Cell-based whole exome sequencing (WES) readouts were combined with 3D-TGA results to build a pharmacogenomic response algorithm. When applied to the GENIE patient database, resultant tumor-specific response landscapes helped to inform an early Universal-RAS clinical trial design for Compound A (see, FIGS. 11A-11C).

Cell lines tested in 3D-TGA (N=132) were assigned response of sensitive (IC50<1 μM), intermediate (IC50≥1 and >25% reduction at 10 μM), and resistant otherwise The dark line on each plot represents the median of the individual curves; Dotted vertical lines match Cmax Compound A drug free-fractions attained in patients 3 and 4 at 160 mg QD p.o.

For 122 of the 3D-TGA cell lines where in-house WES data were also collected, the upper plateau-to-1 uM difference was used to delineate responder from non-responder, and thus train a logistic regression model with 10-fold cross validation. There were 28 features used, one comprised of a consolidated MAPK mutations status (mutations in K/H/NRAS, GNAQ/11, and BRAF class I/II), and 27 other genes representing key pathways. With an AUC of 0.84 on the training cohort and 0.72 on test cohort, the model was then applied to GENIE cohorts (see, FIGS. 11A-11C) where all features were included on the testing panel. For each indication of interest, a kernel density plot of the probability of predicted response for the entire cohort (projected onto the negative y-axis), and the MAPK(+) subset (positive y-axis) shows enrichment for higher probability of response with restriction to MAPK alteration status. FIGS. 11A, 11B and 11C show 3D-TGA Compound A Dose Responses (N=132 Models; 12 Tumor Indications).

The depth of response to Compound A was evaluated across a panel of diverse 3D-TGA tumor models and led to identification of a biomarker signature for therapeutically addressable MAPK pathway addiction. To translate these findings into a relevant clinical application, a response algorithm was developed and applied to the GENIE database, which has cataloged the molecular profiles of over 100,000 cancer patients. Mutational landscapes of patients within GENIE helped identify preclinical models that better represent patient profiles likely to be encountered in the clinic. This approach could, as a general principle, be applied as a tool for improving biomarker discovery and clinical translation of oncology drugs.

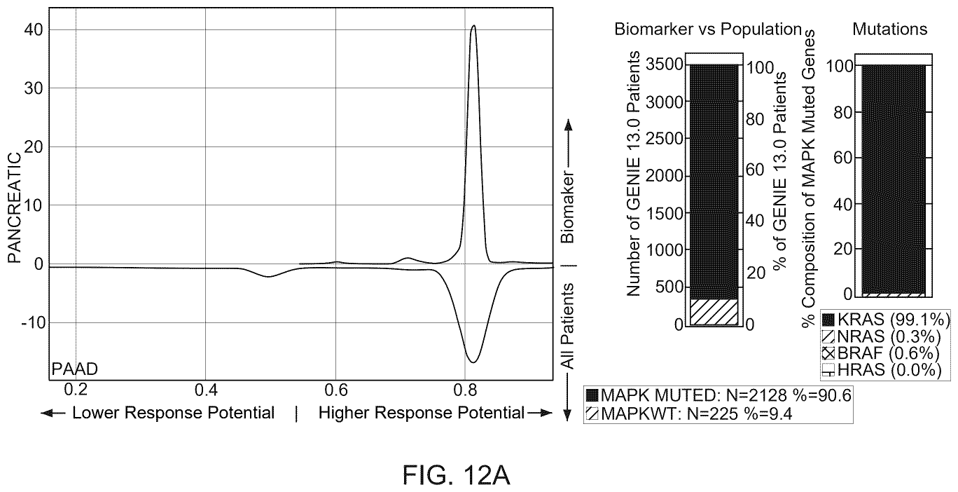

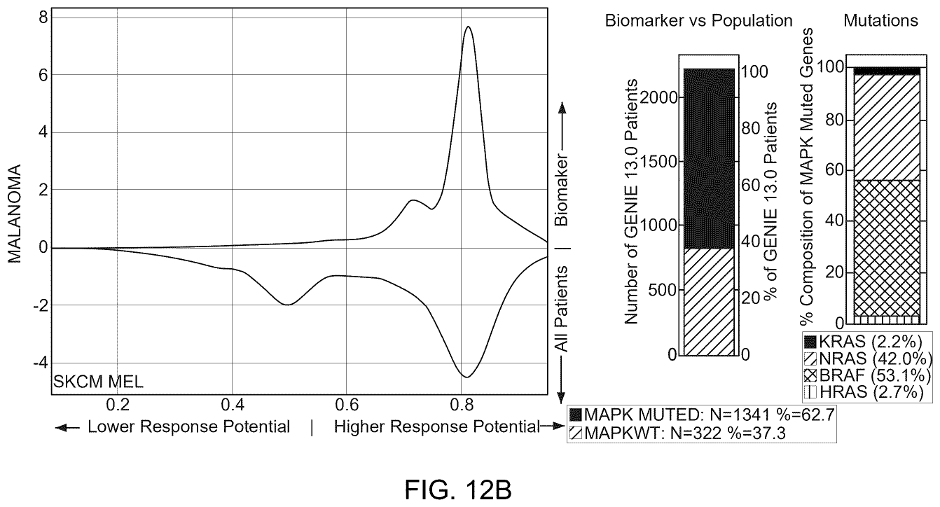

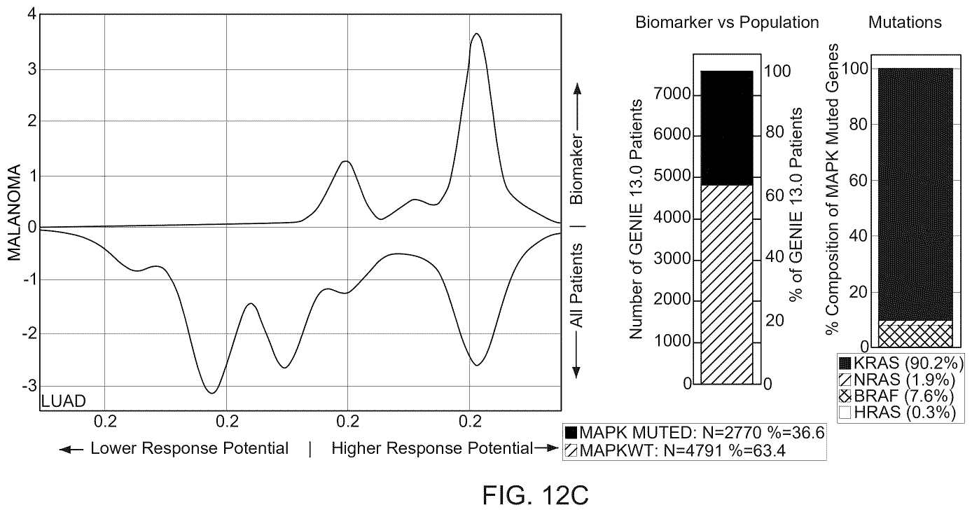

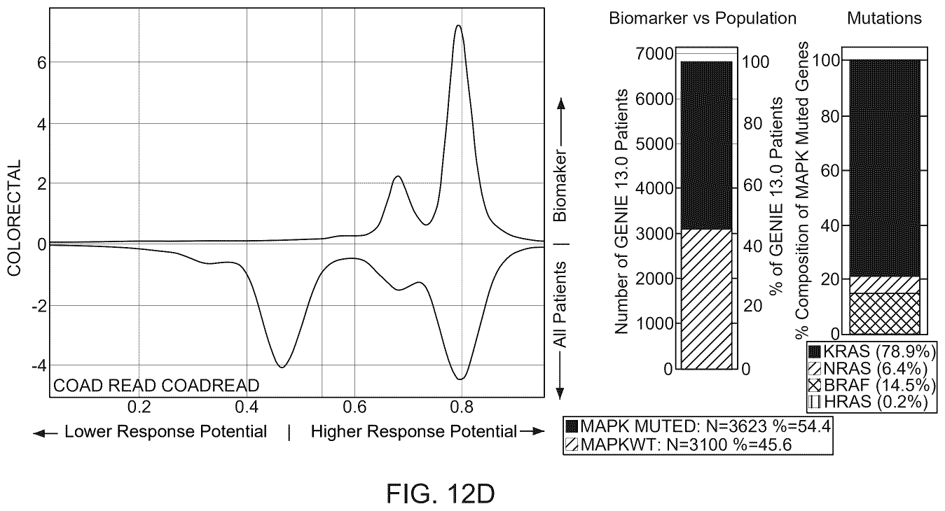

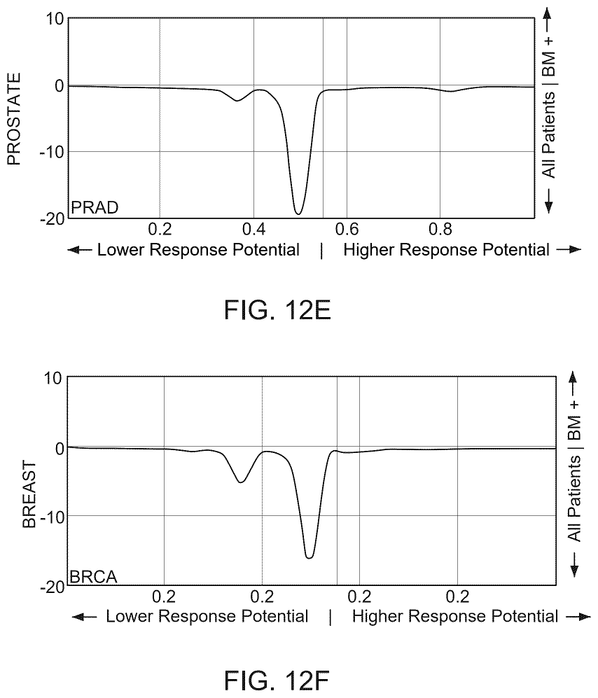

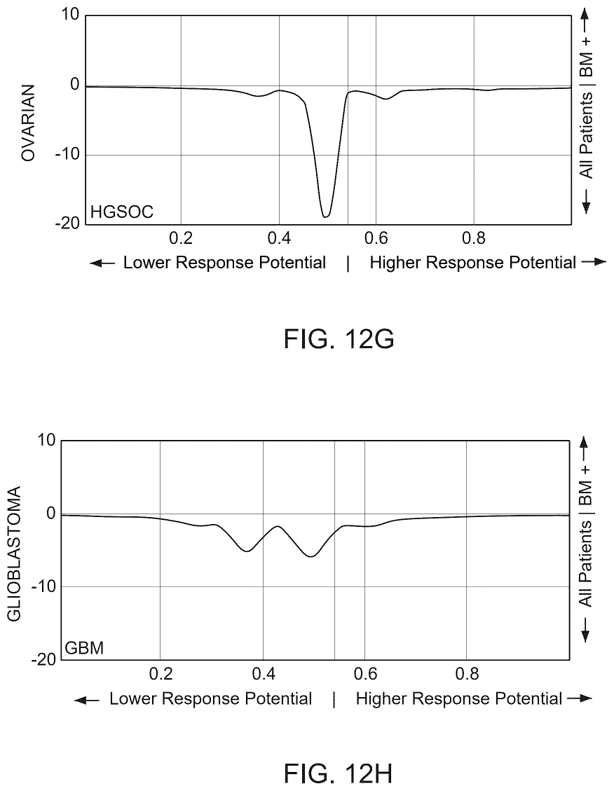

FIGS. 12A-12H show mapping of biomarker sensitive patient populations (GENIE) (MAPK+=GENIE patients per indication with activation mutation in KRAS, NRAS, HRAS, BRAF (class I or II)).

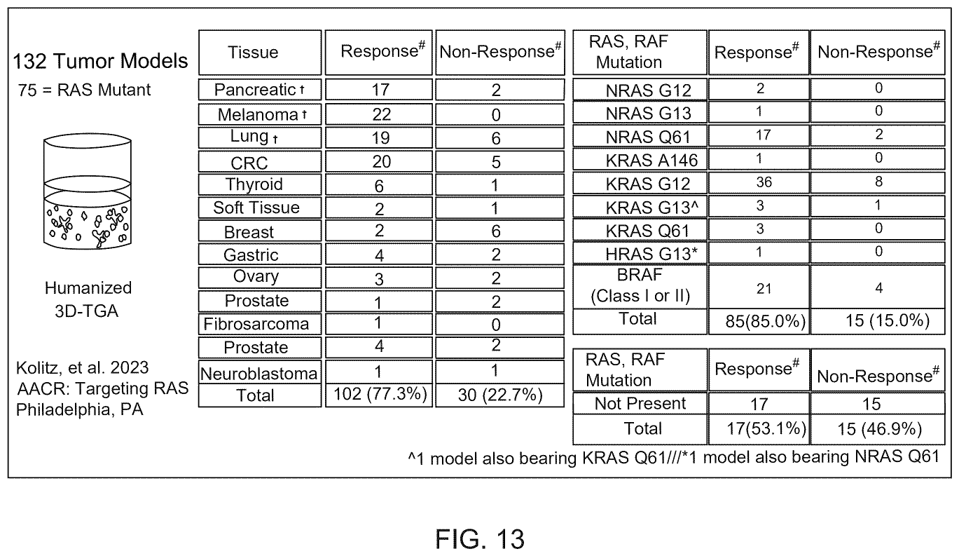

FIG. 13 shows tables of data showing the response to Compound A based on 3D-TGA and other preclinical modeling. Across all RAS-mutant models tested (132 tumor models, 75 of which have a reported RAS mutation), at least one model displayed response to Compound A for each observed RAS mutation, regardless of mutation position or amino acid substitution. No significant preference was observed with respect to response to Compound A across 30 KRAS G12 mutated cell lines, the most commonly mutated position in KRAS, from three major cancer indications including pancreatic, lung and colorectal cancer models.

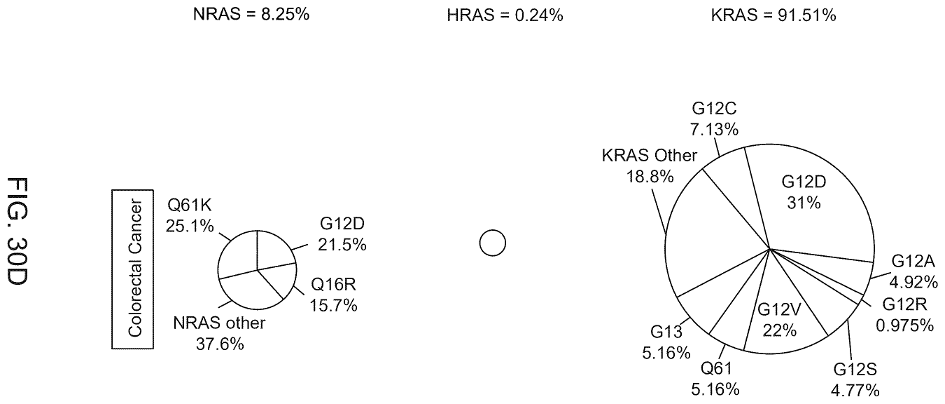

FIGS. 30A, 30B, 30C and 30D are graphs showing the mutation profiles across indications in the GENIE patient database for pancreatic cancer cells with RAS mutations. FIG. 30A shows graphs of mutation profiles representing the distribution of RAS mutations across indications in the GENIE patient database for pancreatic cancer cells with NRAS (less than 1%), HRAS (less than 1%) and KRAS (greater than 99%). FIG. 30B shows graphs of mutation profiles representing the distribution of RAS mutations across indications in the GENIE patient database for lung cancer cells with NRAS (about 3%), HRAS (less than 1%) and KRAS (greater than 97%). FIG. 30C shows graphs of mutation profiles representing the distribution of RAS mutations across indications in the GENIE patient database for melanoma cancer cells with NRAS (about 89%), HRAS (about 5%) and KRAS (about 6%). FIG. 30D shows graphs of mutation profiles representing the distribution of RAS mutations across indications in the GENIE patient database for melanoma cancer cells with NRAS (about 8%), HRAS (less than 1%) and KRAS (about 92%).

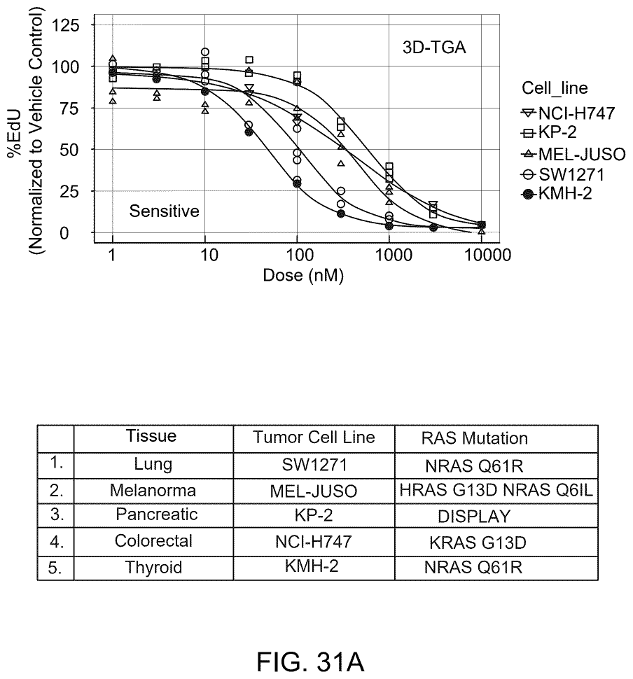

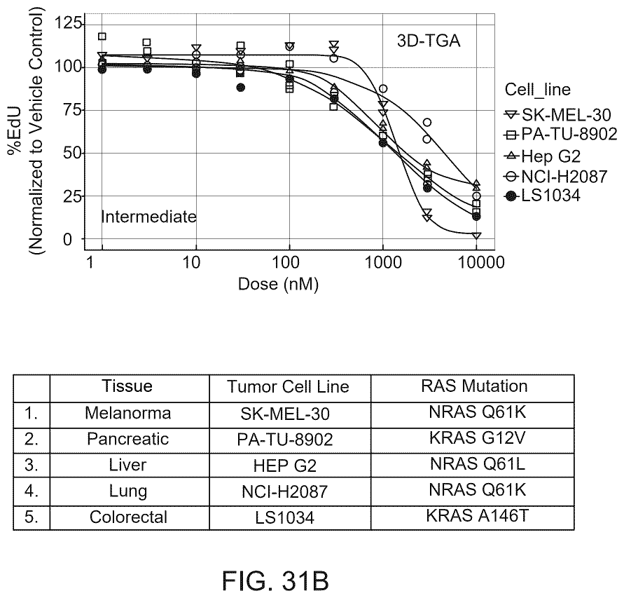

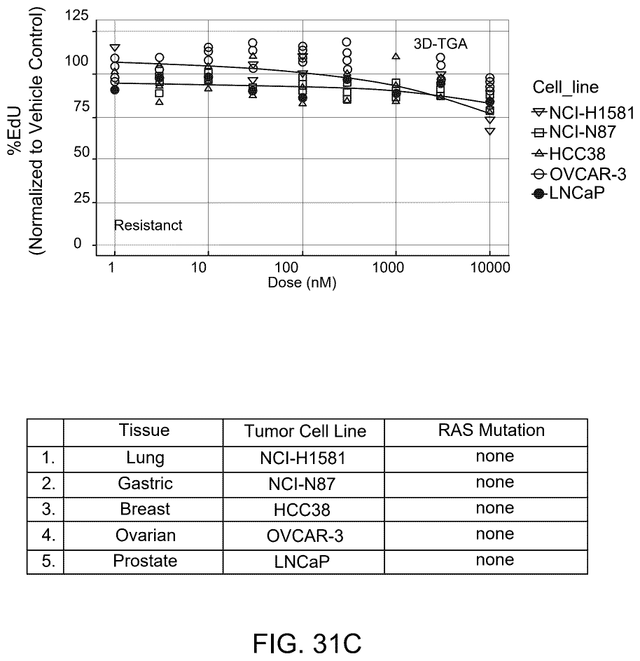

FIGS. 31A, 31B, and 31C show dose-response curves obtained with Compound A in the 3D-TGA model. Cell lines tested in 3D-TGA were assigned response of sensitive (IC50<1 μM), intermediate (IC50≥1 and >25% reduction at 10 μM), and resistant otherwise (3D-TGA). FIG. 31A shows dose-response curves for Compound A in a representative subset of susceptible 132 3d-TGA tumor models (lung SW1271, melanoma MEL-JUSO, pancreatic KP-2, colorectal NCI-H747 and thyroid KMH-2) that were sensitive to Compound A and listed in the corresponding Table below the graph in FIG. 31A. FIG. 31B shows dose-response curves for Compound A in a representative subset of intermediate 132 3d-TGA tumor models (melanoma SK-MEL-30, pancreatic PA-TU-8902, liver HEP G2, lung NCi-H2087 and colorectal LS1034) that exhibited an intermediate sensitivity to Compound A and listed in the corresponding Table below the graph in FIG. 31B. FIG. 31C shows dose-response curves for Compound A in a representative subset of resistant 132 3d-TGA tumor models (lung NCI-H1581, gastric NCI-N87, breast HCC-38, ovarian OVCAR-3 and prostate LNCaP) that exhibited an resistance to Compound A and listed in the corresponding Table below the graph in FIG. 31C.

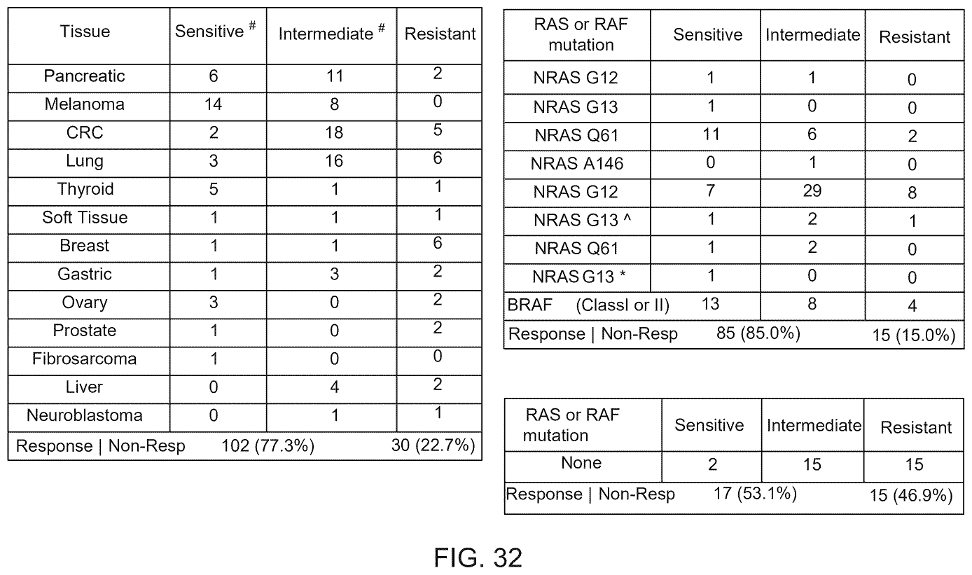

FIG. 32 is a table summarizing the tumor tissue type and RAS mutation status for the humanized 3D tumor growth assay, based on response to Compound A (sensitive, intermediate or resistant).

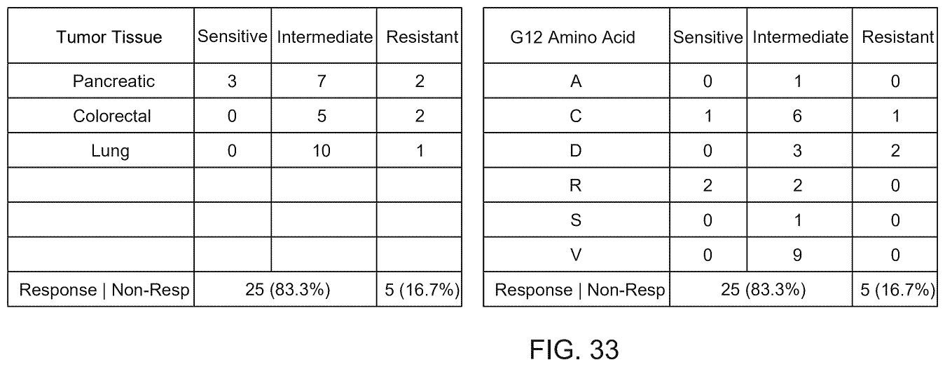

FIG. 33 is a table showing the subset of KRAS G12 variant and associated 3d-TGA response by tumor tissue type and G12 mutation amino acid mutation type.

Compound A was evaluated in 9-point semi-log₁₀ dose response curves from 1 nM to 10 μM across all tumor models in the 3D-TGA. Each model was classified according to the drug dose required to reduce cell division by at least 50% (IC₅₀) as quantified by reduction of tumor cell 5-ethynyl-2′-deoxyuridine (EdU) incorporation. Models were considered sensitive if IC₅₀<1 μM, intermediate if 1-10 μM or at least 25% inhibition at 10 μM, and resistant otherwise. Each category boundary was established based on attainable in vivo Cmax plasma drug free fractions for Compound A and depth of tumor cell growth impairment.

Tumor models displaying activation mutations in KRAS, NRAS, HRAS, BRAF and certain other MAPK pathway activation events were found to be most sensitive to single agent Compound A. There were 40 putative response biomarkers identified, most of which are known to activate the MAPK pathway upstream of MEK or represent parallel pathways that could confer resistance.

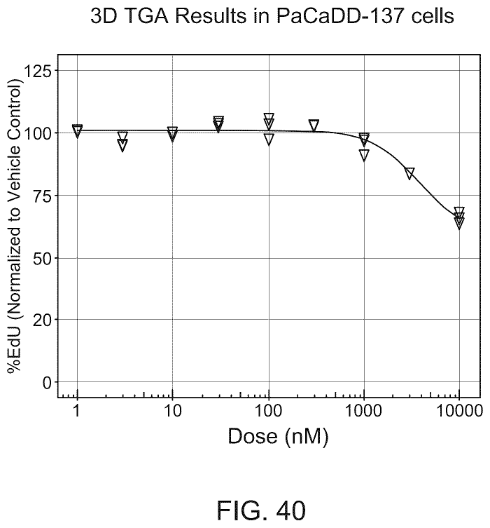

FIG. 40 is a graph showing 3D-TGA Compound A dose responses calculated for PaCaDD-137 cells. The EC50 was calculated as about 3.7 micromolar, and the IC50 was greater than 10 micromolar.

### Example 11

### Oral Dosage Form of Compound A

An oral dosage form of Compound A can be prepared by combining Compound A as the active pharmaceutical ingredient (API) with pharmaceutically acceptable excipients to form a tablet. The intra granulation excipients can include microcrystalline cellulose (e.g., CEOLUS UF-711), crospovidone Type A (e.g., Polyplasdone XL), hydroxypropyl Cellulose HPC-SL, hyroxypropyl cellulose-Low Substituted L-HPC LH-21, and purified water. The extra granulation excipients can include Compound A and magnesium stearate (e.g., hyqual vegetable source). For example, a tablet dosage form is described in the Table 4 below.

TABLE 4

| | |
|---|---|
| Ingredient | % w/w |

Intra Granulation

| | |
|---|---|
| Compound A (API) | 20.00 |
| Microcrystalline Cellulose | 64.25 |
| Crospovidone, Type A | 5.00 |
| Hydroxypropyl Cellulose HPC-SL | 5.00 |
| Hydroxypropyl Cellulose-Low Substituted, | 5.00 |
| L-HPC LH-21 | |
| Sub Total | 99.25 |

Extra Granulation

| | |
|---|---|
| Compound A (API) granulate | 99.25 |
| Magnesium Stearate | 0.75 |
| Sub Total | 100.00 |

### Example 12

### First-in-Human Clinical Trial of Compound A

Chronic inhibitors of MEK have been poorly tolerated, and limited mainly to RAF mutant disease. Compound A is a deep cyclic inhibitor of MEK in the MAPK pathway. Compound A is a novel, oral, dual MEK inhibitor with a unique PK profile, characterized by a high Cmax and a short half-life. The clinical trial is an open-label study designed to evaluate the safety, tolerability, clinical pharmacology, and preliminary efficacy of Compound A when dosed as monotherapy, once daily, in participants with advanced RAS mutant solid tumors, following at least one line of systemic, standard-of-care therapy.

This is a Phase 1/2a, multicenter, open-label, nonrandomized study to characterize the safety and anti-tumor activity of Compound A administered to participants with RAS-mutated or RAS/MAPK pathway activated advanced or metastatic solid tumors as monotherapy and in combination with approved agents. This study begins with Phase 1 Dose Exploration (including monotherapy dose escalation and dose expansion stages). The monotherapy dose exploration is composed of a dose escalation study followed by the accrual of dose expansion cohorts to generate more robust data on Compound A's safety, tolerability, PK/PD, and antitumor activity.

The dose escalation follows an mTPI-2 design. The mTPI-2 dose escalation scheme in this study allows for a minimum of 1 participant and up to 8 participants at each dose level to be evaluated. The evaluation period encompasses the first 21 days after the first administration of Compound A (i.e., the first 21 days of the first 28-day treatment cycle [Cycle 1]), during which safety and tolerability data for each evaluable participant are collected. These data, along with PK and PD data (if available) and cumulative safety and tolerability data collected, inform dose-escalation decisions within the prespecified planned dose range (40 mg to 960 mg). The initially planned cohort dose escalation levels include: 40, 80, 160, 240, and 320 mg QD.

Once adequate safety, PK, PD, and any efficacy data are available, select tolerable and pharmacologically active dose cohorts are expanded by or enrolled to approximately 20 participants to further evaluate safety, tolerability, PK, PD, and anti-tumor activity. Monotherapy dose escalation participants previously treated at a dose level selected as one of the expansion cohort doses may be counted towards the overall enrollment of that expansion cohort. A preferred dose of Compound A exhibits optimal pharmacological characteristics of deep cyclic inhibition (DCI; high Cmax and near-zero drug trough, approximating a 2 h plasma half-life and >90% inhibition of pERK) and provides adequate anti-tumor activity while not exceeding the MTD (if reached during dose escalation).

Initial phase 1/2a clinical data (NCT05585320) demonstrate favorable initial safety along with PK and PD profiles that are consistent with preclinical modeling of deep cyclic inhibition (DCI), including Cmax levels over 2,000 ng/mL (~1 μM drug FF), a median plasma t1/2 of 1.94 hours, and ~90% pharmacodynamic inhibition of pERK.

The study population comprises male and female participants 18 years of age or older with advanced, unresectable, or metastatic RAS-mutated solid tumors who have received at least 1 prior line of systemic therapy for their advanced/metastatic disease. Patients for treatment can have any advanced, unresectable, or metastatic RAS-mutated solid tumors in Phase 1, while Phase 2a will focus on tumor type cohorts: NRAS-mutated melanoma, KRAS-mutated PDAC, KRAS-mutated NSCLC and KRAS-mutated, APC-wildtype CRC.

The cohort dose escalation levels include oral administration of Compound A at doses of 40, 80, 160, 320 mg QD, for up to twelve 28-day cycles. Once daily administration will allow for more than 10 half-lives between doses. Timed serial PK samples are collected on C1D1 and C1D15 and trough PK samples are collected at C1D2, C1D8, C1D16, C2D1 and C3D1.

Table 5 provides clinical data obtained from initial pharmacokinetics (PK) and pharmacodynamics (PD) data from an ongoing phase 1/2a trial (NCT05585320), from certain human subjects enrolled during the first 20 weeks since the first patient was dosed in this Phase 1 clinical trial, showing the calculated half-life based on samples taken at cycle 1 day 1 (C1D1) and cycle 1, day 15 (C1D15), after receiving once-daily oral doses of Compound A at doses of 40 mg, 80 mg, or 160 mg. Each patient was diagnosed with the RAS-mutant cancer diagnosis indicated in Table 5 prior to treatment with Compound A. No dose limiting toxicities (DLT) or severe adverse events (SAE) were observed while dosing these patients through C1D15. No Compound A drug-related adverse events beyond grade 1 were reported in dose levels III or IV. The clinical trial is actively enrolling patients at 320 mg QD p.o. with 2 additional patients already consented (KRAS-G12V pancreatic cancer and KRAS-G12D colorectal cancer). No dose limiting toxicities (DLTs) or serious adverse events (SAEs) were observed in the first 20 weeks since dosing the first patient. Early PK data are approximately dose linear with no Compound A drug accumulation. All patients listed in Table 5 subsequently cleared the dose limiting toxicity (DLT) window.

TABLE 5
Patient Status Summary for Compound A Phase 1 Clinical Trial

| | | RAS | Dose | | C1D1 | C1D15 | Mean | Cmpd A |
|---|---|---|---|---|---|---|---|---|
| # | Patient Cancer | Mutation | Level | Dose | (t_{1/2}) | (t_{1/2}) | (t_{1/2}) | Status |
| 1. | PANCREATIC | KRAS- | I | 40 mg | 1.82 | 2.10 | 1.96 | Off |
| | | G12D | | QD p.o. | hours | hours | hours | Treatment |
| 2. | COLORECTAL | KRAS- | II | 80 mg | 1.41 | 1.43 | 1.42 | Off |
| | | G12V | | QD p.o. | hours | hours | hours | Treatment |
| 3. | COLORECTAL | NRAS- | III | 160 mg | 2.04 | 1.83 | 1.94 | Off |
| | | Q61L | | QD p.o. | hours | hours | hours | Treatment |
| 4. | COLORECTAL | NRAS- | III | 160 mg | 1.91 | 1.97 | 1.94 | On |
| | | Q61K | | QD p.o. | hours | hours | hours | Treatment |
| 5. | PANCREATIC | KRAS- | IV | 320 mg | 2.31 | 2.46 | 2.38 | On |
| | | G12D | | QD p.o. | hours | hours | hours | Treatment |
| 6. | PANCREATIC | KRAS- | IV | 320 mg | 2.04 | 2.27 | 2.16 | On |
| | | G12V | | QD p.o. | hours | hours | hours | Treatment |
| 7. | COLORECTAL | KRAS- | IV | 320 mg | 1.45 | 2.27 | 1.86 | On |
| | | G12S | | QD p.o. | hours | hours | hours | Treatment |

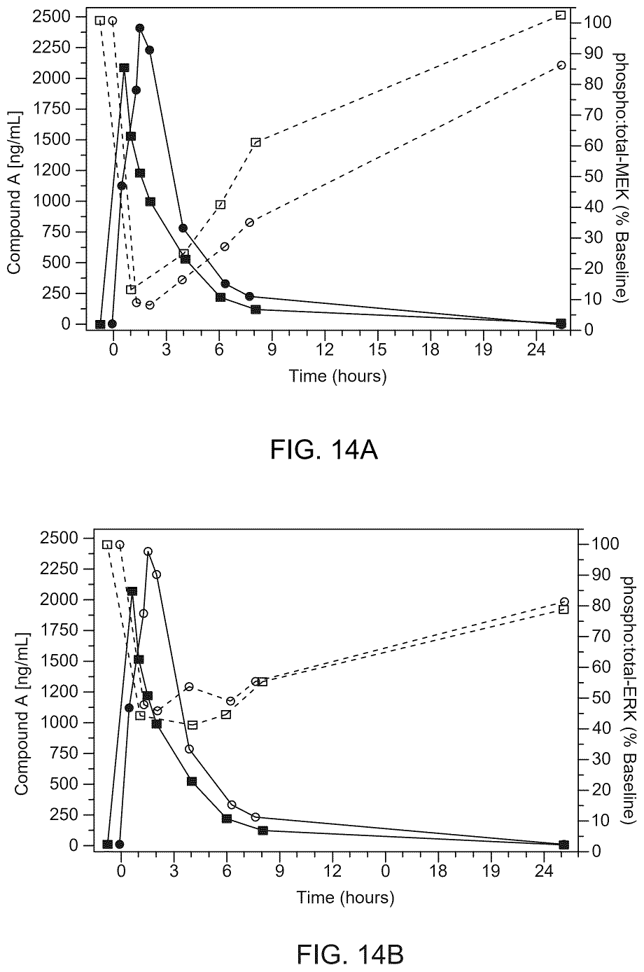

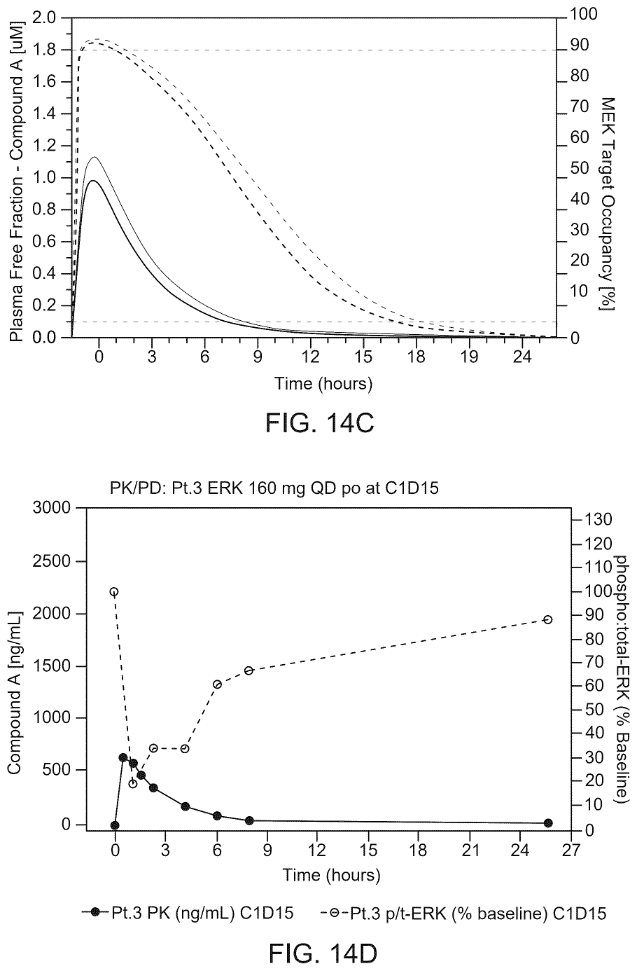

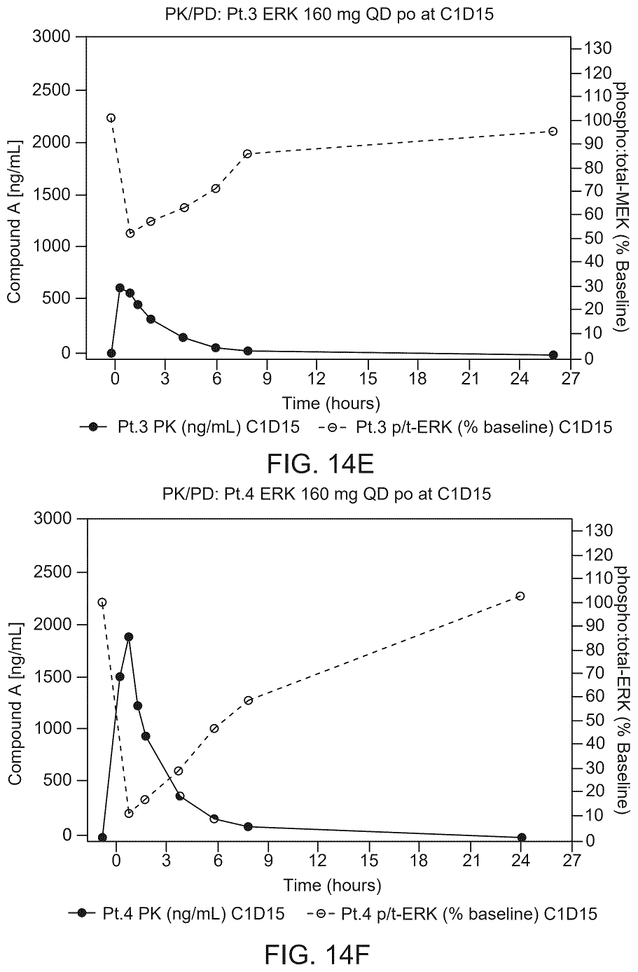

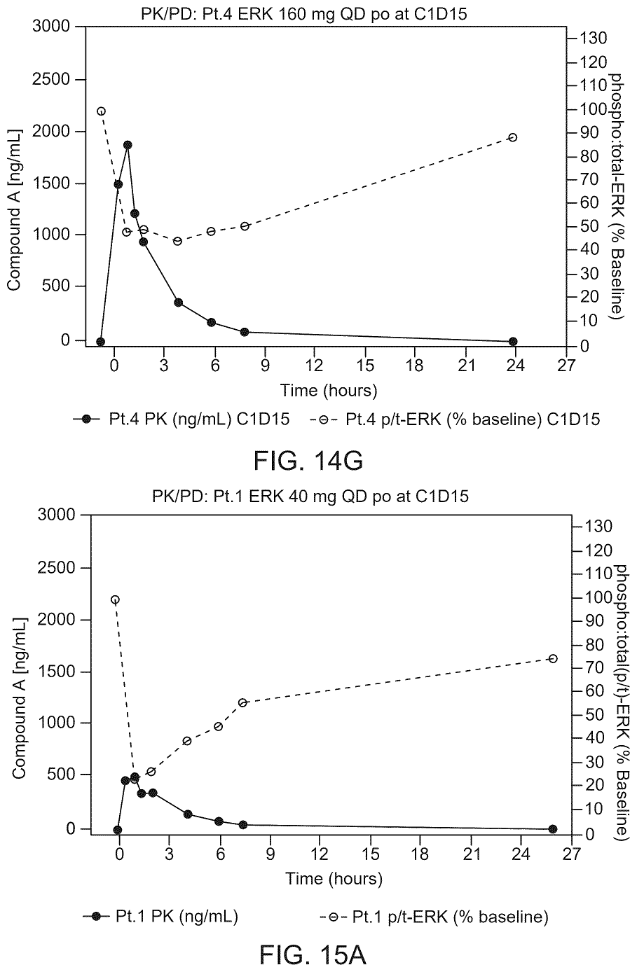

In patients 3 and 4, an approximately 90% pharmacodynamic (PD) inhibition of pERK was observed in patients receiving 160 mg of Compound A administered orally (po) once daily (QD). (Dose Level III, cycle 1, day 1). FIG. 14A shows Compound A PK/PD (pERK) showing MAPK pathway suppression (87-92% reduction p/t-ERK, Cmax≥2,000 ng/mL and Cmin of about 0.1 micromolar free fraction≥6-8 hours). FIG. 14B shows Compound A PK/PD (phosphorylated MEK) data showing suppression of pathway bypass (55-59% reduction p/total-MEK, Cmax≥2,000 ng/mL and Cmin of about 0.1 micromolar free fraction≥6-8 hours). FIG. 14C shows modeled PK/BK, drug free fraction (FF) with a high Cmax (see Hoare, Sam RJ. “Analyzing kinetic binding data.” Assay Guidance Manual) (≥90% MEK occupancy, Cmax 1 micromolar FF, Cmin of about 0.1 micromolar free fraction≥6-8 hours). Referring to FIG. 14A-C:

Dose Level III: Cycle 1 Day 1 (C1D1) PK (solid line), BK (dotted line), and PD (dotted line) for patient 3 (solid line with circles) and 4 (solid line with solid squares), both at 160 mg QD p.o.

PK plasma: pre-dose (0), 0.5, 1, 1.5, 2*, 4, 6, 8, 24 hours; PD plasma: PK-matched without 0.5 or 1.5 hours (*poor sample quality for Pt.4 at 2 hour)

PD method: A549 (KRASG12S) cells were exposed to patient plasma for 2-hours before quantifying phosphorylated and total ERK and MEK

FIG. 14A is a graph showing PK/PD (pERK) for Compound A from patients 3 and 4 after receiving 160 mg administered orally (po) once daily (QD) (cycle 1, day 1), showing Compound A (pERK) MAPK pathway suppression. FIG. 14B is a graph showing PK/PD (pMEK) for Compound A from patients 3 and 4 after receiving 160 mg administered orally (po) once daily (QD) (cycle 1, day 1), shows suppression of pathway bypass. FIG. 14C is a graph showing the modeled PK/BK based on data from patients 3 and 4 after receiving 160 mg administered orally (po) once daily (QD) (cycle 1, day 1), drug free fraction (FF) for Compound A including a high Compound A Cmax.

FIG. 14D is a graph showing PK/PD (pERK) for Compound A from patient 3 after receiving 160 mg administered orally (po) once daily (QD) (cycle 1, day 15). FIG. 14E is a graph showing PK/PD (pMEK) for Compound A from patient 3 after receiving 160 mg administered orally (po) once daily (QD) (cycle 1, day 15). FIG. 14F is a graph showing PK/PD (pERK) for Compound A from patient 4 after receiving 160 mg administered orally (po) once daily (QD) (cycle 1, day 15). FIG. 14G is a graph showing PK/PD (pMEK) for Compound A from patient 4 after receiving 160 mg administered orally (po) once daily (QD) (cycle 1, day 15).

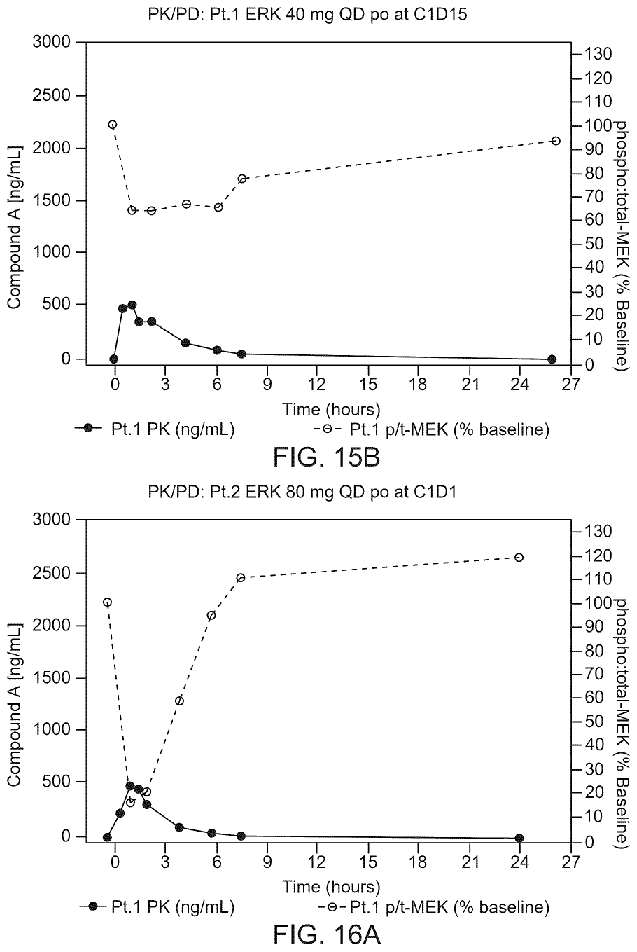

FIG. 15A is a graph showing PK/PD (pERK) for Compound A from patient 1 after receiving 40 mg administered orally (po) once daily (QD) (cycle 1, day 1). FIG. 15B is a graph showing PK/PD (pMEK) for Compound A from patient 1 after receiving 40 mg administered orally (po) once daily (QD) (cycle 1, day 1).

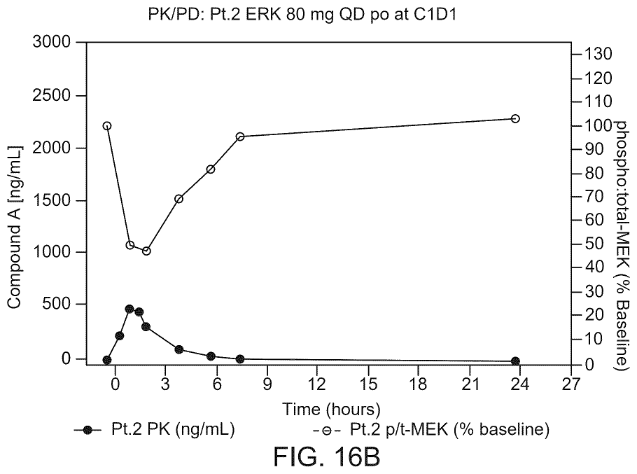

FIG. 16A is a graph showing PK/PD (pERK) for Compound A from patients after receiving 80 mg administered orally (po) once daily (QD) (cycle 1, day 1). FIG. 16B is a graph showing PK/PD (pMEK) for Compound A from patient 2 after receiving 80 mg administered orally (po) once daily (QD) (cycle 1, day 1).

Significant PK Cmax levels (plasma concentration of therapy in a specific area of the body) were observed with Compound A of over 2,000 ng/mL (or approximately 1 uM drug free-fraction at 160 mg once daily oral dose)

Greater than 90 percent PD inhibition of phosphorylated extracellular signal-regulated kinase (pERK) with Compound A compared to pretreatment baseline for patients at the third dose level (160 mg once daily oral)

A median plasma half-life (t_{1/2}) of 1.94 hours observed with Compound A across the first three dose levels evaluable (40 mg, 80 mg and 160 mg once daily oral), in patients with pancreatic and colorectal cancer with different RAS mutations, including KRAS-G12D, the most common mutation presents in pancreatic cancer.

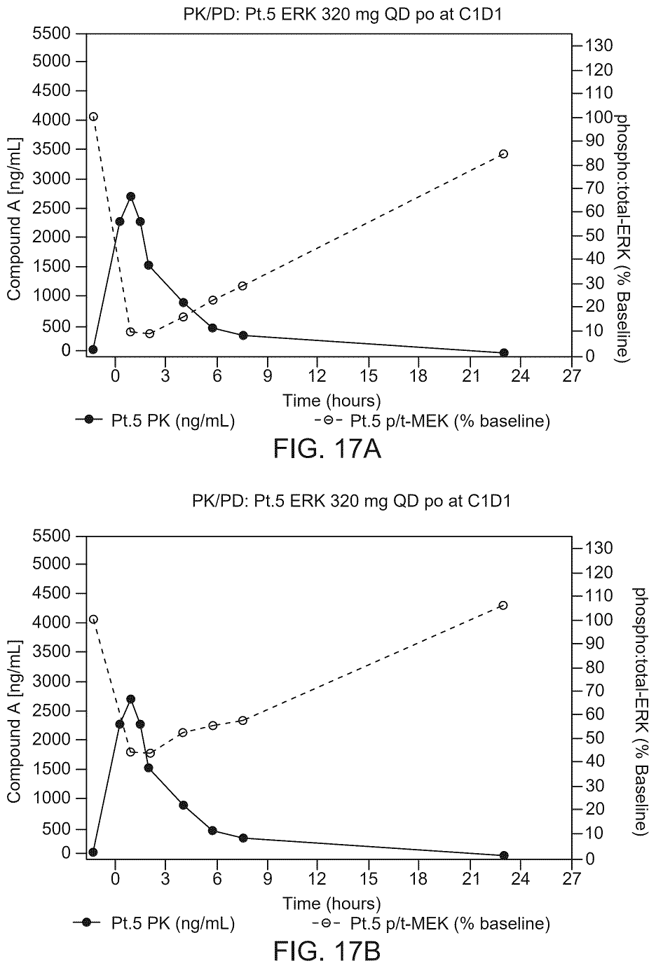

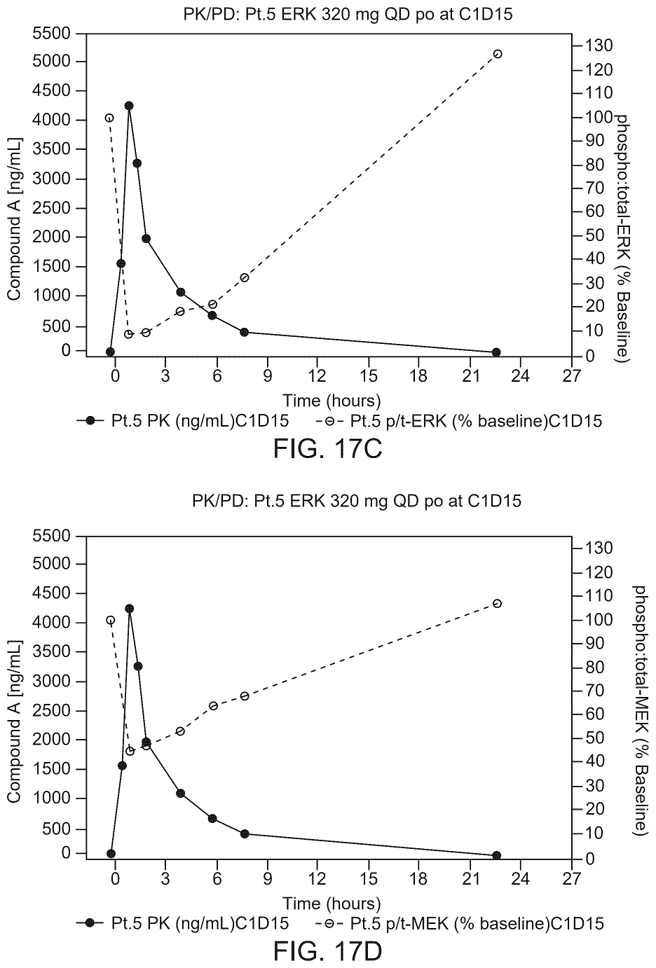

Patient 5 was diagnosed with KRAS-G12D pancreatic cancer and received 320 mg of Compound A once daily, for 15 days in cycle 1. Table 5 provides the corresponding half-life measured at day 1 and day 15. FIG. 17A is a graph showing PK/PD (pERK) for Compound A from patient 5 after receiving 320 mg administered orally (po) once daily (QD) (cycle 1, day 1). FIG. 17B is a graph showing PK/PD (pMEK) for Compound A from patient 5 after receiving 320 mg administered orally (po) once daily (QD) (cycle 1, day 1). FIG. 17C is a graph showing PK/PD (pERK) for Compound A from patient 5 after receiving 320 mg administered orally (po) once daily (QD) (cycle 1, day 15). FIG. 17D is a graph showing PK/PD (pMEK) for Compound A from patient 5 after receiving 320 mg administered orally (po) once daily (QD) (cycle 1, day 15).

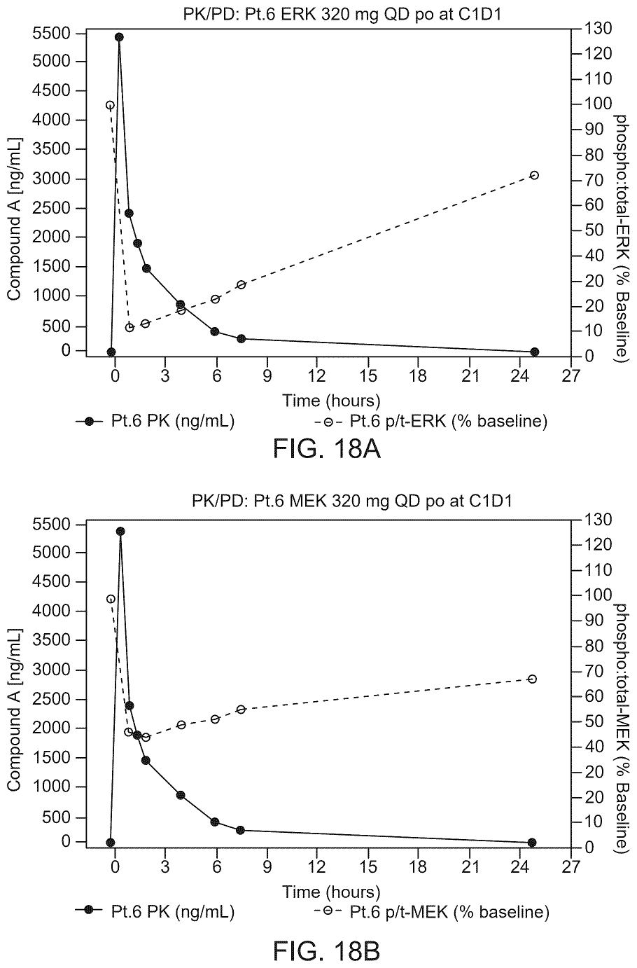

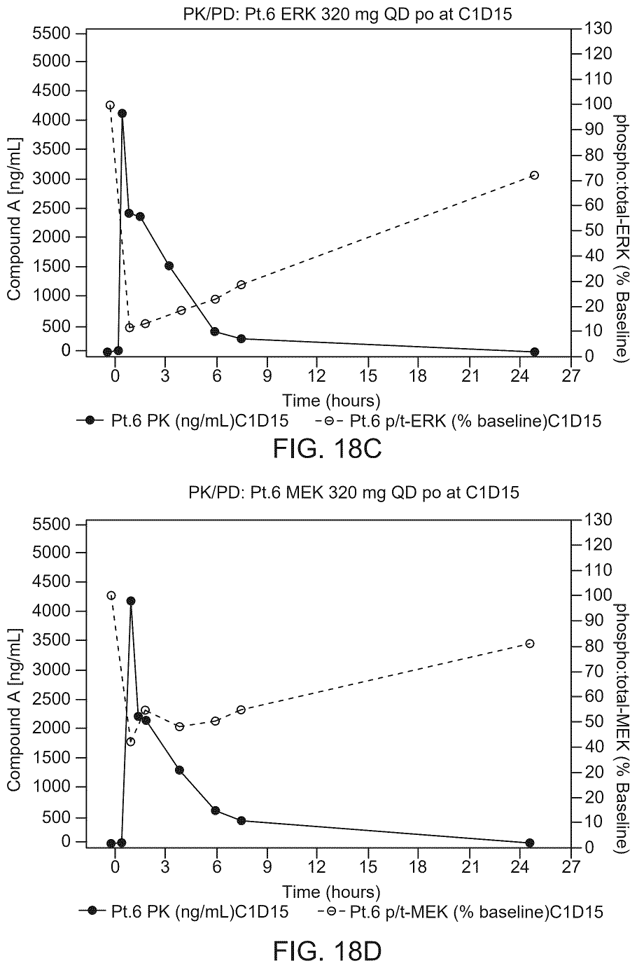

Patient 6 was diagnosed with KRAS-G12V pancreatic cancer and received 320 mg of Compound A once daily, for 15 days in cycle 1. Table 5 provides the corresponding half-life measured at day 1 and day 15. FIG. 18A is a graph showing PK/PD (pERK) for Compound A from patient 6 after receiving 320 mg administered orally (po) once daily (QD) (cycle 1, day 1). FIG. 18B is a graph showing PK/PD (pMEK) for Compound A from patient 6 after receiving 320 mg administered orally (po) once daily (QD) (cycle 1, day 1). FIG. 18C is a graph showing PK/PD (pERK) for Compound A from patient 6 after receiving 320 mg administered orally (po) once daily (QD) (cycle 1, day 15). FIG. 18D is a graph showing PK/PD (pMEK) for Compound A from patient 6 after receiving 320 mg administered orally (po) once daily (QD) (cycle 1, day 15).

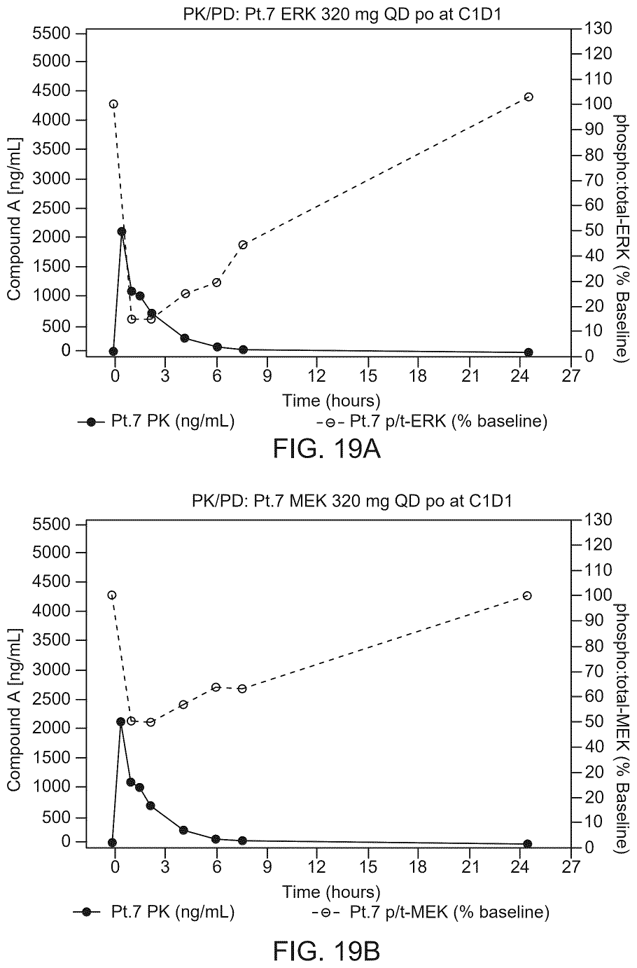

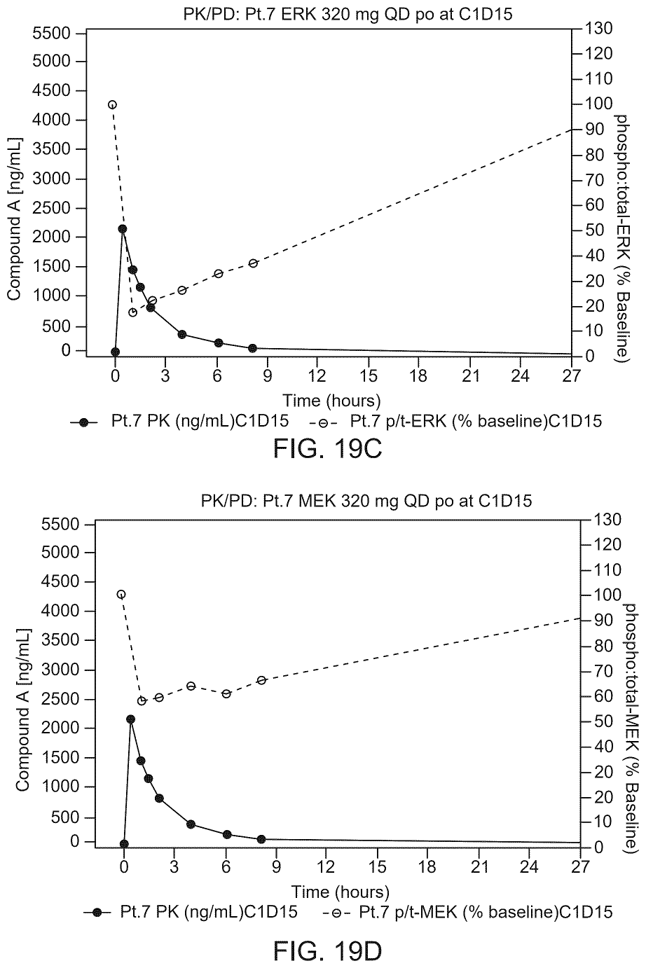

Patient 7 was diagnosed with KRAS-G12S colorectal cancer and received 320 mg of Compound A once daily, for 15 days in cycle 1. Table 5 provides the corresponding half-life measured at day 1 and day 15. FIG. 19A is a graph showing PK/PD (pERK) for Compound A from patient 7 after receiving 320 mg administered orally (po) once daily (QD) (cycle 1, day 1). FIG. 19B is a graph showing PK/PD (pMEK) for Compound A from patient 7 after receiving 320 mg administered orally (po) once daily (QD) (cycle 1, day 1). FIG. 19C is a graph showing PK/PD (pERK) for Compound A from patient 7 after receiving 320 mg administered orally (po) once daily (QD) (cycle 1, day 15). FIG. 19D is a graph showing PK/PD (pMEK) for Compound A from patient 7 after receiving 320 mg administered orally (po) once daily (QD) (cycle 1, day 15).

An additional patient was enrolled at 160 mg QD p.o. (NRAS-G13R Melanoma); independent of DLT dose escalation.

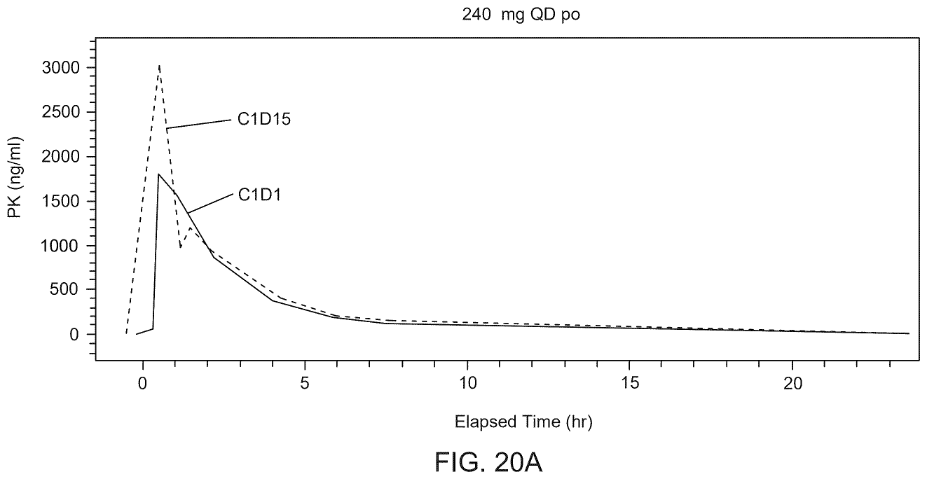

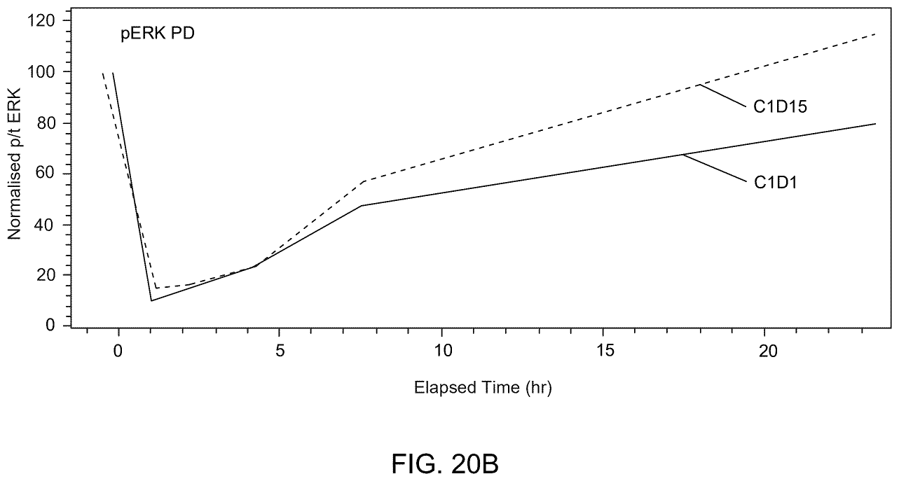

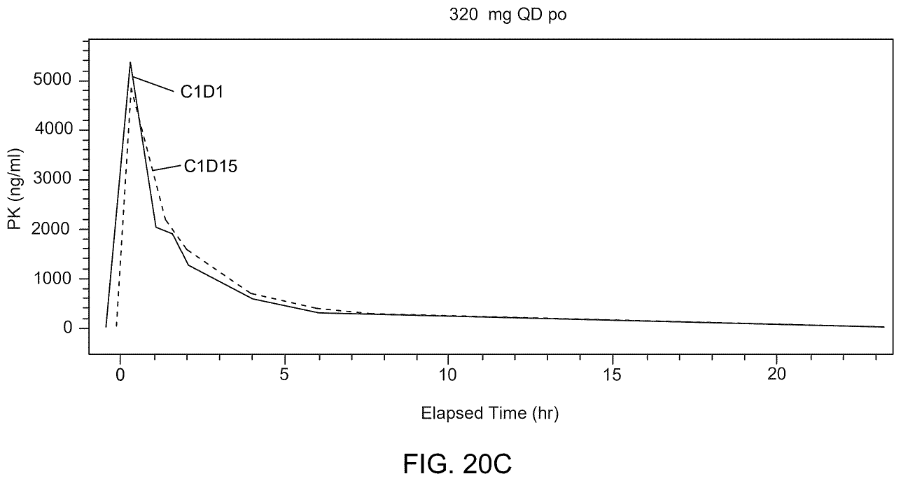

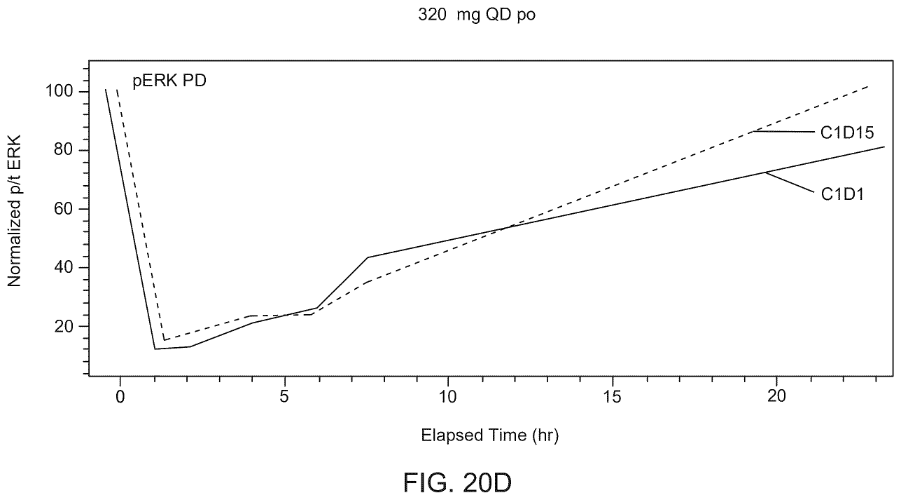

FIG. 20C is a graph showing plasma concentration (PK) for Compound A from patient 16 after receiving 320 mg Compound A administered orally (po) once daily (QD) at cycle 1, day 1 and at cycle 1, day 15. FIG. 20D is a graph showing PK/PD (pERK) for Compound A from patient 16 after receiving 320 mg Compound A administered orally (po) once daily (QD) at cycle 1, day 1 and at cycle 1, day 15.

The cohort dose escalation levels included oral administration of Compound A at a dose of 240 mg QD, for up to twelve 28-day cycles. Once daily administration allowed for more than 10 half-lives between doses. Timed serial PK samples were collected on C1D1 and C1D15 and trough PK samples are collected at C1D2, C1D8, C1D16, C2D1 and C3D1. FIG. 20A is a graph showing plasma concentration (PK) for Compound A from patient 13 after receiving 240 mg Compound A administered orally (po) once daily (QD) at cycle 1, day 1 and at cycle 1, day 15. FIG. 20B is a graph showing PK/PD (pERK) for Compound A from patient 13 after receiving 240 mg Compound A administered orally (po) once daily (QD) at cycle 1, day 1 and at cycle 1, day 15.

This is an example of developing a medicine for a broad population of cancer patients using a universal-RAS therapy in the dose-escalation portion of the Phase 1/2a study for Compound A in patients with advanced solid tumors. The study's Safety Review Committee completed its evaluation and determined that doses up to and including 320 mg (including, for example, 240 mg) once daily were tolerable with no dose limiting toxicities. Enrollment in the Phase 1b expansion portion of the study will commence and is designed to evaluate two dosing cohorts of 12 patients each at an oral dose of 240 mg or 320 mg once daily, with three patients already in the study have so far been dosed at the 320 mg dose level.

This unique therapy aims for Universal-RAS activity: treating patients with any mutation in KRAS, NRAS, or HRAS. As such, there are plans to enroll 21 additional patients in next part of the study to materially add to a dataset for Compound A in patients with RAS-mutant solid tumors.

Compound A achieved significant levels of PK Cmax, demonstrated a half-life of approximately two hours, and was well tolerated with no dose limiting toxicities or serious adverse events observed in patients with pancreatic and colorectal cancer. Upon review of the cumulative data from the eight patients dosed in the Phase 1a study, the SRC endorsed 240 mg daily and 320 mg daily as the dose levels for the expansion cohort and agreed with initiating the Phase 1b expansion.

Compound A is contemplated to achieve universal-RAS activity that selectively impacts cancer cells to a greater extent than healthy cells, through deep cyclic inhibition of the MAPK pathway with once-daily oral dosing. Compound A is currently being evaluated in a Phase 1/2a study in patients with advanced solid tumors harboring RAS mutations for whom there are limited treatment options (NCT05585320).

Compound A was well tolerated with no DLTs or SAEs observed and no drug-related adverse events beyond Grade 1 observed. All patients listed in Table 5 subsequently cleared the dose limiting toxicity (DLT) window. An additional patient was enrolled at 160 mg QD p.o. (NRAS-G13R Melanoma); independent of DLT dose escalation.

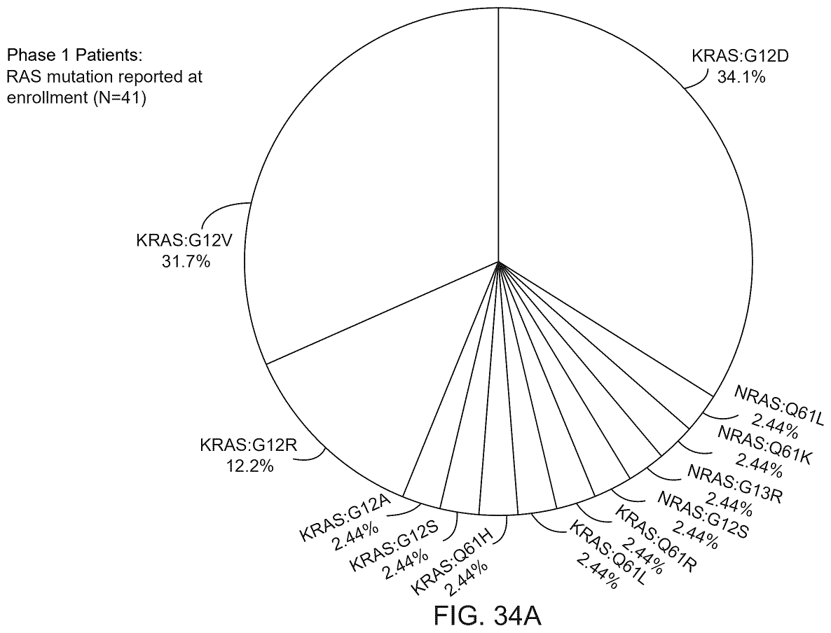

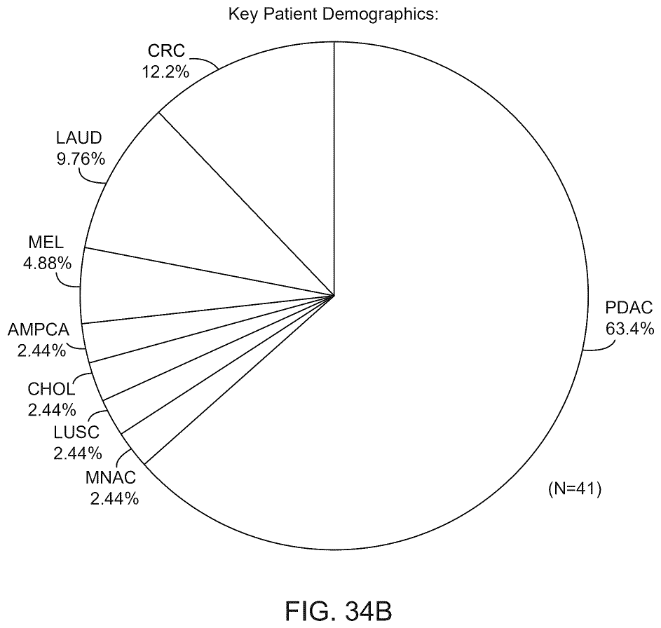

FIG. 34A is a graph showing the RAS mutations reported at enrollment (N=41) for phase 1 human clinical trial patients treated with Compound A. About two-thirds of the patients treated with Compound A as a third or later line of therapy, up to 7th line. RAS mutations were reported in 41 patients diagnosed with various forms of RAS mutated cancer each treated with Compound A (N=41). The 41 patients treated with Compound A included 26 pancreatic ductal adenocarcinoma (PDAC), 5 colorectal cancer (CRC), 4 lung adenocarcinoma (LUAD), 2 melanoma (MEL), 1 lung squamous cell carcinoma (LUSC), 1 ampulla of vater carcinoma (AMPCA), 1 mesonephric adenocarcinoma (MNAC), 1 cholangiocarcinoma (CHOL).

FIG. 34B is a graph showing cancer diagnosis for the patients reported at enrollment (N=41) for phase 1 human clinical trial patients treated with Compound A. A total of 82% of the 41 patients treated with Compound A never had a prior partial response (PR) or complete response (CR) to any prior therapy before treatment with Compound A, and the patients had a range of 1-6 prior lines of therapy before receiving Compound A (median 2 prior lines of therapy). The 41 patients treated with Compound A included 34 patients with prior treatment history, of which only 6 are known to have had a PR in response (no CR's) to any prior treatment for metastatic disease (excludes adjuvant). Among these 34 patients with prior treatment history, only 6 are known to have had a partial response (PR) and none are known to have had a complete response (CR), in response to any prior treatment for metastatic disease (excludes adjuvant), Compound A treatment was the median third line of therapy (range 2nd-7th line).

About two-thirds of the patients treated with Compound A as a third or later line of therapy, up to 7^{th} line. Specifically, among 34 patients treated with Compound A, a total of 32.4% received Compound A as a second line therapy, 38.2% of patients received Compound A as a third line therapy, 14.7% received Compound A as a fourth line of therapy, 8.82% received Compound A as a fifth line of therapy, 2.94% received Compound A as a sixth line of therapy and 2.94% received compound A as a seventh line of therapy. RAS mutations were reported in 41 patients diagnosed with various forms of RAS mutated cancer each treated with Compound A (N=41). The 41 patients treated with Compound A included 26 pancreatic ductal adenocarcinoma (PDAC), 5 colorectal cancer (CRC), 4 lung adenocarcinoma (LUAD), 2 melanoma (MEL), 1 lung squamous cell carcinoma (LUSC), 1 ampulla of vater carcinoma (AMPCA), 1 mesonephric adenocarcinoma (MNAC), 1 cholangiocarcinoma (CHOL).

A total of 82% of the 41 patients treated with Compound A never had a prior partial response (PR) or complete response (CR) to any prior therapy before treatment with Compound A, and the patients had a range of 1-6 prior lines of therapy before receiving Compound A (median 2 prior lines of therapy). The 41 patients treated with Compound A included 34 patients with prior treatment history, of which only 6 are known to have had a PR in response (no CR's) to any prior treatment for metastatic disease (excludes adjuvant). Among these 34 patients with prior treatment history, only 6 are known to have had a partial response (PR) and none are known to have had a complete response (CR), in response to any prior treatment for metastatic disease (excludes adjuvant), Compound A treatment was the median third line of therapy (range 2nd-7th line).

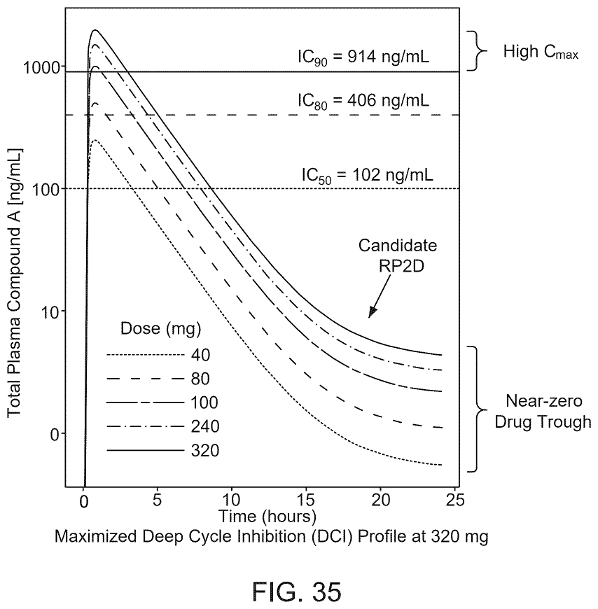

The exploratory PK/PD results of 19 patients treated with Compound A were analyzed (N=19). The Compound A plasma drug concentrations (ng·L) were measured over time after doses of Compound A at 40, 80, 160, 240 and 320 PO QD. FIG. 35 is a graph showing pharmacokinetic (PK) and pharmacodynamic (PD) data obtained from patients treated with Compound A.

Modeled PK profiles were calculated based on measurements of plasma concentrations (ng/mL) of Compound A versus time (h) from 19 patients on a semilogarithmic scale for different dose groups. Approximately dose linear from 40 to 320 mg PO QD with no drug accumulation. A tight relationship was observed between plasma concentrations and phosphorylated ERK (p-ERK) to total ERK (t-ERK) ratios; Longer time above pMEK IC90 at 320 mg (4.0 hr) vs. 240 mg (3.3 hr). Overall, a tight relationship was observed between the plasma concentrations and p-ERK to total ERK ratios, as well as a low variance in PD (p-ERK) profiles at the 320 mg compared to the 240 mg PO QD dose levels. Overall, Compound A was well-tolerated by patients in the clinical trial, with 100% suppression of acquired RAS alterations observed and individual target lesion regression in about half of the patients. Among the patients treated with Compound A:
Best individual lesion regressions: −35.7% at 320 mg in 2 L (vs. −11.4% at 240 mg);
Best RECIST SLD: −18.9% at 320 mg in 2 L (vs. −7.1% at 240 mg);
Longest duration on therapy: 162 days (5+ months) at 240 mg; no TRAEs; and
53% of patients had ≥1 target lesions regress at 320 mg or 240 mg.

A total of 11 patients receiving Compound A had post-baseline scans at 320 mg, 6 patients with post-baseline scans at 240 mg, 3 patients with scans at lower doses, 7 patients have started treatment but not yet scanned, 5 patients are pending data entry and 8 patients progressed before they could receive a post-baseline scan, and 1 was not evaluable. 6 of 20 (30%) patients with completed RECIST scans, showed best sum of longest diameters (SLD) of 0% to −18.9%, and 4 of 20 (20%) showed best SLD less than zero. (Note: 2 patients at 320 mg with −35.7% as best individual lesion regression, both in 2 L).

### Example 13

Compound a as Single Agent and in Combination with Encorafenib for Patients with RAS or RAF Mutant Tumors

Compound A is a novel, oral, dual MEK inhibitor with a unique PK profile, characterized by a high Cmax and a short half-life. Many tumors are addicted to MAPK pathway activation, including the >20% of human tumors with mutations in RAS or RAF. Compound A is an oral once-daily treatment currently in Phase 1 patients with advanced or metastatic RAS-mutant solid tumors [NCT05585320]. To date, drugs disrupting the MAPK pathway have done so chronically, leading to dose-limiting toxicities (DLTs) and poor response durability. In contrast, Compound A was designed to provide deep cyclic inhibition (DCI) of the MAPK pathway via features including a unique pharmacokinetic (PK) profile with high peak plasma drug levels and a near zero, daily drug trough. This promotes pulsatile inhibition of MEK, depriving tumors of sustained signaling of a critical oncogenic pathway while limiting toxicity and durability issues associated with chronic MEK inhibition. In Phase 1a dose escalation, no DLTs were observed, the plasma drug half-life was approximately 2-hours, and pharmacodynamic (PD) data were consistent with DCI. Phase 1b dose expansion is underway.

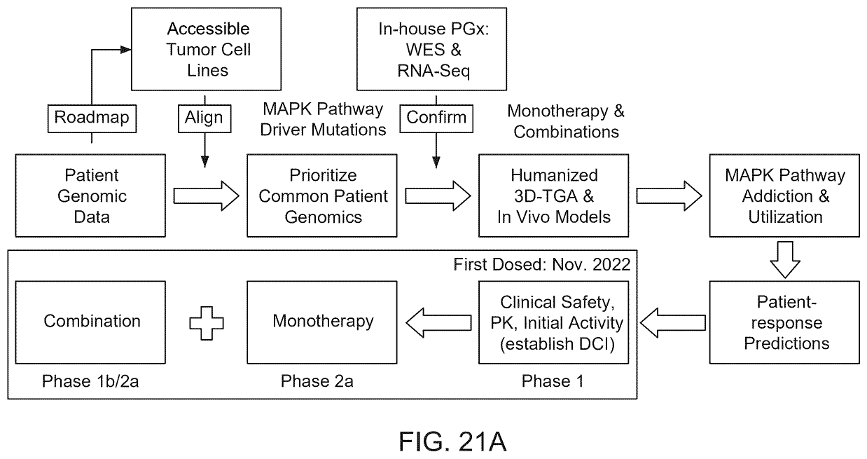

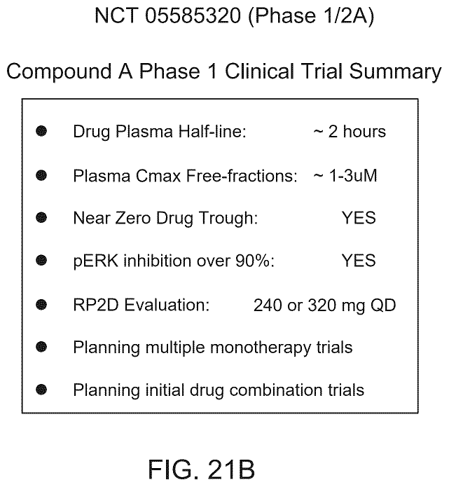

FIG. 21A is a schematic diagram of an end-to-end translational modeling approach in support of Compound A. FIG. 21B is a table providing certain Phase 1 clinical trial summary parameters for Compound A.

Translational efforts are focused on identifying MAPK pathway addiction and sensitivity to Compound A. Tumor models displaying patient-aligned genomic profiles against large patient databases such as AACR Project GENIE1 were tested in humanized 3D tumor growth assays (3D-TGA). Computational modeling based on response and in-house genomic data was used to inform identification of patient populations for Compound A monotherapy and potential combination opportunities.

Compound A is a short-lived dual MEK inhibitor (MEKi) that works through Deep Cyclic Inhibition (DCI) and is currently under evaluation in early clinical development with an initial focus on advanced RAS mutant solid tumors (NCT05585320). Expanded preclinical and translational modeling support broad potential as monotherapy and in combination for MAPK-pathway mutations upstream of MEK. Assessment of Compound A in over 190 patient-aligned 3D tumor models demonstrated diverse responses across a wide range of MAPK-driven tumor types, including those with RAS or RAF mutations.

Using cancer-specific, patient-aligned cell lines, Compound A activity was characterized in the 3D-TGA. Whole exome sequencing was performed to confirm alteration status, and a further subset subjected to RNA sequencing. Pharmacogenomic data were used to generate a model predictive of response to Compound A and identify biomarker-aligned patient subpopulations. Selected model predictions were then tested in subcutaneous tumor xenograft models in female BALB/c nude mice.

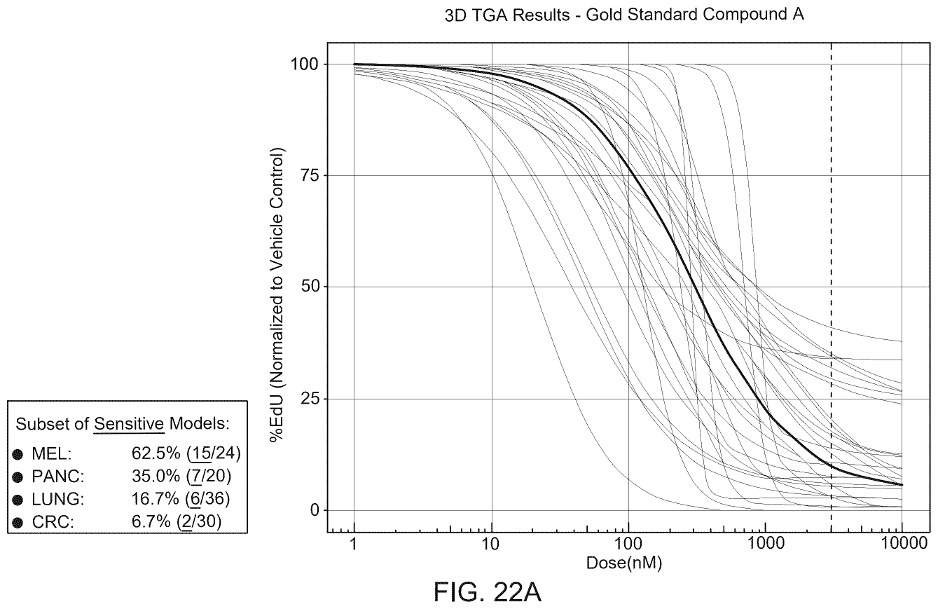

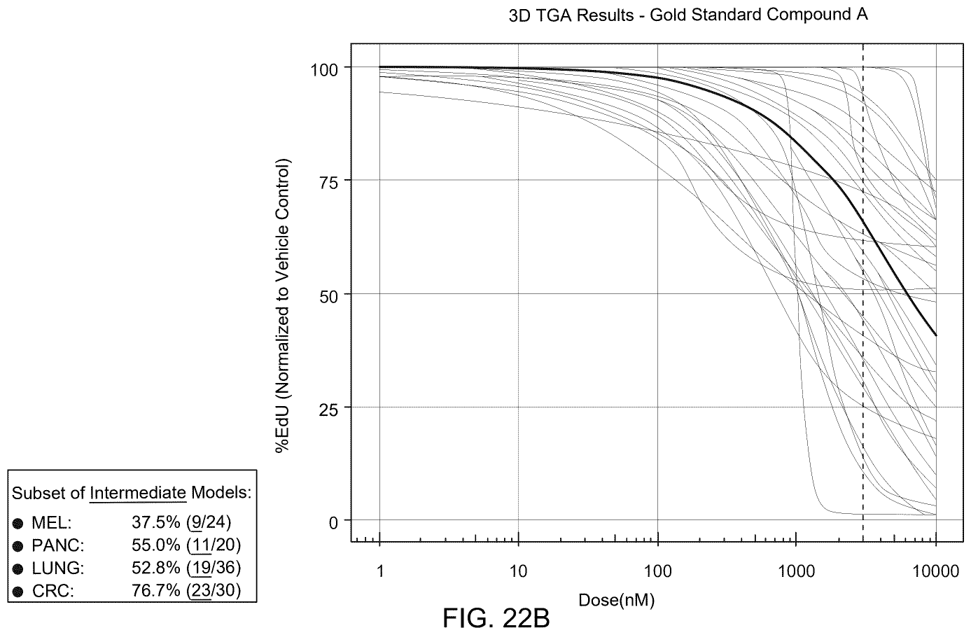

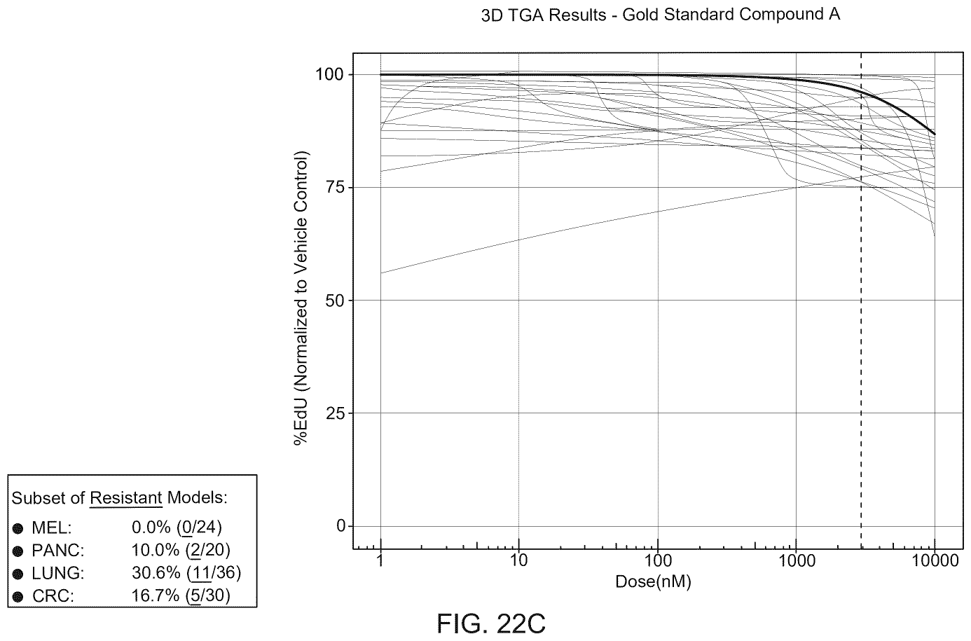

Referring to FIGS. 22A, 22B, and 22C, are graphs obtained from 3D-TGA pharmacogenomics modeling (pharmacology shown). The cell lines tested in 3D-TGA (N=193) were assigned response of sensitive (IC₅₀<1 uM), intermediate (IC50≥1 and >25% reduction at 10 uM), and resistant otherwise. The dark line on each plot represents the median of the individual curves; Dotted vertical line matches Cmax Compound A drug free-fraction levels achieved at 320 mg QD p.o. Major tumor types with activation mutation in the MAPK pathway upstream of MEK (Biomarker Positive): MEL (23/24), PANC (19/20), LUNG (33/36), CRC (28/30). FIG. 22A is a graph showing 3D-TGA Compound A dose responses calculated for sensitive tumor models. FIG. 22B is a graph showing 3D-TGA Compound A dose responses calculated for intermediate tumor models. FIG. 22C is a graph showing 3D-TGA Compound A dose responses calculated for resistant tumor models.

In some embodiments, a therapeutically effective amount of Compound A can be administered for the treatment of a form of melanoma with a RAS or RAF mutation. In some embodiments, a therapeutically effective amount of Compound A can be administered for the treatment of a form of pancreatic cancer with a RAS or RAF mutation. In some embodiments, a therapeutically effective amount of Compound A can be administered for the treatment of a form of lung cancer with a RAS or RAF mutation. In some embodiments, a therapeutically effective amount of Compound A can be administered for the treatment of a form of colorectal cancer with a RAS or RAF mutation.

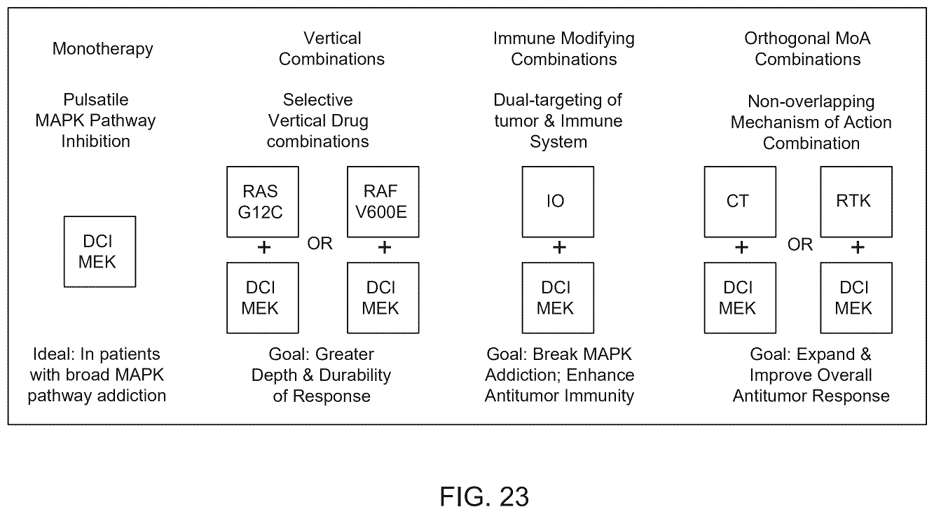

FIG. 23 is a schematic depicting examples of various therapeutic applications of Compound A both as monotherapy and Compound A in combination with other therapies, for the treatment of certain RAS and RAF mutations. Referring to FIG. 23, DCI is an abbreviation for Deep Cyclic Inhibition of the MAP Kinase pathway is a core feature of Immuneering's Dual MEK inhibitor, Compound A, IO is an abbreviation for immune oncology, CT is an abbreviation for Cytotoxic Therapy and RTK is an abbreviation for Receptor Tyrosine Kinase. The deep cyclic inhibition (DCI) of MEK is believed to display strong antitumor activity as monotherapy as well as across broad drug-drug combinations.

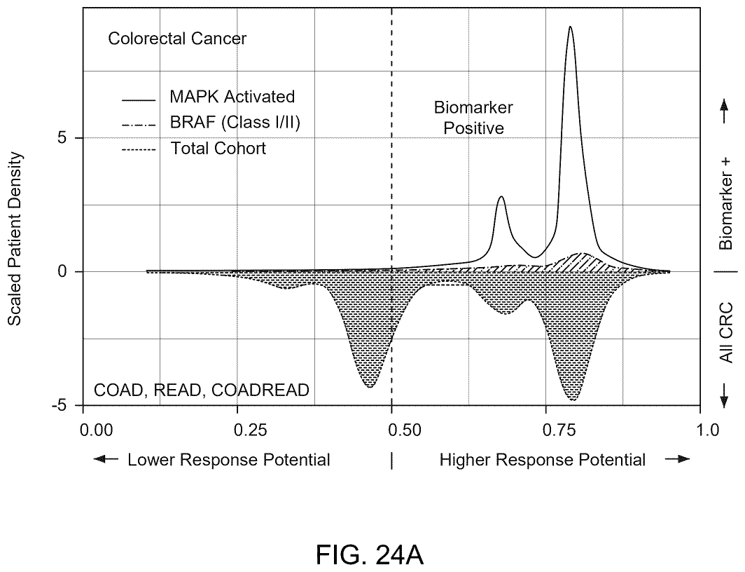

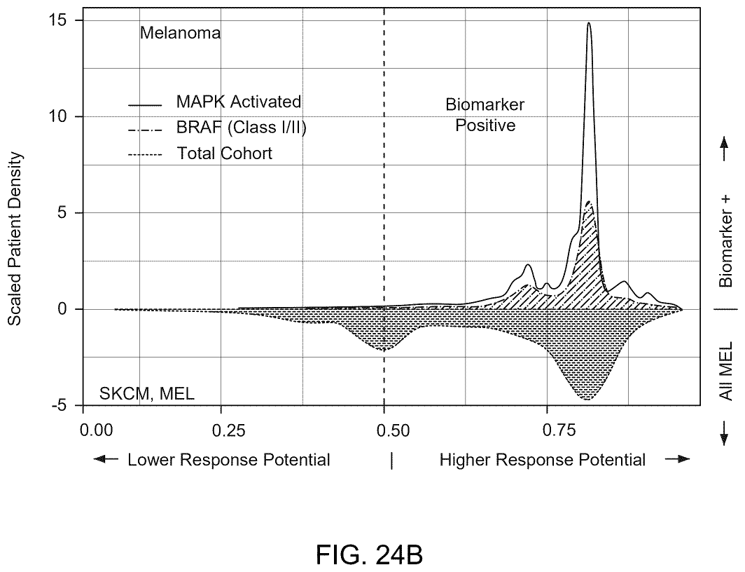

Referring to FIGS. 24A and 24B, each Figure shows for each indication: kernel density plot of probability of predicted response for entire cohort (projected onto the negative y-axis), and biomarker+ subset (positive y-axis). FIGS. 24A and 24B show the BRAF (class I and II) subsets (blue histogram) within the overall MAPK activated population (red line).

In addition to RAS, these and prior data support potential for Compound A treatment in RAF-mutant disease. Here, Compound A was tested alone and in a vertical combination with encorafenib (HT-29 colorectal BRAFV600E xenograft tumor model). The in vivo DCI MEKi combination of Compound A with encorafenib resulted in deeper regressions and superior durability in a head-to-head comparison versus binimetinib plus encorafenib. In an orthogonal combination, 3D modeling revealed Compound A combination potential with cytotoxic agents in pancreatic tumor models (in vivo combination studies are underway). An integrated translational platform with integrated bioinformatics utilized patient-aligned model systems was used to identify key factors that help predict response to Compound A and its unique DCI profile.

Assessment of Compound A across >190 patient-aligned models demonstrated diverse responses across a wide range of MAPK-driven tumor types, including those with RAS or RAF mutations. In addition to RAS, these data suggested additional potential for Compound A in BRAF-mutant disease. Therefore, Compound A was tested alone and with encorafenib in the HT-29 colorectal BRAFV600E mutant xenograft model. Monotherapy with either encorafenib or Compound A displayed superior tumor growth inhibition to binimetinib. Compound A in combination with encorafenib drove deeper regressions and superior durability of response in a head-to-head in vivo comparison versus binimetinib plus encorafenib.

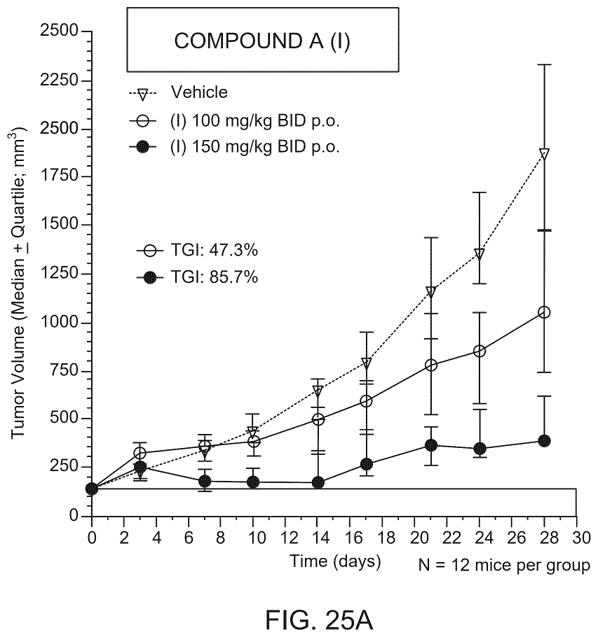

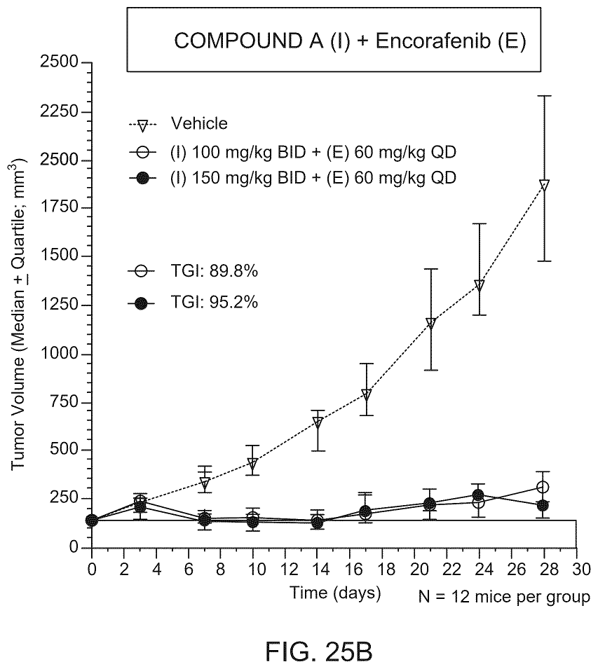

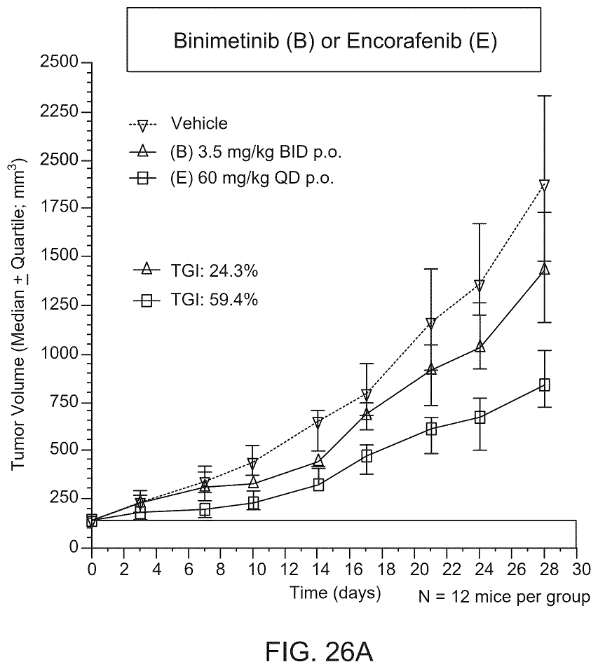

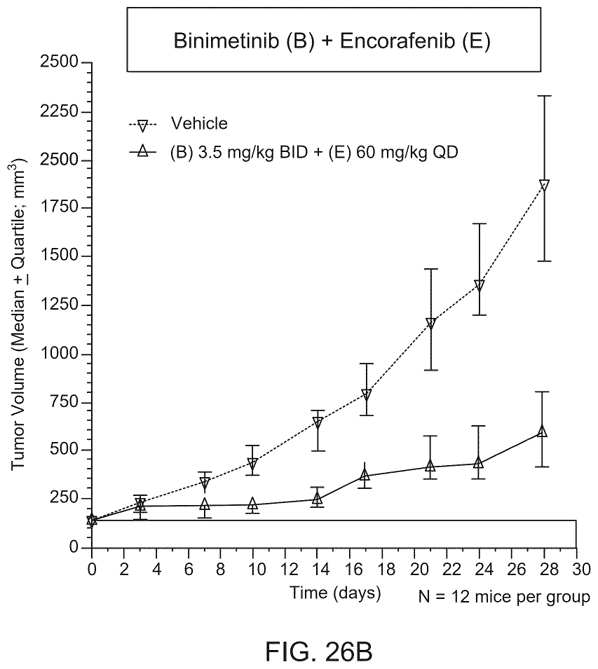

Referring to FIGS. 25A, 25B, 26A and 26B: (1) HT-29 BRAFV600E Colorectal Cancer (CRC) xenograft tumor model was run in athymic nude mice; (2) binimetinib (MEK inhibitor) and encorafenib (BRAF inhibitor) were commercially purchased; (3) Tumor Growth Inhibition (TGI) % was calculated with the equation [1−(Ti−T₀)/(Ci−Co)]×100%; and (4) no median body weight loss was noted. FIG. 25A shows the tumor volume result of Compound A monotherapy treatment; and FIG. 25B shows the tumor volume result of Compound A and encorafenib as a vertical combination (in vivo) experiment of a BRAF+MEK (head-to-head vs. the human dose equivalents of the registered MEKi (binimetinib) and RAFi (encorafenib)). The deep cyclic inhibition (DCI) approach that Compound A establishes demonstrated superior depth and durability as monotherapy (i.e., MEKi (Compound A) vs. MEKi (binimetinib)) and in MEKi+RAFi combinations with encorafenib. FIG. 26A and FIG. 26B show the comparative tumor volume growth measured using binimetinib or encorafenib (FIG. 26A) or binimetinib and encorafenib combined (FIG. 26B).

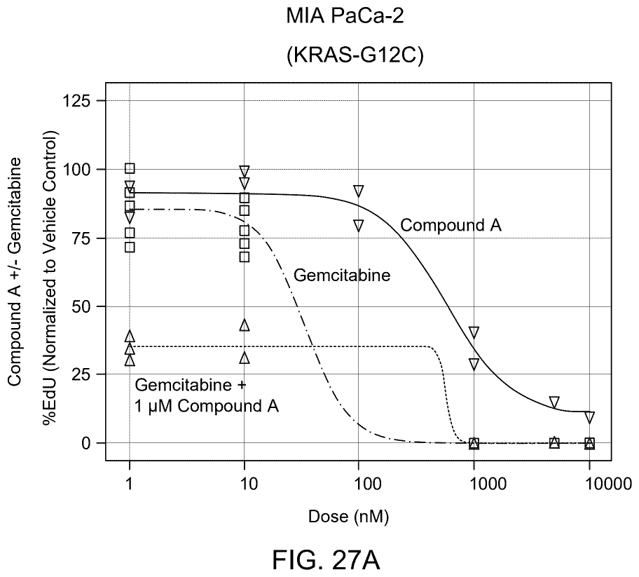

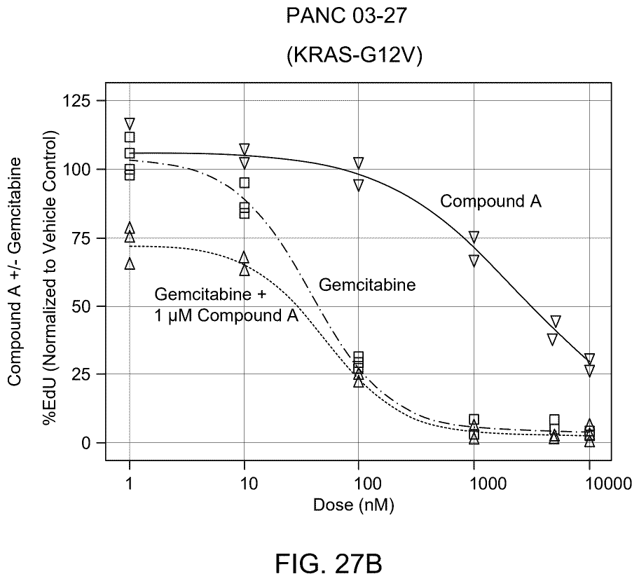

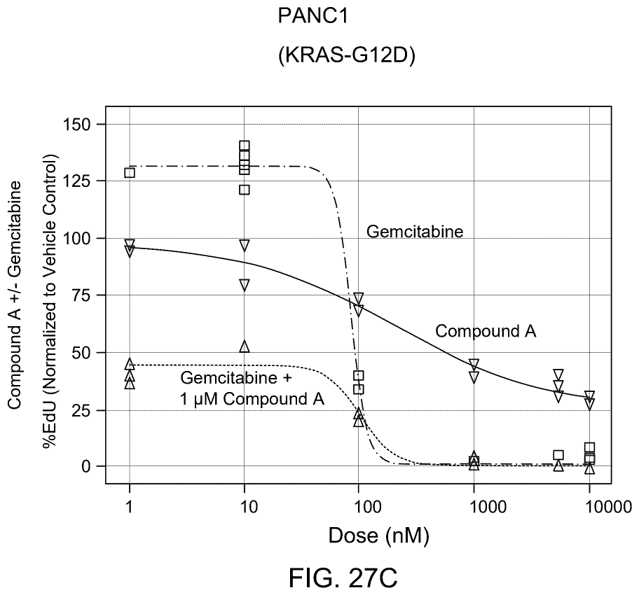

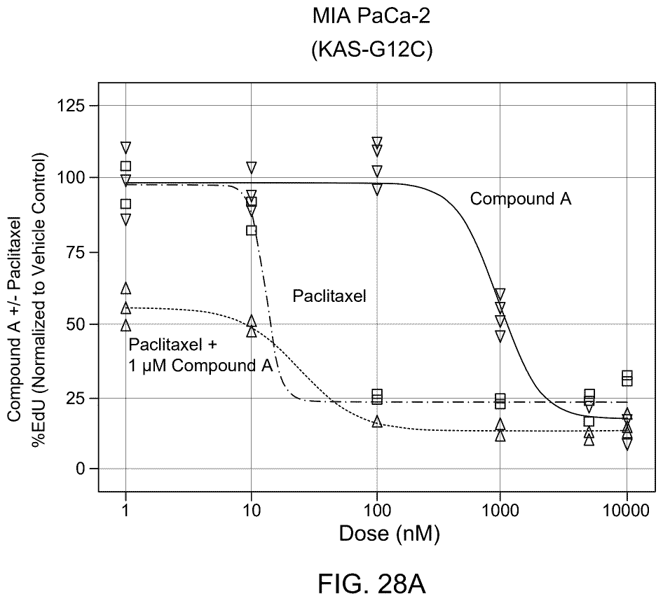

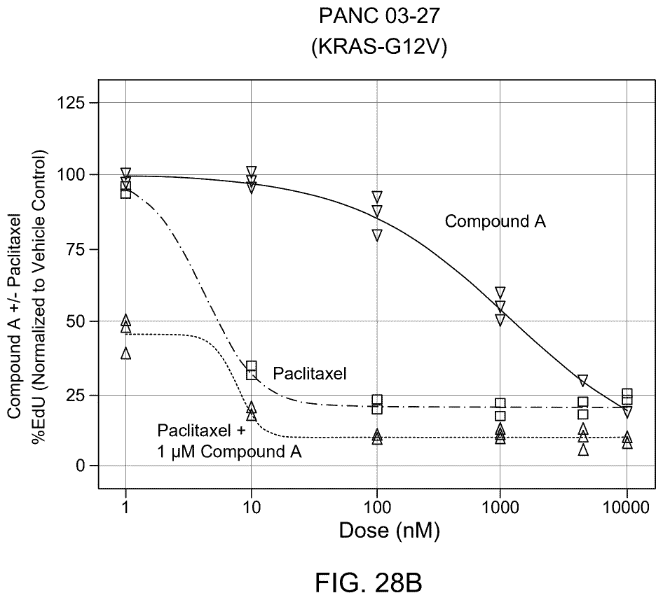

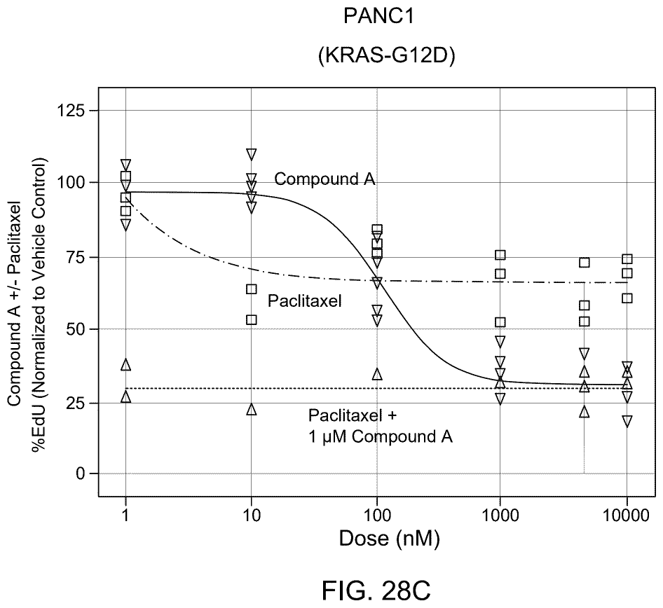

Referring to FIGS. 27A, 27B, 27C, 28A, 28B and 28C: (1) Compound A±CT dose response curves were obtained in the humanized 3D Tumor Growth Assay (3D-TGA); (2) three human pancreatic cancer cell models were selected based on patient alignment scores, where each model's mutational profile mapped to three distinct subsets of GENIE v13.1 patients categorized as pancreatic adenocarcinoma; and (3) gemcitabine and paclitaxel (CT agents commonly used for treatment of pancreatic cancer) were commercially purchased. FIG. 28A is a dose response curves obtained in the humanized 3D Tumor Growth Assay (3D-TGA) with Compound A±paclitaxel with MIA PaCa-2 cells. FIG. 27A is a dose response curves obtained in the humanized 3D Tumor Growth Assay (3D-TGA) with Compound A±gemcitabine with MIA PaCa-2 cells. FIG. 27B is a dose response curves obtained in the humanized 3D Tumor Growth Assay (3D-TGA) with Compound A±gemcitabine with Panc 03-27 (KRAS G12V) cells. FIG. 27C is a dose response curves obtained in the humanized 3D Tumor Growth Assay (3D-TGA) with Compound A±gemcitabine with Panc1 (KRAS-G12D) cells. FIG. 28A is a dose response curves obtained in the humanized 3D Tumor Growth Assay (3D-TGA) with Compound A±paclitaxel with MIA PaCa-2 cells. FIG. 28B is a dose response curves obtained in the humanized 3D Tumor Growth Assay (3D-TGA) with Compound A±paclitaxel with Panc 03-27 (KRAS G12V) cells. FIG. 28C is a dose response curves obtained in the humanized 3D Tumor Growth Assay (3D-TGA) with Compound A±paclitaxel with Panc1 (KRAS-G12D) cells.

We used an integrated platform of translational experiments and informatics to identify patient-aligned model systems, prioritize factors relevant for response to Compound A's unique DCI profile, and elucidate combination opportunities to potentially inform clinical development strategies.

### Example 14

Compound a in Combination with Gemcitabine and Nab-Paclitaxel (mGnP) or in Combination with Modified FOLFIRINOX (mFFX) for Patients with PDAC

In some embodiments, a method of treating pancreatic cancer with Compound A is provided. Specifically, in some embodiments, methods of treating pancreatic ductal adenocarcinoma (PDAC) and other exocrine tumors with compound A are provided.

Table 6 provides a summary of prior clinical trials for the treatment of pancreatic cancer.

TABLE 6
Pancreatic Cancer Clinical Trials

| Clinical Trial | Treatment | Line of Treatment | ORR |
|---|---|---|---|
| Phase III MPACT | Gemcitabine | 1^{st} Line | 7% |
| Phase III MPACT | Gemcitabine + | 1^{st} Line | 23% |
| | nab-paclitaxel | | |
| Phase III | FOLFIRINOX | 1^{st} Line | 32% |
| PRODIGE/ACCORD 11 | | | |
| Phase III NAPOLI-3 | NALIRIFOX | 1^{st} Line | 42% |
| Phase III NAPOLI-1 | Nal-IRI + FU/LV | 2^{nd} Line | 17% |
| Phase III MPACA-3 | mFOLFIRINOX | 2^{nd} Line | 15% |

Compound A is evaluated in a Phase 1/2a, multicenter, open-label, nonrandomized study to characterize the safety and anti-tumor activity of Compound A administered to participants with RAS-mutated or RAS/MAPK pathway activated advanced or metastatic solid tumors as monotherapy and in combination with approved agents. This is a Phase 1/2a, multicenter, open-label, nonrandomized study to characterize the safety and anti-tumor activity of Compound A administered to participants with RAS-mutated or RAS/MAPK pathway activated advanced or metastatic solid tumors as monotherapy and in combination with approved agents. This study is comprised of 2 phases, beginning with Phase 1 Dose Exploration (including monotherapy dose escalation and dose expansion stages, as well as cohorts designed to evaluate the safety and tolerability of Compound A in combination with approved agents) followed by Phase 2a featuring multiple proof-of-concept (POC) generating monotherapy and combination cohorts in malignancies of interest.

Participants are categorized into the following treatment groups (arms):
Treatment Group A: Compound A Monotherapy
Treatment Group B: Compound A in combination with modified gemcitabine and nab-paclitaxel (mGnP) in participants with previously untreated, unresectable advanced or metastatic PDAC
Treatment Group C: Compound A in combination with modified FOLFIRINOX (mFFX) in participants with previously untreated unresectable advanced or metastatic PDAC

Once adequate Compound A monotherapy safety and tolerability data are collected and evaluated, dose escalation cohorts evaluating the safety, tolerability, and PK of Compound A in combination with approved agents is initiated for enrollment in Treatment Groups B and C. In some aspects, the dose of Compound A to be used in combination with the anti-cancer treatments represented in Treatment Groups B and C is 320 mg QD. In addition, Compound A can also be administered in combination with these agents, at 240 mg QD, one dose level lower.

In addition, methods of treatment can include the administration of Compound A in combination with mGnP or mFFX. Approximately 30 participants per indication cohort can be enrolled, based on a cohort-specific Simon's 2-stage design.

Monotherapy Compound A is administered in a once-daily regimen for up to twelve 28-day cycles (48 weeks). If multiple daily dose regimens are pursued (e.g., BID), the total daily dose administered will not exceed the highest tolerable QD dose evaluated to date.

In some methods of treatment, Compound A is administered in combination with approved anti-cancer agents, and Compound A is administered once-daily at the dose that optimally displays DCI, while the combination agents will be administered in commonly used modified regimens that maintain effectiveness while reducing chemotherapy-related toxicities. Combination treatment continues until PD (radiographic or clinical) or other treatment discontinuation criteria are met. Individual components of the chemotherapy regimens can be discontinued if necessary to manage toxicities known to be associated with the agent(s). Likewise, if the entire chemotherapy regimen must be discontinued due to intolerability, participants may continue to receive Compound A as monotherapy until PD or other treatment discontinuation criteria are met. Similarly, participants who achieve a disease response of sufficient depth and duration that may warrant cessation of the approved agents may continue to receive maintenance Compound A until PD or other treatment discontinuation criteria are met. Other recognized maintenance regimens in combination with Compound A may be allowed with approval from the Sponsor MM.

Treatment will be administered in an inpatient or outpatient setting, as deemed appropriate by the Investigator and/or per discussion with the Sponsor. Participant safety will be monitored throughout the study by an SRC established by the Sponsor.

In some embodiments, methods of treatment comprise the administration of Compound A in combination with gemcitabine for the treatment of pancreatic adenocarcinoma in a patient in need thereof. In one aspect, a method of treating pancreatic adenocarcinoma comprises administering to the patient in need thereof Compound A in combination with, before or after, a gemcitabine-based regimen in Table 7.

TABLE 7
Chemotherapy Regimen combinations with Compound A

| | | Gemcitabine-based Regimen |
|---|---|---|
| | | (if fixed dose infusion rate not utilized, administer |
| Regimen | Compound A | gemcitabine 1,000 mg/m² over 30 minutes) |
| 1 | Compound A 320 mg QD | Gemcitabine 600-750 mg/m² IV on Days 1, 8, |
| | (or 240 mg reduced dose) | 15 (fixed dose infusion rate of 10 mg/m²/minute |
| | PO | preferred) |
| | | Repeat every 28 days |
| 2 | Compound A 320 mg QD | Gemcitabine 600-750 mg/m² IV on Day 1 (fixed |
| | (or 240 mg reduced dose) | dose infusion rate of 10 mg/m²/min preferred) |
| | PO | Cisplatin 30 mg/m² IV over 60 minutes on Day |
| | | 1 |
| | | Repeat every 14 days |
| | | (The preferred doublet for tumors with germline |
| | | BRCA mutations) |
| 3 | Compound A 320 mg QD | Gemcitabine 600-750 mg/m² IV on Days 1 and |
| | (or 240 mg reduced dose) | 8 (fixed dose infusion rate of 10 mg/m²/minute |
| | PO | preferred) |
| | | Capecitabine 1,500-1,800 mg/m²/day PO |
| | | divided twice daily on Days 1-14 |
| | | Repeat every 21 days |
| 4 | Compound A 320 mg QD | (dosing from ESPAC-4 in the adjuvant setting) |
| | (or 240 mg reduced dose) | Gemcitabine 1,000 mg/m² IV over 30 minutes |
| | PO | weekly on Days 1, 8, and 156 |
| | | Capecitabine 1,660 mg/m²/day PO divided |
| | | twice daily on Days 1-216 |
| | | Repeat every 28 days |
| 5 | Compound A 320 mg QD | Good performance status: |
| | (or 240 mg reduced dose) | Paclitaxel protein-bound 100-125 mg/m² IV on |
| | PO | Days 1, 8, 15 |
| | | Gemcitabine 600-750 mg/m² IV on Days 1, 8, |
| | | 15 (fixed dose infusion rate of 10 mg/m²/min |
| | | preferred) |
| | | Repeat every 28 days |
| | | Average performance status: |
| | | Paclitaxel protein-bound 125-175 mg/m² IV on |
| | | Day 1 |
| | | Gemcitabine 600-750 mg/m² IV on Day 1 (fixed |
| | | dose infusion rate of 10 mg/m²/min preferred) |
| | | Repeat every 14 days |
| 6 | Compound A 320 mg QD | Gemcitabine 300-400 mg/m² IV on Days 4 and |
| | (or 240 mg reduced dose) | 11 (fixed dose infusion rate of 10 mg/m²/minute |
| | PO | preferred) |
| | | Docetaxel 30-40 mg/m² IV on Days 4 and 11 |
| | | Capecitabine 1,000 mg/m²/day PO divided twice |
| | | daily on Days 1-14 |
| | | Repeat every 21 days |
| 7 | Compound A 320 mg QD | Gemcitabine 600-750 mg/m² IV on Day 1 (fixed |
| | (or 240 mg reduced dose) | dose infusion rate of 10 mg/m²/minute preferred) |
| | PO | Oxaliplatin 85 mg/m² IV over 2 hours on Day 1 |
| | | Repeat every 14 days |

In some embodiments, methods of treatment comprise the administration of Compound A in combination with oxaliplatin and/or fluorouracil for the treatment of pancreatic adenocarcinoma in a patient in need thereof. In one aspect, a method of treating pancreatic adenocarcinoma comprises administering to the patient in need thereof Compound A in combination with, before or after, a fluoropyrimidine-based regimen in Table 8A, a FOLFIRINOX regimen in Table 8B or Table 8C.

TABLE 8A
Chemotherapy Regimen combinations with Compound A

| Regimen | Compound A | Fluoropyrimidine-based Regimen |
|---|---|---|
| 1 | Compound A 320 mg QD | mFOLFOX 6 |
| | (or 240 mg reduced dose) | Oxaliplatin 85 mg/m² IV over 2 hours on Day 1 |
| | PO | Leucovorin 400 mg/m² IV over 2 hours on Day 18 |
| | | Fluorouracil 400 mg/m² IV bolus on Day 18, then fluorouracil |
| | | 2,400 mg/m² IV continuous infusion over 46 hours |
| | | Repeat every 14 days |
| 2 | Compound A 320 mg QD | XELOX or CapeOx |
| | (or 240 mg reduced dose) | Capecitabine 1,500-1,800 mg/m² PO divided twice daily on |
| | PO | Days 1-14, then |
| | | Oxaliplatin 85-100 mg/m² IV over 2 hours on Day 1 |
| | | Repeat every 21 days |
| 3 | Compound A 320 mg QD | FOLFIRINOX |
| | (or 240 mg reduced dose) | Oxaliplatin 75-85 mg/m² IV over 2 hours on Day 1 |
| | PO | Irinotecan 125-180 mg/m² IV over 90 minutes on Day 1 |
| | | Leucovorin 400 mg/m² IV over 2 hours on Day 17 |
| | | Fluorouracil 400 mg/m² IV bolus on Day 17, then fluorouracil |
| | | 2,400 mg/m² IV continuous infusion over 46 hours |
| | | Repeat every 14 days |
| 4 | Compound A 320 mg QD | Liposomal irinotecan (Onivyde ®) plus 5-fluorouracil |
| | (or 240 mg reduced dose) | Liposomal irinotecan 70 mg/m² IV over 90 minutes on Day 1 |
| | PO | (For patients with known homozygous UGT1A1*28 allele |
| | | reduce the initial starting dose to 50 mg/m²) |
| | | Leucovorin 400 mg/m² IV over 2 hours on Day 1 |
| | | Fluorouracil 400 mg/m² IV bolus on Day 1, then |
| | | Fluorouracil 2,400 mg/m² IV continuous infusion over 46 hours |
| | | Repeat every 14 days |

TABLE 8B
FOLFIRINOX (irinotecan, oxaliplatin and fluorouracil) in combination with
Compound A (e.g., 320 mg QD PO or 240 mg QD PO)

| Day | Drug | Dose | Route | Diluent & Rate |
|---|---|---|---|---|
| Day 1 | Glucose 5% | 500 ml | Infusion | Fast Running for Line Flush |
| | Ondansetron | 8 mg | Oral | |
| | Dexamethasone | 8 mg | Oral/Slow bolus/15 min infusion | |
| | Oxaliplatin | 85 mg/m² | IV infusion | 250 ml glucose 5% over 2 hours |
| | Calcium Folinate | 200 mg/m² | IV Infusion | 250 ml Glucose 5% over 2 hours. |
| | (Folinic Acid) | | | After 30 min. start Irinotecan |
| | Irinotecan | 180 mg/m² | IV Infusion | Over 90 minutes in 250 ml |
| | | | | Glucose 5% alongside folinic acid |
| | Glucose 5% | | | 250 ml line flush |
| | 5 fluorouracil | 400 mg/m² | IV bolus | Over 5 minutes |
| | 5 Fluorouracil | 2400 mg/m² | via infusor | Sodium chloride 0.9% over |
| | | | device | 46 hours |

An example of a modified FOLFIRINOX dosing schedule is provided in Table 8C. Oxaliplatin is administered in 250 mL glucose 5% over 2 hours. This is infused concurrently with calcium folinate in 250 mL glucose 5% over 2 hours. The line should then be flushed with glucose 5%.

TABLE 8C
modified FOLFIRINOX (14 day cycle frequency,
for up to 12 cycles) in combination with Compound A
(e.g., 320 mg QD PO or 240 mg QD PO)

| Day | Drug | Dose | Route |
|---|---|---|---|
| 1 | Calcium folinate | 350 mg | IV infusion |
| 1 | Oxaliplatin | 85 mg/m² | IV infusion |
| 1 | Irinotecan | 150 mg/m² | IV infusion |
| 1-2 (46 hours) | Fluorouracil | 2400 mg/m² | IV infusion over 46 hours |

In some embodiments, methods of treatment comprise the administration of Compound A in combination with chemoradiation for the treatment of pancreatic adenocarcinoma in a patient in need thereof. In one aspect, a method of treating pancreatic adenocarcinoma comprises administering to the patient in need thereof Compound A in combination with, before or after, chemoradiation-based regimen in Table 9.

TABLE 9
Chemoradiation Regimen combinations with Compound A

| Regimen | Compound A | Chemoradiation Regimen |
|---|---|---|
| 1 | Compound A 320 mg | Long course chemoradiation |
| | QD (or 240 mg reduced | Total dose 50 Gy in 25 fractions or 50.4 Gy in 28 |
| | dose) PO | fractions |
| | | Concurrent capecitabine 1,650 mg/m² |
| | | Concurrent gemcitabine 300-400 mg/m² IV given |
| | | at fixed dose infusion once weekly (if fixed dose |
| | | infusion rate of 10 mg/m²/minute not utilized, |
| | | administer gemcitabine over 30 minutes) |
| 2 | Compound A 320 mg | Short course chemoradiation |
| | QD (or 240 mg reduced | Total dose 30 Gy in 10 fractions |
| | dose) PO | Concurrent capecitabine 1,650 mg/m² PO in two |
| | | divided doses on each day of radiation or |
| | | Concurrent gemcitabine 300-400 mg/m² IV given |
| | | at fixed rate dose infusion once weekly (if fixed dose |
| | | infusion rate of 10 mg/m²/minute not utilized, |
| | | administer gemcitabine over 30 minutes) |
| 3 | Compound A 320 mg | Hypofractionated chemoradiation |
| | QD (or 240 mg reduced | Total dose 60-67.5 Gy in 15 fractions |
| | dose) PO | Concurrent capecitabine 1,650 mg/m² PO in two |
| | | divided doses on each day of radiation |
| | | Requires image guidance |
| 4 | Compound A 320 mg | Total dose 33-40 Gy in 5 fractions |
| | QD (or 240 mg reduced | Usually requires fiducials |
| | dose) PO | Requires daily image guidance |

### Example 15

Activity of Compound a Alone or in Combination with Chemotherapy in RAS-Altered Pancreatic Cancer Models

Addiction to the MAPK pathway drives a large proportion of cancers, and pancreatic tumors almost universally display activation of the MAPK pathway and most commonly by way of RAS mutations. Compound A, a once-daily oral treatment being evaluated in a Phase 1/2a trial for RAS-mutant solid tumors [NCT05585320], offers a novel deep cyclic inhibition (DCI) approach in disrupting the MAPK pathway at MEK. Traditionally, MAPK-targeted drugs inhibit the pathway chronically and are associated with serious class-effect toxicities and limited durability. In contrast, Compound A's unique pharmacokinetic (PK) profile was designed to drive deep pulsatile inhibition of MEK, with the goal of improving tolerability and providing more durable activity across a broad range of MAPK-driven tumors. Phase 1 dose escalation of Compound A revealed no dose-limiting toxicities, high oral bioavailability, plasma half-life of approximately 2-hours, and pharmacodynamic (PD) data supporting DCI of the MAPK pathway.

Compound A responses in humanized 3D tumor growth assays (3D-TGA), which better predict in vivo tumor response relative to 2D culture, were combined with NGS data using machine learning (ML) to refine a pharmacogenomic response model. Evaluation of databases such as AACR Project GENIE enabled prediction of patient alignment of preclinical models based on genomic profile, identification of patient populations displaying MAPK pathway addiction and projected sensitivity to Compound A mono- or combination therapy. To test combinations with approved chemotherapy agents, Compound A, gemcitabine (GEM) and nab-paclitaxel (PAC) and 5-fluorouracil (5-FU) were evaluated in 3D-TGA and in a pancreatic tumor xenograft model with drugs alone or across multiple combinations.

By examining cell line 3D-TGA response data using in-house whole exome sequencing that was further supported by emergent Phase 1 clinical ctDNA data, a machine learning model was further refined to predict Compound A sensitivity. A starting set of gene features, defined by alteration frequency >5% in the GENIE 15.0 database within the indications of pancreatic adenocarcinoma, colorectal adenocarcinoma, lung adenocarcinoma, and melanoma, was filtered for representation in the dataset and prioritized using ordinal regression. (GENIE v15.0: The AACR Project GENIE Consortium. AACR Project GENIE: Powering Precision Medicine Through An International Consortium, Cancer Discov. 2017 Aug. 7(8):818-831). Model-projected responses were then calculated for GENIE patient profiles in select solid tumor indications. Mutation patterns observed in GENIE patients were also compared with those observed in cell lines to identify preclinical models that best resemble real-world patients. The goal of this effort is to increase the translational fidelity of specific tumor models, aiming to translationally identify patient populations most likely to benefit from Compound A treatment. Here, we focus on model refinement in pancreatic cancer to better understand MAPK pathway addiction vs. utilization.

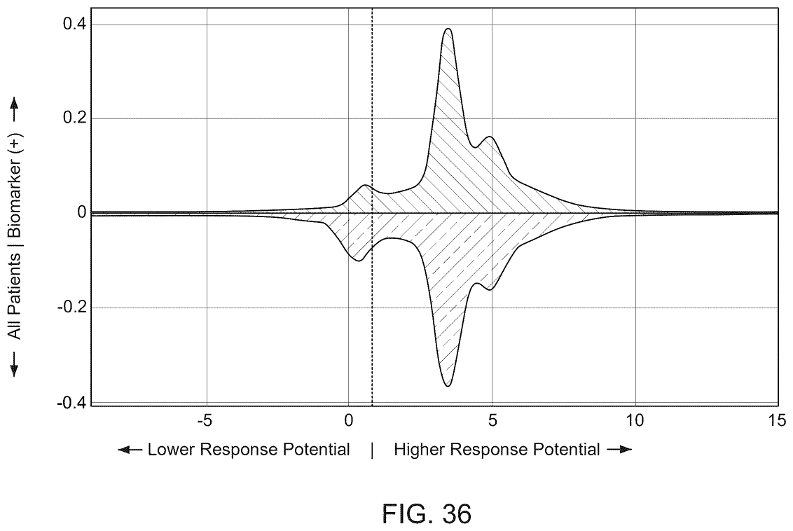

MAPK signaling pathway in PAAD disease and its potential therapeutic sensitivity based on ML model. FIG. 36 is a graph showing the mapping of biomarker sensitive patient populations (GENIE) for pancreatic adenocarcinoma (PAAD). FIG. 36 is a kernel density plot of the probability of MAPK pathway addiction and potentially responsive population projected onto the negative y-axis, and the MAPK biomarker (+) subset projected onto the positive y-axis. Pancreatic cancer displays a strong biomarker (+) subset (91% in this dataset of >4500 pancreatic adenocarcinoma (PAAD) patients in GENIE v15.0 with all model features on the testing panel), suggesting a broad opportunity for mono- and combination therapies with Compound A.

### Example 16

### In Vitro Pharmacology Response Profiles of 12 Pancreatic Tumor Models in Humanized 3D-Tumor Growth Assay (3D-TGA) to Compound A, as Single Agent or Combination Therapy

The humanized 3D tumor growth assay (3D-TGA) is an extracellular matrix (ECM)-based, 3D tumor model that more faithfully mimics the human tumor microenvironment (TME) with the goal of improving translational fidelity to patients. Disruption of the MAPK pathway has specifically demonstrated that 3D models more accurately predict certain aspects of in vivo tumor biology, especially when evaluating molecular activities of drug candidates that target the MAPK pathway. MEK inhibition in the 3D-TGA is useful to compare tumor addiction levels to the MAPK pathway across many models and establish response biomarkers for Compound A. See Casneuf T, et al., “Interleukin-6 is a potential therapeutic target in interleukin-6 dependent, estrogen receptor-a-positive breast cancer,” Breast Cancer (Dove Med Press). 2016; 8:13-27; Onion D, et al. “3-Dimensional Patient-Derived Lung Cancer Assays Reveal Resistance to Standards-of-Care Promoted by Stromal Cells but Sensitivity to Histone Deacetylase Inhibitors,” Mol Cancer Ther. 2016 April; 15(4):753-63; and Sasser A K, et al., “Interleukin-6 is a potent growth factor for ER-alpha-positive human breast cancer,” FASEB J. 2007 Nov. 21(13):3763-70, and Janes M R, et al., “Targeting KRAS Mutant Cancers with a Covalent G12C-Specific Inhibitor,” Cell. 2018 Jan. 25; 172(3):578-589.e17, each incorporated herein by reference in its entirety.

This Example provides the non-clinical evaluation of various tumor models with different KRAS treated with the dual-MEK inhibitor, Compound A, in the 3D-TGA, to evaluate pharmacogenomic responses (sensitivity) as single agent or in combination with chemotherapy agents that are standard of care in the clinic for mPDAC: gemcitabine (an antimetabolite antineoplastic agent interfering with DNA synthesis), paclitaxel (that interferes with the normal function of microtubules during cell division) or 5-FU (that acts in several ways, but principally as a thymidylate synthase (TS) inhibitor and is part of FOLFIRINOX). All drugs were evaluated in 9-point semi-log₁₀ dose response curves from 1 nM to 10 μM across the 12 pancreatic tumor models in the 3D-TGA. For each single drug, an IC₅₀ was calculated (drug dose required to reduce cell division by at least 50% as quantified by reduction of tumor cell 5-ethynyl-2′-deoxyuridine (EdU) incorporation), as well as a synergy score for each combination.

Human tumor cells were maintained in RPMI-1640, supplemented with 10% fetal bovine serum (FBS), 2 mM L-glutamine, 100 U/mL penicillin and 100 μg/mL streptomycin except when additional supplements were recommended by cell banks. Cells were maintained as a 2D monolayer in a humidified incubator at 37° C. with 5% CO₂ and atmospheric air (approximately 21% 02), being passaged once or twice weekly. All cell lines were validated by IDEXX upon receipt and assay setup. See Appendix for full cell line list.

Cells were seeded in 3D cultures in a black-walled, clear-bottom, non-tissue culture treated, 384-well plate (3000 cells per well) using a 1:1 mixture of BME and a custom media and grown in a humidified incubator with 5% CO₂ and 5% O₂, which approximates capillary levels for each gas, at 37° C. After 24 hours, cells were supplemented with a hormone cocktail made in custom RPMI 1640 media (without phenol red, 1.08 g/L D-Glucose, pH 6.8-6.9) with a final concentration of 250 μg/mL DHT, 50 μg/mL E2, 1 ng/mL P and treated with nine (9) concentrations of reference compounds or Compound A ranging from 10 μM to 1 nM for 72 hours. Cells were assessed for cell proliferation using EdU cell proliferation staining. After 48 hours of treatment, EdU was added to wells to a final concentration of 10 μM. After 72 hours, BME plugs were fixed with 3.7-4.0% formaldehyde 20-30 minutes at room temperature and washed twice with PBS. Cells were permeabilized with 30 μL of filtered 0.5% Triton X-100 in PBS for 20 minutes at room temperature. Samples were washed twice gently with PBS. Cells were stained utilizing click-iT chemistry by adding 20 μL of staining cocktail containing 2 mM copper sulfate, 8 μM sulfo-Cy5-azide, and 20 mg/ml ascorbic acid in PBS and incubated 30-60 minutes at room temperature in dark. Samples were washed three times with PBS, stained with 20 μL DAPI 30-60 minutes at room temperature in dark, washed twice with PBS, 20 μL PBS added to wells, and kept in dark approximately 48 hours before imaging. Plates were imaged with the Molecular Devices Pico ImageXpress. Images were segmented and percentage of cells with EdU incorporation was quantified to calculate antiproliferative effects of the drugs.

Pancreatic tumor models displaying different activating mutations in KRAS were tested in the 3D-TGA to evaluate potential synergy between Compound A, a third generation MEK inhibitor, and chemotherapy agents broadly used in the clinical setting, namely gemcitabine, paclitaxel and 5-fluorouracil. In all cases, addition of Compound A to the chemotherapy did not have any antagonistic effect, and in most cell lines, the presence of the physiologically relevant dose of 1 uM Compound A had an additive effect to all chemotherapy agents tested. Similar combinations with Compound A and chemotherapy agents have also been tested in an in vivo setting with the KRAS^{G12C} mutant model, MIA PaCa2, showing even greater results in terms of synergistic effect.

Five (5)-ethynyl-2′-deoxyuridine (EdU) data was represented as percentage EdU positive cells quantified by high content imaging from the Molecular Devices ImageXpress® Pico. Best fit curves were generated and IC₅₀ calculated using the R package dr4pl (Ritz C, et al., “Dose-Response Analysis Using R.,” PLOS ONE [Internet], 2015 Dec. 30 [cited 2018 Jul. 5]; 10(12):e0146021).

In this study, the pharmacogenomic effect of Compound A was evaluated in a selection of 12 pancreatic tumor models with diverse KRAS mutations in the 3D-TGA, alone and in combination with gemcitabine, paclitaxel or 5-fluorouracil. Drug-drug combinations of Compound A with gemcitabine, paclitaxel, and 5-fluorouracil (5-FU) resulted in enhanced tumor growth inhibition across multiple models when compared to chemotherapy alone. Tables 10A-10E show IC50 values obtained from 3D-TGA with certain chemotherapy agents (paclitaxel in Table 10A, gemcitabine in Table 10B, 5-FU in Table 10C, oxaliplatin in Table 10D, SN-38 (irinotecan) in Table 10E), and the percent change in IC50 obtained when combining each chemotherapy agent with Compound A in various DAC cancer cell lines. The IC50. A reduction in IC50 when each chemotherapy agent is combined with Compound A is noted as a “YES” in each table. The IC50 values for the chemotherapy agent are as follows: “A”=<50 nM; “B”=50 nM to <100 nM; “C”=100 nM to <500 nM; “D”=500 nM to 1,000 nM; “E”=1,000 nM.

TABLE 10A
Compound A + Paclitaxel

| | | | | IC50 in 3D-TGA [nM] | | |
|---|---|---|---|---|---|---|
| | | | | | % IC50 change | |
| | PDAC Cell | | | | 1 μM Cmpd A + | IC50 |
| # | Line | Indication | Mutation | Paclitaxel | Paclitaxel | Enhanced |
| 1 | MIA-PaCa2 | PAN | KRAS G12C | A | −32% | YES |
| 2 | Capan-2 | PAN | KRAS G12V | A | −35% | YES |
| 3 | AsPC-1 | PAN | KRAS G12D | A | −92% | YES |
| 4 | PA-TU- | PAN | KRAS G12V | A | −26% | YES |
| | 8988S | | | | | |
| 5 | CFPAC-1 | PAN | KRAS G12V | A | −18% | YES |
| 6 | HPAF-II | PAN | KRAS G12D | A | −(>87%) | YES |
| 7 | Panc 03.27 | PAN | KRAS G12V | A | −(>86%) | YES |
| 8 | PSN-1 | PAN | KRAS G12R | A | 34% | — |
| 9 | Panc 10.05 | PAN | KRAS G12D | A | −18% | YES |
| 10 | Capan-1 | PAN | KRAS G12V | A | −36% | YES |
| 11 | HPAC | PAN | KRAS G12D | A | −43% | YES |
| 12 | PANC-1 | PAN | KRAS G12D | E | −(>100%) | YES |
| 13 | HT-29 | COL | BRAF V600E | A | 22% | YES |
| 14 | RCM-1 | COL | KRAS G12V | C | 87% | YES |
| 15 | SW626 | COL | KRAS G12V | A | −(>89%) | YES |
| 16 | CL-40 | COL | KRAS G12D | A | 71% | YES |
| 17 | SNU-1197 | COL | KRAS G12D | C | 90% | YES |
| 18 | LS1034 | COL | KRAS A146T | E | — | — |

TABLE 10B
Compound A + Gemcitabine

| | | | | IC50 in 3D-TGA [nM] | | |
|---|---|---|---|---|---|---|
| | | | | | % IC50 change | |
| | PDAC Cell | | | | 1 μM Cmpd A + | IC50 |
| # | Line | Indication | Mutation | Gemcitabine | Gemcitabine | Enhanced |
| 1 | MIA-PaCa2 | PAN | KRAS G12C | A | −(>97%) | YES |
| 2 | Capan-2 | PAN | KRAS G12V | C | +103% | — |
| 3 | AsPC-1 | PAN | KRAS G12D | D | −(>100%) | YES |
| 4 | PA-TU- | PAN | KRAS G12V | A | −10% | YES |
| | 8988S | | | | | |
| 5 | CFPAC-1 | PAN | KRAS G12V | A | −23% | YES |
| 6 | HPAF-II | PAN | KRAS G12D | A | − (>95%) | YES |
| 7 | Panc 03.27 | PAN | KRAS G12V | A | −36% | YES |
| 8 | PSN-1 | PAN | KRAS G12R | A | +26% | — |
| 9 | Panc 10.05 | PAN | KRAS G12D | A | −43% | YES |
| 10 | Capan-1 | PAN | KRAS G12V | A | +34% | — |
| 11 | HPAC | PAN | KRAS G12D | A | +156% | — |
| 12 | PANC-1 | PAN | KRAS G12D | B | −(>99%) | YES |
| 13 | HT-29 | COL | BRAF V600E | A | −61% | YES |
| 14 | RCM-1 | COL | KRAS G12V | D | −53% | YES |
| 15 | SW626 | COL | KRAS G12V | C | −(>99%) | YES |
| 16 | CL-40 | COL | KRAS G12D | A | −74% | YES |
| 17 | SNU-1197 | COL | KRAS G12D | C | −21% | YES |
| 18 | LS1034 | COL | KRAS A146T | D | −61% | YES |

TABLE 10C
Compound A + 5-Fluorouracil (5-FU)

| | | | | IC50 in 3D-TGA [nM] | | |
|---|---|---|---|---|---|---|
| | | | | | % IC50 change | |
| | PDAC Cell | | | | 1 μM Cmpd A + | IC50 |
| # | Line | Indication | Mutation | 5-FU | Fluorouracil | Enhanced |
| 1 | MIA-PaCa2 | PAN | KRAS G12C | E | −(>90%) | YES |
| 2 | Capan-2 | PAN | KRAS G12V | E | — | — |
| 3 | AsPC-1 | PAN | KRAS G12D | E | — | — |
| 4 | PA-TU-8988S | PAN | KRAS G12V | E | — | — |
| 5 | CFPAC-1 | PAN | KRAS G12V | E | 5% | — |
| 6 | HPAF-II | PAN | KRAS G12D | E | −(>99%) | YES |
| 7 | Panc 03.27 | PAN | KRAS G12V | E | −(>84%) | YES |
| 8 | PSN-1 | PAN | KRAS G12R | E | — | — |
| 9 | Panc 10.05 | PAN | KRAS G12D | E | — | — |
| 10 | Capan-1 | PAN | KRAS G12V | E | — | — |
| 11 | HPAC | PAN | KRAS G12D | E | — | — |
| 12 | PANC-1 | PAN | KRAS G12D | E | −(>90%) | YES |

TABLE 10D
Compound A + Oxaliplatin

| | | | | IC50 in 3D-TGA [nM] | | |
|---|---|---|---|---|---|---|
| | | | | | % IC50 change | |
| | PDAC Cell | | | | 1 μM Cmpd A + | IC50 |
| # | Line | Indication | Mutation | Oxaliplatin | Oxaliplatin | Enhanced |
| 1 | MIA-PaCa2 | PAN | KRAS G12C | E | −62% | YES |
| 2 | Capan-2 | PAN | KRAS G12V | E | −32% | YES |
| 3 | PA-TU-8988T | PAN | KRAS G12V | E | −(>100%) | YES |
| 4 | CFPAC-1 | PAN | KRAS G12V | E | −17% | YES |
| 5 | HPAF-II | PAN | KRAS G12D | E | −(>100%) | YES |
| 6 | PSN-1 | PAN | KRAS G12R | E | — | — |
| 7 | Panc 10.05 | PAN | KRAS G12D | E | −61% | YES |
| 8 | Capan-1 | PAN | KRAS G12V | C | +50% | — |
| 9 | HPAC | PAN | KRAS G12D | E | — | — |
| 10 | PANC-1 | PAN | KRAS G12D | E | −(>100%) | YES |
| 11 | HT-29 | COL | BRAF V600E | E | +37% | — |
| 12 | RCM-1 | COL | KRAS G12V | D | −45% | YES |
| 13 | SW626 | COL | KRAS G12V | E | −(>100%) | YES |
| 14 | CL-40 | COL | KRAS G12D | C | −74% | YES |
| 15 | SNU-1197 | COL | KRAS G12D | E | +10% | — |
| 16 | LS1034 | COL | KRAS A146T | E | +132% | — |
| 17 | A549 | LUN | KRAS G12S | E | −18% | YES |
| 18 | Calu-6 | LUN | KRAS Q61K | E | +14% | — |
| 19 | NCI-H358 | LUN | KRAS G12C | E | +64% | — |

TABLE 10E
Compound A + SN-38 (Irinotecan)

| | | | | IC50 in 3D-TGA [nM] | | |
|---|---|---|---|---|---|---|
| | | | | | % IC50 change | |
| | PDAC Cell | | | | 1 μM Cmpd A + | IC50 |
| # | Line | Indication | Mutation | SN-38 | SN-38 | Enhanced |
| 1 | MIA-PaCa2 | PAN | KRAS G12C | A | +400% | — |
| 2 | Capan-2 | PAN | KRAS G12V | A | 0% | — |
| 3 | PA-TU- | PAN | KRAS G12V | A | −(>90%) | YES |
| | 8988T | | | | | |
| 4 | CFPAC-1 | PAN | KRAS G12V | A | −75% | YES |
| 5 | HPAF-II | PAN | KRAS G12D | A | −(>89%) | YES |
| 6 | Panc 10.05 | PAN | KRAS G12D | A | −42% | YES |
| 7 | Capan-1 | PAN | KRAS G12V | A | +50% | — |
| 8 | HPAC | PAN | KRAS G12D | A | +269% | — |
| 9 | PANC-1 | PAN | KRAS G12D | A | −(>89%) | YES |
| 10 | HT-29 | COL | BRAF V600E | A | 0% | — |
| 11 | RCM-1 | COL | KRAS G12V | A | −6% | YES |
| 12 | SW626 | COL | KRAS G12V | A | −(>63%) | YES |
| 13 | CL-40 | COL | KRAS G12D | A | −(>52%) | YES |
| 14 | SNU-1197 | COL | KRAS G12D | A | +100% | — |
| 15 | LS1034 | COL | KRAS A146T | A | +123% | — |

In this study, the pharmacogenomic effect of Compound A was evaluated in a selection of various cancer models with diverse KRAS mutations in the 3D-TGA, alone and in combination with various additional oncology therapeutics.

Tables 11A-11B show IC50 values obtained from 3D-TGA with certain KRAS G12C inhibitor compounds (sotorasib in table 11A and adagrasib in table 11B), and the percent change in IC50 obtained when combining each KRAS G12D inhibitor agent with Compound A in various cancer cell lines. A reduction in IC50 when each KRAS G12D inhibitor agent is combined with Compound A is noted as a “YES” in each table. The IC50 values for the agent are as follows: “A”<50 nM; “B”=50 nM to <100 nM; “C”=100 nM to <500 nM; “D”=500 nM to 1,000 nM; “E”=>1,000 nM.

TABLE 11A
Compound A + Sotorasib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Sotorasib | Sotorasib | Enhanced |
| 1 | MIA-PaCa2 | PAN | KRAS G12C | A | −(>98%) | YES |
| 2 | LU99 | LUN | KRAS G12C | E | — | — |
| 3 | NCI-H358 | LUN | KRAS G12C | C | −73% | YES |
| 4 | HCC-44 | LUN | KRAS G12C | E | — | — |
| 5 | NCI-H2030 | LUN | KRAS G12C | A | −(>97%) | YES |
| 6 | NCI-H1792 | LUN | KRAS G12C | E | — | — |
| 7 | NCI-H2122 | LUN | KRAS G12C | E | — | — |

TABLE 11B
Compound A + Adagrasib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Adagrasib | Adagrasib | Enhanced |
| 1 | MIA-PaCa2 | PAN | KRAS G12C | A | −(>95%) | YES |
| 2 | LU99 | LUN | KRAS G12C | E | −(>15%) | YES |
| 3 | NCI-H358 | LUN | KRAS G12C | B | −72% | YES |
| 4 | HCC-44 | LUN | KRAS G12C | E | — | — |
| 5 | NCI-H2030 | LUN | KRAS G12C | A | −(>87%) | YES |
| 6 | NCI-H1792 | LUN | KRAS G12C | E | — | — |
| 7 | NCI-H2122 | LUN | KRAS G12C | E | +(>153%) | — |

Tables 12A-121B show IC50 values obtained from 3D-TGA with certain CDK-4-6 inhibitor compounds (abemaciclib in table 12A and palbociclib in table 121B), and the percent change in % C50 obtained when combining each CDK-4-6 inhibitor agent with Compound A in various cancer cell lines. A reduction in IC50 when each agent is combined with Compound A is noted as a “YES” in each table. The IC50 values for the agent are as follows: “A”=50 nM; “B”=50 nM to <100 nM; “C”=100 nM to <500 nM; “D”=500 nM to 1,000 nM; “E”=1,000 nM.

TABLE 12A
Compound A + Abemaciclib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Abemaciclib | Abemaciclib | Enhanced |
| 1 | MIA PaCa-2 | PAN | KRAS G12C | E | −(>100%) | YES |
| 2 | CFPAC-1 | PAN | KRAS G12V | D | −32% | YES |
| 3 | A-427 | LUN | KRAS G12D | C | −52% | YES |
| 4 | A549 | LUN | KRAS G12S | C | +115% | — |
| 5 | Calu-6 | LUN | KRAS Q61K | E | −(>100%) | YES |
| 6 | HT-29 | COL | BRAF V600E | E | −(>98%) | YES |

TABLE 12B
Compound A + Palbociclib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Palbociclib | Palbociclib | Enhanced |
| 1 | MIA PaCa-2 | PAN | KRAS G12C | E | −(>100%) | YES |
| 2 | CFPAC-1 | PAN | KRAS G12V | C | −12% | YES |
| 3 | A-427 | LUN | KRAS G12D | D | −63% | YES |
| 4 | A549 | LUN | KRAS G12S | C | +36% | — |
| 5 | Calu-6 | LUN | KRAS Q61K | E | −(>100%) | YES |
| 6 | HT-29 | COL | BRAF V600E | E | −97% | YES |

Tables 13A-13D show IC50 values obtained from 3D-TGA with certain EGFR inhibitor compounds (afatinib in Table 13A, erlotinib in Table 131B, gefitinib in Table 13C and osimertinib in Table 131D), and the percent change in IC50 obtained when combining each EGFR inhibitor agent with Compound A in various cancer cell lines. A reduction in IC50 when each agent is combined with Compound A is noted as a “YES” in each table. The IC50 values for the EGFR inhibitor agent are as follows: “A”=50 nM; “B”=50 nM to <100 nM; “C”=100 nM to <500 nM; “D”=500 nM to 1,000 nM; “E”=1,000 nM.

TABLE 13A
Compound A + Afatinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Afatinib | Afatinib | Enhanced |
| 1 | MIA | PAN | KRAS G12C | E | −(>100%) | YES |
| | PACA-2 | | | | | |
| 2 | NCI-H508 | COL | BRAF G596R | D | −90% | YES |
| 3 | SK-CO-1 | COL | KRAS G12V | E | −98% | YES |
| 4 | LS411N | COL | BRAF V600E | E | — | — |
| 5 | NCI-H358 | LUN | KRAS G12C | E | −71% | YES |
| 6 | NCI-H820 | LUN | EGFR T790M | E | −99% | YES |
| 7 | NCI-H1975 | LUN | EGFR L858R | E | −75% | YES |
| 8 | NCI-H1650 | LUN | EGFR | E | — | — |
| | | | E746_A750 | | | |
| | | | del | | | |
| 9 | NCI-H2172 | LUN | EGFR L858R | E | — | — |
| 10 | NCI-H1792 | LUN | KRAS G12C | E | — | — |
| 11 | HCC827 | LUN | EGFR | A | −63% | YES |
| | | | E746_A750 | | | |
| | | | del | | | |

TABLE 13B
Compound A + Erlotinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Erlotinib | Erlotinib | Enhanced |
| 1 | MIA | PAN | KRAS G12C | E | −(>100%) | YES |
| | PACA-2 | | | | | |
| 2 | NCI-H508 | COL | BRAF G596R | E | −48% | YES |
| 3 | SK-CO-1 | COL | KRAS G12V | E | −(>89%) | YES |
| 4 | LS411N | COL | BRAF V600E | E | — | — |
| 5 | NCI-H358 | LUN | KRAS G12C | E | −(>32%) | YES |
| 6 | NCI-H820 | LUN | EGFR T790M | E | −92% | YES |
| 7 | NCI-H1975 | LUN | EGFR L858R | E | −(>19%) | YES |
| 8 | NCI-H1650 | LUN | EGFR | E | — | — |
| | | | E746_A750 del | | | |
| 9 | NCI-H2172 | LUN | EGFR L858R | E | — | — |
| 10 | NCI-H1792 | LUN | KRAS G12C | E | — | — |
| 11 | HCC827 | LUN | EGFR | A | +629% | — |
| | | | E746_A750 del | | | |

TABLE 13C
Compound A + Gefitinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Gefitinib | Gefitinib | Enhanced |
| 1 | MIA | PAN | KRAS G12C | E | −(>100%) | YES |
| | PACA-2 | | | | | |
| 2 | NCI-H508 | COL | BRAF G596R | E | −43% | YES |
| 3 | SK-CO-1 | COL | KRAS G12V | E | −84% | YES |
| 4 | LS411N | COL | BRAF V600E | E | — | — |
| 5 | NCI-H358 | LUN | KRAS G12C | E | — | — |
| 6 | NCI-H820 | LUN | EGFR T790M | E | −96% | YES |
| 7 | NCI-H1975 | LUN | EGFR L858R | E | — | — |
| 8 | NCI-H1650 | LUN | EGFR | E | μ− | — |
| | | | E746_A750 | | | |
| | | | del | | | |
| 9 | NCI-H2172 | LUN | EGFR L858R | E | — | — |
| 10 | NCI-H1792 | LUN | KRAS G12C | E | — | — |
| 11 | HCC827 | LUN | EGFR | A | +97% | — |
| | | | E746_A750 | | | |
| | | | del | | | |

TABLE 13D
Compound A + Osimertinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Osimertinib | Osimertinib | Enhanced |
| 1 | MIA | PAN | KRAS G12C | E | −(>100%) | YES |
| | PACA-2 | | | | | |
| 2 | NCI-H508 | COL | BRAF G596R | E | −(>80%) | YES |
| 3 | SK-CO-1 | COL | KRAS G12V | E | −(>80%) | YES |
| 4 | LS411N | COL | BRAF V600E | E | — | — |
| 5 | NCI-H358 | LUN | KRAS G12C | E | — | — |
| 6 | NCI-H820 | LUN | EGFR T790M | E | −99% | YES |
| 7 | NCI-H1975 | LUN | EGFR L858R | E | −83% | YES |
| 8 | NCI-H1650 | LUN | EGFR | E | — | — |
| | | | E746_A750 del | | | |
| 9 | NCI-H2172 | LUN | EGFR L858R | E | — | — |
| 10 | NCI-H1792 | LUN | KRAS G12C | E | — | — |
| 11 | HCC827 | LUN | EGFR | A | +(>100%) | — |
| | | | E746_A750 del | | | |

Table 14 shows IC50 values obtained from 3D-TGA with certain BRAF inhibitor compound encorafenib and the percent change in CC50 obtained when combining encorafenib with Compound A in various cancer cell lines. A reduction in IC50 when encorafenib is combined with Compound A is noted as a “YES” in each table. The IC50 values for the EGFR inhibitor agent are as follows: “A”=<50 nM; “B”=50 nM to <100 nM; “C” 100 nM to <500 nM; “D”=500 nM to 1,000 nM; “E”=>1,000 nM.

TABLE 14
Compound A + Encorafenib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Encorafenib | Encorafenib | Enhanced |
| 1 | A375 | MEL | BRAF V600E | A | −(>29%) | YES |
| 2 | HT-29 | COL | BRAF V600E | E | −(>99%) | YES |
| 3 | LS411N | COL | BRAF V600E | E | — | — |

Tables 15A-15D show IC50 values obtained from 3D-TGA with certain ALK inhibitor compounds (alectinib in Table 15A, brigatinib in Table 15B, crizotinib in Table 15C and loratinib in Table 151%), and the percent change in IC50 obtained when combining each ALK inhibitor agent with Compound A in various cancer cell lines. A reduction in C50 when each agent is combined with Compound A is noted as a “YES” in each table. The IC50 values for the ALK inhibitor agent are as follows: “A”=50 nM; “B”=50 nM to <100 nM; “C”=100 nM to <500 nM; “D”=500 nM to 1,000 nM; “E”=1,000 nM.

TABLE 15A
Compound A + Alectinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Alectinib | Alectinib | Enhanced |
| 1 | NCI- | LUN | NRAS Q61K, | E | −(>79%) | YES |
| | H2087 | | ALK S553A | | | |
| 2 | SW900 | LUN | KRAS G12V, | E | −(>100%) | YES |
| | | | ALK G103R | | | |
| 3 | CORL23 | LUN | KRAS G12V, | E | — | — |
| | | | ALK R317T | | | |

TABLE 15B
Compound A + Brigatinib

| | | | | IC50 in 3D-TGA [nM] | | |
|---|---|---|---|---|---|---|
| | | | | | % IC50 change | |
| | | | | | 1 μM Cmd A + | IC50 |
| # | Cell Line | Indication | Mutation | Brigatinib | Brigatinib | Enhanced |
| 1 | NCI- | LUN | NRAS Q61K, | E | −86% | YES |
| | H2087 | | ALK S553A | | | |
| 2 | CORL23 | LUN | KRAS G12V, | E | −13% | YES |
| | | | ALK R317T | | | |

TABLE 15C
Compound A + Crizotinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Crizotinib | Crizotinib | Enhanced |
| 1 | NCI- | LUN | NRAS Q61K, | E | −53% | YES |
| | H2087 | | ALK S553A | | | |
| 2 | CORL23 | LUN | KRAS G12V, | E | −30% | YES |
| | | | ALK R317T | | | |
| 3 | SW900 | LUN | KRAS G12V, | E | −(>100%) | YES |
| | | | ALK G103R | | | |

TABLE 15D
Compound A + Lorlatinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Lorlatinib | Lorlatinib | Enhanced |
| 1 | NCI- | LUN | NRAS Q61K, | E | −(>82%) | YES |
| | H2087 | | ALK S553A | | | |
| 2 | CORL23 | LUN | KRAS G12V, | E | — | — |
| | | | ALK R317T | | | |

Tables 16A-16D show IC50 values obtained from 3D-TGA with hematologic oncology agents azacitidine (Table 16A), decitabine (Table 16B), daunorubicin (Table 16C), and bortezomib (Table 16D) and the percent change in IC50 obtained when combining each agent with Compound A in various cancer cell lines. A reduction in IC50 when each agent is combined with Compound A is noted as a “YES” in each table. The IC50 values for the ALK inhibitor agent are as follows: “A”=50 nM; “B”=50 nM to <100 nM; “C”=100 nM to <500 nM; “D”=500 nM to 1,000 nM; “E”=1,000 nM.

TABLE 16A
Compound A + Azacitidine

| | | | | IC50 in 3D-TGA [nM] | | |
|---|---|---|---|---|---|---|
| | | | | | % IC50 change | |
| | Cell | | | | 1 μM Cmpd A + | IC50 |
| # | Line | Indication | Mutation | Azacitidine | Azacitidine | Enhanced |
| 1 | NB4 | AML | KRAS Amp | E | −78% | YES |
| 2 | HL-60 | AML | NRAS Q61L | E | −44% | YES |
| 3 | OCI- | AML | NRAS Q61L | E | +112% | — |
| | AML3 | | | | | |
| 4 | PL-21 | AML | KRAS A146V | D | −(>100%) | YES |
| 5 | KHM-1B | MM | NRAS G12C/ | C | −36% | YES |
| | | | KRAS G12C | | | |
| 6 | TF-1 | AML | NRAS Q61P | E | −(<100%) | YES |
| 7 | THP-1 | AML | NRAS G12D | E | −(<100%) | YES |

TABLE 16B
Compound A + Decitabine

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Decitabine | Decitabine | Enhanced |
| 1 | NB4 | AML | KRAS Amp | E | 149.1 | YES |
| 2 | HL-60 | AML | NRAS Q61L | E | — | — |
| 3 | OCI-AML3 | AML | NRAS Q61L | E | +(>250%) | — |
| 4 | PL-21 | AML | KRAS A146V | E | −(>100%) | YES |
| 5 | TF-1 | AML | NRAS Q61P | A | −(>96%) | YES |
| 6 | THP-1 | AML | NRAS G12D | E | −(>100%) | YES |

TABLE 16C
Compound A + Daunorubicin

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Daunorubicin | Daunorubicin | Enhanced |
| 1 | NB4 | AML | KRAS Amp | 63.2 | −45% | YES |
| 2 | HL-60 | AML | NRAS Q61L | 67.6 | −38% | YES |
| 3 | OCI-AML3 | AML | NRAS Q61L | 28 | +25% | — |
| 4 | PL-21 | AML | KRAS A146V | 64.1 | −(>98%) | YES |
| 5 | TF-1 | AML | NRAS Q61P | 56.2 | −(>98%) | YES |
| 6 | THP-1 | AML | NRAS G12D | 119.2 | −(>99%) | YES |

TABLE 16D
Compound A + Bortezomib

| | | | | IC50 in 3D-TGA [nM] | | |
|---|---|---|---|---|---|---|
| | | | | | 1 μM Cmpd A + | IC50 |
| # | Cell Line | Indication | Mutation | Bortezomib | Bortezomib | Enhanced |
| 1 | RPMI- | MM | KRAS G12A | C | −75% | YES |
| | 8226 | | | | | |
| 2 | KHM-1B | MM | NRAS G12C/ | D | −98% | YES |
| | | | KRAS G12C | | | |
| 3 | L-363 | MM | NRAS Q61H | E | −1% | YES |
| 4 | MM.1S | MM | KRAS G12A | B | −69% | YES |

Table 17 shows IC50 values obtained from 3D-TGA with certain FAK inhibitor compound ifebemetinib and the percent change in IC50 obtained when combining ifebemetinib with Compound A in various cancer cell lines. A reduction in C50 when ifebemetinib is combined with Compound A is noted as a “YES” in each table. The IC50 values for the EGFR inhibitor agent are as follows: “A”=<50 nM; “B”=50 nM to <100 nM; “C”=100 nM to <500 nM; “D”=500 nM to 1,000 nM; “E”=>1,000 nM.

TABLE 17
Compound A + Ifebemetinib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Ifebemetinib | Ifebemetinib | Enhanced |
| 1 | MIA PaCa-2 | PAN | KRAS G12C | E | −(>100%) | YES |
| 2 | HPAC | PAN | KRAS G12D | E | — | — |
| 3 | HPAF-II | PAN | KRAS G12D | E | −(>100%) | YES |
| 4 | AsPC-1 | PAN | KRAS G12D | E | — | — |
| 5 | Panc 10.05 | PAN | KRAS G12D | E | −(>99%) | YES |
| 6 | PANC-1 | PAN | KRAS G12D | E | −(>100%) | YES |
| 7 | LU99 | LUN | KRAS G12C | E | — | — |
| 8 | A-427 | LUN | KRAS G12D | E | −(>22%) | YES |
| 9 | NCI-H2030 | LUN | KRAS G12C | D | −(>100%) | YES |
| 10 | HCC-44 | LUN | KRAS G12C | E | — | — |
| 11 | T3M-10 | LUN | KRAS G12D | E | −(>99%) | YES |
| 12 | NCI-H2122 | LUN | KRAS G12C | E | — | — |
| 13 | NCI-H1792 | LUN | KRAS G12C | E | — | — |
| 14 | NCI-H358 | LUN | KRAS G12C | E | −(>27%) | YES |
| 15 | COLO 678 | COL | KRAS G12D | E | — | — |
| 16 | CL-40 | COL | KRAS G12D | E | −(>97%) | YES |

Table 18 shows IC50 values obtained from 3D-TGA with certain PARP inhibitor compound talazoparib and the percent change in IC50 obtained when combining talazoparib with Compound A in various cancer cell lines. A reduction in IC50 when talazoparib is combined with Compound A is noted as a “YES” in each table. The IC50 values for the EGFR inhibitor agent are as follows: “A”=<50 nM; “B”=50 nM to <100 nM; “C”=100 nM to <500 nM; “D”=500 nM to 1,000 nM; “E”=>1,000 nM.

TABLE 18
Compound A + Talazoparib

| | | | | IC50 in 3D-TGA [nM] | | |
|---|---|---|---|---|---|---|
| | | | | | % IC50 change | |
| | | | | | 1 μM Cmpd A + | IC50 |
| # | Cell Line | Indication | Mutation | Talazoparib | Talazoparib | Enhanced |
| 1 | MIA PACA-2 | PAN | KRAS G12C | E | −93% | YES |
| 2 | Capan-1 | PAN | KRAS G12V | E | — | — |
| 3 | A549 | LUN | KRAS G12S | E | +321% | — |
| 4 | NCI-H1792 | LUN | KRAS G12C | D | −46% | YES |
| 5 | NCI-H1734 | LUN | KRAS G13C | E | −25% | YES |
| 6 | HL-60 | AML | NRAS Q61L | E | −13% | YES |
| 7 | THP-1 | AML | NRAS G12D | E | — | — |

1-40. (canceled)

41. A method of treating a BRAF mutated colorectal cancer in combination with a kinase inhibitor, the method comprising administering Compound A 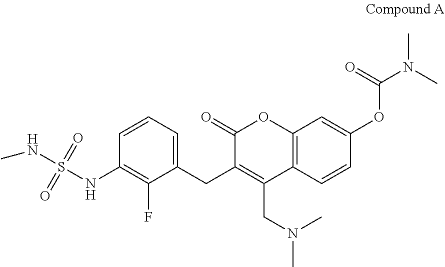
or a pharmaceutically acceptable salt thereof to a patient in need thereof at a dose providing 320 mg or 240 mg of Compound A once daily.

42. The method of claim 41, wherein a total of 320 mg of Compound A is orally administered (po) once daily (QD) to treat the BRAF mutated colorectal cancer.

43. The method of claim 42, wherein the patient is diagnosed with metastatic colorectal cancer with a BRAF V600E mutation.

44. The method of claim 43, wherein the kinase inhibitor is encorafenib.

45. The method of claim 44, wherein the method further comprises administering 300 mg encorafenib once daily to the patient in need thereof.

46. The method of claim 41, wherein a total of 240 mg of Compound A is orally administered (po) once daily (QD) to treat the BRAF mutated colorectal cancer.

47. The method of claim 46, wherein the patient is diagnosed with metastatic colorectal cancer with a BRAF V600E mutation.

48. The method of claim 47, wherein the kinase inhibitor is encorafenib.

49. The method of claim 48, wherein the method further comprises administering 300 mg encorafenib once daily to the patient in need thereof.
